(19) United States
(12) Patent Application Publication
(10) Pub. No.: US 2023/0385450 A1
(43) Pub. Date: Nov. 30, 2023

(54) HUMAN-CENTRIC HEALTH RECORD SYSTEM AND RELATED METHODS

(71) Applicant: Luc Bessette, Montreal (CA)

(72) Inventors: Luc Bessette, Montreal (CA); Yves Leborgne, Montreal (CA); Francois Desloges, Montreal (CA); Mathieu Rousseau, Montreal (CA)

(21) Appl. No.: 17/994,128

(22) PCT Filed: May 25, 2021
(86) PCT No.: PCT/CA2021/050704

§ 371 Date: Nov. 25, 2022

Related U.S. Application Data
(60) Provisional application No. 63/147,080, filed on Feb. 8, 2021.
(60) Provisional application No. 63/110,191, filed on Nov. 5, 2020.
(60) Provisional application No. 63/029,643, filed on May 25, 2020.

(30) Foreign Application Priority Data
May 25, 2020 (CA) 3081531
Nov. 5, 2020 (CA) 3098242
Feb. 8, 2021 (CA) 3108555

(51) Int. Cl. G06F 21/62; G16H 10/60

(52) U.S. Cl. CPC G06F 21/6254; G16H 10/60; G06F 2221/2115

(57) ABSTRACT

A method to manage exposure of confidential user information in a user data record to a third party, the method comprising providing an electronic record system managed by a server arrangement, the server arrangement being configured to interact with a user device associated with the user, implementing with the server arrangement a data privacy configurator including a GUI allowing the user at the user device to specify data access parameters, the data access parameters distinguishing between first data in the user data record that the user is willing to expose to a third party from second data in the user data record that the user is not willing to expose to the third party, in response to a request received at the server arrangement to communicate user data from the user data record to the third party, processing the user data record according to the data access parameters to allow the third party to access the first data while precluding the third party to access the second data.

### FIELD OF THE INVENTION

The invention generally relates to electronic health record systems and in particular to Human-Centric health record systems and related methods. While most of the technical concepts disclosed in this document are directed to the management of medical data, at least some of those concepts have broader applications and can be used outside the health care industry such as the banking industry, the legal industry or any other field where the management of confidential information is necessary. Accordingly, the invention is not limited to health-care related uses and can be embodied in systems and methods that manage user information which is not linked to medicine.

### BACKGROUND

Medicine has been for a long time a long time a top-down activity, where all activities are dictated by the capabilities and restrictions of care providers, and where the patient ends up being a passive voice in the decisions and chain of events. The doctor has the knowledge and the office, the patient has the waiting room. The patient's medical information is gathered and stored in the doctor's or hospital record. The patient must wait to get results and then wait again for a doctor that is part of his health management organization to be available. Pertinent medical data are the product of medical interventions and patient driven medical data is often not deemed contributive. Physicians are doing medical research—patients are studied.

The data and influence are currently in the hands of the few who are collecting the information. Cloaked with the screen of confidentiality, the information is rarely shared nor aggregated in a cooperative way. For instance, health records associated with a patient may be stored at various locations, such as at an electronic health record (EHR) system associated with a physician, as part of a government regulated or state-owned health record system, pharmacies, health maintenance organizations (HMOs) and the like. A problem with such EHR systems is that a patient's health records are dispersed across multiple sources (e.g., multiple computer systems) and are not easily accessible if a patient wants a comprehensive record of his/her medical history.

As a patient's health records are located at multiple sources, this can further be a problem if a patient visits a new physician outside of the patient's regular physician's office and/or receives care outside of his [symbol] ally accessible care delivery network, as the new physician does not have a complete understanding [symbol] e patient's medical history. Moreover, any medical information regarding the new physician's assessments of the patient is not added to the patient's file at the regular physician's office and/or the file that is part of the patient's normally accessible care delivery network.

Another problem with current patients' electronic health records (EHRs) is that as these records contain confidential and/or privileged information, the communication of the health records and gaining access to health information is not done in a risk-free way such that confidentiality is maintained. A process of authentication of a user trying to remotely access health records is, therefore, a key component of the safety of an electronic record system. The process of authentication of the user consists of validating that a user trying to access the confidential and/or privileged data corresponds to the user who is authorized to consult the confidential and/or privileged data and not unauthorized users. Some authentication systems have tried to remedy this situation and provide safer authentication. However, these authentication processes require a succession of steps (e.g., an increased number of passwords, an increased complexity of passwords, etc.) rendering the process less user-friendly, and not necessarily rendering the authentication significantly safer.

A further problem with patients' health records being located at multiple sources is that if third parties (e.g., agents) would like access to patient information for various reasons they are required to obtain consent from the patient for each source and then access each of the various sources that store patients' health records separately to obtain a more complete medical history of the patients. Obtaining consent from patients for multiple sources and then accessing multiple sources can be tedious especially where patient information is desired for a large group of patients.

In addition, when patients get medical results done (e.g., tests, lab work, imaging, etc.) they are typically not informed if their medical results have been processed, completed or reviewed, unless their physician contacts them. This often leaves patients in a state of uncertainty and anxiety regarding their medical results and/or the completion status.

A patient may also make use of various portable and wearable devices, which measure various physical conditions. The data from these various devices are typically stored in a distributed computing environment (e.g., the “cloud”) which is separate from the other sources containing health records associated with the patient, which results in yet another source of health records associated with the patient, further compounding fragmentation of the health-related information. [symbol] ght of the above, there is a need in the industry for providing an improved EHR system and improved methods relating to EHR systems that alleviates, at least in part, the deficiencies with existing EHR systems.

### SUMMARY OF THE INVENTION

As embodied and broadly described herein, the invention provides a method to manage exposure of confidential user information in a user data record to a third party, the method comprising providing an electronic record system managed by a server arrangement, the server arrangement being configured to interact with a user device associated with the user, implementing with the server arrangement a data privacy configurator including a GUI allowing the user at the user device to specify data access parameters, the data access parameters distinguishing between first data in the user data record that the user is willing to expose to a third party from second data in the user data record that the user is not willing to expose to the third party, in response to a request received at the server arrangement to communicate user data from the user data record to the third party, processing the user data record according to the data access parameters to allow the third party to access the first data while precluding the third party to access the second data.

### BRIEF DESCRIPTION OF THE DRAWINGS

A detailed description of embodiments of the invention is provided below, by way of example only, with reference to the accompanying drawings, in which:

FIG. 1A illustrates a block diagram of an example Human-Centric electronic health record (EHR) system connected to various other EHR systems and to a computing entity associated with a patient in accordance with an embodiment of the invention.

FIG. 1B illustrates a block diagram of an example EHR network comprising a central EHR system in accordance with an embodiment of the invention.

FIG. 1C illustrates a block diagram of an example EHR network comprising various EHR systems and a computing entity associated with a patient connected to the EHR network in accordance with an embodiment of the invention.

FIG. 2A illustrates a block diagram of a Human-Centric EHR system in accordance with a specific example of implementation.

FIG. 2B illustrates a block diagram of a physician's EHR system in accordance with a specific example of implementation.

FIGS. 3A to 3C illustrates examples of data record structures in accordance with embodiments of the inventions.

FIG. 4A illustrates a flowchart of a process, which may be implemented by the physician's EHR system of FIGS. 1A-1C in accordance with a specific example of implementation.

FIG. 4B illustrates a flowchart of a process, implemented by the EHR system in accordance with a specific non-limiting example of implementation.

FIG. 4C illustrates a flowchart of a process, implemented by the central EHR system in accordance with a specific non-limiting example of implementation.

FIG. 5A illustrates a block diagram of an EHR network comprising a single EHR system in accordance with a specific non-limiting example of implementation.

FIG. 5B illustrates a block diagram of an EHR network comprising a plurality of EHR systems accessible by a server in accordance with a specific non-limiting example of implementation.

FIG. 5C illustrates a block diagram of an EHR network comprising a plurality of EHR systems in accordance with a specific non-limiting example of implementation.

FIG. 6 illustrates a block diagram of an example of an EHR system that is able to provide patients with medical results upon authorization in accordance with an embodiment of the invention.

FIG. 7A illustrates a flowchart of a process for determining if the EHR system should be notified of receipt of medical results at a physician's office in accordance with a specific non-limiting example of implementation.

FIG. 7B illustrates a flowchart of a process for transmitting medical results to the EHR system in accordance with a specific non-limiting example of implementation.

FIG. 8A illustrates a flowchart of a process for sending a notification to the patient's computing entity in accordance with a specific non-limiting example of implementation.

FIG. 8B illustrates a flowchart of a process for sending medical results associated with the patient to the patient's computing entity in accordance with a specific non-limiting example of implementation.

FIG. 9 illustrates a block diagram of an example of an EHR system that is able to receive and obtain auxiliary medical related information in accordance with an embodiment of the invention.

FIG. 10 illustrates a flowchart of a process for making a health-related determination in accordance with a specific non-limiting example of implementation.

FIG. 11 illustrates a block diagram of an example of a Human-Centric EHR system that provides health records associated with a patient to a third-party EHR system in accordance with an embodiment of the invention.

FIG. 12A illustrates a flowchart of a first process for providing one or more health records associated with a patient to a third-party in accordance with a specific non-limiting example of implementation.

FIG. 12B illustrates a flowchart of a second process for providing one or more health records associated with a patient to a third-party in accordance with a specific non-limiting example of implementation.

FIG. 13A illustrates a block diagram of an example of an EHR system that provides health records associated with a patient to a third-party EHR system in accordance with another embodiment of the invention.

FIG. 13B illustrates a flowchart of a third process for providing one or more health records associated with a patient to a third-party in accordance with a specific non-limiting example of implementation.

FIG. 14 illustrates a block diagram of an example of an EHR system for providing patient anonymized [symbol] th records to a third-party computing entity in accordance with an embodiment of the invention.

FIG. 15 illustrates a flowchart of a process for providing patient anonymized health records to a third-party in accordance with a specific non-limiting example of implementation.

FIGS. 16A, 16B, 16C, 16D, 16E and 16F illustrate specific and non-limiting examples of information that may be displayed on a graphical user interface (GUI) of a patient's computing entity in accordance with specific examples of implementation.

FIGS. 17A, 17B and 17C illustrate specific and non-limiting examples of notifications in accordance with specific examples of implementation.

FIG. 18 illustrates a block diagram of an EHR system connected to various other EHR systems and to a computing entity associated with a patient in accordance with an embodiment of the invention.

FIGS. 19A, 19B, 19C, 19D and 19E illustrate specific and non-limiting examples of information that may be displayed on a graphical user interface (GUI) of a physician's computing entity in accordance with specific examples of implementation.

FIGS. 20A and 20B illustrate flowcharts of processes for creating a care support group in accordance with a specific non-limiting example of implementation.

FIG. 21 illustrates a data structure for a care support group in accordance with a specific non-limiting example of implementation.

FIG. 22 is a block diagram showing a system according to another embodiment of the disclosure, allowing a secure user authentication.

FIGS. 23 to 25D show flowcharts of methods of operation for the system depicted in FIG. 22 and GUI examples associated with those methods;

FIGS. 26A to 26D are block diagrams showing variants of the system illustrated in FIG. 22.

FIGS. 27A to 27G show Graphical User Interfaces (GUI) through which users interact with the system [symbol] FIG. 22, in particular for performing user authentication.

FIG. 28 is a flowchart describing a method performed by the system of FIG. 22.

FIG. 29 is a flowchart describing a method performed by the system of FIG. 22, according to a variant.

FIG. 30 is a method to implement the system of FIG. 22.

FIGS. 31 to 33D show flowcharts of alternative methods performed by the system of FIG. 22 and associated GUI examples.

FIG. 34 is a block diagram illustrating a network architecture where the EHR system interfaces with other networks, which are not interoperable with the EHR system.

FIG. 35 is a flow diagram of a process illustrating the interaction between the EHR system and an external network to communicate medical data;

FIG. 36 is flow diagram of a process for accessing medical data from the EHR system, which resides on an external network;

FIG. 37 is a flow diagram illustrating the operation of a software function to generate and dynamically maintain a medical information index.

FIGS. 38 to 41 are examples of Graphical User Interfaces (GUIs) allowing a patient to set up a data privacy management function at the EHR system.

FIG. 42 is a flow diagram of a process for a patient to set up the data privacy settings.

FIG. 43 is a block diagram of a user profile of a user of the EHR system FIG. 44 is a flowchart illustrating the operation of a theme-based filter to selectively mask medical information to preserve its confidentiality.

FIG. 45 is a flowchart of a process for managing the access of authorized users, such as treating physicians to the medical information of the patient.

FIG. 46 is a flowchart of a further process for managing the access of authorized users to the medical information of the patient.

FIG. 47 is a block diagram of a machine-readable data structure mapping medical data items to respective identifiers.

FIG. 48 is an example of a user interface where the patient can selectively mask medical information on the basis of certain themes.

FIG. 49 is block diagram of the elements of the Human-Centric EHR system showing a consent manager associated with a user profile.

FIG. 50 is a flowchart of a process to manage requests from external entities to access medical data.

FIG. 51 is flowchart of specific steps of the process shown at FIG. 50.

FIG. 52 is a diagram showing conceptually the data structure of a consent data element.

FIG. 53 is an example of a user interface to manage consents.

FIG. 54 is a further example of a GUI for managing consents.

FIG. 55 is yet another example of a GUI for managing consents.

FIG. 56 is a flowchart of a process to manage the validity of consents.

FIG. 57 illustrates an options hierarchy to set admissibility criteria.

FIG. 58 is a flowchart of a process to send to a third-party medical data.

FIG. 59 is a process to notify a user if anomalous result is identified in medical data sent to a third party.

FIG. 60 is a flowchart of a process to provide to a user, recommendations about the suitability of the user medical data for particular research projects.

FIG. 61 is block diagram of a networked arrangement allowing the EHR system to communicate with research organizations through an online portal.

FIG. 62 is a flowchart of a process that receives the information defining the parameters of a research project and tries to match the research project to individual subscribers of the EHR system.

FIG. 63 is a representation of a GUI allowing a user to manage participation in a research project.

FIG. 64 is a block diagram of an EHR system with a functionality to perform medical test results management and reconciliation, according to a specific example of implementation of the invention.

FIG. 65 is a block diagram of a test reconciliation functional block.

FIG. 66 is an example of GUI having a test selection control and a tracking control.

FIG. 67 is another example of a GUI having a notification control and an input control.

FIG. 68 is a flowchart of a process to set the parameters of a test request record and tracking parameters.

FIG. 69 is a flowchart of test reconciliation process.

FIG. 70 is a block diagram of a system for performing online purchase of a ticket for travel and configured to perform verification of on an immunization status.

FIG. 71 is a flowchart of a process performed by the system shown at FIG. 70.

FIG. 72 is an illustration of a mobile showing a notification allowing a user to authorize or not authorize the communication of an immunization status to a third party.

FIG. 73 is a block diagram of a system for performing online purchase of a ticket for travel, which is a [symbol] nt of the system shown at FIG. 70.

FIG. 74 are screen shots of displays illustrating a QR code to be read by a mobile to trigger the immunization status determination process.

FIG. 75 is a block diagram of a check-in kiosk with functionality to perform verification of the immunization status of travelers.

FIG. 76 is a flowchart of a process performed by the kiosk at FIG. 75.

FIG. 77a is a block diagram of another variant of the system to verify the immunization status of a user.

FIG. 77b is a block diagram of an app to determine the immunization status of a user.

FIG. 78 is a block diagram of a network arrangement showing the Human-Centric EHR system interacting with a drug dispensing entity.

FIG. 79 is a more detailed block diagram of the network arrangement shown in FIG. 78 showing in greater detail the structure of the drug dispensing entity.

FIG. 80 is a block diagram showing software functionalities of the Human-Centric EHR system to manage drug prescriptions.

FIG. 81 is a representation of a GUI allowing a patient to set preferences in relation to drug prescription management.

FIG. 82 is a flowchart of a process implementing the GUI shown in FIG. 81.

FIG. 83 is a flowchart of a process illustrating a drug prescription follow-up service.

FIG. 84 is an illustration of a mobile showing a notification providing a reminder to a patient in relation to a drug prescription.

FIG. 85 is another example of a notification to the patient in relation to prescribed drugs side effects.

FIG. 86 is a flowchart of a process implemented in relation to patient notifications about drug prescriptions.

FIG. 87 is a flowchart for a drug prescription refill reminder and renewal reminder service to the patient.

FIG. 88a is an illustration of a notification on the patient mobile in relation to the drug prescription renewal reminder service.

FIG. 88b is a block diagram illustrating the relationship between different software modules allowing to schedule an appointment for the renewal of a drug prescription.

FIG. 88c is a block diagram illustrating software modules invoked when the patient performs a transaction with the drug dispensing entity.

FIG. 89 is a flowchart of a process illustrating the various steps performed in the course of a transaction with the drug dispensing entity to fulfill a drug prescription.

FIG. 90 is a flowchart of a process to filter messages from the drug prescription entity to the patent.

FIG. 91 is a block diagram illustrating software modules or functionalities to manage interactions between the patient and entities in domains external to the Human-Centric EHR system 102, such as a drug dispensing entity, a lab services entity and an imaging services entity.

FIG. 92 is a flowchart of a process between a patient and a lab services entity implemented by the arrangement shown in FIG. 91.

FIG. 93 is an illustration of a notification mechanism allowing the patient to set an appointment with the lab services entity.

FIG. 94 is a block diagram of software functionalities of the Human-Centric EHR system 102, illustrating a drug prescription reconciliation and security management function.

FIG. 95 is a flowchart illustrating a process implemented by the arrangement shown in FIG. 94. [symbol] to be expressly understood that the description and drawings are only for the purpose of illustrating certain embodiments of the invention and are an aid for understanding. They are not intended to be a definition of the limits of the invention.

### DETAILED DESCRIPTION

Although the term electronic health record (EHR) system is used in this document, this term may also be referred to as an electronic health or medical record system or any other suitable termed system, computing entity or computing platform.

Any feature of any embodiment discussed herein may be combined with any feature of any other embodiment discussed herein in some examples of implementation.

Any of the steps of the processes discussed herein in may be done in different orders, the steps of process discussed herein may be combined and some steps of the processes discussed herein may be omitted in some examples of implementation.

The use of the term “patient” is used herein to refer to a person or an individual and is not intended to be limiting. For example, term “patient” could be used to include a legal guardian acting on behalf of the patient. “Patient” and “user” are terms that sometimes are being used interchangeably.

In the embodiments discussed herein the term “physician” is used, in other examples of implementation other types of medical professions, such as dentists, optometrists, pharmacists, nurses, nurse practitioners, physician assistants and/or any other suitable medical professional may be used synonymously with the term “physician”. The term “physician” may include any medical or health related “practitioner”, where the practitioner include may include any person that has access to read and/or write records to a patient's record.

Although reference is made in the examples above where interaction takes place with a government-managed EHR system and/or network, in other examples, other EHR systems and/or networks associated with other organizations (e.g., commercial organizations, government health care systems, HMOs, etc.) may synonymously be used. [symbol] also appreciated that the term database when referenced in this document could be a single structured [symbol] that includes information, or it could reference a collection of databases that could have multiple records or tables that can work jointly or independently of each other. In other words, the reference to database in this document may be to indicate the function of storage or reception of information such as patient records, summary medical records, prescription information, drug information, patient information, insurance information, data records, health records, medical records, etc. in one or more database, one or more tables and/or one or more records, where the databases, tables, and/or records are stored in one or more computer readable memories.

It is further appreciated that the term “EHR system” may be interpreted to include any computer-based system that runs or accesses application software that provides the functionality of electronic record systems described herein. For instance, the application software may be running on the EHR system itself or may be running on a separate computer system or server arrangement that the EHR system accesses (e.g., over a data network).

Certain additional elements that may be needed for operation of some embodiments have not been described or illustrated as they are assumed to be within the purview of those of ordinary skill in the art. Moreover, certain embodiments may be free of, may lack and/or may function without any element that is not specifically disclosed herein.

A description of examples of implementation of a Human-Centric EHR system and its functions, variants and uses to support other medical-related applications are provided below. The description will initially lay out the structure of the EHR system and some of its basic functionalities. Specific functionalities and variants will be described subsequently. The description of those specific functionalities and variants will build upon the description of the basic architecture of the EHR system.

Human-Centric EHR System Architecture and Basic Functionalities

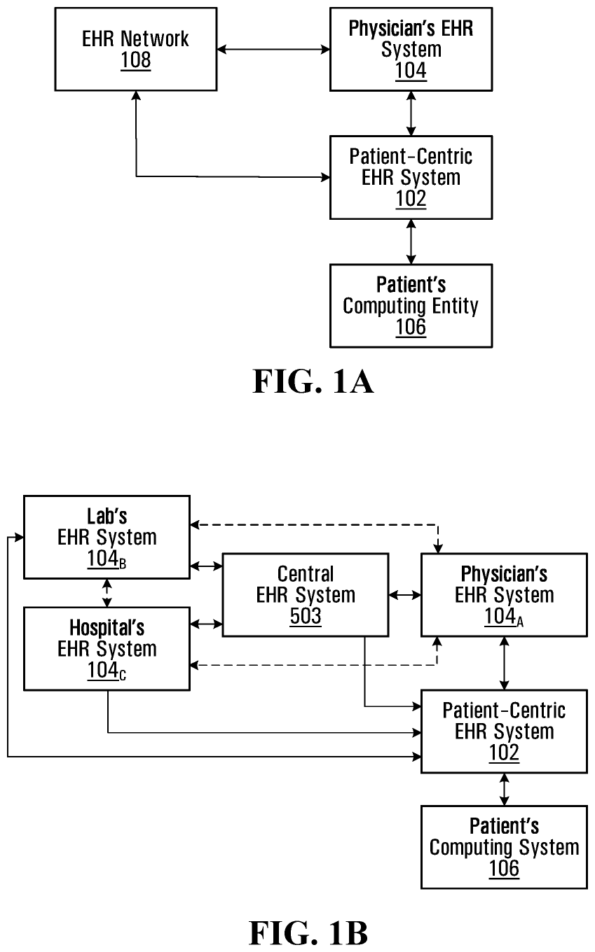

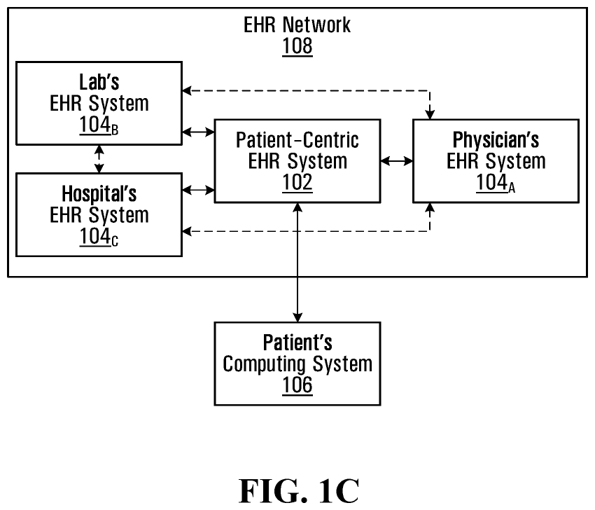

FIG. 1A illustrates a block diagram of an example Human-Centric electronic health record (EHR) system 102 connected to various other EHR systems and to a computing entity associated with a patient in accordance with an embodiment of the invention. As illustrated the Human-Centric EHR system 102 is connected to the patient's computing entity 106 (e.g., a computing entity associated with a patient) and to a physician's EHR system 104 (e.g., an EHR system associated with a physician) which is connected to an EHR network 108. Although in FIG. 1 only a single Human-Centric EHR system 102, a single [symbol] ician's EHR systems 104, a single EHR network 108 and a single patient's computing entity 106 are [symbol] vn, this is for illustration purposes only, as one or more Human-Centric EHR systems, one or more physician's EHR systems, one or more EHR networks and one or more patient's computing entities may be used in implementing the various embodiments described herein.

The various connections between the Human-Centric EHR system 102, the patient's computing entity 106, the physician's EHR system 104 and/or the EHR network 108 may include one or more connections over one or more data networks (e.g., the Internet, local area network (LAN), or a wide area network (WAN), cellular networks, other wired or wireless networks and/or any other suitable data network). In other words, the Human-Centric EHR system 102, the patient's computing entity 106, the physician's EHR systems 104 and/or the EHR network 108 may be nodes in one or more data networks linked by communication paths. The various connections between the Human-Centric EHR system 102, the patient's computing entity 106, physician's EHR systems 104 and/or the EHR network 108 may allow for the communication (e.g., transmission and/or reception) of various information and/or data between the various systems and/or devices. The various information and/or data exchanged may be notifications, data records, health records, medical records and medical related information general. The term “health record” is used throughout this document to refer to medical or health related information assembled in some cohesive way.

In general terms, the Human Centric EHR system 102 allows for a patient to have centralized storage of his/her health records by obtaining the health records associated with the patient from various sources and also allows for the patient to have some control over his/her health records. In other words, “Human Centric” refers to the Human Centric EHR system 102 possibly offering a patient with fine-grained control over visibility of health records associated with the patient. The control of the health records associated with the patient may include who can view the health records and/or what aspects of the health record can be viewed. In particular, some health records may have a ‘for your eyes only’ nature and be annotated as such. In that case, the Human Centric EHR system 102 may only make the information available when the patient has identified himself strongly as being the recipient. Furthermore, the ‘for your eyes only’ annotation may cause the health record being becoming ‘invisible’ after a set delay after the patient has seen the content. For instance, the health record may become ‘invisible’ by revoking any and all access rights that could exist for the record, for other users other than the patient himself/herself. Also, the record may become destroyed from volatile memory and then may only be viewed again if the patient himself/herself requests the records after a two-factor authentication at the device (e.g., fingerprint or other biometric identifier and user identifier such as a passcode). These and other aspects of the Human [symbol] ric EHR system 102 should likely become more readily apparent to the person skilled in the art [symbol] ghout this document.

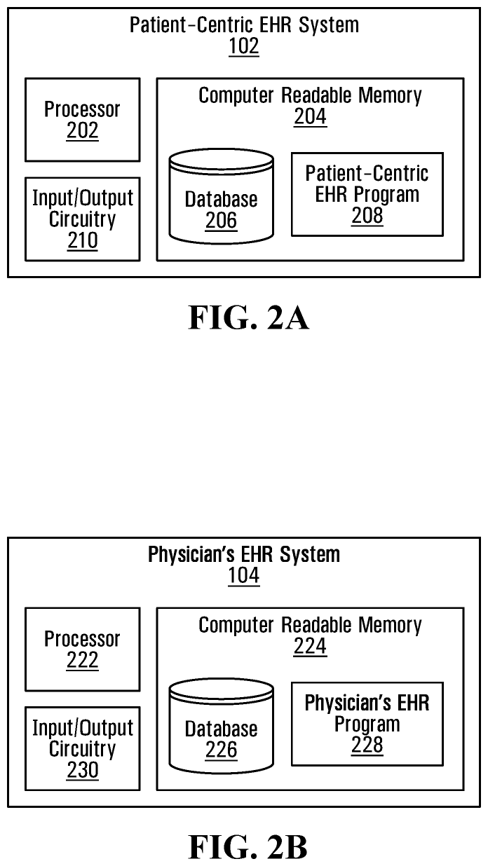

FIG. 2A is a block diagram of the Human-Centric EHR system 102 in accordance with a specific example of implementation. In general terms, the Human-Centric EHR system 102 may be a single computing entity or a distributed computing environment (e.g., server arrangements) that stores health records associated with patients. As illustrated, the Human-Centric EHR system 102 comprises at least one processor 202, input/output circuitry 210 and at least one computer readable memory 204 comprising at least one database 206 and a Human-Centric EHR program 208 (e.g., program code, application software, etc). The at least one processor 202, input/output circuitry 210 and at least one computer readable memory 204 may be connected by various data buses and in the case of a distributed computing environment may be interconnected by one or more data networks. The at least one database 206 stores health records associated with a plurality of patients. The Human-Centric EHR program 208 comprises program code which when executed by the at least one processor 202 causes the Human-Centric EHR system 102 to perform various operations and functions. The input/output circuitry 210 is used to connect the Human-Centric EHR system 102 to one or more data networks and to other peripheral devices (e.g., keyboard, mouse, monitor/display, printer and/or any other suitable device).

FIG. 2B illustrates a block diagram of the physician's EHR system 104 (e.g., an EHR system associated with a physician and/or any other health related practitioner) in accordance with a specific example of implementation. In general terms, the physician's EHR system 104 may be a single computing entity or a distributed computing environment (e.g., server arrangements) that stores health records associated with patients. For instance, the physician's EHR system 104 may be of the type that is typically found in a physician's office for maintaining health records of patients but with additional functionality as should likely become more readily apparent to the person skilled in the art throughout this document. As illustrated, the physician's EHR system 104 comprises at least one processor 222, input/output circuitry 230 and at least one computer readable memory 224 comprising at least one database 226 and at least one physician's EHR program 228. The at least one processor 222, input/output circuitry 230 and at least one computer readable memory 224 may be connected by various databases and in the case of a distributed computing environment may be interconnected by one or more data networks. The at least one database 226 stores one or more health records associated with a patient and stores a plurality of health records associated with a plurality of patients. The physician's at least one EHR program 228 comprises program code which when executed by the processor 222 causes the physician's EHR system 104 to perform various operations. The input/output circuitry 230 may be used to connect the physician's EHR system [symbol] to one or more data networks and to other peripheral devices (e.g., keyboard, mouse, monitor/display, [symbol] er and/or any other suitable device).

The patient's computing entity 106 (e.g., a computing entity associated with a patient) may be any portable or non-portable computing device, such as a cellphone, a tablet, a smart watch, a laptop/notebook computer, a computer or any other suitable computing device. The patient's computing entity 106 may comprise program code that when executed causes the patient's computing entity 106 to perform various operations, such as execute application software. In some cases, the patient's computing entity 106 runs application software that is associated with the Human-Centric EHR system 102. The application software may include client-side program code used to interact with the Human-Centric EHR system 102 having server-side program code. For example, client-side program code may include JavaScript that invokes AJAX queries to the server-side program code of the Human-Centric EHR system 102. In another example of implementation, the client-side program may be distributed as an app that can be downloaded from an app store such as the Apple app store or an Android app store.

The patient's computing entity 106 may include a display/screen or it can be connected to a display/screen on which a graphical user interface (GUI) may be implemented. The GUI may be conditioned based on the program code (e.g., application software) and/or various data received from the Human-Centric EHR system 102.

An EHR network 108 depicted in FIG. 1C may comprises one or more EHR systems. As indicated previously, an EHR system may be a single computing entity or a distributed computing environment (e.g., server arrangements). For instance, the one or more EHR systems of the EHR network 108 may be similar to the physician's EHR system 104 and/or the Human-Centric EHR system 102 described elsewhere in this document. The one or more EHR systems of the EHR network 108 may include one or more systems such as: health maintenance organization's (HMO's) EHR system; other physicians' EHR systems; EHR systems associated with pharmacies; EHR systems associated with dentists; EHR systems associated with optometrists; EHR systems associated with other medical facilities (e.g., laboratories such as testing and imagining labs); government regulated or state-owned health record system; and/or any other suitable system. By way of example, in Quebec, the government regulated health record system is the DSQ (Dossier de santé du Québec) and in some embodiments, the EHR network 108 comprises the DSQ. As such, the EHR network 108 may include a central EHR system 503, as shown in FIG. 1B, implemented as a single computing entity or as a distributed computing environment (e.g., server arrangements) that typically includes at least one processor, input/output circuitry and computer readable [symbol] ory including a database and program code. It is appreciated that the central EHR system 503 may [symbol] government regulated or state-owned EHR system or HMO's EHR system and/or any other suitable EHR system.

In some cases, the EHR network 108 may store the medical information by using a summary medical record system, such as of the type disclosed in Canadian Patent No. 2,329,598 or PCT International Publication No. WO 2015/031980, both of which are incorporated herein by reference.

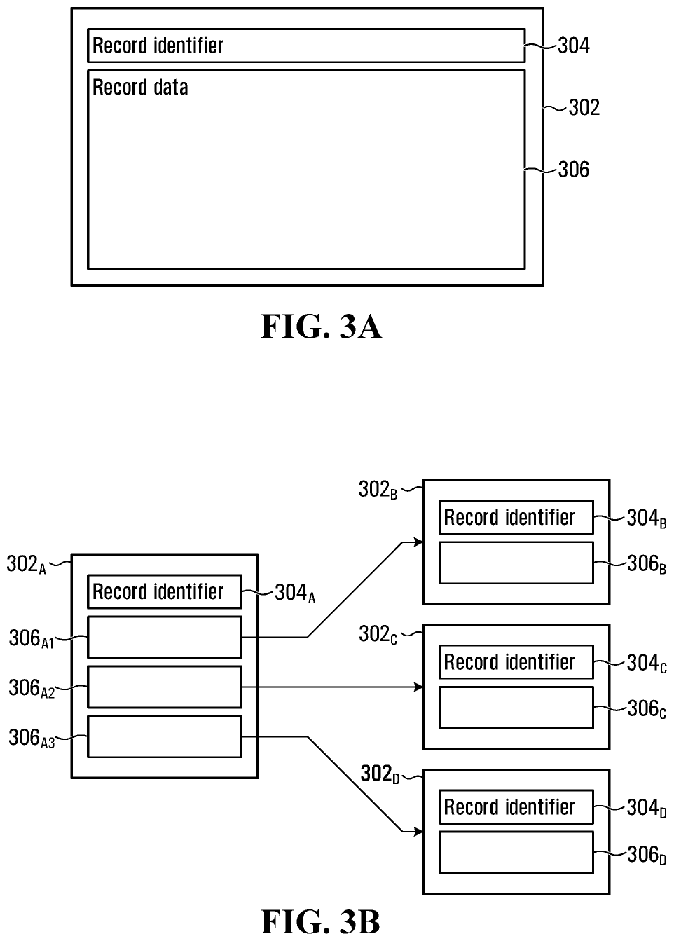

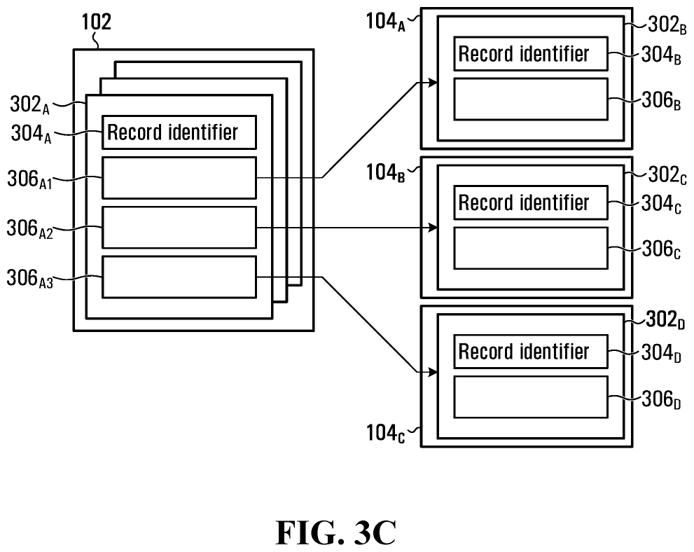

FIGS. 3A to 3C illustrate examples of data records 302 in accordance with embodiments of the invention. It will be understood that the representation is conceptual to facilitate the understanding of the way the information is organized, and it is not intended to be limiting in terms of how the data conveying that information is being structured or how the information is shown on a display device.

The data record 302 includes a record identifier 304 and record data 306. The record identifier 304 is used to identify the specific data record 302 and to distinguish it from the data records of other patients. The record data 306 is associated with the record identifier 304 such that information relevant to a specific patient identified by the record identifier 304 is stored in the record data 306 of the data record 302. The record data 306 may be physically stored in one centralized location or may be stored in a distributed fashion with a mechanism to link the various data components together so all the useful information can be retrieved when required. As such, the record data 306 may include data/information and/or may point to locations where data/information may be stored.

By way of example, FIG. 3B illustrates a data record 302_{A} having a record identifier 304_{A} and a plurality of records 306_{A} 306_{B} 306_{C} where each of the records 306_{A} 306_{B} 306_{C} includes a respective pointer pointing to respective ones of the data records 302_{B} 302_{C} 302_{D}. Each of the data records 302_{B} 302_{C} 302_{D} has a respective identifier 304B 304_{C} 304_{D} and respective data record 306_{B} 306_{C} 306_{D}. It is appreciated that in these examples the record data 306 may be a combination of data that is stored in one location and also includes links and/or pointers to data stored in a remote location, which can be accessed when required by downloading the data from the remote location pointed to by the one or more links.

By way of another example, FIG. 3C illustrates an implementation similar to that of FIG. 3B, where data record 302_{A} has a record identifier 304_{A} and a plurality of records 306_{A} 306_{B} 306_{C} where each of the records 306_{A} 306_{B} 306_{C} includes a respective pointer pointing to respective ones of the data records 302_{B} 302_{C} 302_{D}. However, as shown in FIG. 3C, the data records 302_{B} 302_{C} 302_{D} are respectively stored on [symbol] rate EHR systems, namely, a physician's EHR system 104_{A}, a laboratory EHR system 104_{B} and a [symbol] tal EHR system 104_{C} (e.g., such as shown in FIG. 1C). As it will be discussed later in this document, this implementation is useful in that it limits the amount of medical information stored on the EHR system 102, which is desirable for privacy reasons. While the data can still be accessed by authorized users from the EHR system 102 by following the links to the actual data sources 104_{A}, 104_{B} and 104_{C}, a data breach on the EHR system 102 will not provide access to the medical data itself. In this fashion the data is better protected.

For clarity, the data record 302 may be a health record of the type used (e.g., accessed, stored, obtained, communicated, transmitted and/or received) by the various systems and devices discussed in this document. The record data 306 of the data record 302 may include links and/or pointers to other data records of the type of the data record 302 by pointing to these other data records' identifiers and where these other data records' record data includes the additional information associated with the data record 302.

In the embodiments where the data record 302 is a health record, the data record 302 may include information such as: prescribed medication, delivered medication, laboratory results, pathology reports, consultation reports, imaging reports and images themselves, ECG (electrocardiogram) reports or the images themselves, surgical or procedure reports with or without images, allergies or medication intolerances, hospitalization summaries, physician summaries and/or any other suitable information. The data record 302 may also include patient identification information such as the patient's name, address, date of birth, health card number, primary physician, the issuing physician that prescribed the medication or medical test, and/or any other suitable information. The information stored in the data record 302 is not limited to the non-exhaustive list given above, a person skilled in the art would understand that other types of patient health and/or medical information may also be stored in the data record 302 in the various embodiments disclosed in this document.

In some embodiments, where the data record 302 is a health record stored on the EHR network 108, the data record 302 may include a summary component that includes information such as summaries of: Administrative Data, Permanent Biological Data, Significant Antecedents, Current Medical Conditions, Biological Data, Prescribed and/or Delivered Medications, Laboratory Results, Pathology Reports, Consultation Reports, Imaging Reports and Images, ECG reports and/or ECG Images, Surgical or Procedure Reports, Allergies and/or Medication Intolerances, Hospitalization Summaries or Physician Summaries. Furthermore, each summary may be associated with a pointer, which points to more complete [symbol] mation provided by the summary. By way of a specific and non-limiting example, the ECG reports [symbol] mary may list pointers to where the ECG images are actually stored. Similarly, different laboratory reports, images, prescribed prescriptions, and so forth, may be at different nodes of the data network and the summary records contains links that point to the different nodes in the network that store the complete information. As such, the person skilled in the art would clearly understand that any number of combinations of different types of data records where some data records are stored on various nodes (e.g., various EHR systems) of the EHR network 108 could exist. As various implementations of the data records are known in the art, data records do not need to be described in detail because they are well within the reach of a person skilled in the art.

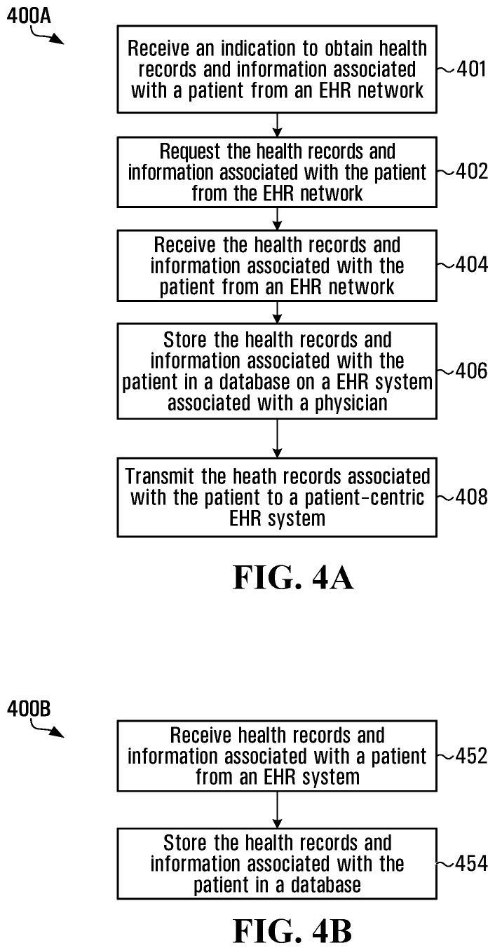

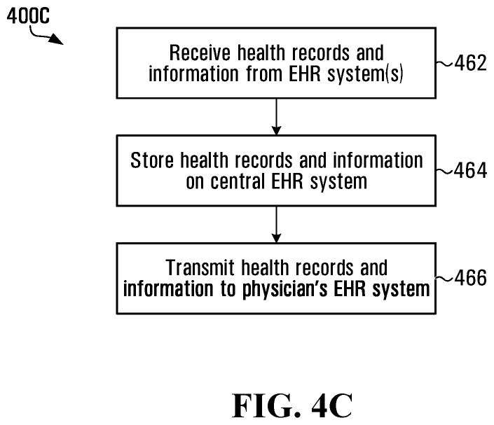

FIG. 4A illustrates a flowchart of a process 400A, which may be implemented by the physician's EHR system 104 in accordance with a specific example of implementation. At step 401 the physician's EHR system 104 receives an indication (e.g., a request) to obtain health records associated with a patient from the EHR network 108. In some cases, at step 401, the Human-Centric EHR system 102 initiates the process to obtain health records of the patient from the EHR network 108 by transmitting a request to the physician's EHR system 104. In these cases, the patient provides authorization to the Human-Centric EHR system 102 to retrieve the patient's health records from various sources. This may include the patient creating an account with the Human-Centric EHR system 102, which may be done via the patient's computing entity 106. In some cases, the patient uses a web browser running on the patient's computing entity 106 to connect to a website associated with the Human-Centric EHR system 102. In other cases, the patient may download an application that runs on the patient's computing entity 106 in order to connect to the Human-Centric EHR system 102 to create an account. In the account creation process, the patient may have to provide information about himself/herself, such information may include: name, address, date of birth, place of birth, health card number, social insurance number, email, user name, password, biometric identifier and/or any other suitable information. The Human-Centric EHR system 102 may then use the information received from the patient to authenticate that the person making the account is in fact that person. Regardless of the means that the patient creates an account with the Human-Centric EHR system 102, upon creation of an account and authorization from the patient, the Human-Centric EHR system 102 may initiate the process of obtaining the health records associated with the patient. Once the account is created, the Human-Centric EHR system 102 may maintain a record associated with the patient, where the record includes an account identifier (e.g., an account name/number, health card number, email and/or any suitable identifier) and credentials which may be used to authenticate the patient (e.g., a password, biometric information and/or any other suitable credentials). [symbol] her cases, at step 401, the indication to obtain health records associated with a patient from an EHR network 108 may be initiated by the physician, such as when a patient is at the physician's office and requests for his/her health records to be added to the Human-Centric EHR system 102. Similar to the case above, an account may be created at the Human-Centric EHR system 102. For example, the physician may create an account with the Human-Centric EHR system 102 upon verbal confirmation from the patient. In some cases, the patient may already have an account created with the Human-Centric EHR system 102 and visits the physician so that the physician via the physician's EHR system 104 may request the health records associated with the patient from the EHR network 108. Similar to how the patient has an account with the Human-Centric EHR system 102, physicians may also have accounts with the Human-Centric EHR system 102. For example, a physician may have an account with the Human-Centric EHR system 102 so that the physician may communicate via the physician's EHR system 104 with the Human-Centric EHR system 102. In such case, the Human-Centric EHR system 102 would have accounts associated with various physicians, where each account includes at least one identifier and at least one credential, such that a physician via the physician's EHR system 104 may then use the identifier and/or the credential in connecting to the Human-Centric EHR system 102.

In some instances, the health records of the patient available in the EHR network 108, such as an HMO and/or government maintained and ran EHR network and/or system, may be extracted when the physician consults the health records of the patient by logging into the EHR network. When the physician accesses the health records, the data in the records, which is owned by the patient, can be legitimately transferred to the Human-Centric EHR system 102. In this fashion, the Human-Centric EHR system 102 is populated with medical data every time the HMO and/or government managed EHR system is accessed by a physician. Over time, the HMO and/or government managed EHR system may be completely replicated into the Human-Centric EHR system 102. In other cases, the Human-Centric EHR system 102 may replicate the index (e.g., a list of pointers that point to specific records in the nodes of the EHR network 108) of the EHR network 108. In other words, meta-information may be replicated without replicating the data itself. The EHR network 108 is not limited to HMO and/or government maintained and ran EHR networks and/or systems but may be any suitable EHR network(s) and/or system(s) managed by one or more suitable organizations.

The record replication mechanism, which is performed every time the physician accesses the government and/or HMO managed EHR system 108, ensures that the information available in the Human-Centric EHR system 102 is maintained up to date. If changes have been made to the EHR record in the HMO [symbol] or government-managed system, those changes are automatically carried over into the Human- [symbol] ric EHR system 102.

At step 402, the physician's EHR system 104 requests the health records associated with the patient from the EHR network 108. This may include the physician's EHR system 104 connecting to the EHR network 108, in which the physician has an account with. For example, the physician may provide his/her username and password and/or hardware token such that the physician's EHR system 104 is able to connect to the EHR network 108. The process of EHR systems connecting to the EHR network 108 is known in art, for example as is disclosed in PCT International Publication No. WO 2015/031980, the contents of which are incorporated herein by reference. As various means for connecting to the EHR network 108 are known in the art, the means for connecting by the physician's EHR system 104 to the EHR network 108 does not need to be described in detail because such means are well within the reach of a person skilled in the art. The requests for the health records associated with the patient from the EHR network 108 may include an identifier associated with the patient, which the EHR network 108 may use to obtain the health records associated with the patient. The requests for the health records associated with the patient from the EHR network 108 may also include an identifier of the physician's EHR system 104 making the request.

Even more specifically, the physician accesses the government-managed or HMO-managed EHR system during a consultation, which access includes downloading the medical data to the physician's computer to display it on the physician's display. The software that manages the replication of the medical data makes a copy of the information sent from the EHR government-managed or HMO-managed system to the Human-Centric EHR system. The software can also be designed to parse the information made available when the file access was requested to determine if additional data needs to be obtained such as to obtain a complete copy of the patient record. For example, if the medical data stored in the government-managed EHR system includes a summary portion that uses links allowing access to additional medical data, the software is configured to parse the file and recognize the links and invoke them such that the additional medical information is also sent by the HMO and/or government-managed EHR system to the physician. That additional medical information is then also copied and integrated into the Human-Centric EHR system 102.

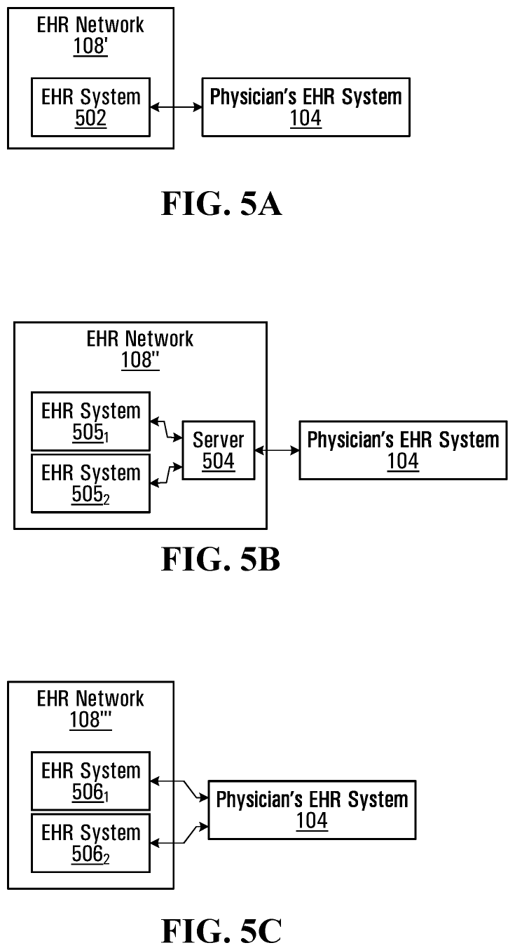

FIG. 5A illustrates a block diagram of an EHR network 108′ comprising a single EHR system 502 in accordance with a specific non-limiting example of implementation. The EHR network 108′ is a specific non-limiting example of implementation of the EHR network 108 (discussed elsewhere in this document). [symbol] is example, the single EHR system 502 stores the health records associated with the patient. In the [symbol] ext of this example, at step 402, the physician's EHR system 104 requests the health records associated with the patient from the single EHR system 502. In turn, the single EHR system 502 transmits the health records associated with the patient to the physician's EHR system 104.

FIG. 5B illustrates a block diagram of an EHR network 108″ comprising a plurality of EHR systems 505₁ 505₂ accessible by a server 504 in accordance with a specific non-limiting example of implementation. The server 504 refers to one or more computing entities (e.g., machines) on which a server program runs that waits for requests from other computing entities (e.g., physician's EHR system 104) and responds to them. The server 504 may be a record aggregation system. The EHR network 108″ is a specific non-limiting example of implementation of the EHR network 108 (discussed elsewhere in this document). In this example, the EHR network 108″ comprises a plurality of EHR systems 505₁ 505₂, such that the health records associated with the patient is distributed amongst the plurality of EHR systems 505₁ 505₂. In other words, the health records associated with the patient are distributed amongst the EHR network 108″ such that at least part of the health records associated with the patient are stored on each of the EHR systems 505₁ 505₂. In the context of this example, at step 402, the physician's EHR system 104 requests the health records associated with the patient from the centralized server 504 associated with the EHR network 108″. In turn, the server 504 communicates with the plurality of EHR systems 505₁ 505₂ to obtain the requested health records associated with the patient, which may include the server 504 querying the plurality of EHR systems 505₁ 505₂. Then the server 504 transmits the health records associated with the patient to the physician's EHR system 104. The server 504 in some cases may be an EHR system which stores at least part of the health records associated with the patient. More specifically, the server 504 may be the central EHR system 503.

FIG. 5C illustrates a block diagram of the EHR network 108′″ comprising a plurality of EHR systems 506₁ 506₂ in accordance with a specific non-limiting example of implementation. The EHR network 108′″ is a specific non-limiting example of implementation of the EHR network 108 (discussed elsewhere in this document). In this example, the EHR network 108′″ comprises a plurality of EHR systems 506₁ 506₂, such that the health records associated with the patient is distributed amongst the plurality of EHR systems 506₁ 506₂. In other words, the health records associated with the patient is distributed amongst the EHR network 108′″ such that at least part of the health records associated with the patient is stored on each of the EHR systems 506₁ 506₂. In the context of this example, at step 402, the physician's EHR system 104 requests health records associated with the patient from each of the plurality [symbol] HR systems 506₁ 506₂. In turn, each of the plurality of EHR systems 506₁ 506₂ transmits health [symbol] rds associated with the patient to the physician's EHR system 104.

The various EHR systems 502, 505₁ 505₂ 506₁ and 506₂ may be of the type of EHR system that is discussed elsewhere in this document. Although in both FIG. 5B and FIG. 5C two EHR systems 505₁ and 505₂ or 506₁ and 506₂ are shown, the EHR network 108 may comprise more than two EHR systems. The configuration of the EHR network 108 illustrated in FIGS. 5A, 5B and 5C is intended to not be limiting and various other configurations of the EHR network 108 are possible in other embodiments. Although in FIGS. 5A, 5B and 5C the physician's EHR system 104 is shown connecting directly to the EHR network 108, in other examples the Human-Centric EHR system 102 may connect directly to the EHR network 108. Any communication with the EHR network 108 from other systems and/or devices as discussed in this document may be communication with any of the systems and/or devices that are included in the EHR network 108, for example, such as those shown in FIGS. 5A-5C.

Turning back to FIG. 4A, at step 404, the physician's EHR system 104 receives the health records associated with the patient from the EHR network 108. In other words, at step 404, the physician's EHR system 104 obtains the health records associated with the patient, in response to requesting the health records associated with the patient from the EHR network 108.

At step 406, the health records associated with the patient may then be stored in the physician's EHR system 104. For example, the obtained health records associated with the patient may be stored in the database 226 of the physician's EHR system 104 in association with the patient. In some cases, the database 226 of the physician's EHR system 104 may have additional medical information relating to the patient. In other words, at least part of the health records associated with the patient may be stored in the database 226 of the physician's EHR system 104 prior to the physician's EHR system 104 making the request to the EHR network 108 to obtain the remaining parts of the health records associated with the patient. In other cases, the physician's EHR system 104 and the EHR network 108 may have at least in part duplicate information relating to the health records associated with the patient.

Then at step 408, the health records associated with the patient is transmitted to the Human-Centric EHR system 102. The Human-Centric EHR system 102 receives the health records associated with the patient and stores these health records in one or more records in the database 206 in association with the patient. In other words, the health records associated with the patient may be stored in a manner that is associated with the patient's account. Such a configuration may allow for the patient to access his/her health records [symbol] onnecting to the Human-Centric EHR system 102 (e.g., via a web browser or an application running [symbol] e patient's computing entity 106) and log into the patient's account. By allowing the patient to access his/her health records on the Human-Centric EHR system 102 the patient may be able to control various aspects of his/her health records. For instance, the patient's account may have a profile associated with it, where the profile has various settings, parameters and/or options that the patient can configure. Moreover, where the health records associated with the patient are obtained from multiple sources (e.g., multiple nodes/systems of the EHR network 108) and then stored in the Human-Centric EHR system 102, this may allow for a centralized storage of the health records associated with the patient.

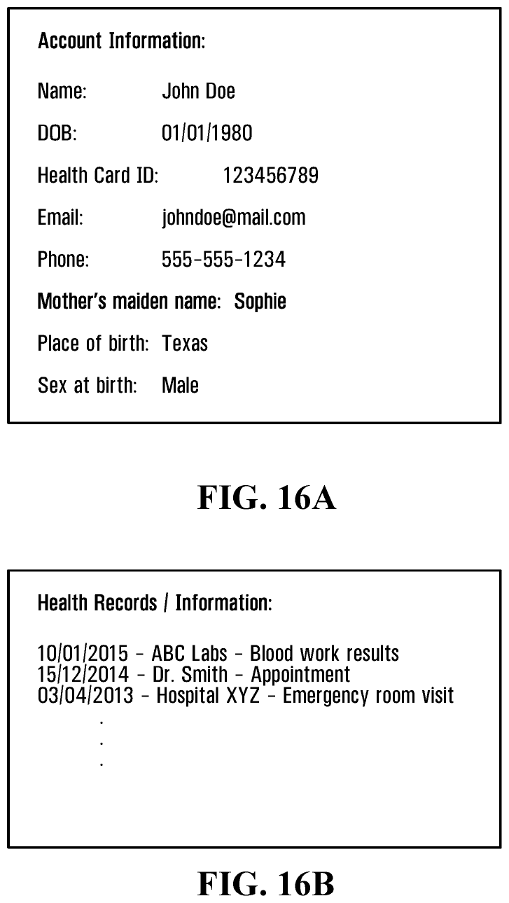

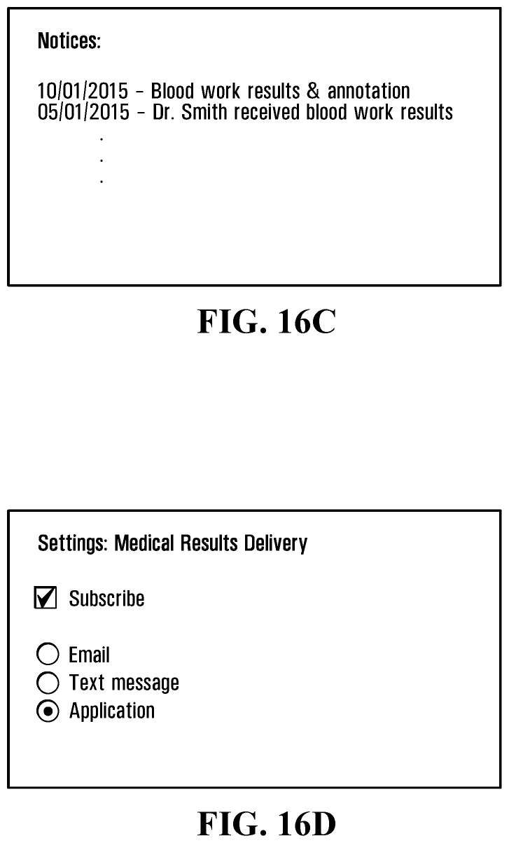

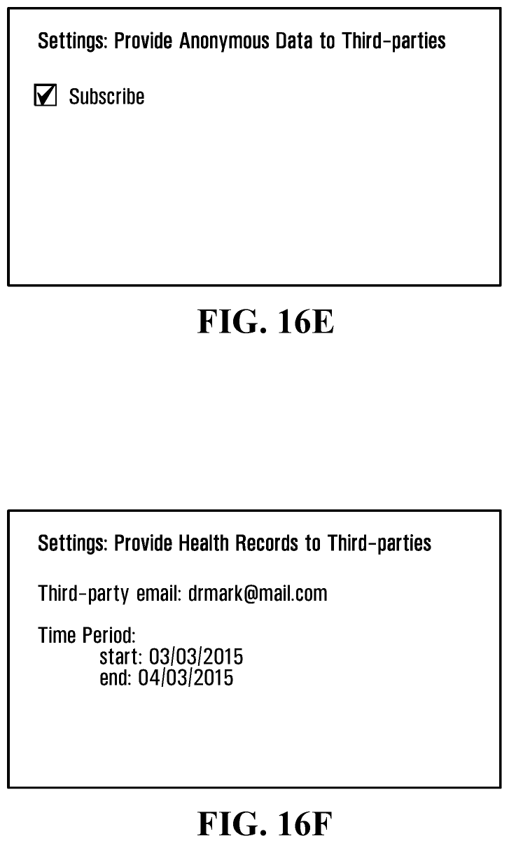

FIGS. 16A-16F illustrate specific and non-limiting examples of information that may be displayed on the GUI of the patient's computing entity 106 in accordance with specific and non-limiting examples of implementation. FIG. 16A illustrates an example of account information associated with the patient. FIG. 16B illustrates a plurality of health records obtained from multiple sources. As shown in this example, the plurality of health records includes laboratory results from ABC Labs, annotations from an appointment with Dr. Smith and annotations from a hospital visit to Hospital XYZ. The patient via the patient's computing entity 106 may be able to connect to the Human-Centric EHR system 102 after step 408 of process 400A and view the plurality of health records associated with the patient (e.g., as shown in FIG. 16B). The patient may also be able receive various notifications from the Human-Centric EHR system 102, as shown in FIG. 16C and discussed elsewhere in this document. The patient may also be able to control various aspects of the plurality of health records stored on the Human-Centric EHR system 102 and associated with the patient for example as is shown in FIGS. 16D, 16E and 16F.

FIG. 4B illustrates a flowchart of a process 400B which may be implemented by the physician's EHR system 104 in accordance with a specific example of implementation. In this example, the physician's EHR system 104 registers with the EHR network 108 which may include registering with the central EHR system 503 (or the server 504, as shown in FIG. 5B) or one or more EHR systems (e.g., such as those shown in FIGS. 5A and 5C). The registration process may include the physician's EHR system 104 providing to the EHR network 108, central EHR system 503 and/or various EHR systems a list of patients, where the list of patients includes patients that the physician provides medical services to. The list of patients may include for each patient on the list the patient's name, date of birth, health identifier number and/or any other suitable information. Once registered, the physician's EHR system 104 is subscribed to receive records associated with the list of provided patients. That is, when the EHR network 108, central EHR system 503 and/or various EHR systems updates a health record associated with a patient, the updated record and/or information associated with the updated record is transmitted to the [symbol] ician's EHR system 104. At step 452, the physician's EHR system 104 receives the health record and [symbol] , at step 454, stores the health record in association with the patient's record on the physician's EHR system 104. In other words, the physician's EHR system 104 automatically receives the health records for all of its patients, regardless of where a specific patient is registered with the Human-Centric EHR system 102. The physician's EHR system 104 may then transmit the records of the patients that are registered with the Human-Centric EHR system 102 to the Human-Centric EHR system 102, in a similar manner as discussed elsewhere in this document.

FIG. 4C illustrates a flowchart of a process 400C which may be implemented by the central EHR system 503 in accordance with a specific example of implementation. The process 400C is now discussed with reference to FIG. 1B. At step 462, the central EHR system 503 receives from one or more EHR systems (e.g., the physician's EHR system 104_{A}, the laboratory EHR system 104_{B} and the hospital EHR system 104_{C}, etc.) one or more health records. The received health records may be transmitted to the central EHR system 503 upon request from the central EHR system 503 to the one or more EHR systems or may automatically be transmitted to the central EHR system 503 from the one or more EHR systems, for example, when a record is updated or based on a period of time (e.g., on a daily basis). The central EHR system 503 may process the received records and, at step 464, store the records in association with the corresponding patients that the records correspond thereto in a database of the central EHR system 503. At step 466, the central EHR system 503 may then transmit health records on the central EHR system 503 to the physician's EHR system 104. For instance, the central EHR system 503 may have a list of registered EHR systems that are to receive patient records. The central EHR system 503 may then process in response to receipt of the records from step 462 and if any of those records are associated with a patient that is associated with the subscribing physician's EHR system 104, then the central EHR system 503 transmits those records to the subscribing physician's EHR system 104.

Although in the embodiments illustrated above, the Human-Centric EHR system 102 receives the health records associated with the patient via the physician's EHR system 104, in other cases the Human-Centric EHR system 102 may be connected directly to the EHR network 108 to request and obtain the health records associated with the patient. For instance, the Human-Centric EHR system 102 may request the health records associated with the patient directly from the EHR network 108 in a similar fashion to that discussed in regard to step 402; the Human-Centric EHR system 102 may receive the health records associated with the patient directly from the EHR network 108 in a similar fashion to that discussed in regard to step 404; and the Human-Centric EHR system 102 may store the health records associated with the patient in a similar fashion to that discussed in regard to step 406. [symbol] example, as shown in FIG. 1C, the Human-Centric EHR system 102 may be part of the EHR network 108 such that it is connected to various EHR systems (e.g., a physician's EHR system 104_{A}, a laboratory EHR system 104_{B} and a hospital EHR system 104_{C}) of the EHR network 108. In other cases, the Human-Centric EHR system 102 may be connected to and communicates with the central EHR system 503, where the central EHR system 503 is connected to and communicates with various EHR systems (e.g., the physician's EHR system 104_{A}, the laboratory EHR system 104_{B} and the hospital EHR system 104_{C}).

It is appreciated that FIGS. 1A to 1C are examples of possible configurations and interconnections of the Human-Centric EHR system 102 and the EHR network 108 and that other configuration are possible.

The Human-Centric EHR system 102 may also maintain a record of the various physicians' EHR systems that the patient has medical records associated with.

The technique for populating an EHR system such as the physician's EHR system 104 and/or the Human-Centric EHR system 102 may be implemented in various ways. One technique for populating an EHR system with health records associated with respective patients includes querying the centralized EHR network 108 storing health records about multiple patients. The querying may be performed in the context of a physician dispensing medical services to a first patient of the multiple patients. For example, this may take place when the first patient is at the physician's office seeking a medical consult (e.g., an appointment with the physician). This medical consult may be to seek medical advice/services and/or may be for the purpose of populating the EHR system with health records associated with the first patient. The querying in this example includes accessing the health record of the first patient from the centralized EHR network 108. It may also include accessing the health record of the first patient from physician's EHR system 104, for example if the physician's EHR system 104 has additional health records about the first patient that are not on the centralized EHR network 108. This technique could then include copying some or all of the information in the health record of the first patient on the centralized EHR network 108 to the EHR system to create a health record for the first patient in the electronic health record system. This copying may include copying some or all of the information in the health record of the first patient on the centralized EHR network 108 to the physician's EHR system 104 and/or the Human-Centric EHR system 102. The physician's EHR system 104 may transmit some or all of the information in the health record of the first patient on the centralized EHR network 108 to the Human-Centric EHR system 102 for storage. [symbol] querying and copying step may then be repeated for a second patient of the multiple patients to [symbol] ss and copy the health record of the second patient from the centralized EHR network 108. The querying and copying step may then be repeated numerous times such as for a third patient, a fourth patient and so on, of the multiple patients. For example, this may be done each time that the physician is dispensing medical services to a patient of the multiple patients.

It is appreciated that the physician may dispense medical services to only a subset of patients of the multiple patients. In other words, the centralized EHR network 108 stores health records about multiple patients and the physician may only access and copy the health records of the patients that the physician is dispensing medical services thereto. This is the case because the centralized EHR network 108 would typically also store health records associated with patients that are not patients of the physician but of other physicians.

By way of example, each time the physician dispenses medical services to the first patient, the querying of the centralized EHR network 108 may be repeated to access and copy any new health records associated with first patient to the EHR system. This constitutes an update of the first patient's record on the EHR system based on any new records of the first patient on the centralized EHR network 108. In other cases, the querying of the centralized EHR network 108 may be done to access and update the first patient's health records on the EHR system upon request from the physician and/or the physician's EHR system 104. For example, when the physician desires to obtain the new health records associated with the first patient from the centralized EHR network 108 and/or when physician's EHR system 104 has been programmed to access the centralized EHR network 108 such as on a regular interval (e.g., daily, weekly, month basis and/or any other suitable timeframe). In even further cases, the centralized EHR network 108 may be programmed to push the new health records of the first patient to the electronic health record system such as on a regular interval (e.g., daily, weekly, month basis and/or any other suitable timeframe).

It is appreciated that the aforementioned electronic health record system that was populated with health records associated with respective patients could provide an account to the first patient such that the first patient can access the health record associated therewith on the electronic health record system. An account may also be provided to the second patient, and so forth. The account provided may be as discussed elsewhere in this document. This account may be an account to the physician's EHR system 104 and/or the Human-Centric EHR system 102. [symbol] me embodiments, the EHR network 108 may be implemented to include the use of block-chains. A [symbol] k-chain is a distributed non-centralized database that maintains a continuously growing list of data records that each refers to previous items on the list, which may be referred to as a ledger. More specifically, the block-chain includes blocks that hold timestamped data records. Each block also includes a hash of the prior block, linking the blocks together. The linked blocks form a chain, each additional block reinforcing those before it, thus giving the database type its name. In this embodiment, various nodes would include at least a partial copy of the block chain.

Medical Results Delivery Mechanism

This section discusses a functionality of the Human-Centric EHR system that communicates medical information to the user (patient) in a seamless and secure fashion.

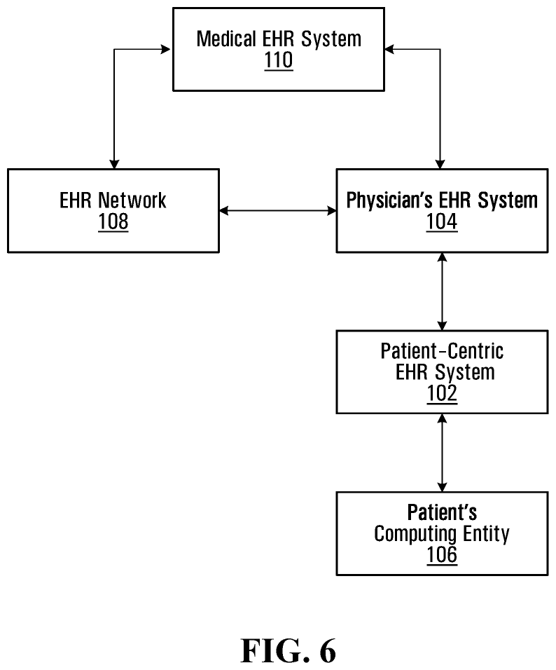

FIG. 6 illustrates a block diagram of an example of the Human-Centric EHR system 102 that is able to provide patients with medical results upon authorization in accordance with an embodiment of the invention. As illustrated, the Human-Centric EHR system 102 is connected to the patient computing entity 106 and to the physician's EHR systems 104 which is connected to EHR network 108. Also, a medical EHR system 110 is illustrated as being connected to both the EHR network 108 and to the physician's EHR systems 104. Although in the embodiment shown the medical EHR system is connected to both the EHR network 108 and the physician's EHR system 104, in other embodiments the medical EHR system 110 may only be connected to one of the EHR network 108 and physician's EHR system 104. It is appreciated that other connections are possible in other embodiments.

The various connections between the Human-Centric EHR system 102, the patient's computing entity 106, the physician's EHR system 104, the medical EHR system 110 and/or the EHR network 108 may be similar to the various connections between systems and devices, as discussed elsewhere in this document. The various connections between the Human-Centric EHR system 102, the patient's computing entity 106, the physician's EHR system 104, the medical EHR system 110 and/or the EHR network 108 may include one or more connections over one or more data networks. The various connections between the Human-Centric EHR system 102, the patient's computing entity 106, the physician's EHR system 104, the medical EHR system 110 and/or the EHR network 108 may allow for the communication (e.g., transmission and/or reception) of various information, such as information of medical nature between the various systems and/or devices. The various information and/or data exchanged may be a “notification” or a “health record” of the type discussed elsewhere in this document. A notification would typically be [symbol] nnouncement on the computing entity 106 to inform the patient that medical information is available [symbol] e consulted. For privacy reasons a notification would not convey information of medical nature, however it is not excluded that a notification may include some if not all of the medical information it refers to.

The medical EHR system 110 may be one or more computer systems associated with a laboratory, testing facility, imaging facility or any other suitable facility that provides medical results associated with a test or image of a patient (e.g., an EHR system associated with a laboratory, testing facility and/or imaging facility). The medical result may include test results, such as blood test results, urine test results; imaging results, such as an X-ray image results or CAT (computerized axial tomography) scan results; CT (computerized tomography) scan; ECG images and/or reports; or any other suitable laboratory, imagining or test result and/or reports. The medical results may be provided in a health record associated with the patient.

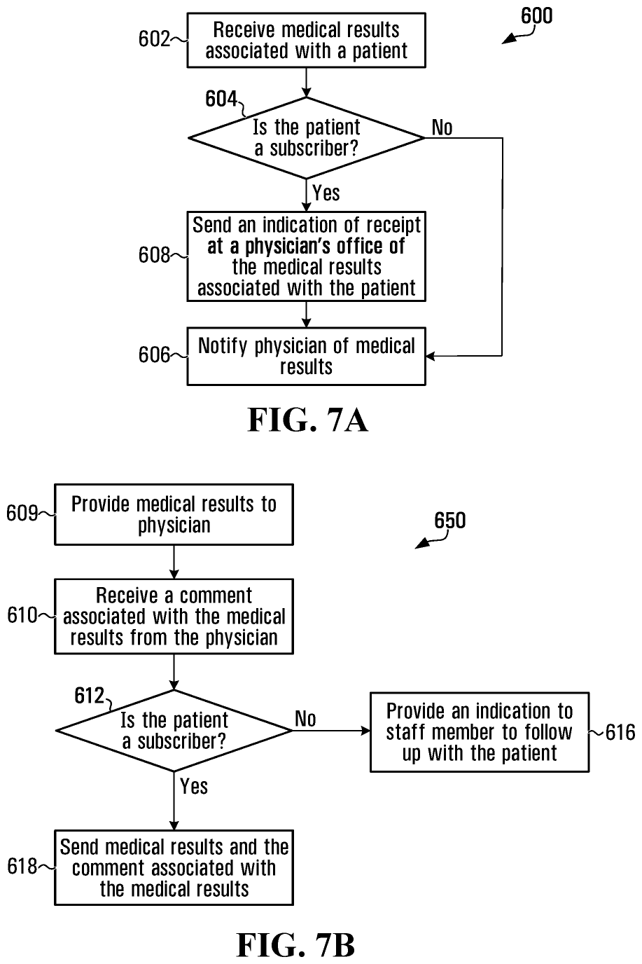

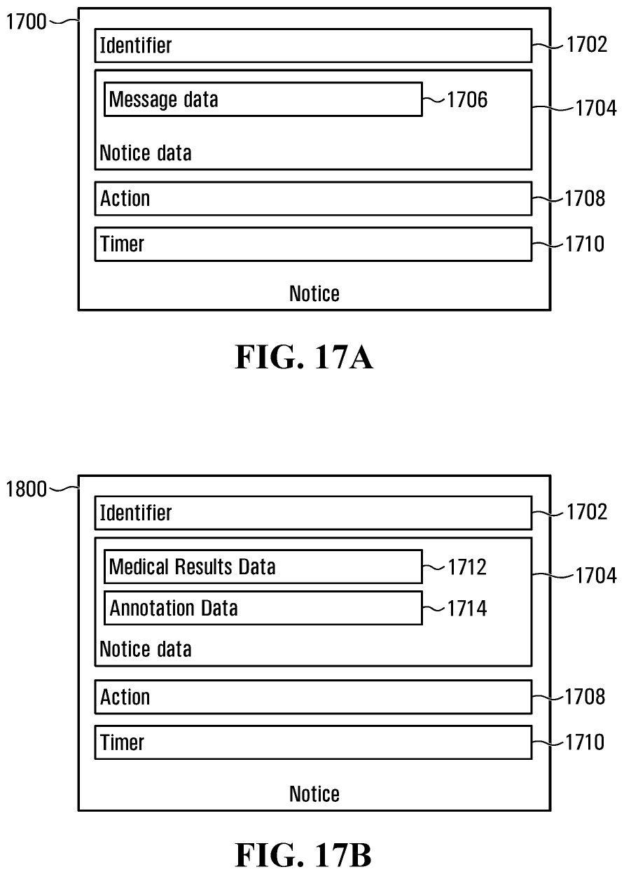

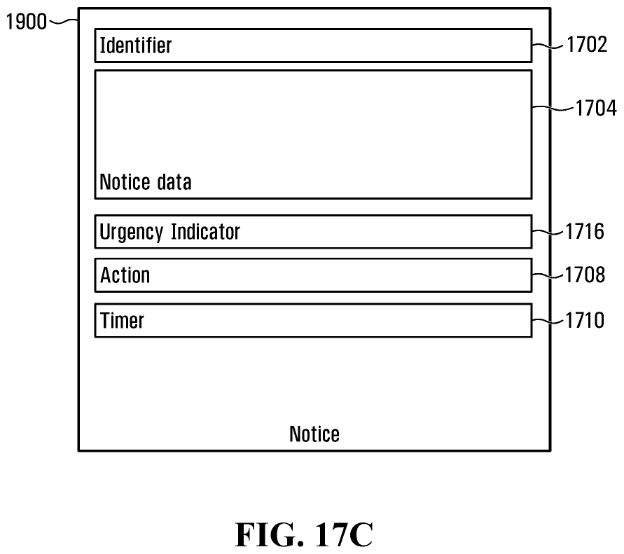

FIG. 7A illustrates a flowchart of a process 600, which may be implemented by the physician's EHR system 104 for determining if the Human-Centric EHR system 102 should be notified of receipt of medical results at a physician's office in accordance with a specific non-limiting example of implementation. Prior to process 600, it is appreciated that a patient may have visited the physician and the physician requested that the patient get medical results done (e.g., testing or lab-work done). After the patient visits the facility to get the medical testing or lab-work done, the results are entered into a medical EHR system 110. The Human-Centric EHR system 102 may receive an indication from the physician's EHR system 104 that medical testing or lab-work has been prescribed and the Human-Centric EHR system 102 may send a notification to the patient's computing entity 106 to get medical results done. The Human-Centric EHR system 102 may use this indication to setup a reminder notification if the patient does not get the medical testing or lab-work done within a certain timeframe. The notifications are discussed elsewhere in this document, such as the discussion in association with FIGS. 17A and 17B.

At step 602, medical results associated with a patient are received at the physician's EHR system 104. At this step, the medical results may be received from the medical EHR system 110 or may be received from the EHR network 108 (e.g., the central EHR system 503 and/or any other EHR system). For instance, in some cases, the medical EHR system 110 may send the medical results to the EHR network 108 and the physician's EHR system 104 queries the EHR network 108 to obtain the medical results or the EHR network 108 transmits the medical results to the physician's EHR system 104 upon receipt from the medical EHR system 110. In other cases, the medical results may be provided in hardcopy and a user [symbol] rs and/or scans the medical results into the EHR network 108 and/or physician's EHR system 104. [symbol] rdless of how the medical results are received, at this step the medical results may be stored in a health record associated with the patient.

At step 604, the patient information associated with the medical results is processed to see if the patient is a subscriber to the Human-Centric EHR system 102 and/or a subscriber to a medical results delivery service. For example, prior to the physician's EHR system 104 receiving the medical results associated with the patient, the patient may have logged into his/her account and subscribed to the medical results delivery service. As such, the Human-Centric EHR system 102 may manage a subscribing list of the patients that subscribe to the medical results delivery service. FIG. 16D illustrates a specific and non-limiting example of a profile page of the patient's account, which provides the options to subscribe to the medical results delivery service. As illustrated, the Human-Centric EHR system 102 may also allow for the patient to select the means for receiving notifications (e.g., email, text messages and application). In other cases, the patient may not have the option to select the mode for receiving notifications; for example, in the case where the patient's computing entity 106 is running application software associated with the Human-Centric EHR system 102 the notifications may be received via the application software.

Turning back to FIG. 7A, at step 604, the physician's EHR system 104 may query the Human-Centric EHR system 102 to determine if the patient associated with the medical results is on the subscriber list or not. In this case, the Human-Centric EHR system 102 maintains a subscriber list which lists the subscribers (e.g., various patients) and the physician's EHR system 104 may provide the Human-Centric EHR system 102 with an identifier associated with the patient (e.g., a medical card number, an account identifier or any other suitable identifier) that can then be used by the Human-Centric EHR system 102 to make a determination of whether the patient associated with the identifier is a subscriber or not. In other cases, the physician's EHR system 104 may query the Human-Centric EHR system 102 for a subscriber list comprising identifiers associated with patients that are a part of the service and then can determine by processing the subscriber list against an identifier associated with the patient. In this case, if a match is found then the patient associated with the medical results is a subscriber and if a match is not found then the patient associated with the medical results is not a subscriber. Yet, in other cases, the physician's EHR system 104 may include in the medical records associated with the patient that the patient is a subscriber to the medical results delivery service. Regardless of how it is determined that the patient is a subscriber, at this step a determination is made (e.g., at the physician's EHR system 104 and/or the Human-Centric EHR system 102) to determine whether a specific patient is a subscriber or not. [symbol] ay of example, the interaction between the physician's EHR system 104 and the Human-Centric [symbol] system 102 to determine if a specific patient is a subscriber may be implemented as follows. The physician's EHR system 104 queries the Human-Centric EHR system 102 by sending a request to see if the Human-Centric EHR system 102 already has health records associated with a specific patient. The request may include providing a patient's name, gender, date of birth, date of death, social insurance number, a medical card identifier and/or any other suitable identifier. The Human-Centric EHR system processes the request and in the case that a match is found (i.e., the Human-Centric EHR system has health records associated with the requested patient), the Human-Centric EHR system 102 may then communicate to the physician's EHR system 104 that the Human-Centric EHR system 102 has health records associated with the requested patient and that this specific patient may be identified by a specific identifier (e.g., a proxy identifier). This specific identifier may be specific to the requesting physician's EHR system 104. In other words, each physician's EHR system in a plurality of physician's EHR system that may communicate with the Human-Centric EHR system 102 would have a specific identifier for a specific patient, where the specific identifier is different for each of the physician's EHR systems. In the case that a match is not found, the Human-Centric EHR system 102 may still provide the specific identifier for the requested patient and an indication that the Human-Centric EHR system 102 currently does not have any health records associated with the requested patient. It is appreciated that by providing the specific identifier even though the Human-Centric EHR system 102 does not currently have any health records associated with the requested patient that the Human-Centric EHR system 102 may be later able to identify the patient when other physician's EHR systems try to request health records associated with the patient. Such a configuration between the Human-Centric EHR system 102 and physician's EHR system 104 may allow for when the physician's EHR system 104 provides the Human-Centric EHR system 102 with health records associated with the patient, that the physician's EHR system 104 provides the specific identifier of the patient along with a unique identifier associated with the physician's EHR system 104 (e.g., a doubleton).

If the patient is a subscriber to the service, then at step 608 an indication that the medical results have been received at the physician's office is transmitted from the physician's EHR system 104 to the Human-Centric EHR system 102. At step 606, the physician's EHR system 104 may then notify the physician of the received medical results. If the patient is not a subscriber, then the physician's EHR system 104 may then notify the physician of the received medical results (step 606). In other cases, an optional note may be made in a record associated with the patient that an indication was not sent to the Human-Centric EHR system 102. In other words, a note that the patient was not informed of receipt of the medical results at the [symbol] ician's office. In other cases, when the patient is a subscriber, an optional note may be made that [symbol] nt was informed that the medical results were received at the physician's office.

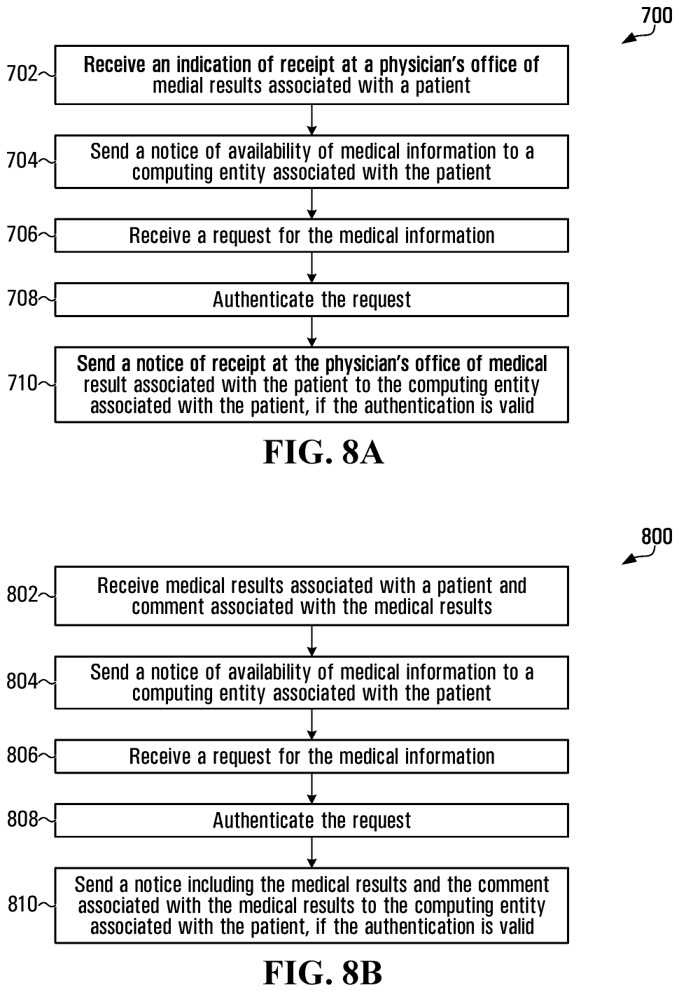

FIG. 8A illustrates a flowchart of a process 700 which may be implemented by the Human-Centric EHR system 102 for sending a notification to the patient's computing entity 106 in accordance with a specific non-limiting example of implementation. At step 702, the Human-Centric EHR system 102 receives from the physician's EHR system 104 an indication that that medical results associated with a patient that subscribes to the notification system of Human-Centric EHR system 102 (i.e., a subscriber) have been received at the physician's office. Step 702 may take places after step 608 of process 600 and in general terms is an indication that medical results are available in the physician's EHR system 104 for review by the physician. At this step the Human-Centric EHR system 102 may store the indication that that medical results associated with the patient have been received at the physician's office in a record associated with the subscribing patient. It is appreciated that there may be a distinction between a patient and a subscriber (or a subscribing patient), as the physician's EHR system 104 would typically have electronic health records associated with different patients, and the physician's EHR system 104 may provide the electronic health records to the Human-Centric EHR system 102 regardless of whether the patient subscribes to the notifications of the Human-Centric EHR system 102.

At step 704, the Human-Centric EHR system 102 then sends a notification to the subscribing patient's computing entity 106 associated with the subscribing patient to notify the subscribing patient that medical information is available. The notification that medical information is available may depend on the type of delivery method that the subscribing patient desires to obtain notifications by. For example, the subscribing patient may provide an email address to receive the notification in the form of an email; a cell-phone number to receive the notification as a text message. In other cases, where the subscribing patient's computing entity 106 runs an application associated with the Human-Centric EHR system 102 and the application may provide the notification in the form of a pop-up box or window type notification or a red circle in a top corner of an icon associated with the application running on the subscribing patient's computing entity 106. Regardless of the means for providing the notification, the notification may only note that medical information is available for viewing at the Human-Centric EHR system 102 and not provide any specific details regarding the medical information. In other words, patient intervention would typically be required to make the actual medical information visible to the user of the subscribing patient's computing entity 106, as discussed in the steps below. [symbol] tep 706 a request for the medical information from the subscribing patient's computing entity 106 is [symbol] ived and the request may include authentication information. In the cases where the notification is provided via email or text-message, the subscribing patient via the subscribing patient's computing entity 106 may login to the Human-Centric EHR system 102 (e.g., through the use of a web browser) to obtain the medical information. For instance, the subscribing patient may provide a user account identifier and a password. In the cases where the notification is provided in the form of a pop-up box or window type notification in the application running on the subscribing patient's computing entity 106, the subscribing patient may provide a user account identifier, a password and/or biometric authentication information (e.g., a fingerprint via a fingerprint reader on the patient's computing entity 106) via the application. In other words, at this step the subscribing patient's computing entity 106 requests from the Human-Centric EHR system 102 access to medical information by providing authentication information.

At step 708, the authentication information included in the request for the medical information is authenticated (e.g., by comparing the authentication information against a record associated with the patient that stores the credential information). Then at step 710 if the authentication information is associated with the subscribing patient, the Human-Centric EHR system 102 then transmits a notification providing the medical information to the subscribing patient's computing entity 106. In this case, the medical information is a notification that medical results have been received at his/her physician's office but has not yet been reviewed.

In some cases, step 704 may be omitted from the process 700. In these cases, a notification of the availability of medical information is not transmitted to the subscribing patient's computing entity 106 and the Human-Centric EHR system 102 waits for a request from the subscribing patient's computing entity 106.

In other cases, step 708 may be omitted from the process 700 and prior to step 704 the subscribing patient's computing entity 106 provides authentication information and if the authentication information is associated with the subscribing patient, then a notification may be sent at step 704. In other words, notifications are not sent unless the subscribing patient has authenticated his/her patient's computing entity 106 to the Human-Centric EHR system 102 in a manner that indicates he/she is currently using the device and would like notifications to be received.

FIG. 7B illustrates a flowchart of a process 650 which may be implemented by the physician's EHR system 104 for transmitting medical results to the Human-Centric EHR system 102 in accordance with a [symbol] ific non-limiting example of implementation. After step 606 of the process 600, the medical results [symbol] be provided to a physician at step 609 of process 650. In other words, after the physician's EHR system receives the medical results associated with the patient, the physician is able to view the medical results and, at step 609, the medical results associated with the patient are provided to the physician. For example, the display/screen of the physician's EHR system 104 may provide the physician with the results and the physician may provide a comment (e.g., an annotation) via an input device such as a keyboard, mouse and/or touch screen. It is appreciated that the terms “comment” and “annotation” may be used interchangeably in this document, where appropriate, to refer to any annotation, comment, observation and/or explanation associated with a medical result. At step 610, the physician provides a comment regarding the medical results and the physician's EHR system 104 receives the comment and stores the comment in association with the medical results in a health record associated with the patient. At step 612, the physician's EHR system 104 then checks to see if the patient is a subscriber (step 612 of process 650 is similar to step 604 of process 600). If the patient is a subscriber, then at step 618 the medical results associated with the patient and the comment associated with the medical results for the patient are sent to the Human-Centric EHR system 102 from the physician's EHR system 104 (step 610 of process 650 is similar to step 608 of process 600; however, instead of providing an indication of the medical results, the medical results themselves are transmitted). It is appreciated that transmission of the medical results may include the actual medical results and/or an explanation of the medical result, which may be transmitted in the form of an electronic health record. It is further appreciated that instead of transmission of the medical results that an explanation of the medical results may be transmitted instead. If the patient is not a subscriber, then at step 616, an indication would typically be provided to a staff member (e.g., nurse, receptionist, etc.) to follow-up with the patient (e.g., give them a call or send them an email) to book an appointment to come in and see his/her medical results, if appropriate in the circumstances. Optionally, a note may be made in the health record associated with the patient that the medical results was not sent to the Human-Centric EHR system 102 to indicate that the patient was not notified of the medical results. In other cases, an optional note may be made in the health record associated with the patient that the patient has been informed of the medical results.

FIG. 8B illustrates a flowchart of a process 800 which may be implemented by the Human-Centric EHR system 102 for sending medical results associated with the subscribing patient to the subscribing patient's computing entity 106 in accordance with a specific non-limiting example of implementation. At step 802 the Human-Centric EHR system 102 receives from the physician's EHR system 104 medical results associated with the subscribing patient. Step 802 may take places after step 618 of process 650 and in general terms the medical results associated with the subscribing patient that are received from the [symbol] ician's EHR system 104 include a comment from the physician in addition to the medical results or [symbol] the comment. The medical results received may also include one or more action items (discussed elsewhere in this document). At this step the Human-Centric EHR system 102 may store the medical results (including any comments and/or action items) associated with the subscribing patient in a record associated with the subscribing patient.

At step 804, the Human-Centric EHR system 102 then sends a notification to the subscribing patient's computing entity 106 associated with the subscribing patient that medical information is available. The notification that medical information is available may depend on the type of delivery method that the subscribing patient desires to obtain notifications by. For example, the subscribing patient may provide an email address to receive the notification in the form of an email; a cell-phone number to receive the indication as a text message. In other cases, where the subscribing patient's computing entity 106 runs an application associated with the Human-Centric EHR system 102, the application may provide the notification in the form of a pop-up box or window type notification or a red circle in a top corner of an icon associated with the application running on the subscribing patient's computing entity 106. Regardless of the means for providing the notification, the notification may only note that medical information is available for viewing at the Human-Centric EHR system 102 and not provide any specific details regarding the medical information. (Step 804 of process 800 is similar to step 704 of process 700).

At step 806 a request for the medical information from the subscribing patient's computing entity 106 is received and the request may include authentication information. In the cases where the notification is provided via email or text-message, the subscribing patient via the subscribing patient's computing entity 106 may login to the Human-Centric EHR system 102 to obtain the medical information. For instance, the subscribing patient may provide a user account identifier and a password. In the cases where the notification is provided in the form of a pop-up box or window type notification in the application running on the subscribing patient's computing entity 106, the subscribing patient may provide a user account identifier, a password and/or biometric authentication information. In other words, at this step the subscribing patient's computing entity 106 requests from the Human-Centric EHR system 102 access to medical information by providing authentication information. (Step 806 of process 800 is similar to step 706 of process 700).

At step 808, the authentication information included in the request for the medical information is authenticated for example by comparing the authentication information against a record associated with the subscribing patient. (Step 808 of process 800 is similar to step 706 of process 700). Then, at step 810, [symbol] e authentication information is associated with the subscribing patient, the Human-Centric EHR [symbol] m 102 then transmits a notification to the subscribing patient's computing entity 106, the notification includes the medical information. In this case, the medical information is the medical results. If the medical results contain a comment from the physician, the comment from the physician regarding the medical results may also be included in this notification.

FIG. 16C illustrates a specific and non-limiting example where the patient via the patient's computing entity 106 may then view these notifications. As illustrated, a first notification is provided that indicates that medical results have been received at the physician's office and a second notification is provided that includes the medical results and an annotation.

In some cases, step 804 may be omitted from the process 800. In these cases, a notification of the availability of medical information is not transmitted to the subscribing patient's computing entity 106 and the Human-Centric EHR system 102 waits for a request from the subscribing patient's computing entity 106.

In other cases, step 808 may be omitted from the process 800 and prior to step 804 the subscribing patient's computing entity 106 provides authentication information and if the authentication information is associated with the subscribing patient, then a notification may be sent at step 804. In other words, notifications are not sent unless the subscribing patient has authenticated his/her patient's computing entity 106 to the Human-Centric EHR system 102 in a manner that indicates he/she is currently using the device and would like notifications to be received.

In some embodiments, the medical results provided in the notification may be transmitted to the patient's computing entity 106 without any comment by the physician. In such case, the physician's EHR system 104 may automatically on creation of a medical record at the physician's EHR system 104 or on receipt of a medical record including medical results (e.g., from the EHR network 108), transmit the medical record including the medical results to the Human-Centric EHR system 102. The Human-Centric EHR system 102 may then notify the patient's computing entity 106 of the medical results in a similar fashion as discussed elsewhere in this document.

In other embodiments, the medical results transmitted to the patient's computing entity 106 may not contain the medical results received from the medical facility (e.g., lab, imaging facility, etc.) but includes only a comment or annotation from the physician. In other words, the medical results may not actually be [symbol] mitted by the physician's EHR system 104 and/or the Human-Centric EHR system 102 and in these [symbol] s, annotations of the medical results may be provided instead. In such case, the process for transmitting the comment on the medical results to the patient's computing entity 106 from the physician's EHR system 104 via the Human-Centric EHR system 102 may be similar to the process discussed elsewhere in this document in relation to transmitting the medical results to the patient's computing entity 106 from the physician's EHR system 104 via the Human-Centric EHR system 102.

At step 810, the notification of the medical results associated with the subscribing patient is transmitted to the subscribing patient's computing entity 106 may also include an action item. In other words, the notification may be flagged as requiring patient interaction or not, as well as what type of action is required. For instance, the action item may be a follow-up appointment with the physician, a prescription prescribed by the physician or any other suitable action.

In the case that the action item is an appointment with the physician, the patient may be able to accept or reject an appointment provided via the notification that provided the patient's computing entity 106 with the medical results and the action item. In some cases, the appointment in the notification is a fixed time and date and the patient has the option of accepting or rejecting the time and date. Then the Human-Centric EHR system 102 receives the acceptance or rejection of the appointment from the patient's computing entity 106. The Human-Centric EHR system 102 may then store the acceptance or rejection in the records associated with the patient at the Human-Centric EHR system 102. The Human-Centric EHR system 102 may also transmit the acceptance or rejection of the appointment to the physician's EHR system 104 and in this case, the physician's EHR system 104 may then store the acceptance or rejection in the medical records associated with the patient and/or patient scheduling software. In other cases, the action item in the notification may provide for a means for the patient to schedule an appointment. For example, the action item may provide a link to the physician's website that allows the patient via the patient's computing entity 106 to book an appointment online. By way of another example, the action item may include an email address or phone number that the patient may then use to schedule an appointment.

In the case that the action item is a prescription, the action item may be provided in various forms. For example, the patient may be able to take the patient's computing entity 106 to a pharmacy and show the prescription to the pharmacy for the fulfillment of the prescription; the action item may indicate a pharmacy where the prescription may be picked-up; the action item may indicate that the prescription has been registered with the government regulated and/or state-owned or HMO EHR system and that a [symbol] macy that has access to this system may fulfill the prescription. Upon fulfillment of the prescription, [symbol] Human-Centric EHR system 102, the EHR network 108 and/or the physician's EHR system 104 may receive acknowledgment of the fulfilled prescription. In other words, the Human-Centric EHR system 102 and/or the physician's EHR system 104 may receive notification that an action item has been completed by the patient.

In some embodiments, Human-Centric EHR system 102 receives a read-receipt from the patient's computing entity 106 after the patient via the patient's computing entity 106 views a notification (e.g., the medical results associated with the patient). The Human-Centric EHR system 102 may then store the notification of the read-receipt in the records associated with the patient at the Human-Centric EHR system 102. The Human-Centric EHR system 102 may also transmit the read-receipt to the physician's EHR system 104 and in this case, the physician's EHR system 104 may then store the read-receipt in the medical records associated with the patient. It is appreciated that such a process may be used to inform the physician if the patient has received medical results, a comment associated with the results and/or any follow up actions.

The notification may be flagged by the Human-Centric EHR system 102 with an expiry date-time stamp. If there is a request for the medical information associated with the notification after the time stamp, the request may be ignored by the Human-Centric EHR system 102 In the case that the patient's computing entity 106 is running application software associated with the Human-Centric EHR system 102 this software may remove notifications that have expired. In other cases, the medical results provided in the notification may expire after a set period of time. The notifications are discussed elsewhere in this document, such as the discussion in association with FIGS. 17A and 17B.

It is appreciated that the steps of process 700 and 800 may be considered a “pull” (or “client-pull”) based notification system instead of a “push” based notification system. To maintain confidentiality of the patient's medical results it is desirable for the medical results delivery system to be provided in a “pull” based fashion. If the patient's computing entity 106 is in the hands of a third-party, and the notification system is a “push” based notification system the confidentiality of the patient's medical results may be breached if the third-party views the notification. From a human perspective a “pull” can be made to look like a “push” but generally speaking is more desirable in terms of maintain confidentiality of the patient's medical results. For instance, when the Human-Centric EHR system 102 receives a “pull” request, prior to sending the information requested, the Human-Centric EHR system 102 verifies that the patient's computing entity 106 is verified to pull. Although in some embodiments, the patient via the patient's [symbol] puting entity 106 initiates communication (e.g., a client-pull) with the Human-Centric EHR system [symbol] to obtain various information (e.g., to get records, annotations, notifications, at the like); in alternative embodiments, push notifications (e.g., email, push notification services, server-push systems, and/or any other suitable push notification) may be used. In other words, the only “push” that may be done in such a configuration is that a push of a communication indicating that there is some information available to be accessed without providing any details on the information available. Afterwards, a “pull” may be used to retrieve the available information once authentication has been done (e.g., verifying identity with a biometric reader).

The processes discussed above (e.g., the processes 700 and 800) provide for an identity management process. This identity management process allows for patients to receive communicates from the Human-Centric EHR system 102 in a confidential way after the patient's identity has been authenticated. By providing a system where the patient is authenticated by the Human-Centric EHR system 102 prior to communications possibly containing confidential information allows for a secure manner for communicating patient health related information.

It is also appreciated that after steps 710 or 810, that after the patient view the medical records and/or medical results on the patient's computing entity 106, that the patient's computing entity 106 may be configured to delete the medical records and/or medical results both in volatile and in persistent memory.

Although in the embodiment above, the physician's EHR system 104 provides the notifications and medical results to the Human-Centric EHR system 102 and it is the Human-Centric EHR system 102 that provides the patient with the notifications and medical results, in other embodiments, the physician's EHR system 104 may notify the patient directly without the use of the Human-Centric EHR system 102. In other words, aspects of the Human-Centric EHR system 102 may be incorporated into the physician's EHR system 104. As such, in some embodiment, some or all of the functionality of the Human-Centric EHR system 102 discussed in this document may be implemented on the physician's EHR system 104.

In other embodiments, other forms of information exchange capability between the patient and the physician may be possible. In other words, the Human-Centric EHR system 102 may provide for a means for facilitating the exchange of information between the physician via the physician's EHR system 104 and the patient's computing entity 106. [symbol] re 17A and 17B illustrate more detailed examples of a first notification 1700 and second notification [symbol] ) in accordance with a specific and non-limiting example of implementation. As illustrated, the notifications 1700 and 1800 include an identifier 1702, notification data 1704, an action 1708 and a timer 1710. The identifier 1702 is used to identify the patient's computing entity 106, such that the notification is communicated from the Human-Centric EHR system 102 to patient's computing entity 106. The identifier 1702 may include an IP address, an email address, an account identifier, a telephone number and/or any other suitable identifier. The notification data 1704 may be used to communicate various information and data, such as: messages, medical results, annotations and/or any other suitable information. As illustrated, the notification data 1704 may include message data 1706, medical results data 1712, and/or an annotation data 1714. The message data 1706 may include a text-based and/or HTML based message used to provide information to the patient's computing entity 106. The medical results data 1712 may include the medical results and/or health records. The annotation data 1714 may be used to provide physician's annotation regarding the medical results. The action data 1708 may be used to convey an action item from the Human-Centric EHR system 102 to the patient's computing entity 106. Such actions may include an allow/deny action, an acknowledge action (e.g., a delivery or read receipt action), an authentication action and/or any other suitable action.

It is appreciated that notifications 1700 and 1800 are the general mechanism by which the patient associated with the patient's computing entity 106 becomes aware of any changes of his/her medical record. In fact, the first notification 1700 may also include a token (or a key), where the token may be used by the patient's computing entity 106 in making a pull to Human-Centric EHR system 102 requesting further information and resulting in the patient's computing entity 106 receiving a second communication such as a second notification 1800 including the further information, such as the medical information.

The notification 1700 is now discussed in the context of the example of the Human-Centric EHR system 102 communicating a notification of authorization to the patient's computing entity 106. This notification of authorization typically is sent first, prior to sending any further notifications, to authenticate that the person at the patient's computing entity 106 is in fact the patient. In this case, the message data 1706 may include a message that indicates to the patient that the Human-Centric EHR system 102 has further information and/or a notification for him/her. The timer 1710 may be an expiry timer, for which the notification is set to expire. In other words, the notification may no longer be accessible by the patient (e.g., the notification if removed from the notifications application window on the application software running of the patient's computing entity 106) after a set period of time. In this example, the action 1708 [symbol] ides an action item for the request for an identifier (e.g., a password, biometric information and/or [symbol] other suitable identifier) via the GUI of the patient's computing entity 106. The action item when acknowledged by the patient may send a communication from the patient's computing entity 106 to the Human-Centric EHR system 102. In this case, the communication includes the identifier. The Human-Centric EHR system 102 may process the received identifier to determine if the person at the patient's computing entity 106 is in fact the patient. This may be done by processing the received identifier against a record storing the patient's identifier. If the patient is authenticated, then further notifications may be sent from the Human-Centric EHR system 102 to the patient's computing entity 106. This may also be done by the Human-Centric EHR system 102 communicating a token (or a key) in the first notification 1700 to the patient's computing entity 106 and then the validating the token in the request from the patient's computing entity 106 such that the Human-Centric EHR system 102 verifies that the token is the token that was previously sent.

The notification 1700 is now discussed in the context of the example of the Human-Centric EHR system 102 communicating a notification to get medical results done. In this example, the message data 1706 includes a message that indicates to the patient to go to a medical facility to get medical results done. The action 1708 in this example includes an action item for the patient to acknowledge via the GUI of the patient's computing entity 106 that he/she went to the medical facility to get the testing/lab-work done. The timer 1710 may be a reminder to the patient to get the medical testing or lab work done or may be an expiry timer for which the notification is set to expire, and the patient can no longer get medical testing/lab-work done. For instance, if the patient does not go to get the testing/lab-work done after a set period of time, the Human-Centric EHR system 102 may issue another notification and/or a reminder. Also, if the patient does not go and get the testing/lab-work done after a set period of time, the notification may no longer be accessible by the patient (e.g., the notification if removed from the notifications application window on the application software running of the patient's computing entity 106).

The action item when acknowledged by the patient may send a communication from the patient's computing entity 106 to the Human-Centric EHR system 102. The Human-Centric EHR system 102 may process the receipt of an action item to setup other notifications and/or reminders. For example, after the patient acknowledges that he/she got the testing/lab-work done, then the Human-Centric EHR system 102 may process this acknowledgement against the type of testing/lab-work done to determine the typical timeframe for the medical results for this testing/lab-work. Then, if the Human-Centric EHR system 102 does not receive acknowledgement from the physician's EHR systems 104 that the medical results have [symbol] received at the physician's office, the Human-Centric EHR system 102 may issue a notification to [symbol] patient's computing entity 106 and/or physician's EHR systems 104 that the medical results were not received.

For example, if a patient gets a blood count test done, this typically only takes a couple of hours and if the patient via the patient's computing entity 106 has not received a notification that the test results are done within a 24-to-48-hour period, this may be an indication that the test results were lost, or the blood work was not done. In other words, the Human-Centric EHR system 102 may be able to determine if test/lab results are missing or not completed.

Turning now to FIG. 17C, a notification 1900, which is similar to the notifications 1700 and 1800 is shown; however, the notification 1900 may be a communication that is made from the medical EHR system 110 to the physician's EHR system 104, either directly or via the EHR network 108. The notification 1900 may be a communication that is sent from medical EHR system 110, where the notification data 1704 include medical result associated with a patient. The notification 1900 may include an urgency indicator to indicate that the medical results are time sensitive and should be reviewed by the physician urgently. The incoming notifications at the physician's EHR system 104 may be processed and when a notification includes an urgency indicator; it may be then brought to attention to physician via the physician's EHR system 104 in a more urgent manner than non-urgent notifications. For example, after the patient visits the lab and the lab technician enters in the medical results from the patient's lab tests, the notification 1900 may be communicated from the medical EHR system 110 to the physician's EHR system 104, and in this example the medical results include an outcome/observation that requires urgent review by the attending physician or the primary physician of the patient, as the results of the test indicate an outcome that could have immediate affect on the patient's health condition. The timer 1700 of the notification 1900 may include a “time-out”, so that the issuing lab technician associated with the medical EHR system 110 is notified via the action 1708 which results in the physician's EHR system 104 sending a notification to the medical EHR system 110 that the physician has not seen the medical results within a specific amount of time.

Referring back to FIG. 17A, the notification 1700 is now discussed in the context of the example of the Human-Centric EHR system 102 communicating a first notification of the availability of medical information to the patient's computing entity 106. In this case, the message data 1706 includes a message that indicates to the patient that his/her medical results have arrived at the physician's office. The message data 1706 may also include an indication that a notification will be provided later. The timer 1710 may [symbol] sed to as reminder so the patient can follow-up with his/her physician if he/she does not receive a [symbol] ication with the medical results in a set period of time. The timer 1710 may also be an expiry timer for which the notification is set to expire and the patient can no longer view this notification.

The notification 1800 is now discussed in the context of the example of the Human-Centric EHR system 102 communicating a second notification of the availability of medical information to the patient's computing entity 106. In this case, the medical results data 1712 conveys the medical results and the annotation data 1714 includes a physician's comment or annotation regarding the medical results. The action 1708 may be used for various actions items such as acknowledgment of receipt of the notification, to schedule an appointment and/or to provide a prescription. The timer 1710 may be an expiry timer for which the notification is set to expire and the patient can no longer view this notification.

The physician should report the medical results to the patient in a specific time-frame and the Human-Centric EHR system 102 may be able to record or track the time-frame that it takes each physician to report the medical results. In other words, the Human-Centric EHR system 102 may monitor and record the time frames of the various timers and actions to produce reports that indicate the average medical facility processing time, the physician's time to review the medical results and/or any other suitable report. This data may be useful in determine which medical facilities process results faster than others and/or to determine which physicians review medical results faster than others.

The message data 1706, medical results data 1712 and the annotation data 1714 may specific that the notification or the information provided in the notification is “for your eyes only” (or a similar word having the same connotation), as it is appreciated that the patient may be receiving medical results and/or information that is sensitive in nature.

Notification 1700 and/or notification 1800 may also be used in various other embodiments to obtain permission for an action from the patient via the patient's computing entity 106. For example, to receive authorization from the patient to provide health records to a third-party, to receive authorization from the patient to have his/her health records used in a study, and/or to receive authorization from the patient any other suitable action.

In some specific examples of implementation, the notifications may include a must acknowledge field, an acknowledged by field and an expiry field. The must acknowledge field requires a receipt from the patient's computing entity 106. The acknowledged by field identifies who acknowledged and may include [symbol] lentifier of the party that acknowledged the receipt of the notification. The expiry field is used to set [symbol] xpiry timer.

This notification system may be useful when multiple test/lab results are being done, as it may allow for a means to monitor multiple test/lab results.

It is appreciated that timers may be used in various ways in the various embodiments described herein. In general, any communication or notification (e.g., the notifications 1700, 1800 and 1900) may include a timer 1710. Although the timer 1710 is discussed herein in various examples, more generally a timer may be used to notify patients, physician's, practitioners, and/or any other suitable person of an action that is required by him/her (e.g., view the notification, view the information contained in the notification, follow the action in the notification), any missed actions (e.g., a reminder), may be used for notifications to become expired and no long visible by the user.

A delegate, such as a patient's legal guardian and/or any other suitable person, having a delegate's computing entity (similar to the patient's computing entity 106) may be setup for receiving notifications with the Human-Centric EHR system 102. The delegate's computing entity may receive notifications instead of the patient's computing entity 106 or the patient's computing entity 106 and the delegate's computing entity may both receive notifications. This may be useful where the patient is disabled or has limited mobility and needs regular assistance from another person.

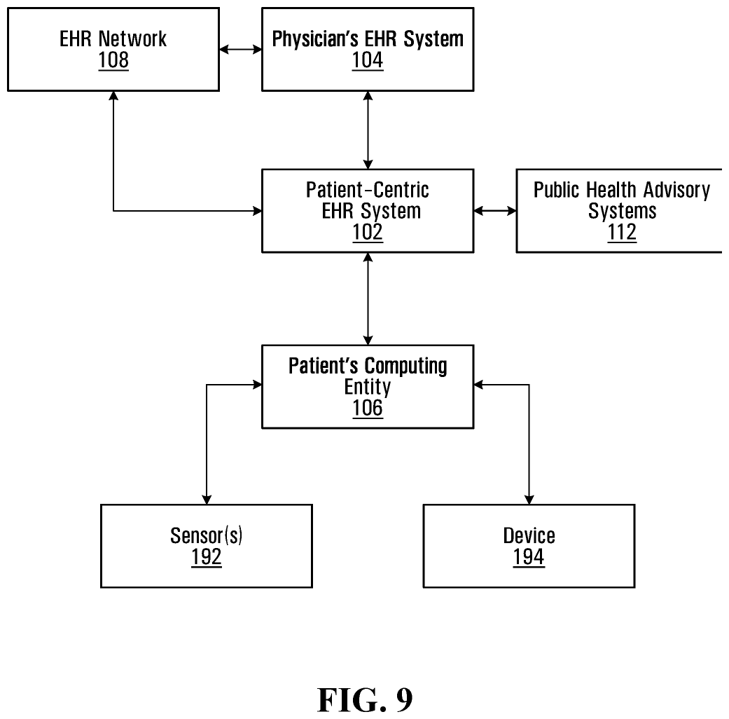

FIG. 9 illustrates a block diagram of an example of the Human-Centric EHR system 102, where the Human-Centric EHR system 102 is able to receive and obtain auxiliary medical related information. The Human-Centric EHR system 102 may be connected to the patient computing entity 106, to the physician's EHR systems 104, to the EHR network 108 and/or to one or more systems that provide public health advisories 112. As illustrated, the patient computing entity 106 is connected to one or more sensors 192. In some cases, the patient computing entity 106 is also connected to an external device 194. The various connections between the Human-Centric EHR system 102, the patient's computing entity 106, the physician's EHR system 104, the medical EHR system 110 (not illustrated in FIG. 9), the EHR network 108, the public health advisor systems 112, one or more sensors 192 and/or the external device 194 may include one or more connections over one or more data networks. The various connections between the Human-Centric EHR system 102, the patient's computing entity 106, the physician's EHR system 104, the medical EHR system 110, the EHR network 108, the public health advisor systems 112, one or more sensors 192 and/or the external device 194 may allow for the communication (e.g., transmission and/or [symbol] ption) of various information and/or data between the various systems and/or devices. In some [symbol] odiments the connection between the one or more sensors 192 and/or the external device 194 with the patient's computing entity 106 may be a wired (e.g., USB, USB-C, Ethernet, Firewire™ or any other suitable connection) or wireless connection (e.g., Bluetooth™, ZigBee™, Wi-Fi™, NFC or any other suitable connection).

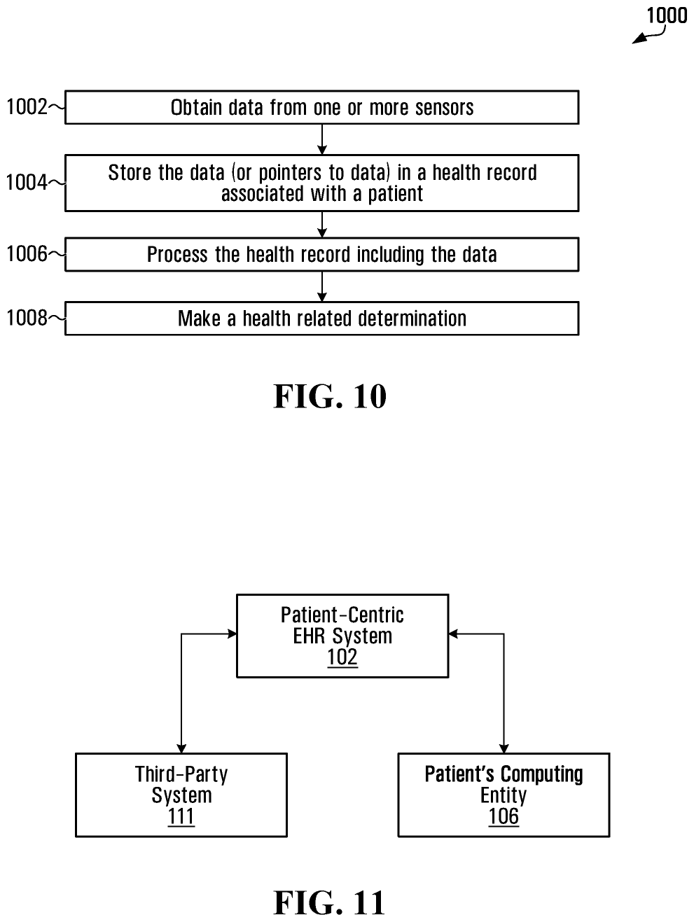

FIG. 10 illustrates a flowchart of a process 1000 which may be implemented at the Human-Centric EHR system 102 for making a health-related determination at least in part by processing data obtained from one or more sensors 192 in combination with health records associated with a patient. At step 1002, data from one or more sensors 192 is obtained. This may include monitoring data obtained from the one or more sensors 192 where these sensors 192 measure a physical condition of a patient and where the monitoring of the data from the one or more sensors is done over a plurality of time points. These one or more sensors 1002 may be of various types, including: blood pressure monitors, blood glucose monitors, heart rate monitors, accelerometers, GPSs, barometer, altimeter, ambient light level detector, spectrum analyzer, any other sensors located in a portable computing device (e.g., smart-watch, portable phone, tablet, PDA, or any other suitable portable computing device), and/or any other suitable sensor or computing device. At this step, data from public health advisory systems 112 may also be obtained, which may include various types of information such as smog warnings, heatwaves and/or any other suitable information.

At step 1004 the data from the one or more sensors 192 is stored in association with a health record associated with a patient. It is appreciated that the data from the one or more sensors may be collected over multiple time points to form longitudinal data. This longitudinal data associated with the patient may track a physical condition of a patient over a period of time. This longitudinal data may also include environmental factors such as atmospheric pressure, UV exposure and/or any other suitable environmental factors. This longitudinal data associated with the patient may be stored in one or more health records on the Human-Centric EHR system 102. The Human-Centric EHR system 102 may also contain a continuum of heath records associated with the patient obtained from various EHR systems and/or the EHR network 108. In other words, the medical records may have been obtained from a plurality of EHR systems and/or other sources. This longitudinal data in combination with the continuum of heath records associated with the patient may allow for processing of this information to make a health-related determination for the patient, such as may be done at step 1006. In general, at step 1006, health records including the data from the sensors 192 are processed. This may include processing the data from the sensors 192 against the medical records associated with the patient or processing the health [symbol] rds associated with the patient where these health records are augmented by the data from the sensors [symbol] This processing may also include tracking trends in the longitudinal data. Then at step 1008, at least in part by processing the health record including the data from the sensors a health-related determination is made. The health-related determination may be made when a threshold level is detected. The detection of the threshold level being reached may be determined by computer processing. The detection of the threshold level being reached may also be verified by a third-party agent (e.g., a person). The health-related determination may include sending a notification to the patient via the patient's computing entity 106. The health-related determination may include a notification being sent to the patient's primary physician, assuming that the patient has named a primary physician. In the case that a third-party agent verifies the threshold level being reached the agent may initiate the communication of the notification to the patient and/or to the physician to indicate the unsafe condition. In the case that the notification from a heath related determination is sent to the physician's EHR system 104, the physician may then be notified via the physician's EHR system 104 to review the notification and then make a determination to notify the patient, which may then be sent from the physician's EHR system 104 to the patient's computing entity 106 via the Human-Centric EHR system 102.

The process 1000 should likely become more readily apparent in view of the following examples:
Blood pressure monitor example: In this example the sensor 192 is a sensor that monitors blood pressure. The sensor 192 is in communication with the patient's computing entity 106 and the patient's computing entity 106 communicates blood pressure data to the Human-Centric EHR system 102, such that the Human-Centric EHR system 102 may monitor this data. The patient's health records indicate that the patient has high blood pressure and the Human-Centric EHR system 102 based on processing the patient's health records determines that if the blood pressure of the patient rise above a certain threshold that the patient via the patient's computing entity 106 and/or the patient's physician via the physician's EHR system 102 should be notified as this may be an indication of a possible medical issue.
Blood glucose monitor example: In this example the sensor 192 is a sensor that monitors blood glucose levels. Various blood glucose monitors are currently available in the market (e.g., MyGlucoHealth Blood Glucose Meter with Bluetooth Technology). The sensor 192 is in communication with the patient's computing entity 106 and the patient's computing entity 106 communicates blood glucose data to the Human-Centric EHR system 102, such that the Human-Centric EHR system 102 may monitor this data. The patient's health records indicate that the patient has diabetes and the Human-Centric EHR system 102 based on processing the patient's health records determines that if the blood glucose level of the patient raises above a certain threshold that the patient via the patient's computing entity 106 and/or the patient's physician via the physician's EHR system 102 should be notified as this may be an indication of a possible medical issue.
Heart rate monitors & exercise equipment example: In this example the sensor 192 is a sensor that monitors heart rate. Various heart rate monitors are currently available (e.g., OMsignal Biometric Smartwear). The sensor 192 is in communication with the patient's computing entity 106 and the patient's computing entity 106 communicates heart rate data to the Human-Centric EHR system 102, such that the Human-Centric EHR system 102 may monitor this data. In this example, the device 194 generally speaking is a piece of exercise equipment and in particular is a treadmill. Prior to starting to run on the treadmill the patient identifies the sensor 192 and device 194 to the Human-Centric EHR system 102. The Human-Centric EHR system 102 based on the patient's health records make a determination that the patient should run at a specific pace and may control the speed of the treadmill. As the patient starts to run on the treadmill, his/her heart rate increase and in this example the heart rate is too high and the Human-Centric EHR system 102 makes the determination by processing the heart rate data in combination with the patient's health records that the speed of the treadmill should be reduced. In other words, in this example, the Human-Centric EHR system 102 may make a recommendation of a treadmill speed based on the patient's health records and may adjust the speed in accordance with changes in heart rate.
Chronic obstructive pulmonary disease, GPS & public health advisory example: In this example the sensor 192 is a GPS sensor which may be part of the patient's computing entity 106. The sensor 192 is in communication with the patient's computing entity 106 and the patient's computing entity 106 communicates GPS location data to the Human-Centric EHR system 102, such that the Human-Centric EHR system 102 may monitor this data. The patient's health records indicate that the patient has chronic obstructive pulmonary disease (COPD) and the Human-Centric EHR system 102 based on processing the patient's health record determines that the Human-Centric system should obtain public health advisories for the public health advisory system 112. Then the Human-Centric EHR system 102 monitors the location of the patient and processes the public health advisories to determine if the patient is in a high smog area and if such is the case, notifies the patient via the patient's computing entity 106 and/or the patient's physician via the physician's EHR system 102.
The above examples are intended to be non-limiting and various other examples of using the sensors 192 in combination with the patient's health records obtained for multiple EHR system and/or sources to make health related determinations are possible.

The Human-Centric EHR system may be provided with logic that can process the information received from the various biometric sensors and generate calibration data sent to wearable or stationary devices intended to provide guidance to the patient regarding physical activity. For example, the program logic can interpret the physiological information received such as to set a safe level of activity that the user should not exceed in order to avoid a health risk, such as a heart attack, stroke or other. To be more specific, the program logic will process the physiological information such as the heart rate, blood pressure and blood glucose, among others to derive the safe level for physical activities. This processing can be performed by mapping the physiological data with predetermined degrees of “safe level” exercise intensity, such as exercise duration, speed of running, maximal heart level rate during the exercise, etc. In a possible refinement, the program logic can use other information from the patient record, which is co-related to the physiological information to further refine the “safe level” for the user based on the user's medical history.

The calibration data can be sent to the physiological sensor that generated the physiological data when that sensor is used to provide physical activity guidance to the user, such as the degree of daily physical activity desired. The calibration data will thus determine the amount of activity (calories burned) and the intensity.

In one specific example, the calibration data can be sent to an exercise machine, such as a treadmill, to set the operational parameters of the treadmill for an exercise session for the user, such as the maximal running speed, duration, elevation, etc. A similar approach can be considered for another kind of exercise equipment such a muscle building machine.

In other embodiments instead of the Human-Centric EHR system 102 processing the health record associated with the patient and the auxiliary medical related information (e.g., data from sensors 192, device 194, public health advisory system 112, and/or any other suitable information) the Human-Centric EHR system 102 may transmit the health record associated with the patient and the auxiliary medical related information to a third-party system (e.g., an agent) which may then process the health record and auxiliary medical related information to make the health related determination in a similar fashion as that [symbol] ssed above. In such case, after the third-party system makes the health-related determination, the [symbol] party system may transmit the health-related determination to Human-Centric EHR system 102 which may forward the health-related determination to the physician's EHR system 104 and/or the patient's computing entity 106.

It is appreciated that the health-related determination may be made by a computer-based decision support agent that contains logic that can make a health-related determination. More specifically, the decision support agent may process the health record associated with the patient and the auxiliary medical related information in relation to rules, fact, fact and rules, models, algorithms, search, procedural code, analytic techniques, inference engine and/or any other suitable technique or logic to make the health-related determination that may add value to the patient. In some cases, the decision support agent is an artificial intelligence and/or expert system that may emulate the decision-making ability of a human expert.

Although in the examples above the health record associated with the patient and the auxiliary medical related information was processed to make a health-related determination, in other embodiments the health record associated with the patient may be processed on its own (without the auxiliary medical related information) to make a health-related determination in a manner similar to that discussed above.

Third-Party Health Record Access Mechanism

FIG. 11 illustrates a block diagram of an example of the Human-Centric EHR system 102, where the Human-Centric EHR system 102 is able to provide a health record associated with a patient to a third-party system 111 upon authorization of the patient at the patient computing entity 106 (e.g., a computing entity associated with the patient) in accordance with an embodiment of the invention. The third-party system 111 may be an EHR system similar to the physician's EHR system 104, discussed elsewhere in this document. As illustrated, the Human-Centric EHR system 102 is connected to the patient's computing entity 106 and to a third-party systems 111 (e.g., an system associated with a third-party). The various connections between the Human-Centric EHR system 102, the patient's computing entity 106 and/or the third-party system 111 may include one or more connections over one or more data networks. The various connections between the Human-Centric EHR system 102, the patient's computing entity 106 and/or the third-party system 111 may allow for the communication (e.g., transmission and/or reception) of various information and/or data between the various systems and/or devices. The various information and/or data exchanged may be a “notification” or a “health record” of the type discussed elsewhere in this document. The third-party system 111 may be viewed as an agent. For example, it may [symbol] system that examines records associated with the patient. The agent may be able to examine or [symbol] ess the records and make various determinations and/or annotations to one or more of the records. It is appreciated that the agent may be a person at the third-party system 111 that reviews the health records and then runs additional tests or processes on the health records to make the various determinations and/or annotations to one or more of the records. In some embodiments, the agent may be a computer-based agent comprising the computer-based decision support agent (discussed elsewhere in this document) that may process the health records and make various determinations and/or annotations to one or more of the records.

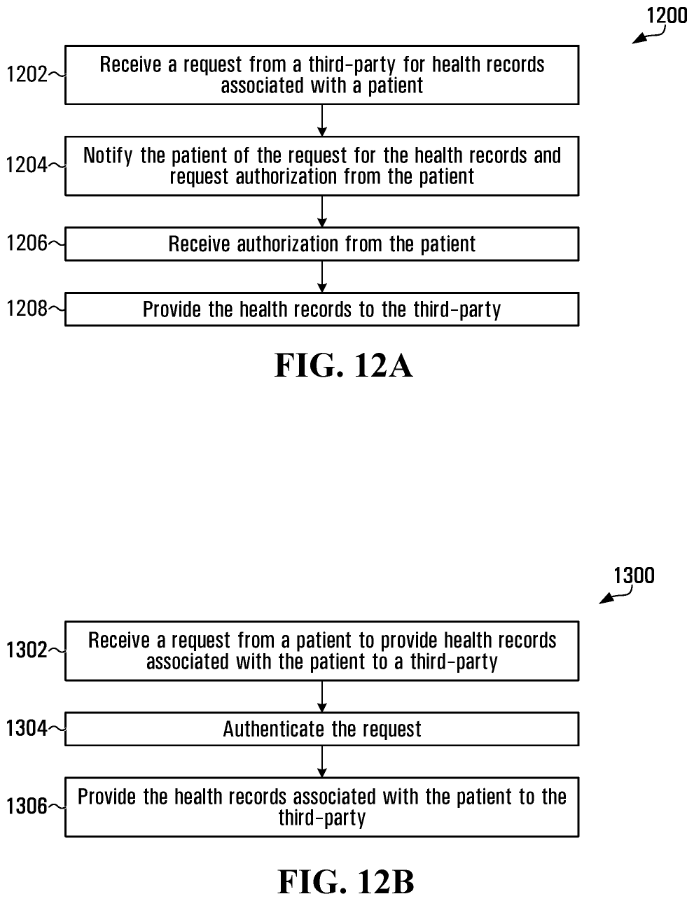

FIG. 12A illustrates a flowchart of a first process 1200 for providing one or more health records associated with a patient to the third-party system 111. At step 1202 a request from a third-party system 111 for one or more health records associated with a patient is received at the Human-Centric EHR system 102. The request may be initiated from the third-party system 111. At step 1204, the patient associated with the heath record which was requested is notified of the request for the one or more health records via the patient's computing entity 106. The Human-Centric EHR system may also request that the patient via the patient's computing entity 106 provide authorization to provide the third-party system 111 with the one or more health records. In this example, the patient's computing entity 106 is provided with the notification (similar to the notifications discussed elsewhere in this document) and the request for authorization (e.g., an action item). At step 1206, the Human-Centric EHR system 102 receives the authorization from the patient via the patient's computing entity 106. Then, at step 1208, the one or more health records associated with the patient are provided by the Human-Centric EHR system 102 to the third-party via the third-party system 111. If the authorization of the patient at step 1206 fails or the patient declines to authorize the Human-Centric EHR system 102 to provide the records to the third-party system 111, the process 1200 would stop (i.e., no records would be provided at step 1208).

Steps 1204 may be similar to step 704 discussed above, in that a notification is sent to the patient via the patient's computing entity 106. The notification may not provide any indication of the contents of the notification other than indicating that some action/information is available. The patient may then authenticate himself/herself at step 1206 (e.g., similar to step 706) and the Human-Centric EHR system 102 would then authenticate the request (e.g., similar to step 708). Once the patient's computing entity 106 is authenticated to the Human-Centric EHR system 102 a common proxy identifier may be used to communicate data between the patient's computing entity 106 and the Human-Centric EHR system 102. Then the patient's computing entity 106 may send the request to the Human-Centric EHR system 102 to [symbol] orize the patient's computing entity 106 to provide one or more records associated with the patient to [symbol] third-party system 111.

FIG. 12B illustrates a flowchart of a second process 1300 for providing one or more health records associated with a patient to a third-party. The process 1300 is similar to process 1200; however, the process 1300 may be initiated by the patient while the process 1200 may be initiated by the third-party. At step 1302 a request from a patient via the patient's computing entity 106 is received at the Human-Centric EHR system 102. The request is for the Human-Centric EHR system 102 to provide a health record associated with the patient to a third party. This request from the patient may occur after the patient has authenticated himself/herself to Human-Centric EHR system 102, as discussed above. In this example, the third-party is associated with the third-party system 111. At step 1304, the request is authenticated and then, at step 1306, the one or more health records associated with the patient are provided to the third-party system 111. Step 1304 and 1306 are similar to steps 1206 and 1208 discussed above. If the authorization of the patient at step 1304 fails, the process 1300 would stop (i.e., no records would be provided at step 1306).

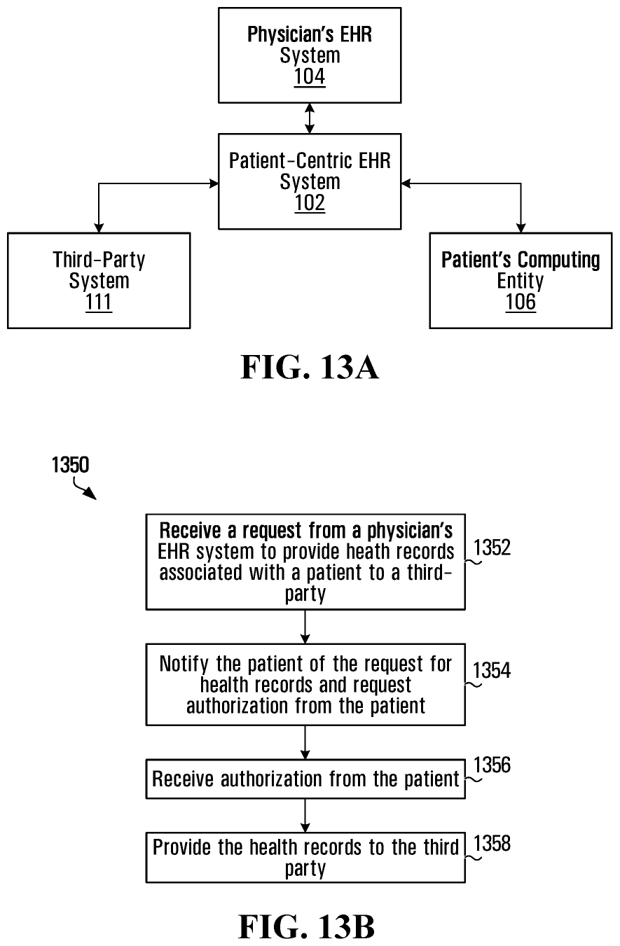

FIG. 13B illustrates a flowchart of a third process 1350 for providing one or more health records associated with the patient to the third-party system 111. The process 1350 is similar to that of the process 1200; however, the request to provide the one or more health records associated with the patient to the third-party system 111 is initiated by the physician at the physician's EHR system 104. For instance, the process 1350 may be implemented on the Human-Centric EHR system 102 shown in FIG. 13A, which illustrates a block diagram of an example of the Human-Centric EHR system 102 shown in FIG. 11, but where the physician's EHR system 104 is in communication with the Human-Centric EHR system 102 for initiating the request to provide the one or more health records associated with the patient to the third-party system 111. Turning back to the process 1350, at step 1352 a request from the physician's EHR system 104 to provide one or more health records associated with a patient to the third-party system 111 is received at the Human-Centric EHR system 102. At step 1354, the patient associated with the heath record which was requested is notified of the request for the one or more health records via the patient's computing entity 106. The Human-Centric EHR system may also request that the patient via patient's computing entity 106 provide authorization to provide the third-party system 111 with the one or more health records. Step 1354 may be implemented in a similar way as step 1204 discussed above. At step 1356, the Human-Centric EHR system 102 receives the authorization from the patient via the patient's computing entity 106. Step 1356 may be implemented in a similar way as step 1206 discussed above. Then, at step 1358, the one or more health records associated with the patient are provided by the [symbol] ian-Centric EHR system 102 to the third-party system 111. Step 1358 may be implemented in a [symbol] lar way as step 1208 discussed above. If the authorization of the patient at step 1356 fails or the patient declines to authorize the Human-Centric EHR system 102 to provide the records to the third-party system 111, the process 1350 would stop (i.e., no records would be provided at step 1358).

Also, although not illustrated in FIGS. 12A, 12B and 13B, the patient may at a later time withdraw authorization to the third-party to have access to the health records associated with the patient. For instance, the patient via the patient's computing entity 106 may authenticate himself/herself to the Human-Centric EHR system 102 (as discussed elsewhere in this document), afterwards the patient via the patient's computing entity 106 may send a request to the Human-Centric EHR system 102 to remove or revoke access to a third-party to one or more of the health records that the third-party previously had access thereto. In other words, the patient with the patient's computing entity 108 may connect to the Human-Centric EHR system 102 to control who has visibility access to his/her entire health record or to specific records.

The processes 1200,1300 and 1350 are illustrated further by way of a specific and non-limiting example. In this example, the Human-Centric EHR system 102 has an agent company providing ‘on-call’ physicians that can read one or more health records and provide interpretation/information. The patient may first subscribe to the agent and then the patient may request a second opinion on one or more health records (e.g., such as at step 1302). For instance, the patient may subscribe directly to the third-party system 111 or may subscribe to the third-party system 111 via the Human-Centric EHR system 102. The third-party system 111 is notified via the Human-Centric EHR system 102, assigns a third-party physician, and issues a notification to authorize (e.g., patient-physician pairing) via the Human-Centric EHR system 102. The patient receives the notification (e.g., such as at step 1204). The notification includes an action which requires confirmation, and the patient confirms (e.g., such as step 1206). The third-party physician via the third-party system 111 receives a notification with the one or more health records attached (e.g., such as at step 1208 or 1306). For example, the third-party physician may connect to the third-party system 111 and/or to the Human-Centric EHR system 102 via the third-party system 111 to gain access to the patient's health records. Then the third-party physician may add a determination and/or an annotation to the one or more health records. When a determination and/or annotation is added to the one or more health record associated with the patient on the Human-Centric EHR system 102, the patient at the patient's computing entity 106 may receive notification from the Human-Centric EHR system 102, with the attached third-party physician's determination and/or annotation. The primary [symbol] ician, of the patient, via the physician's EHR system 104 may also be notified when a determination [symbol] or an annotation is added to a health record associated with the patient.

Similarly, the example above instead of the health records associated with the patient being provided to a physician for review, the health records may be provided to a third-party system 111 for processing the patient's records to make a health-related determination. For example, the determination may be to identify some genetic predisposition that the patient has and/or any other suitable medical condition. In other words, the patient may provide at least some of his/her health records to the third-party system 111 for processing or continuous monitoring, such that the system may make a health-related determination and then provide such heath related determination to the patient. For instance, the health-related determination may be made by the computer-based decision support agent that contains logic that can make a health-related determination, discussed elsewhere in this document. The computer-based decision support agent may also be used for continuous monitoring of the patient's health record and when a possible medical condition is detected, cause a notification to be sent to the patient and/or the patient's physician.

The processes 1200,1300 and 1350 are illustrated further by way of a specific and non-limiting example. In this example, the patient is visiting family in Australia and the patient has a care need. The patient finds a third-party physician and would like to provide this third-party physician with his health records. The patient via the patient's computing entity 106 grants the third-party physician/the third-party physician's clinic access rights to his health records on the Human-Centric EHR system 102. The patient via the patient's computing entity 106 may connect or log into the Human-Centric EHR system 102 and select the third-party that the patient desires to share his health records with (e.g., such as at step 1302), as is shown in example illustrated in FIG. 16F. The patient may be able to set a start and end date (and time) or an expiry date (and time) for which the third-party physician can have access to the patient's health records. The patient may also be able to select (e.g., flag) which health records that the patient would like to have provided to the third-party physician. After the Human-Centric EHR system 102 receives this request from the patient to grant the third-party physician access rights to the selected health records, the Human-Centric EHR system 102 notifies the third-party physician via the third-party system 111. Although the third-party system 111 in some embodiments is an EHR system, in other cases this system 111 may not have EHR capabilities but may be any portable or non-portable computing device and/or entity, such as a cellphone, a tablet, a smart watch, a laptop/notebook computer, a computer or any other suitable computing device. In this example the third-party system 111 associated with the third-party physician receives a notification from the Human-Centric EHR system 102. This notification may [symbol] the form of a secure email granting the third-party physician access to the Human-Centric EHR [symbol] m 102 to access the selected health records associated with the patient. In this example, the third-party physician's access to the Human-Centric EHR system 102 would be limited to the health records associated with that patient only and may be limited to health records that the patient chooses to share with the third-party physician. The third-party physician logs-in with his third-party computing device 111 and has access to whatever parts the patient has selected as accessible by the third-party physician. As such, the patient is able to provide access right to all of his health records or to a portion of his health records. In this example, before the third-party physician can actually see the health records associated with the patient, the patient's computing entity 106 receives a notification with an allow or deny confirmation option (e.g., such as at step 1204). This option allows for the patient to deny access, for example if the patient is not in the presence of the third-party physician. If the patient allows access (e.g., such as at step 1206), for example, when the patient has the benefit of visually verifying the identity of the third-party physician, the third-party computing device 111 is then provided with the health records associate with the patient that the patient has selected to be accessible by the third-party physician. In this example, the patient may use biometric authentication (or any other suitable means of authentication) on the patient's computing entity 106 in order to confirm that the actual patient or their legal proxy, as opposed to the person who has the device in hand, is authorizing the access. The combination of a notification mechanism and a robust authentication on the patient's computing entity side, especially when that computing entity is a mobile, is a security element that is difficult to bypass. It is not possible for a non-authorized party that may gain access to the mobile of the user to enable through the notification access to the medical information, unless the authentication mechanism of the mobile unlocks the mobile first and enables the notification to receive user input. For example, if the mobile is stolen, the mobile will receive the notification that could appear on the screen, but notification would not respond to inputs, hence it would not be possible send an authorization at step 1206 to provide access to the health records.

In yet another example, the sharing of data to a third party may be performed according to a variant of the process described in FIG. 12B. Assume for the purpose of this example that the user accesses the user data, which can be medical data or other confidential data such as financial data, legal data or other. In the case of financial data, that data can consist of several pages of financial information such as different banking accounts. The access by the user to the financial information is performed according to anyone of the methods discussed throughout this document. Once the user has accessed the account and reached the page of financial information that he or she wants to share with a third party, the user can activate a control on a GUI on the user computing entity 106 to initiate the sharing process. [symbol] specific example of implementation, the sharing process is configured such that the presence of the [symbol] on the information will be shared with is necessary. That presence can be physical presence, namely [symbol] the user and the third party are in close proximity with each other or virtual presence, where both the user and the third party are on a video conference where each person can see the other.

The third party is directed to access a network location defined by a certain address, via a web browser. With reference to FIG. 11, assume the third party resides at the third-party system 111 and accesses the network location from the third-party system 111. Specifically, the third party would type in the browser address window the specified network address that will be received at the system 102. In response to this request, the system 102 will generate a random code that is sent to the web browser opened at the third-party system 111. In a specific example of implementation, that random code can appear on the browser display window as a QR code, as a bar-code or as any other machine-readable code. Once this code is generated and displayed on the browser window, it is kept in the memory of the system 102 for a certain period of time, which can be minutes or hours. Passed this time period, the code is de-activated and will no longer produce any information transfer.

Assuming the code is within its validity period, the user's computing entity, which is assumed to be a mobile with a camera, scans the QR code. The scanned QR code is then transmitted to the system 102 and then matched with the original code that was generated on the web browser page.

The system 102 monitors at all times the system state of all the interactions with the various computing entities 106 and is aware of the actual record or page of the user account that was displayed when the sharing command was initiated. That page or record is then sent to the web browser on which the QR code was initially displayed.

Optionally, before actually displaying the information on the web browser, the system 102 may send a notification, as described elsewhere in the description, which includes a response mechanism allowing the user to confirm that the information should be made available to a third party. In response to an acknowledgement of the user via the response mechanism the information is shared.

The QR code generated on the web browser can also be read in the context of a video conference, where the third party with the browser showing the QR code is displayed on the video screen and then the user scans this code on its video screen to complete the process. [symbol] itional examples of functionalities that further facilitate the management of the access of health [symbol] rds to a third party are discussed later in this disclosure. Those examples introduce additional automation and selection granularity to provide the patient with a fine control over the access of the health records to a third party.

Care Support Group

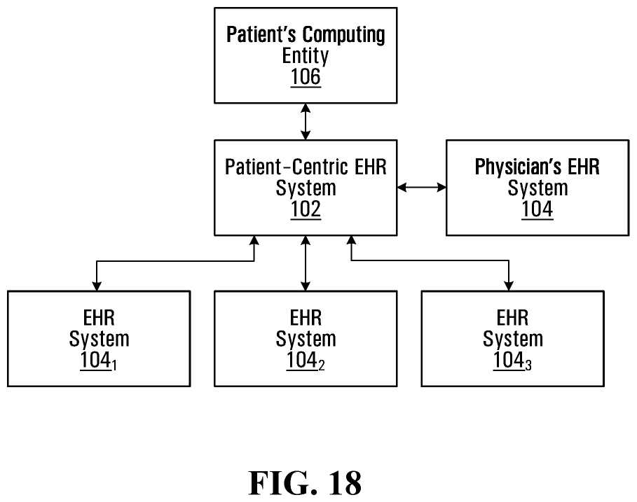

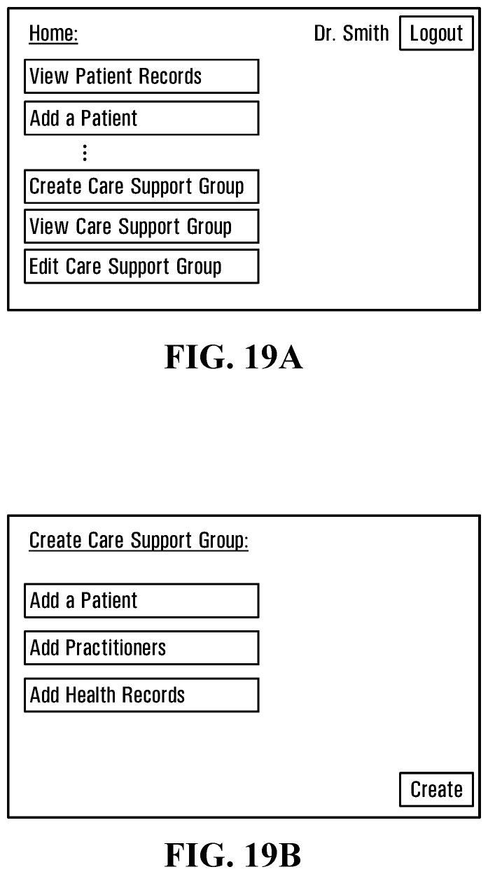

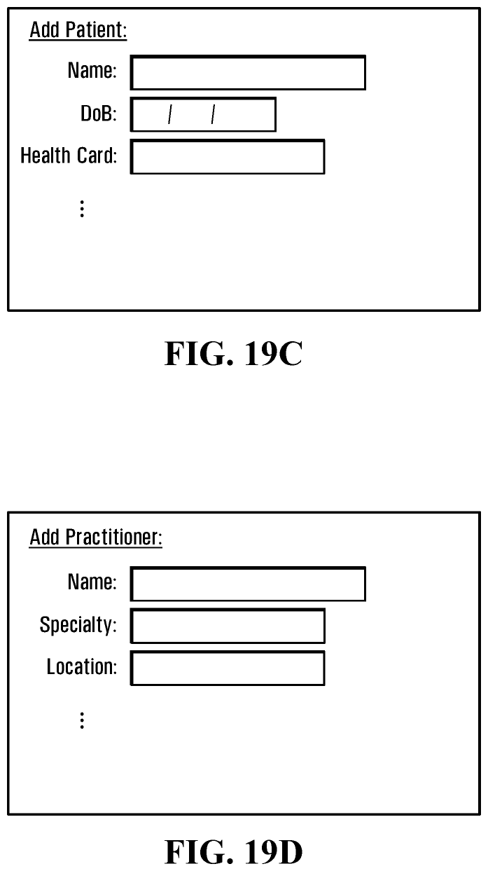

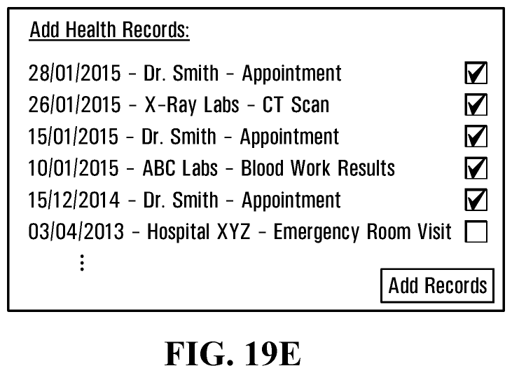

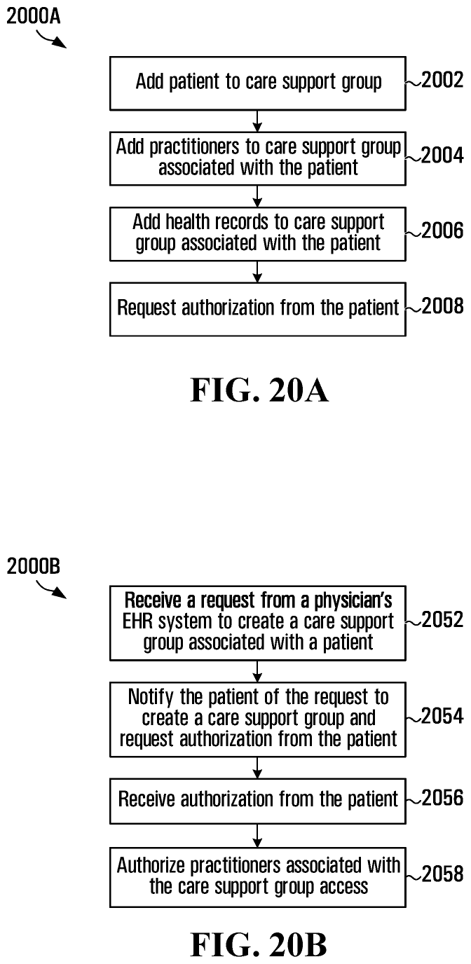

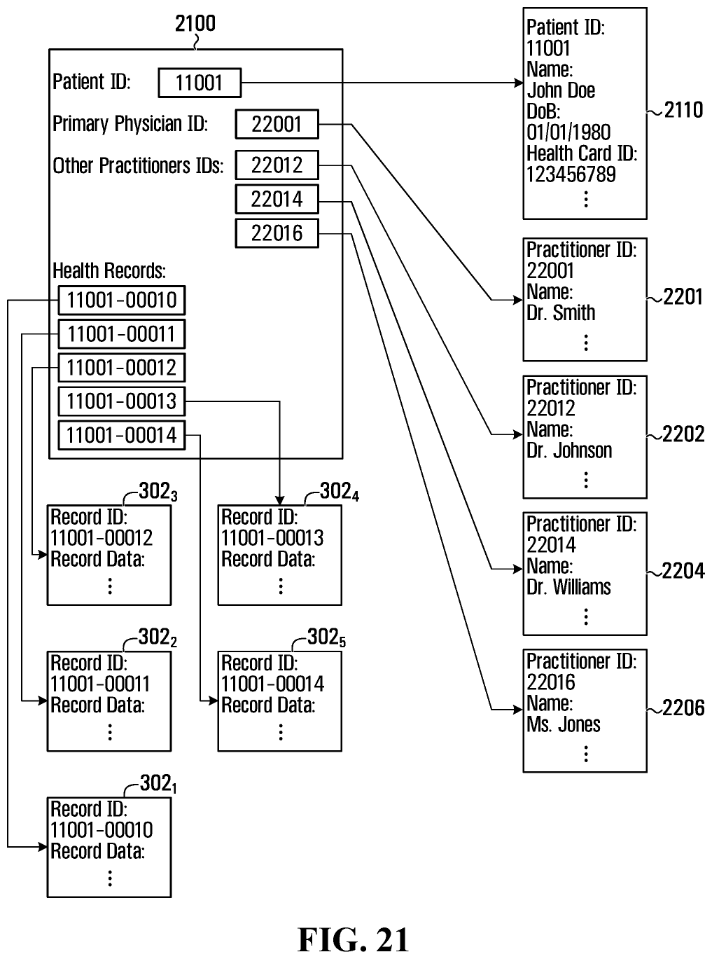

A care support group system will now be described with reference to FIGS. 18, 19A to 19E, 20A, 20B and 21. In general, the care support group may be considered a subset of the health records associated with a patient that is accessible and updatable by a select group of practitioners. FIG. 18 illustrates a block diagram of an example of the Human-Centric EHR system 102 connected to a primary physician's EHR system 104, a plurality of EHR systems 104₁ 104₂ 104₃ associated with other practitioners and to the patient's computing entity 106 in accordance with an embodiment of the invention. In this embodiment, the primary physician via the physician's EHR system 104 is able to create a care support group around a patient and a situation associated with the patient via the Human-Centric EHR system 102. FIGS. 19A to 19E illustrate specific and non-limiting examples of information that may be displayed on a graphical user interface (GUI) of a physician's computing entity in the process of creating the care support group in accordance with specific and non-limiting examples of implementation. FIGS. 20A and 20B illustrate flowcharts of processes 2000A and 2000B for creating the care support group in accordance with a specific non-limiting example of implementation. FIG. 21 illustrates an example of a computer readable data structure 2100 for the care support group that may be stored on the Human-Centric EHR system 102 in accordance with a specific non-limiting example of implementation.

It is appreciated that the primary physician associated with the patient may want to share various health records and information associated with a patient in order to provide care to the patient. For instance, the primary physician may want to create a therapeutic team for the collaboration in the treatment of a specific condition (e.g., condition, disorder, infection, disability, injury, situation and/or any other suitable condition or situation) associated with the patient. For instance, the medical condition may have specific symptoms and signs that the physician has identified which may be determined based on testing results (e.g., blood tests, urine tests, x-ray images, and/or any other suitable test). As such, the physician may want to further investigate and/or treat the medical condition. Regardless of the specific condition or situation for creating the care support group around, the physician may interact with the Human-Centric EHR system 102 to create the care support group further described below. [symbol] iecific and non-limiting example of the care support group (or circle of care) will now be described. [symbol] is example, the patient John Doe was diagnosed as having cancer by his primary physician Dr. Smith. As John Doe chooses to undergo various treatments based on the advice of his primary physician, Dr. Smith chooses to interact with the Human-Centric EHR system 102 via his physician's EHR system 104 to create a care support group during the treatment of John Doe's cancer. As such, the primary physician Dr. Smith connects to the Human-Centric EHR system 102 which may include him logging in with the physician's EHR system 104 to the Human-Centric EHR system 102 by providing his account identifier and credential. After logging in to the Human-Centric EHR system 102, the GUI of the display device associated with the primary physician's EHR system 104 may be conditioned to display the information shown in FIG. 19A. As shown, FIG. 19A provides Dr. Smith with various interface controls to allow him to select and view health records associated with a patient, add a patient to the Human-Centric EHR system 102, create a care support group, view a care support group and edit a care support group. Other interface controls may be available in other embodiments.

The primary physician Dr. Smith may select the interface control to create a new care support group and then may be provided with the information shown in FIG. 19B, which includes interface controls for adding a patient, adding practitioners. Other interface controls may be included in other embodiments. Afterwards, the primary physician Dr. Smith may create the care support group according to the process 2000A in FIG. 20A. For instance, the primary physician Dr. Smith may create the care support group by adding a patient to the care support group (step 2002), adding one or more practitioners to the care support group associated with the patient (step 2004) and adding one or more health records to the care support group associated with the patient (step 2006). The order of steps 2002, 2004, 2006 may vary in various embodiments. The Human-Centric EHR system 102 receives the requests from the physician's EHR system 104 to create and add aforementioned aspects to the care support group, processes the request and creates the care support group accordingly which may include creating the data structure 2100. The data structure 2100 is for illustration purposes only and may vary in various implementations of the care support group.

To further illustrate step 2002, the primary physician Dr. Smith may select the interface control to add a patient to the care support group and then may be provided with the information shown in FIG. 19C. FIG. 19C illustrates an example of information shown on the GUI of the primary physician's EHR system 104 for adding a patient to the care support group. As shown, the primary physician Dr. Smith may search for a patient by his/her name, date of birth, health card number and/or any other suitable identifier (e.g., address, phone number, to name a few). Then the primary physician Dr. Smith may add [symbol] patient, which in this example is John Doe, to the care support group. The Human-Centric EHR [symbol] m 102 may provide the primary physician's EHR system 104 with a listing of patients. Adding the patient to the care support group may include selecting John Doe's name (or other suitable identifier) from the listing shown on the GUI of potential patients selectable by the physician.

To further illustrate step 2004, the primary physician Dr. Smith may select the interface control to add practitioners to the care support group and then may be provided with the information shown in FIG. 19D. FIG. 19D illustrates an example of information shown on the GUI of the primary physician's EHR system 104 for adding one or more practitioners to the care support group. As shown, the primary physician Dr. Smith may search for a practitioner by his/her name, specialty, location and/or any other suitable identifier (e.g., clinic name, hospital name, to name a few). The Human-Centric EHR system 102 may provide the primary physician's EHR system 104 with a listing of practitioners. Then the primary physician Dr. Smith may add a desired practitioner to the care support group. Adding the practitioner to the care support group may include selecting the practitioner's name (or other suitable identifier) from the listing shown on the GUI of potential practitioners selectable by the physician. The primary physician Dr. Smith may add more than one practitioner to the care support group at this step. In this example, the primary physician Dr. Smith adds an oncologist Dr. Johnson, a radiologist Dr. Williams and a nurse practitioner Ms. Jones.

To further illustrate step 2006, the primary physician Dr. Smith may select the interface control to add one or more health records to the care support group and then may be provided with the information shown in FIG. 19E. FIG. 19E illustrates an example of information shown on the GUI of the primary physician's EHR system 104 for adding one or more health records associated with the patient to the care support group. The primary physician Dr. Smith may be provided with a listing of all or a select number of the patient John Smith's health records that are stored on the Human-Centric EHR system 102 and then is provided the opportunity to select the health records that the primary physician Dr. Smith believes to be relevant for being added to the care support group for access by the other practitioners associated with the care support group. The selection of the health records for being accessible to the care support group may include the use of a search and/or selection field for selecting specific health records meeting a specific criterion. For example, the primary physician Dr. Smith may be able to select records according to: a date range (e.g., health records before a certain date, after a certain date, between two dates); a location (e.g., a specific clinic, hospital, laboratory, imaging facility, city, province/state, country); a practitioner (e.g., a doctor's or practitioner's name) a condition associated with the record (e.g., disorder, infection, disability, injury, situation and/or any other suitable condition or situation); testing results (e.g., blood tests, urine [symbol] x-ray images, and/or any other suitable test or image type); and/or any other suitable field for [symbol] ting health records.

Once the primary physician Dr. Smith creates the care support group, prior to the other practitioners (which in this example are Dr. Johnson, Dr. Williams and Ms. Jones) are granted access to the care support group the patient may have to grant authorization. At step 2008, the primary physician Dr. Smith requests authorization from the patient to create the care support group. The authorization may be done through the Human-Centric EHR system 102 in which case upon creation of the care support group the patient is notified via the Human-Centric EHR system 102 of the care support group and is asked to grant access, which may be done according to the process 2000B. In other cases, the authorization may be a verbal authorization by the patient to the physician during the consultation, in which case the primary physician may indicate to the Human-Centric EHR system 102 that the patient has provided consent and the other practitioners may now have access to the care support group.

Turning to FIG. 21, the example care support group data structure 2100 stored on the Human-Centric EHR system 102 is shown. As shown, the data structure 2100 includes a primary physician identifier 22001 which is used as a pointer to point to a data record 2201 associated with the primary physician Dr. Smith. When the primary physician Dr. Smith creates the care support group (as discussed above) the Human-Centric EHR system 102 may create the care support group data structure 2100 and add the primary physician's identifier 22001 upon creation (e.g., creates the pointer). The data record 2201 may include information associated with the primary physician Dr. Smith such that when the primary physician Dr. Smith connects and logs in to the Human-Centric EHR system 102 via his EHR system 104, he has access to the care support group associated with the data structure 2100.

The data structure 2100 also includes a patient identifier 11001 which is used as a pointer to point to a data record 2110 associated with the patient John Doe. When the primary physician Dr. Smith created the care support group (as discussed above) and associates the patient to the care support group, the Human-Centric EHR system 102 may add the patient identifier 11001 to the data structure 2100 (e.g., creates the pointer). It is appreciated that the Human-Centric EHR system 102 may maintain a list of the patients that are registered with the Human-Centric EHR system 102 and/or that have health records stored therein. As such, the Human-Centric EHR system 102 may have a list of patients where each of the patients is associated with a unique identifier, which may be used in the storage of data and health records associated with each patient. The data record 2110 may include information associated with the patient John Smith such that when the patient John Smith connects and logs in to the Human-Centric EHR system 102 via his [symbol] puting entity 106, he has access to the care support group associated with the data structure 2100. The [symbol] record 2110 may include information pertaining to the patient John Smith's health records such as storage of the health records themselves and/or pointers to where the health records associated with the patient may be accessed.

The data structure 2100 also includes other practitioners' identifiers 22012, 22014, 22016 which are used as pointers to point to respective data records 2202 2204 2206 associated with Dr. Johnson, Dr. Williams and Ms. Jones, respectively. When the primary physician Dr. Smith created the care support group (as discussed above) and associates the other practitioners to the care support group, the Human-Centric EHR system 102 may add the other practitioners' identifiers 22012, 22014 and 22016 to the data structure 2100 (e.g., creates the pointers). It is appreciated that the Human-Centric EHR system 102 may maintain a list of the practitioners that are registered with the Human-Centric EHR system 102. As such, the Human-Centric EHR system 102 may have a list of all of the practitioners where each of the practitioners is associated with a unique identifier, where the unique identifiers may be used for allowing respective practitioners access to various health records associated with various patients. More specifically, the practitioners' identifiers may be used such that respective practitioners have access to the care support group. The data records 2202, 2204 and 2206 may include information associated with the respective practitioners such that when one of the practitioners connects and logs in (e.g., by providing his/her account identifier and credential) to the Human-Centric EHR system 102 via his/her EHR system 104₁, 104₂ or 104₃, he/she has access to the care support group associated with the data structure 2100.

The data structure 2100 also includes health record identifiers 11001-00010, 11001-00011, 11001-00012, 11001-00013, 11001-00014 and 11001-00015, which are used as pointers that point to respective health records 302₁, 302₂, 302₃, 302₄ and 302₅, respectively. When the primary physician Dr. Smith created the care support group (as discussed above) and associates patient's health records to the care support group, the Human-Centric EHR system 102 may add the health record identifiers 11001-00010, 11001-00011, 11001-00012,11001-00013, 11001-00014 and 11001-00015 to the data structure 2100. These health records 302₁, 302₂, 302₃, 302₄ and 302₅ may be of the type discussed elsewhere in this document.

Turning now to FIG. 20B, the patient may grant access to the selected practitioners to the care support group according to the process 2000B. At step 2052, the Human-Centric EHR system 102 receives the request from the physician's EHR system 104 to create the care support group associated with the patient (e.g., step 2008 of process 2000A). At step 2054, the Human-Centric EHR system 102 then notifies the patient John Doe via his computing entity 106 of the request to create the care support group and request [symbol] orization from the patient. This step of notification and authorization may be done according to the [symbol] us methods and process discussed elsewhere in the document, which may include sending a notification which does not provide any confidential information prior to sending the request for authorization. At step 2056, the Human-Centric EHR system 102 receives the authorization from the patient, and the Human-Centric EHR system 102 authorizes the practitioners associated with the care support group access to patient's health records associated with the care support group. Then at step 2058, in response to the patient's authorization for the creation of the care support group, then each of the practitioners associated with the care support group may have access to the care support group and the associated health records of the patient made available through the care support group.

It is appreciated that the patient may deny access to the creation of the care support group and in such case access would not be granted to the practitioners. In other cases, the patient have the ability in granting the authorization to select which practitioners may be added to the care support group and/or which health records may be added to the care support group. For example, the patient may be provided on the GUI of his computing entity 106 with a list of the other practitioner that the primary physician has added to the care support group and/or a list of the health records that the primary physician has added to the care support group. Then, the patient may be able to select the various practitioners and/or health records that are made available to the care support group.

After the care support group has been created, the patient (e.g., John Doe) may be able to log into the Human-Centric EHR system 102 and add additional practitioners and/or health records to the care support group. For example, if the patient John Doe sees another doctor Dr. Anderson, then John Doe may then add Dr. Anderson to his care support group such that Dr. Anderson may be able to log in to the Human-Centric EHR system 102 and have access to the care support group. By way of another example, if the Human-Centric EHR system 102 receives additional health records associated with the patient, the patient may be notified of the additional health records, which the patient may add to the care support group. In general, the patient may be able to add health records to the care support group by selecting any of the health records stored on the Human-Centric EHR system 102 such that the practitioners associated with the care support group have access to the added health records.

In some embodiments, the patient may have the ability to specify which health records to be accessible to which of the practitioners associated with the care support group and may have the ability to place restrictions on the practitioners that have access to the care support group. The practitioners added to the care support group may be added with specific time limitations (e.g., corresponding to a treatment and/or [symbol] very period) and the patient may be able to specify the time periods that one or more of the [symbol] titioners has access to the care support group. The patient may also have the ability to be able to log in to the Human-Centric EHR system 102 and remove practitioners and/or health records from the care support group.

After step 2058 each of the practitioners may be sent a notification from the Human-Centric EHR system 102 that the care support group has been created, that the patient has granted authorization and/or that they now have access to the care support group. In this example, the practitioners Dr. Smith, Dr. Johnson, Dr. Williams and Ms. Jones may then be able to connect to the Human-Centric EHR system 102 to have access to the care support group. The practitioners Dr. Smith, Dr. Johnson, Dr. Williams and Ms. Jones may connect via the respective EHR systems 104, 104₁, 104₂ and 104₃. It is appreciated that the practitioners Dr. Smith, Dr. Johnson, Dr. Williams and Ms. Jones may not be tied to a specific system or device and may connect via various EHR systems or devices to the Human-Centric EHR system 102 using their account identifiers and credentials.

Each of the practitioners may be able to view the health records associated with the patient that is associated with the care support group. For example, prior to the patient John Doe having a consult with Dr. Johnson, Dr. Johnson may connect to the Human-Centric EHR system 102 and view the various records of the care support group associated with the patient John Doe.

Each of the practitioners may be able to add annotations to a specific health record in the group of the health records associated with the care support group. Each of the practitioners may be able to add additional health records to the group of health records associated with the patient that is associated with the care support group. For example, after the patient John Doe has a consult with Dr. Johnson, Dr. Johnson may then add an annotation and/or a new health record to the care support group associated with the patient John Doe, which may detail the Dr. Johnson assessment the patient and/or the patient's current health records.

Continuing with this example, Dr. Johnson may add a treatment regime for the patient John Doe and may add this to a new health record associated the care support group associated with the patient John Doe. Then in administering the treatment regime, the nurse practitioner Ms. Jones may access the care support group associated with the patient John Doe to view the treatment regime which may include a treatment dosage and schedule for the treatment of John Doe's cancer. Ms. Jones may add an annotation and/or new health record to the care support group associated with the patient John Doe after administering the cancer [symbol] ment. After adding the annotation and/or new health record related to the patient being administered [symbol] reatment, a notification may be sent to Dr. Johnson via the Human-Centric EHR system 102 such that Dr. Johnson is notified that the patient is undergoing the prescribed treatment.

It is appreciated that the annotation and/or new health record added to the care support group associated with the patient John Doe may also include the addition of notifications and/or action items (which are also discussed elsewhere in this document). For example, Dr. Johnson in adding the treatment regime may add an action item to notify the nurse practitioner that John Doe is going to undergo treatment and that an appointment should be schedule. By way of another example, Dr. Johnson in adding the treatment regime may add an action item such that he will be notified of the patient's treatment. In other words, action items and/or notifications may be used by the practitioners to schedule treatments, prescribe prescriptions/treatments, to schedule appointments, setup reminders, inter-practitioner communication, to share laboratory testing and/or imaging results, and/or any other suitable means.

It is also appreciated that the same patient may be part of more than one care support group, as the care support group may be created regarding a specific condition. For example, the patient may have a care support group for the treatment of cancer and another care support group for the treatment of diabetes, where the practitioners in each care support group are different and have different access to the patient's health records. In some cases, the primary physician may be the same in multiple care support groups and some practitioners may be associated with multiple cares support groups associated with the same patient.

Although in this example the plurality of EHR systems 104₁ 104₂ 104₃ associated with other practitioners have EHR functionality, in other embodiments, the systems 104₁ 104₂ 104₃ associated with other practitioners do not have EHR capability are may be any suitable computing system such as a computer, laptop, tablet, mobile phone and/or any other suitable device. In such case, the systems 104₁ 104₂ 104₃ associated with other practitioners may connect to the Human-Centric EHR system 102 via a web browser, application software running on the systems 104₁ 104₂ 104₃ and/or any other suitable means.

Although in this example, the primary physician Dr. Smith creates the care support group, in other embodiments, the patient or other practitioners may create the care support group in a similar fashion to that described herein.

Although in this example, the primary physician Dr. Smith added specific practitioners, in other examples the primary physician Dr. Smith may have added a facility to the care support group. For example, the [symbol] ity added to the care support group may be a laboratory or testing facility, which can then view the [symbol] prescribed by any of the practitioners in the care support group and then add the test results to the care support group. In other words, access to the care support group may be assigned to specific entities such as hospitals, clinics, laboratories and/or any other suitable entity.

In other examples, the primary physician Dr. Smith may also add the computer-based decision support agent (discussed elsewhere in this document) to assess the patient's health record such that the practitioners associated with the care support group may view the results from the decision support agent, which may then be used for the treatment of the patient and/or discussed among the practitioners.

It is further appreciated that the care support group may also be used for clinical research.

Anonymized Health Record Access Mechanism

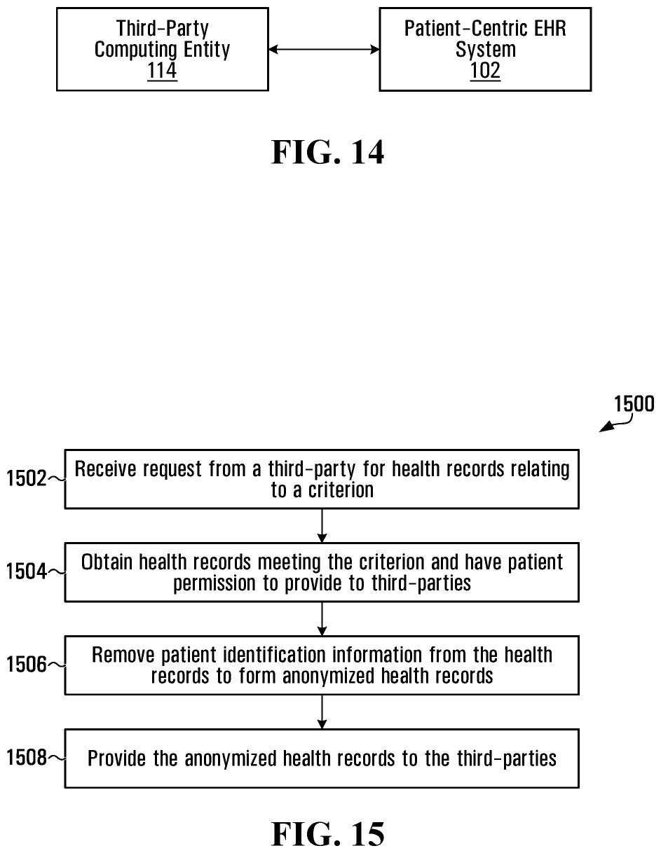

FIG. 14 illustrates a block diagram of an example of the Human-Centric EHR system 102 connected to an agent or third-party computing entity 114, such that the Human-Centric EHR system 102 may provide patient anonymized health records to a third-party computing entity 114. In other cases, the health records provided to the third-party computing entity 114 are not anonymized and may be similar to the various other embodiments discussed in this document. The connection between the Human-Centric EHR system 102 and the third-party computing entity 114 may include one or more connections over one or more data networks. The connection between the Human-Centric EHR system 102 and the third-party computing entity 114 may allow for the communication (e.g., transmission and/or reception) of various information and/or data between the various systems and/or devices. The various information and/or data exchanged may be a “notification”, “notifications” a “health record” and/or “health records” of the type discussed elsewhere in this document.

FIG. 15 illustrates a flowchart of a process 1500 for providing patient anonymized health records to the third-party computing entity 114. At step 1502, the Human-Centric EHR system 102 receives a request from a third-party for health records relating to a criterion or criteria. In this example, the third party is the third-party computing entity 114. Then, at step 1504, the Human-Centric EHR system 102 obtains health records meeting the criterion or criteria, by processes the criterion or criteria against the database 206 of health records to obtain health records meeting the criterion or criteria and where each obtained record has permission from the respective patient associated with the record for the record to be provided to third-parties. For instance, when patients register with the Human-Centric EHR system 102 they may be [symbol] d if they are willing to provide anonymized data from their health record to third-parties. In other [symbol] is, a patient may be able to deny access to third-parties to his/her health record. For example, as is shown in FIG. 16E, there may be a settings page associated with the patient's account with the Human-Centric EHR system 102, such that the patient can view this page once he/she is logged into the Human-Centric EHR system 102. Then at step 1506, the patient identification information is removed from the health records to form a cohort of anonymized health records. At step 1508, the cohort of anonymized health records is provided to the third-party computing entity 114.

In some embodiments, the third-party computing entity 114 is associated with an agent that is associated with the Human-Centric EHR system 106. For example, the operator or the Human-Centric EHR system 106 could have agreements with agents to provide agents with access to various aspects of the data stored in the Human-Centric EHR system 106. For example, in one case, an agent could be registered to obtain a cohort of patient health records meeting certain criteria. The Human-Centric EHR system 102 upon a query could provide anonymized data to the third-party computing entity 114, such that the agent could conduct research with the cohort of patient health records. The patient can allow/deny being discovered as part of a query for cohorts. In order for the Human-Centric EHR system 102 to allow for the privacy of patient health information, the patient may be able to configure via the patient's computing entity 106 whether he/she is allowed to be discovered by queries from third-parties. If the patient desires for his/her medical information to not be provided to third-parties, even if the patient matches the criteria of the query, his/her anonymized data is not returned to the third-party agent.

In some embodiments, if patient agrees to provide anonymized data to third-parties, each time his/her name is picked in a query (e.g., on a per a study basis), he/she may have the option to opt-out of the cohort. For example, each time his/her name is picked in a query, he/she may receive a notification (such as the type discussed elsewhere in this document) and accept or deny his/her health records to be included in the cohort. In other words, the Human-Centric EHR system 102 could be configured to allow the patient to revoke his/her consent at any time.

In some embodiments, the agent is able to select a specific anonymized patient from the cohort of patient health records to conduct further health analysis (diagnostics) or establish patient membership in a specific research topic. For example, if a specific anonymized patient, he/she may receive a notification (such as the type discussed elsewhere in this document) and accept or deny his/her health record to be included in the further health analysis (diagnostics) or establish patient membership in a specific research topic [symbol] ome embodiments, the agent could be a ‘blood pressure’ monitor software-as-a-service component, that could alert the patient (and physician) of an abnormal situation over time. The patient again may subscribe, permit notifications, and the physician can do the same for certain ‘thresholds’ defined by the agent. The ‘blood pressure monitor’ may not need to be co-resident with the Human-Centric EHR system 102, but may make use of notifications and notification-lists to habilitate and regulate the flow of information device-agent-medical record-physician.

In some embodiments, the Human-Centric EHR system 102 could have agreement with agents for ‘travel related risks’. In this case, the patient subscribes for notifications from the agent and authorizes the agent to use his/her information to monitor potential health hazards as he moves around the globe. In this case, the data is not anonymized, since it is used directly for a specific patient.

The Human-Centric EHR system 102 may include in the account of the patient a record of the various studies and/or third parties that he/she participated in, which may be used for compensating the patient for participating. The patient may be compensated monetarily or may be compensated by receiving various information and/or data from the study that may be useful to the patient. For instance, the Human-Centric EHR system 102 may notify the patient via the patient's computing entity 106 with a notification that a third-party is requesting to use his/her data and provide him/her with the available compensation, if the patient accepts to provide his/her health record. If the patient acknowledges by selecting the allow option in the notification, then the patient's account may be compensated.

The term “agent” may be used to refer to a third-party system that may be able to receive one or more health records associated with a patient, where the health records may or may not be anonymized. As such, the patient may provide one or more health records to an agent where the agent could provide monitoring, processing or computational service of heath records. It is appreciated that the agent may include the computer-based decision support agent discussed elsewhere in this document.

In another embodiment, the Human-Centric EHR system 102 may be used to provide a decision aid for physicians. For example, the physician at the physician's EHR system 104 may be able to configure various parameters of a health record associated with a patient on the Human-Centric EHR system 102. Such parameters may include which record and which information is made available to third parties. As such the physician may provide a health record associated with a patient or various parts of the patient's health record that the physician would like to provide to a third-party such as an agent (e.g., the agent at [symbol] third-party system 111 or third-party computing entity 114). More specifically, the physician may [symbol] ide one or more workflows to be done by the agent on the physician's behalf. Furthermore, the physician may be able to configure the “anomaly” detection thresholds of various observations to suit his/her practice. Also, an agent may be able to examine a patient's entire records collection and evaluate observations against each other and against the patient's health condition, to try to determine whether some adverse condition is possible given the patient's observation results and other information available therein. In some cases, the agent is another physician that the primary physician would like to have his/her patient's health record reviewed by.

User Authentication

This section refers to functionalities of the Human-Centric EHR network 108 to authenticate users such that access to medical information is provided to legitimate users only.

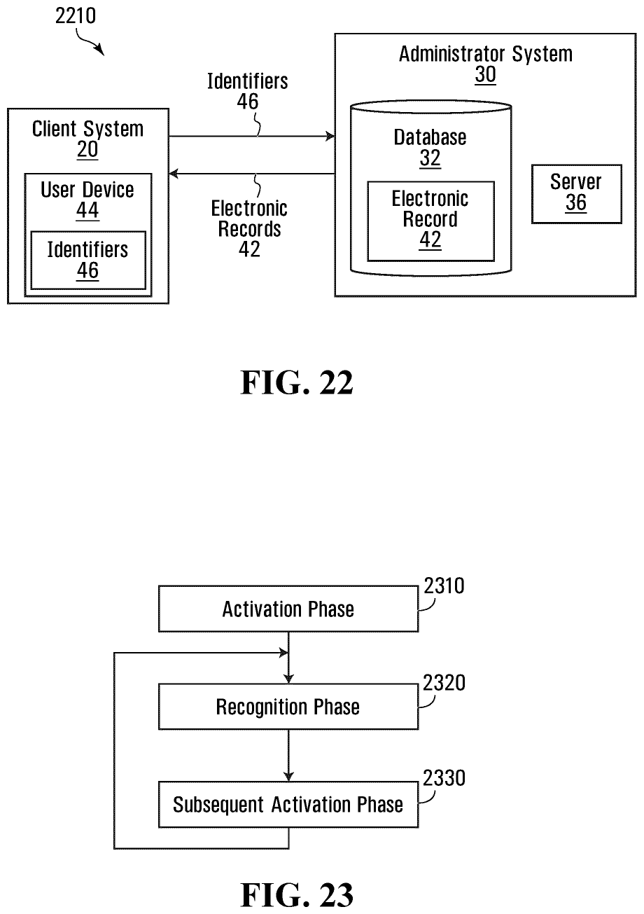

The accessibility and portability of the EHR networks 108 raise issues regarding confidentiality of the health records, as accurately identifying a patient remotely accessing health records is challenging. In some embodiments, the authentication system 2210 shown in FIG. 22 is part of an EHR network 108.

The authentication system 2210 allows a user 40 to connect to a database 32 managed by an administrator system 30 or facility, using a client system 20. In this embodiment, the authentication system 2210 ensures that client system 20 is owned and operated by the rightful user 40, therefore preventing unauthorized users to access the database 32 of the administrator system 30.

In a specific embodiment, the user 40 is a patient. The administrator system that could be partially or entirely located in a medical clinic in order to access electronic records 42 that could be electronic health records. If the EHR network 108 is entirely managed by a single medical clinic, the administrator system 30, including the servers 36, may be located at the clinic. If the EHR network 108 is managed by a plurality of medical clinics, the administrator facility 30 may be distributed between the clinics and the servers 36 may be located at one of the clinics or elsewhere. The servers 36 may be, for example, cloud based.

In the embodiments discussed herein the term “medical clinic” is used. Note, “medical clinic” may include paramedical services such as dental clinics, optometry clinics, pharmacies and/or any other [symbol] ble medical/paramedical service provided and may be used synonymously with the term “medical [symbol] c”.

In a specific example of implementation, the Human-Centric EHR system 102 shown in FIG. 1A may implement, the administrator system 30 in FIG. 22, while the client system 20 in FIG. 22 may be implemented by the Patient's computing entity 106 shown in FIG. 1A.

More specifically, in this example, the administrator system 30 comprises a processing unit, input/output ports, and computer readable memory comprising the database 32 and an administrator authentication program. For instance, the administrator system 30 may comprise a server 36 and optionally one or more computers connected to the server 36. The administrator system 30 may have a distributed architecture where the various elements of the administrator system 30 communicate with each other via data links. The database 32 stores one or more electronic records 42 associated with a user 40 and at least one authorized user device 44 having identifiers 46. The administrator authentication program comprises program code which when executed by the processing unit causes the administrator system to perform various operations. The administrator system 30 connects to the client system 20 via one or more data networks.

The client system 20 typically comprises a processing unit, input/output ports, and computer-readable memory, a client authentication program, and identifiers 46, such as authentication information uniquely identifying the user. The processing unit, input/output ports and computer readable memory of the client system 20 may be connected to each other by various data buses and in the case of a distributed computing environment may be interconnected by one or more data networks. The client authentication program comprises program code which when executed by the processing unit causes the client system to perform various operations, such as managing a user authentication operation involving the identifiers 46.

The client system 20 can communicate with the administrator system 30 to access information in the database 32. In particular, the client system may access a given one of the electronic records 42 of the database 32. The communication between the client system 20 and the administrator system 30 may be initiated by any one of the systems 20, 30. The administrator system 30 may transmit the given one of the electronic records 42 of the database 32 to the client system 20 if the identifiers 46 of the client system 20 are recognized by the administrator authentication program as being authorized to give access to a particular one of the electronic records 42. In particular, the client system 20 transmits the identifiers to the administrator system 30 and the administrator system 30 compares the identifiers of the client system [symbol] the identifiers of the authorized user device associated with the given one of the electronic records. [symbol] ere is a match, the administrator system 30 recognizes the client system 20 as belonging to the user 40 and the given one of the records is transmitted to the client system 20. If there is no match, the client system 20 is not recognized as being an authorized user device and no access to the information in the database 32 is allowed.

As shown in FIG. 23, in one embodiment, the authentication system 2210 performs a process including an activation phase 2310, wherein the user 40 of the client system 20, which typically is a mobile, is authenticated, the mobile of the user 40 communicates with the administrator system 30 and transmits authentication information to the administrator system 30, the administrator system 30 associates the authentication information 46 of the device 44 of the authorized user 40, and the administrator system 30 stores the authentication information of the device 44 into the computer readable memory of the administrator system 30. In some embodiments, activation phase 2310 is followed by a recognition phase 2320. In this phase, the user 40 may authenticate himself/herself to remotely access his/her personal data, acknowledge correspondence from a physicist, request an appointment, etc., and optionally enter into the subsequent activation phase 2330, using the device 44, to activate a new device 144.

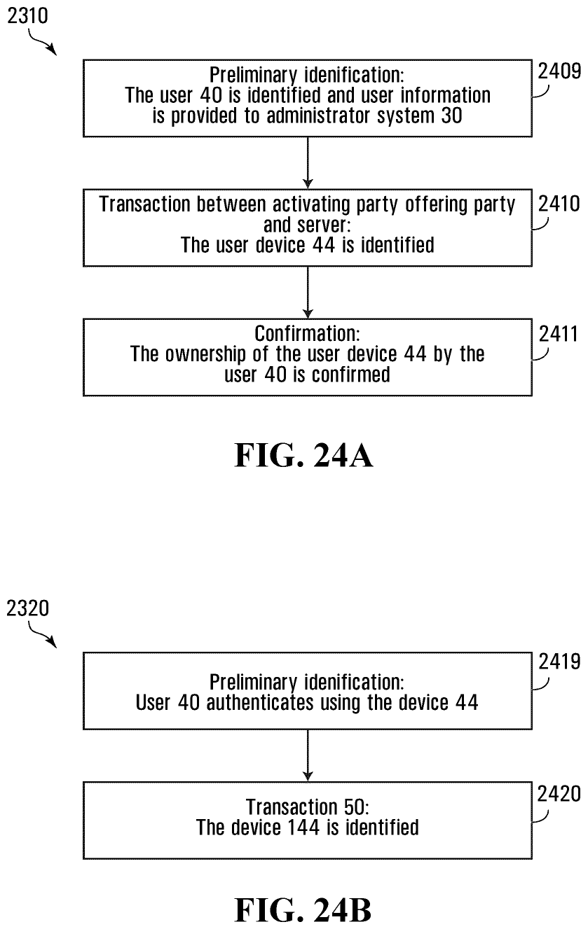

The activation phase 2310 is a phase wherein authentication information about the device 44 is saved into servers 36 of the administrator system 30 and identified as being allowed to access a particular one(s) of the electronic records 42 of the database 32. As shown in FIGS. 24A and 24B, in some embodiments, the activation phase 2310 may comprise step 2409, which is a preliminary identification, and step 2410, which is a transaction 50. Activation phase may also comprise a confirmation step 2411 wherein the ownership of the user device 44 by the user 40 is confirmed.

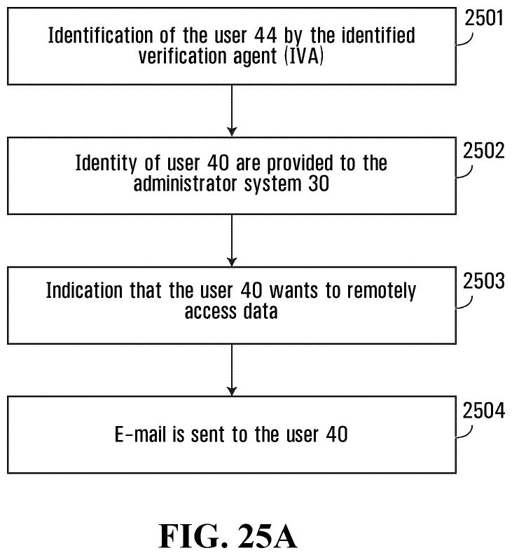

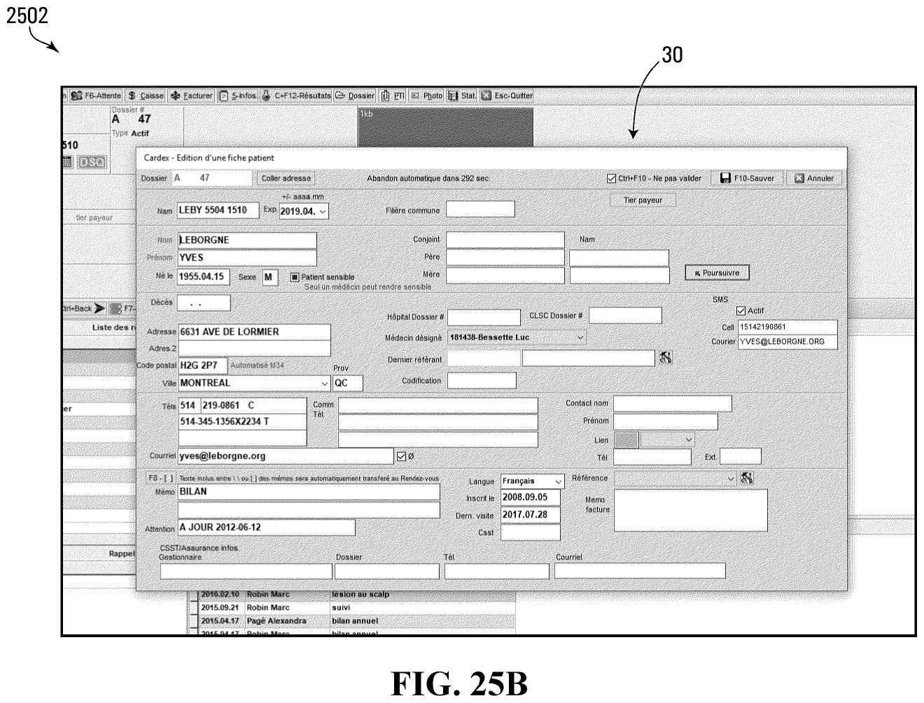

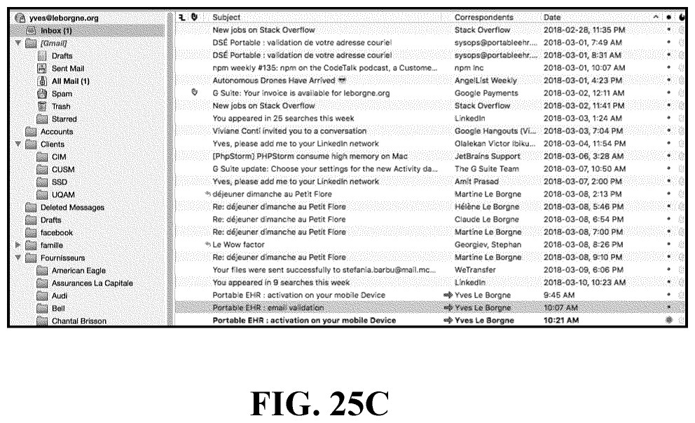

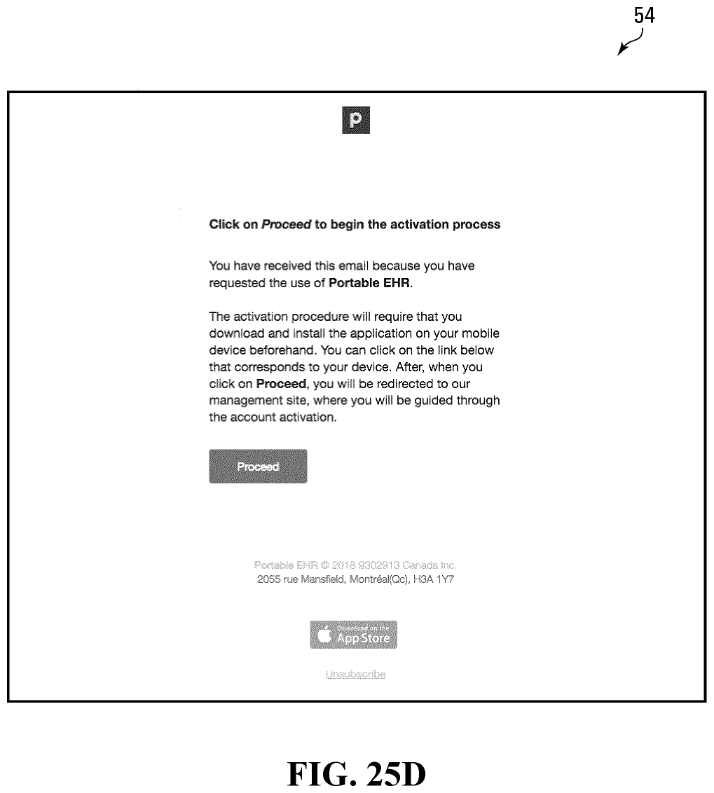

At step 2409, the user 40 is identified and information provided by the user is transmitted to the administrator system 30. In this embodiment, the identification of the user 40 is accomplished by an identity verification agent (IVA). An example of steps of the preliminary identification is depicted in FIG. 25A. FIG. 25B illustrates a GUI that an IVA can manually fill with information specific to the user, such as the name, address, email and other relevant information. As discussed below, that information is then recorded into the administrator system 30. The IVA, who can be a receptionist at a medical clinic first visually identifies the user 40 at step 2501, for example by using identification document (ID) and by comparing features depicted on the ID to features of the face of the user 40 to verify that the ID is legitimately owned by the user 40 and/or by verifying information (e.g., name, birth date, social security number) printed on the ID to ensure that the user 40 is who he says he is. Secondly, [symbol] dentity of the user 40 is recorded into the administrator system 30 at step 2502 (FIG. 25A-B). In some [symbol] s, the user 40 may enter its own identity into the administrator system 30, while in other cases, the IVA may enter the user 40 identity information into the administrator system 30. For example, in a preferred embodiment, the IVA enters an email address and cell phone number of the user 40 into the database 32 of the administrator system 30. Optionally, the IVA may indicate by checking a radio box that the user wants to access his data remotely at step 2503. Subsequently, at step 2504, the administrator system 30 sends an email to the user 40 using information provided by the user 40 as means to start the activation phase 2310. While steps 2501, 2502, 2503, 2504 are completed, the user 40 may install an application or software 48 on the device 44.

Alternatively, the preliminary identification of the user 40 at step 2409 may be made automatically by a machine belonging to the administrator system 30, using an ID scan, face recognition, digital prints, or the like. At step 2502, the user 40 may enter identity information into the machine. Step 2504 happens subsequently.

At step 2410, the transaction 50 that takes place, ensures that the device 44 is indeed owned by the user 40 and allows the device 44 to transmit identifiers 46 to the servers 36 of the administrator system 30, which records the identifiers 46 and associates them to the electronic records 42. As such, the transaction 50 involves multiple parties comprising an activating party 52, an offering party 54 and the servers 36 of the administrator system 30.

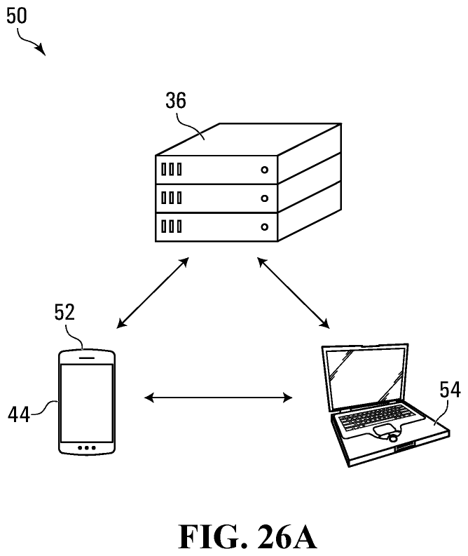

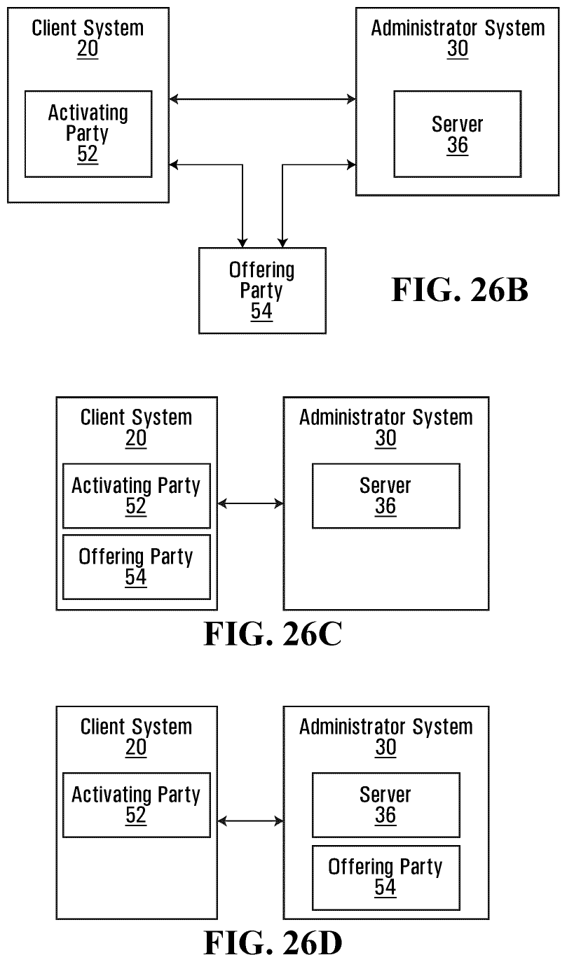

FIGS. 26A-D show non-limiting examples of activating party 52, offering party 54 and server 36 that are involved within a transaction 50 including related GUIs allowing users to interact with the respective devices to complete the process. In this case, the device 44 may be considered as the activating party 52. The offering party 54 be, in some cases, a device owned by the user 40; in some cases, a device manipulated by the IVA; in some cases, the machine belonging to the administrator system 30; and in some cases, a device that is also part of the administrator system 30.

The activation process can be triggered by the device 44 via an app that runs on the device 44. For instance, that app may be downloaded by the user from an app store of installed via another source. The activation screen of the device 44 is shown at FIG. 25D. The user presses on the “Proceed” button to initiate the process. [symbol] ay to ensure that the device 44 is owned by the entitled user 40 is to control the offering party 54 and [symbol] ires a physical proximity between the offering party 54 and the activating party 52 for the transaction 50 to succeed. As such, in cases where the offering party 54 is a device manipulated by the IVA, this may allow the IVA to ensure that the device 44 is indeed owned by the user 40, who was previously identified at step 2409. Similarly, in cases where the offering party 54 is the machine belonging to the administrator system 30, this may allow the administrator system 30 to ensure that the device 44 is indeed owned by the user 40, who was previously identified at step 2409.

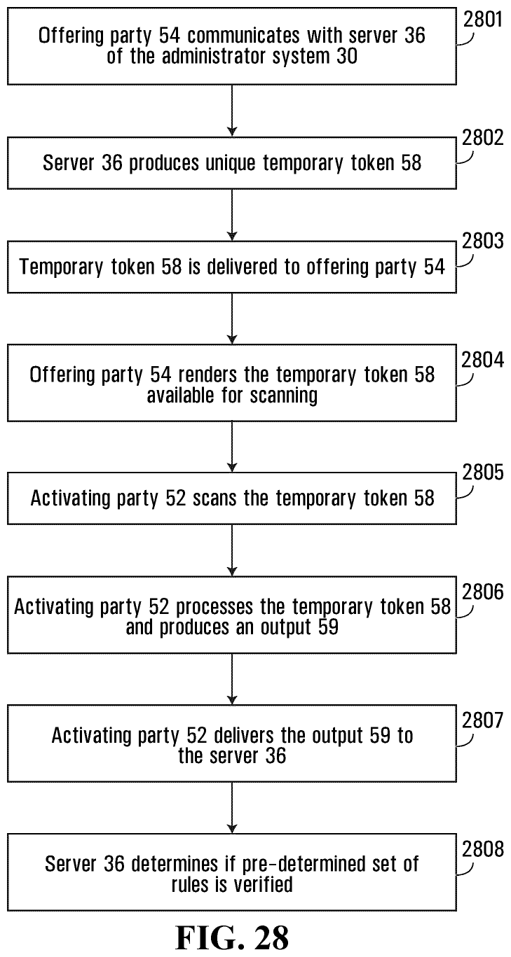

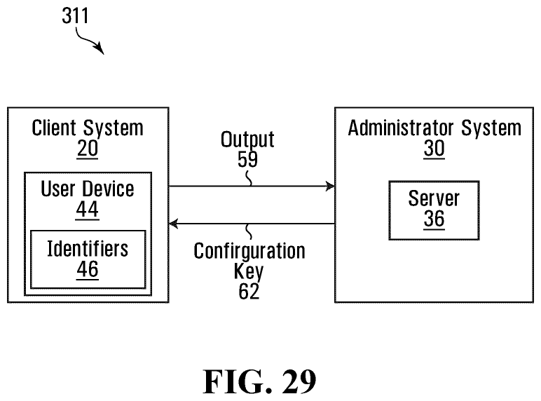

FIG. 28 is a flowchart of the transaction 50 according to this embodiment. The offering party 54 first communicates with the server 36 of the administrator system 30 to initiate the transaction 50 at step 2801. At step 2802, the server 36 produces a unique temporary token 58, which is then delivered to the offering party 54 at step 2803.

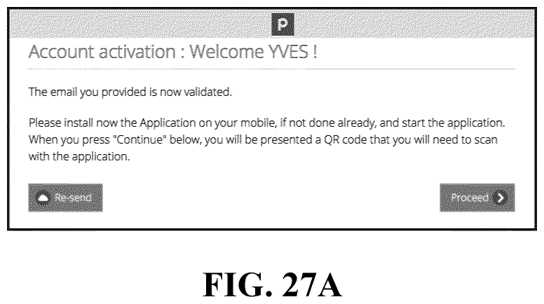

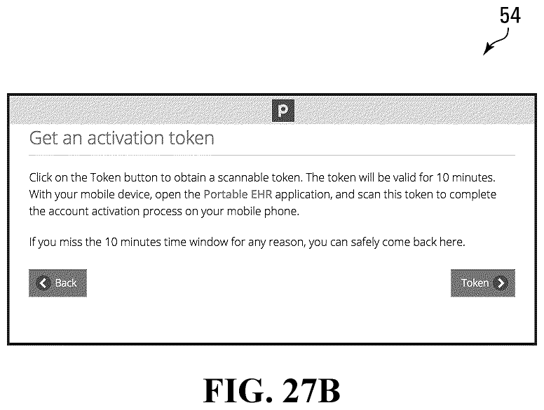

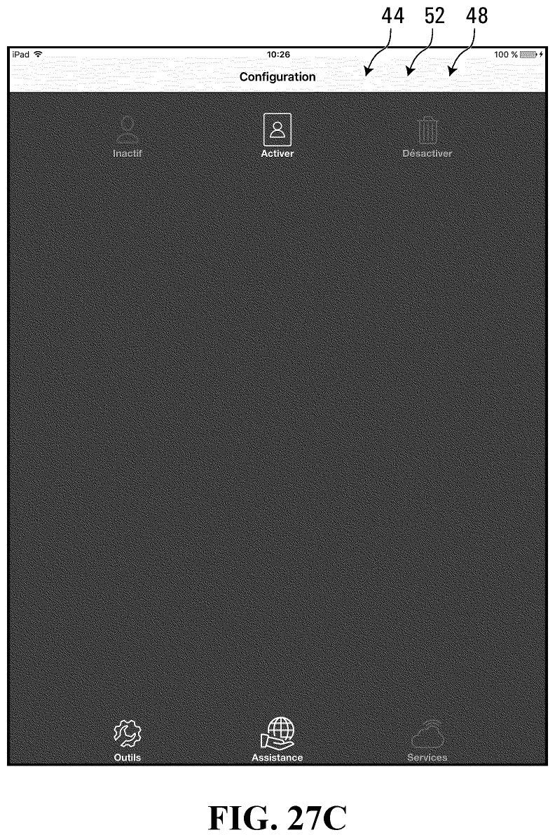

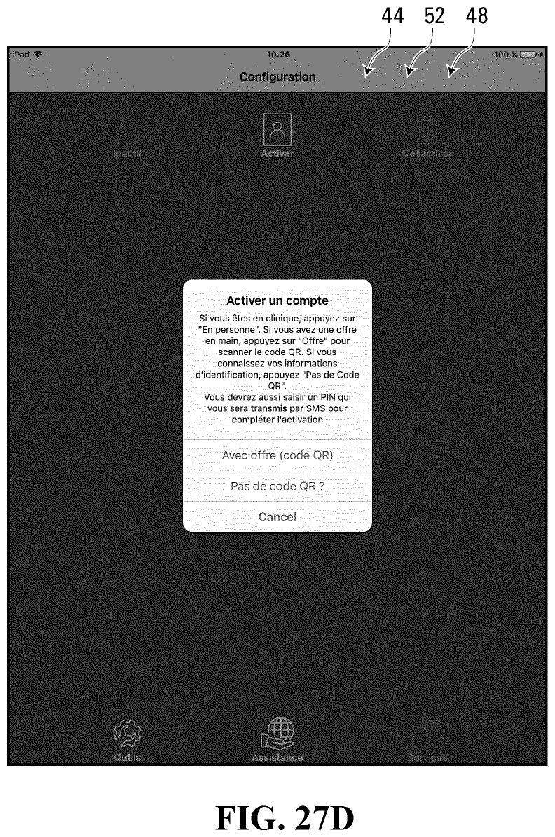

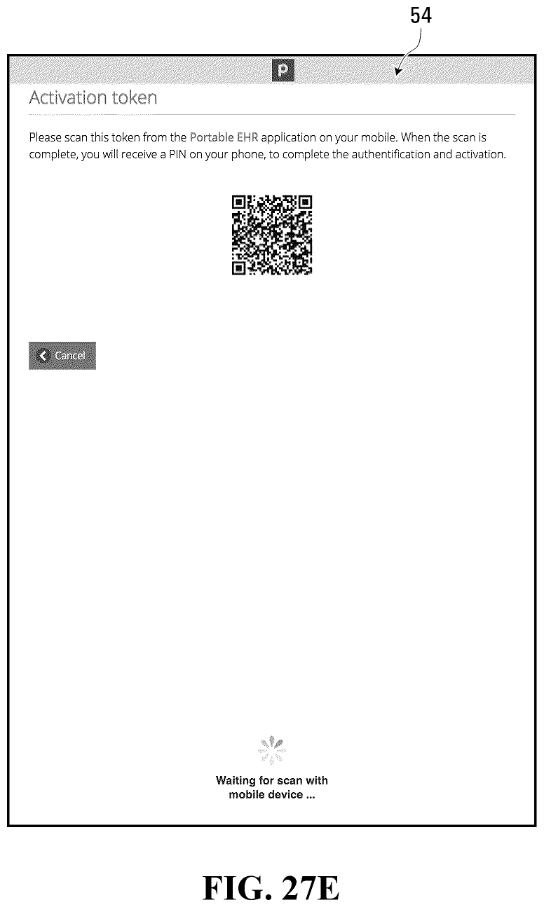

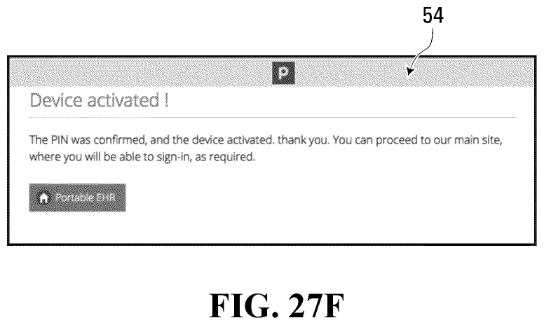

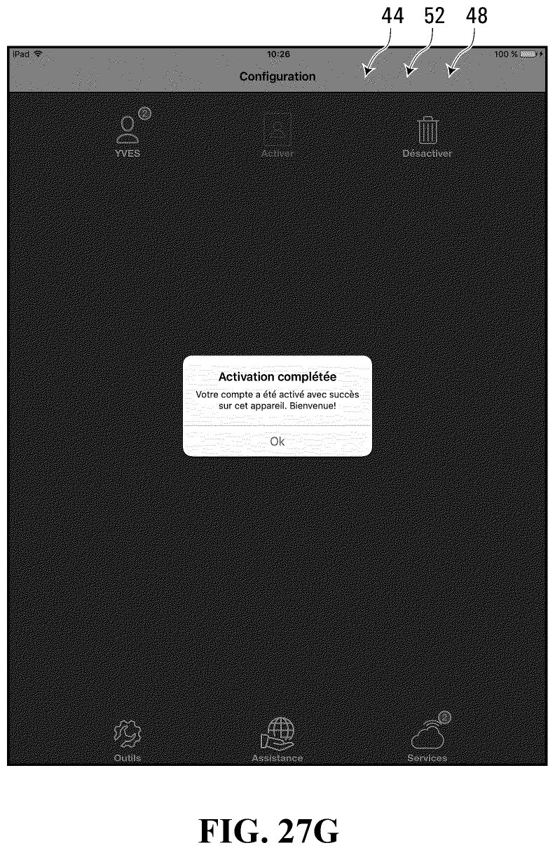

FIG. 27B illustrates a GUI on the device 44 showing controls to trigger the generation of the temporary token. The temporary token 58 may be, for example, a QR code, an alphanumeric digital key, etc. Then, at step 2804, the offering party 54 renders the temporary token 58 available for scanning by the device 44 of the activating party 52. For example, in cases where the temporary token 58 is a QR code and the offering party 54 is a smartphone, a tablet or a computer, the temporary token 58 may be made available for scanning by being displayed on a display of the offering party 54. An example of a QR code that can be scanned by the device 44 is shown at FIG. 27E. As another example, in cases where the temporary token 58 is an alphanumeric digital key and the offering party 54 comprises an NFC emitter, the temporary token 58 may be made available for scanning by enabling the NFC emitter of the offering party 54 and pushing the temporary token 58 to other devices connected through the NFC connection. Subsequently, at step 2805, the activating party 52 scans the temporary token 58 through the application 48 of the device 44. The activating party 52 may then process the scanned temporary token 58 at step 2806 to produce an output 59. The output depends on the temporary token 58. In some cases, the output 59 is identical to the temporary token 58, while in the other case the output 59 differs: for example, for a temporary token 58 being a QR code, the output 59 may be an alphanumerical key. The output 59 may also comprise a signature of the activating party 52, e.g., the identifiers 46 of the device 44. At step 2807, the activating party 52 transmits the output 59 to the server 36, using the application 38. While in the present case, the activating party 52 transmits the output 59 directly to the server 36, in some embodiments, the activating party 52 transmits the output 59 indirectly to the server 36, e.g., by delivering the output 59 to the offering party 54, which may then deliver the output 59 to the server 36. At step 2808, the server 36 processes the output 59 and determines if a pre-determined set of rules of the [symbol] action 50 is being met. If the pre-determined set of rules is met, the identifiers 46 of the client device [symbol] ay be registered into the server 36 and the transaction 50 is completed. If the pre-determined set of rules are not being met, the identifiers 46 of the client device 44 are not registered and the transaction 50 is aborted.

As previously mentioned, the temporary token 58 may be of any suitable form. For example, in some cases, the temporary token 58 may be an image such as a QR code, or any other image. In some cases, the temporary token may be a numerical key or an alphanumerical link. In some cases, the temporary token may be a hyperlink.

In some examples, if the step 2801 of the transaction 50 is repeated, the temporary token 58 may be deactivated and a new temporary token may be activated. Every remaining step of the transaction 50 may also be repeated to complete the transaction 50. Also, the pre-determined set of rules of the transaction 50 may comprise a rule stating that the temporary token 58 must be activated, to ensure that no more than one valid temporary token 58 is available and to reduce the risk that unauthorized users take part in the transaction and corrupt the authentication system 2210.

In this case, also, the temporary token 58 may have a limited and pre-determined lifetime. Once the lifetime becomes expired, the temporary token 58 may be deactivated by the server 36. Also, the pre-determined set of rules of the transaction 50 may comprise a rule stating that the temporary token 58 must be activated and its lifetime must not be expired. To complete the transaction 50, a new temporary token 58 must be delivered by the server.

In some embodiments, the transaction 50 determines an outcome of the activation phase 2310. If the transaction 50 is completed, the server 36 activates the identifiers 46 of the device 44, i.e., the server 36 registers the identifiers 46 as being authorized to access the given one of the electronic records 42 of the database 32. In this case, the activation phase 2310 is a success. Otherwise, the activation phase 2310 is a failure.

In other embodiments, the transaction 50 alone does not determine the outcome of the activation phase 2310. If the transaction 50 is completed, other steps still have to be completed to determine the outcome of the activation phase 2310. For example, in some embodiments, as shown by FIG. 24A the activation phase 2310 further comprises step 2411, which is a confirmation. At step 2411, the confirmation 60 further ensures that the device 44 is indeed owned by the user 40 previously identified at step 2409. The [symbol] irmation may take various forms. For example, in some embodiments, the server 36 of the [symbol] inistrator system 30 transmits a confirmation key 62 to the device 44 using a communication means adherent than a communication means used during the transaction 50.

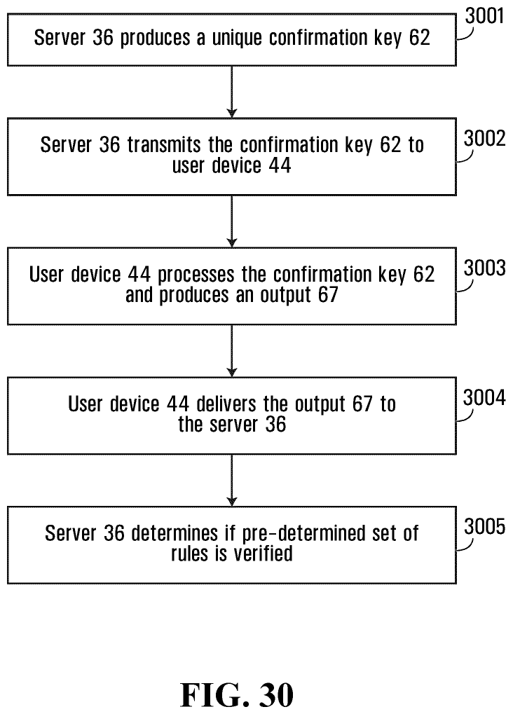

As shown in FIG. 30, the confirmation may comprise the step 3001, at which the server 36 produces the confirmation key 62. In the present case, the confirmation key 62 is alphanumerical. In other embodiments, the confirmation key 62 may be a hyperlink. At step 3002, the server 36 transmits the confirmation key 62 to the device 44. In some cases, the server 36 of the administrator system 30 may transmit the confirmation key 62 to the device 44 by short messaging service (SMS), while in some cases, the server 36 may transmit the confirmation key 62 to the device 44 by email, in every case using the cell phone number or the email address provided by the user 40. An example of the GUI when email is used is shown at FIG. 25C. In some cases, also, the confirmation key 62 is provided directly to the device 44, while in other cases the confirmation key 62 is provided to the user 40, and the user 40 needs to scan or enter the confirmation key 62 into the application 48 installed on the device 44. Then, at step 3003, the application 48 of the device 44 processes the confirmation key 62 and produces an output 67. Similar to step 2806 of the transaction 50, the output 67 depends on the confirmation key 62. In some cases, the output 67 is identical to the confirmation key 62, while in other cases the output 67 differs. The output 67 may also comprise the identifiers 46 of the device 44. At step 3004, the device 44 delivers the output 67 to the server 36. At step 3005, the server 36 processes the output 67 and determines if a pre-determined set of rules of the confirmation 60 is being met. If the pre-determined set of rules is verified, the identifiers 46 of the client device 44 may be confirmed and registered into the server 36 as associated to the user 40. If the pre-determined set of rules is not met, the identifiers 46 of the client device 44 are not confirmed and the confirmation 60 fails.

The confirmation key 62 may be a temporary token delivered by the server 36 of the administrator system 30, i.e., may have a limited and pre-determined lifetime. After the lifetime has expired, the temporary token may be deactivated. Also, the pre-determined set of rules of the server may comprise a rule stating that the temporary token must be activated in a timely manner. To complete the confirmation 60, a new temporary token must be delivered by the server 36.

In other embodiments, the confirmation key 62 may be a permanent token delivered by the server 36 of the administrator system 30, i.e., may have unlimited lifetime. [symbol] ther examples, the key 62 may be voided by the administrator system 30 if some rule has been [symbol] ted, for example, three unsuccessful attempts, administrator revoking the token, key or other identifier forcing a clean restart of the process.

The pre-determined set of rules of the confirmation 60 may comprise a rule stating that, in order to complete the confirmation 60, the identifiers 46 provided during the transaction 50 and the confirmation 60 must be compared and must be identical. Another rule of the predetermined set of rules of the confirmation may be that, in order to complete the confirmation 60, the output 67 transmitted by the application 48 to the server 36 at step 3004 must correspond to the confirmation key 62 delivered by the server 36 at step 3002.

In some embodiments, if the identifiers 46 transmitted during steps 2410 and 2411 are not consistent, the given one of the electronic records 42 may be blocked such that they remain inaccessible and/or a notification may be sent to the user 40.

Once the activation phase 2310 of the device 44 is completed, the user 40 may access data belonging to the given one of the electronic records 42, transmit such data, modify such data, process transactions, request services, etc., using the application 48 installed on the device 44.

While the activation phase 2310 may ensure that the device 44 belongs to the entitled user 40, it may not ensure that the device 44 is being manipulated by the rightful user 40 at the moment when access to the electronic records 42 is requested by the application 48 of the device 44.

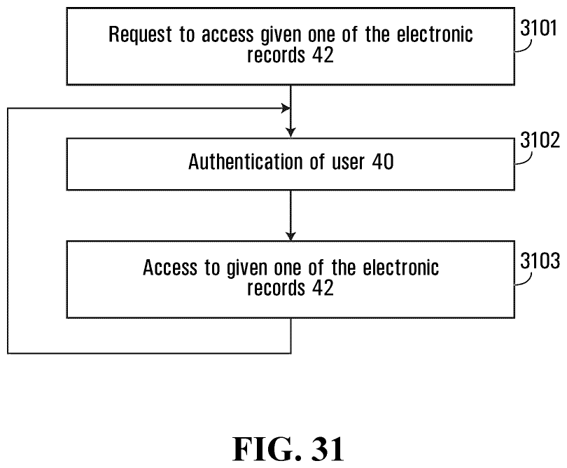

FIG. 31 shows that, in some embodiments, the application 48 requires authentication of the user 44 prior to accessing the given one of the electronic records 42. In some embodiments, after the application 48 is put into sleep or into background process, for example while another application 48 is being used, or after a time delay has expired, the user 44 may be required to authenticate prior to re-accessing the application 48 or the given one of the electronic records 42. If the authentication is not accomplished or fails, the application 48 may then be blocked, thus preventing the user 40 to use the application 48 and access the given one of the electronic records 42. For example, in some embodiments, the application 48 may request that the user 40 authenticate using face recognition, iris scan, fingerprint, pattern, password and/or pin. In some embodiments, the application 48 may request that the user 40 authenticate using the safest technology available on the device 44: for example, if face recognition and/or iris scan are available, authentication is accomplished using one of these technologies; otherwise, if fingerprint is [symbol] able, authentication is accomplished using fingerprint; otherwise, if pattern is available, [symbol] entication is accomplished using pattern; otherwise, if password is available, authentication is accomplished using password; otherwise, if pin is available, authentication is accomplished using pin; otherwise, the application 48 cannot be unlocked and remains inaccessible. Any other authentication technology may also be used.

In some embodiments, the device 44 is a first device and the user 40 may initiate a subsequent activation phase 2330 to allow access to personal data of the user 40 from a second device 144 having identifiers 146.

The subsequent activation phase 2330 is somewhat similar to the activation phase 2310. It is a phase wherein identifiers of the second device 144 are saved into servers 36 of the administrator system 30 and identified as being allowed to access the given one of the electronic records 42 of the database 32, in a manner which ensures that the user 40 is entitled to access the given one of the electronic records 42 of the database, and that the second device 144 is indeed owned by the user 40 and not by someone else. First, the user 40 must install the application 48 onto the second device 144. Then, as shown in FIG. 24B, the subsequent activation phase 2330 may comprise step 2419, which is preliminary identification, and step 2420, which is a transaction 150.

At step 2419, the user 40 is identified and information provided by the user is transmitted to the administrator system 20. As previously discussed, the application 48 may require that the user 40 authenticate prior to accessing the application 48. As such, in this embodiment, preliminary identification is simply accomplished by authenticating and accessing the application 48. In some embodiments, also, the application 48 may ask yet another similar authentication when the user 40 selects an option to activate a new device in the application of the first device 44.

At step 2420, the transaction 150 takes place, ensures that the device 44 is indeed owned by the user 40 and allows the second device 144 to send identifiers 146 to the servers 36 of the administrator system 30, which records the identifiers 146 and associates them to the electronic records 42. The transaction 150 is somewhat similar to the transaction 50 and involves an activating party 152, an offering party 154 and the servers 36 of the administrator system 30. [symbol] me cases, the activating party 152 may be the new device 144, the offering party may be a device [symbol] ed by the user 40, a device manipulated by the IVA, or a device that is also part of the administrator system 30, and the transaction 150 takes place in the same way as the transaction 50.

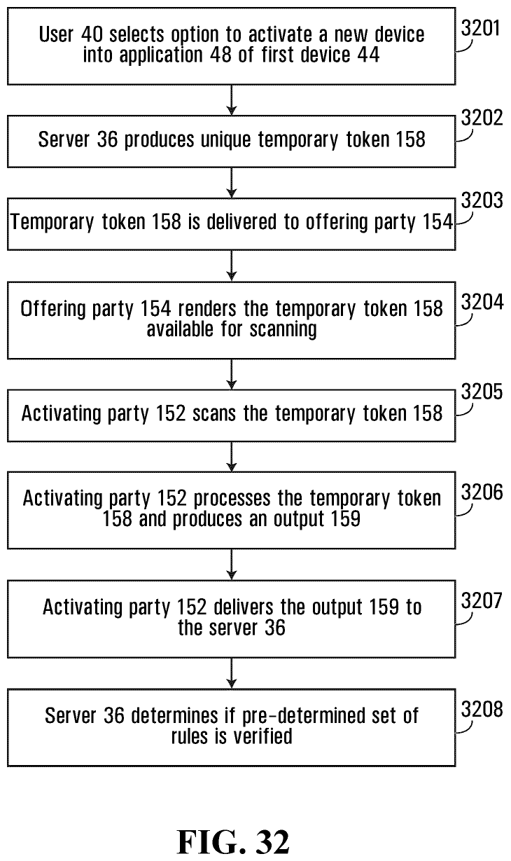

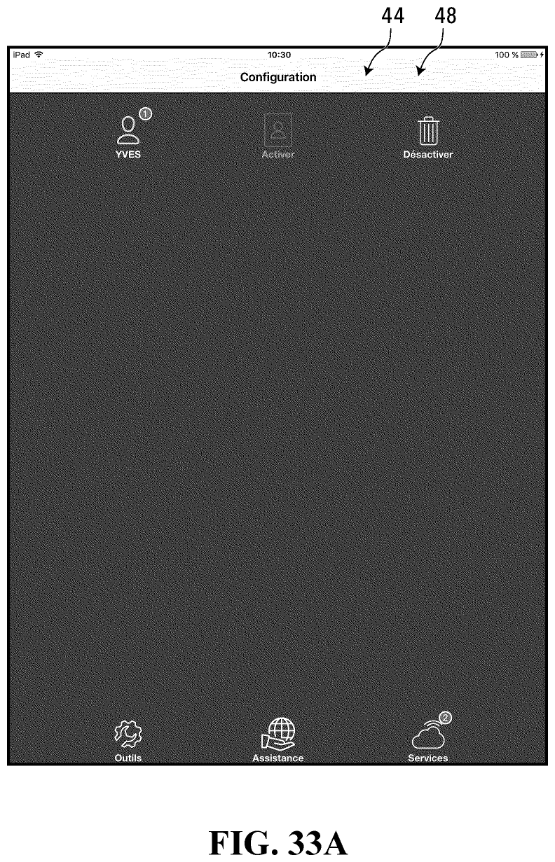

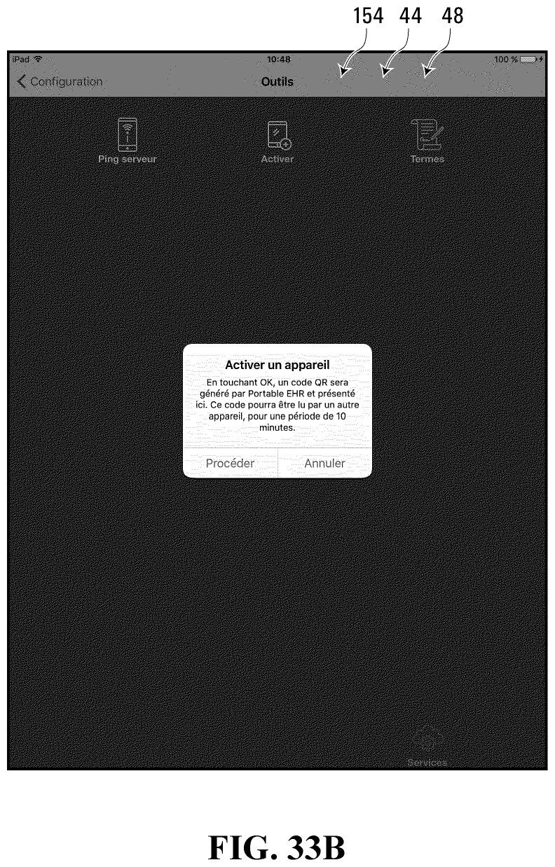

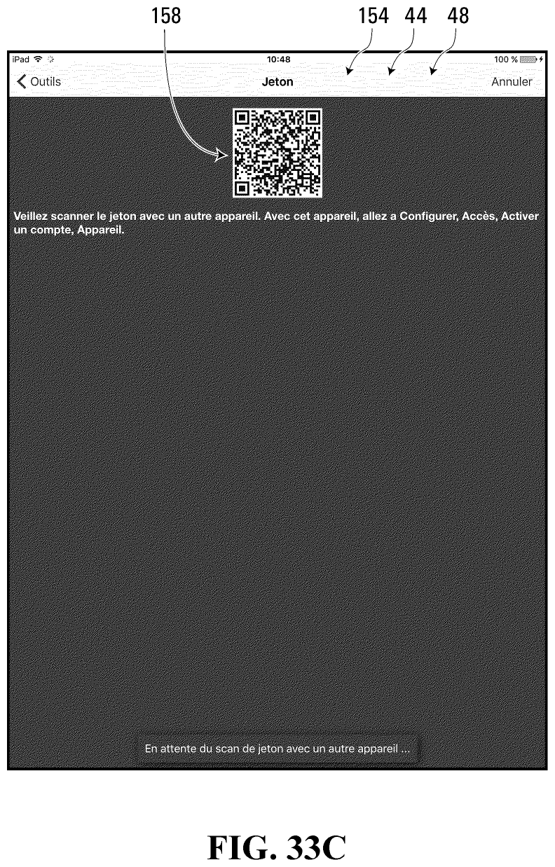

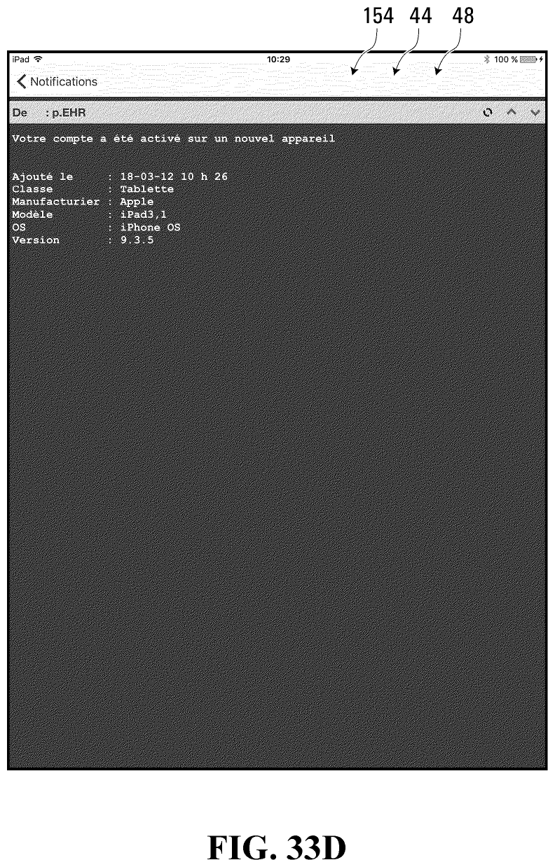

In this case, the activating party 152 is the new device 144 and the offering party 154 is the device 44. As shown in FIG. 32, the user 40 first initiates the transaction 150 by selecting the option to activate a new device 144 in the application of the first device 44, at step 3201. The offering party 154 then communicates with the server 36 of the administrator system 30 to initiate the transaction 150. The following steps of the transaction 150 are identical to steps of the transaction 50.

The transaction 150 may determine an outcome of the subsequent activation phase 2330. If the transaction 150 is completed, the server 36 activates the identifiers 146 of the new device 144, i.e., the server 36 registers the identifiers 146 as being authorized to access the given one of the electronic records 42 of the database 32. In this case, the activation phase 120 is a success. Otherwise, the subsequent activation phase 2330 is a failure.

In other cases, the transaction 150 alone does not determine the outcome of the activation phase 120. If the transaction 150 is completed, other steps still have to be completed to determine the outcome of the subsequent activation phase 2330. For instance, the activation phase 120 may further comprise step 321, which is a confirmation somewhat identical to the confirmation 60 but regarding the new device 144 instead of the device 44.

The above-described arrangement is effective from a data privacy and confidentiality perspective as it securely controls access to the data. For instance, the activation phase has the advantage of confirming the user identity by requiring a valid IP piece, such as a photo bearing government issued ID. The registration of the user device 44 at the server 36 links the user identity with a unique user device 44. In other words, since during the registration process the user must unlock the user device to perform the various operations such as scanning the QR code, the unlocking of the user device 44 by a user whose identity has been confirmed via the ID, demonstrates that the user device 44 is owned by the user and does not belong to someone else. In other words, it is not likely that the user will be able to register at the server 36 against the user ID, someone else's device.

During data access, the app running on the user device 44 requires user authentication to operate. In the case of a user device having a biometric authentication capability, the user needs to first unlock the user [symbol] ce 44, and then unlock the app such that a data access operation can be performed. Since the user [symbol] rd at the server 36 is associated with a unique identifier of the user device 44, the data access request is directed to the user record linked to the unique user device identifier.

Note that the device activation process and the general manner in which the user interacts with the device 44 and the server 36 is not limited to healthcare related data and can be used with any user-related information in the financial field and legal field, among others. Since no passwords are being used, it is difficult for an unauthorized third party to gain access to credentials sufficient to access the data. Accordingly, this implementation offers a secure data storage and access mechanism.

Managing Medical Data Access Over Interconnected Networks.

In previous examples of implementation, the Human-Centric EHR system stores the medical information of patients, such as lab results, imaging results etc. In other words, medical data, such as a lab result received from a testing lab is copied at the server of the Human-Centric EHR and retained there. If the patient accesses the Human-Centric EHR system to view the data, the patient is actually viewing the copy of the data stored locally in the server and not the data at the original source.

A possible disadvantage of that approach is the need to carefully preserve the confidentiality of the medical data that is being collected over time at the server of the Human-Centric EHR system. Medical data is very sensitive and to preserve it requires robust safeguards against hacking. That is costly and there is always some risk of a data breach.

Another challenge when accessing medical information distributed across different networks is that those networks often are not designed to interoperate. Each network has its own protocols, data structures and ways of classifying the information. Trying to make those networks work in a seamless fashion to provide a satisfactory user experience, where the patient can quickly and conveniently access the medical information of interest is difficult.

This example of implementation the invention illustrates a different approach where the data remains at the original source location and the Human-Centric EHR system is configured to respond to patient's requests to view document by managing access to the data at the original source location. In this fashion no sensitive data is stored at the server of the Human-Centric EHR, thus reducing significantly the risk of [symbol] sure of confidential user data. In addition, by limiting the interaction between networks at the level of [symbol] ss management of the data, the networks can be made to work together in order to provide one the one hand satisfactory patient access and on the other hand there is no need provide a deep level of integration. Each network can work as originally designed, and only a thin layer of interoperability is necessary for the information flow to occur in the intended fashion.

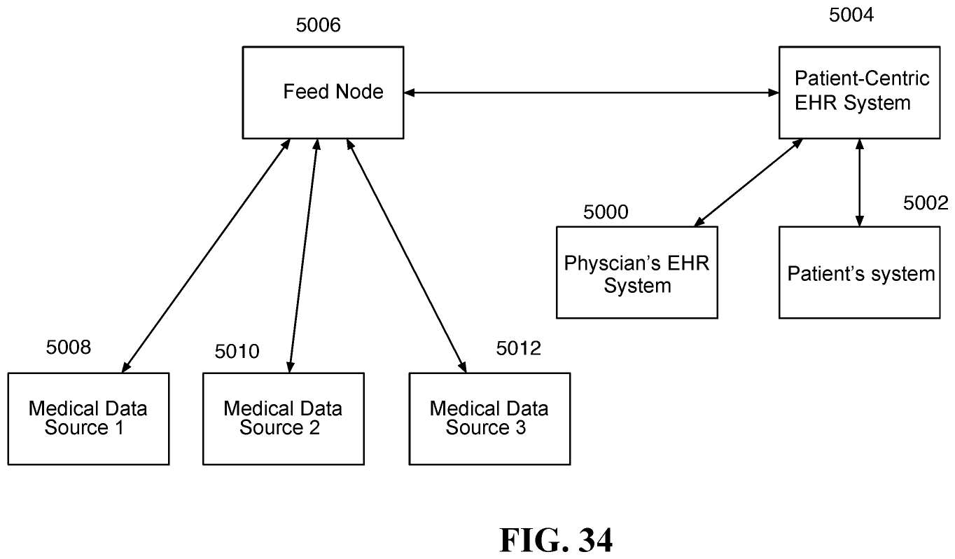

The architecture of the system is shown at FIG. 34. It includes the Human-Centric EHR system 5004 which is arranged largely in the same fashion as discussed in the earlier examples, with the exception of the new functionalities discussed below. The Human-Centric EHR system 5004 communicates with Patient's system 5002 which in most forms of implementation is mobile. The Human-Centric EHR system 5004 also communicates with the physician's EHR system 5000 to provide the functionalities generally described earlier.

The Human-Centric EHR system 5004 communicates with a range of medical data sources 5008, 5010 and 5012 which reside at respective nodes, via a Feed Node 5006. The medical data sources 5008, 5010 and 5012 are not part, strictly speaking of the Human-Centric EHR system 5004. Those medical data sources are independent networks in themselves or part of independent networks. So, while they can communicate with the Human-Centric EHR system 5004, they can also communicate and perform data transactions with a range of other entities that are unrelated to the Human-Centric EHR system 5004. Those entities are not shown in the drawings for clarity. Accordingly, the medical data sources 5008, 5010 and 5012 implement their own data protection and user authentication schemes. What this means is that if a patient wants to access medical data on medical data source 1 at 5008, the patient will access the server associated with the medical data source 1, for example via a web browser, authenticate himself through password or biometric features to gain access to the data. The same process would then need to be repeated at medical data source 2 at 5010 and medical data source 3 at 5012 that hold other medical data for the patient. This is inconvenient for the patient as he needs to remember different passwords and the person needs to perform the access process multiple times to get access to multiple documents, not to mention the possible confusion in terms of where different documents reside.

In a typical implementation, medical source 1 at 5008, medical source 2 at 5010 and medical source 3 at 5012 are associated with entities that generate medical information or more generally process medical information, such as a testing lab, a radiology or other imaging service lab, a hospital and a drug dispensing entity, such as a pharmacy, among others. Accordingly, a patient that is prescribed a blood [symbol] by a physician would go to the testing facility of the lab where a blood sample is taken, the blood [symbol] le processed, and results generated and entered into the database of the lab.

The results are communicated to the Human-Centric EHR system 5004 by the medical data source, say source 1 at 5008 through the Feed Node 5006 that has a functionality to enable the patient to look at a future time, at the medical data without the need to directly access and authenticate himself with a password or biometric feature a the medical data source 1 at 5008. In practice, the Feed Node 5006 can be configured as a server that is remote of the nodes implementing the medical data source 1 at 5008, the medical data source 2 at 5010 and the medical data source 3 at 5012. In this instance, each of the nodes implementing the medical data source 1 at 5008, the medical data source 2 at 5010 and the medical data source 3 at 5012 communicate with the Feed Node 5006 over communication links and via standard communication protocols. In this form of implementation, the services at the Feed Node 5006 exist as a number of different instances, each instance being associated with a given medical data source.

Alternatively, the Feed Node 5006 can be implemented as software that executes on the server of each medical data source. This approach is preferred from the perspective of cost as there is no need to install a separate hardware but may be more complex to implement as it requires installing software on a range of different medical data source nodes.

In yet another form of implementation, the Feed Node 5006 can be part of the Human-Centric EHR system 5004, in other words the software providing the functionality of the Feed Node 5006 is part of the overall software package at the server implementing the Human-Centric EHR system 5004. In such example, the medical data source nodes communicate directly with the server implementing the Human-Centric EHR system 5004.

The role of the Feed Node 5006, in each form of implementation is to maintain a trusted relation with the respective medical data sources, such that requests to access medical information channeled through the Feed Node 5006 are deemed legitimate and no interaction with the patient is required to authenticate the request for the medical data. In this fashion the patient, only need to authenticate himself to the Human-Centric EHR system 5004 by the mechanisms discussed earlier in this disclosure.

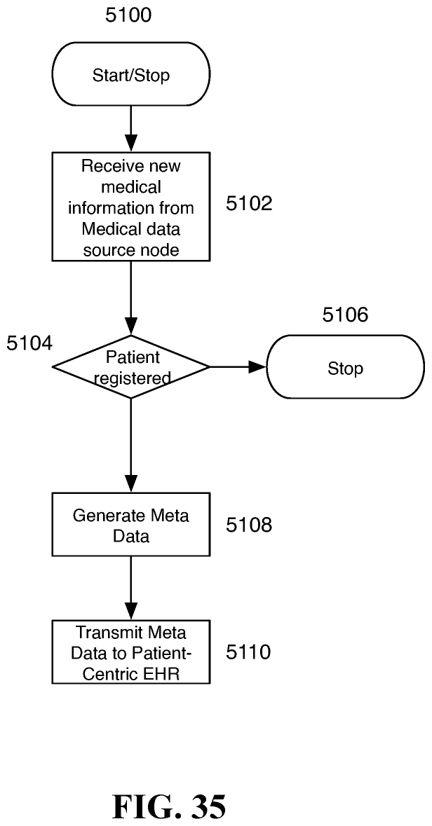

FIG. 35 is a flowchart illustrating the operation of the Feed Node 5006 when new medical data is generated at the medical data source 1 5008, with the understanding that the same process is performed in connection with the other medical data sources. [symbol] process starts at step S100. At step S102 the Feed Node 5006 receives metadata indicating that new medical information is produced at the data source. The metadata would typically include an identifier of the patient to whom the medical data pertains. The identifier can be a name of the patient and/or other identification information such as to uniquely identify the individual. More generally, the metadata can include a range of information categories. The following are examples:
1. An identifier of the message. The identifier can be any suitable alphanumerical string that uniquely identifies the message among other messages.
2. The identifier of the patient, such as the patient name or any identifier that can be mapped to the patient.
3. The dispatch profile of the message. The dispatch profile indicates how the message should be sent to the patient and/or whether the message is urgent or not urgent or any other urgency related categorization. For example, if the message relates to medical information that denotes an urgent condition, such as abnormal results, then the dispatch profile will contain this information.
4. The identifier of the practitioner. The practitioner can be the treating physician of the patient or the physician that has requested the particular test or procedure that is associated with the metadata.
5. A mechanism to trigger an answer from the patient, such as agreement or consent to enroll the patient in a clinical research trial or some follow-up procedure that stems from medical information being communicated to the patient. For example, the patient may indicate that he or she wishes to receive medical news about the condition associated with the medical information or advertisement on products or services associated with the medical information. The answer from the patient is retained at the Human-Centric EHR system 5004 once the patient responds to the inquiry
6. Data that defines the process for accessing the medical information. When a user wants to access the medical information, as it will be described in greater detail below, a request for access will invoke this process that will enable the user to see the medical information.

Advantageously, the metadata holds no personal private medical information. For example, in the case of a blood test, no test values are conveyed by the metadata. This approach protects the confidentiality of the patient information which is accessible only at the location pointed to by the metadata. [symbol] tep 5104 the Feed Node 5006 will query the Human-Centric EHR 5004 to determine if the patient is [symbol] tered by, for example, by searching in the list or registered users the patient name. If no match is round, in other words, the patient for whom medical results are available is not a subscriber of the Human-Centric EHR system 5004, the process terminates at 5106.

However, if a match is identified in the list of registered users the Feed Node 5006 will generate the metadata associated with the medical information to enable the patient to access it via the patient's system 5002 and the Human-Centric EHR system 5004.

Typically, the identifier of the message that is conveyed by the metadata maps to a location which can be a physical one or a logical one, where the medical data resides such that it can be retrieved at a future time. Typically, the location of the medical data would be supplied by the medical data source node when the later communicates with the Feed Node 5006 to submit the medical data.

The metadata can be supplied by the medical data source node when it communicates with the Feed Node 5006. Alternatively, the Feed Node 5006 can generate some elements of the metadata. One possibility is to provide the Feed Node with logic to parse medical data and identify its nature.

The metadata thus generated is stored at the Feed Node 5006 location. Alternatively, it can be transmitted to the medical data source node. In such case, nothing is stored on the Feed Node 5006 that acts as a content-based router. In addition, the metadata is sent to the Human-Centric EHR system 5004, where it can be used to update the patient record such that the patient can access the medical data. In addition to the metadata, the transmission would include an identifier of the patient to enable the Human-Centric EHR system 5004 to match the metadata to a subscribing patient.

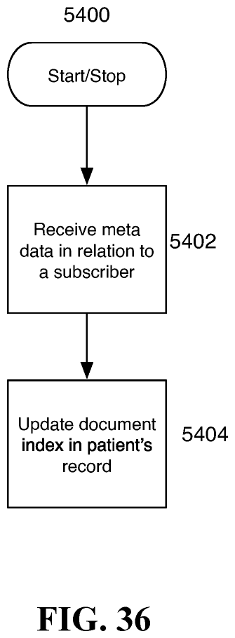

The process at the Human-Centric EHR 5004 is illustrated by the flowchart at FIG. 36. The process starts at 5400. At step S402, the Human-Centric EHR 5004 receives the metadata in connection with a medical data from the Feed Node 5006. At step S404, the Human-Centric EHR will identify on the basis of the patient identifier the record of the subscribing patient to which it belongs and record it there. The recording operation preferably includes writing the metadata or elements thereof in an index such as to constitute over time a summary medical record identifying the health care service events dispensed to the patient for which a document exists and can be consulted. The index can be developed by logic at the Human-Centric EHR system 5004 to identify from the metadata the nature of the service event or the medical information and put that item in an index, thus allowing the patient or a service provider to [symbol] kly access the information of interest. For instance, the index may be constructed by grouping the [symbol] ical information in classes or categories such as “blood tests”, “imaging results”, etc., the individual entries being classified by date, whether the results are normal or any other suitable factor. For convenience, individual entries can appear to the user over a GUI as hyperlinks or other suitable GUI controls. When the user clicks on a hyperlink the process for displaying the medical data identified by the metadata is triggered, as discussed below.

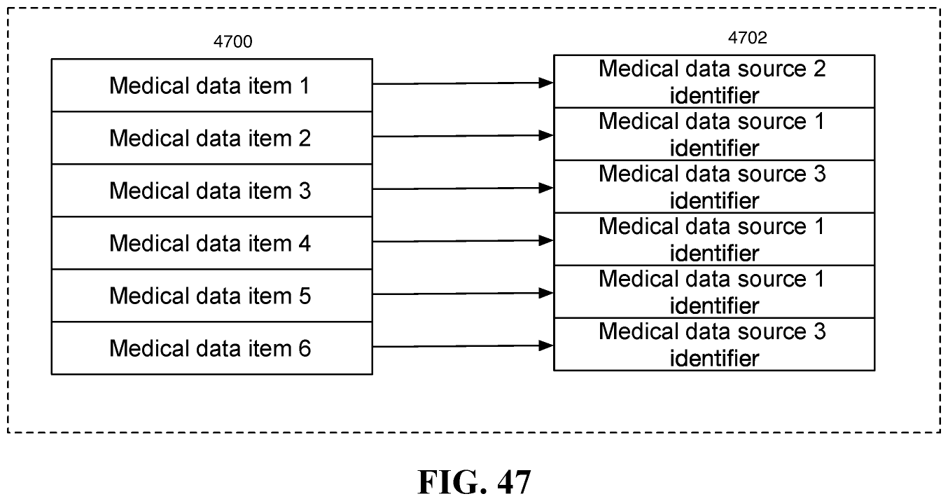

FIG. 47 illustrates a data structure mapping the identifiers of the medical information across the different networks such that the medical information can be accessed. The index or summary of the medical data that the patient sees is depicted at 4700. In the drawing, it is presented as a simple list of medical data items, such as blood tests, imaging results and others, but the organization of the information may be more complex, including a categorization of the information in different classes, as discussed above. What the patient would see on the screen of the mobile is then a list of medical data items, each item corresponding to medical data that is stored at either one of the medical data sources 1, 2 or 3. As indicated previously, the medical data item can be in the form of a visual object that hides the process necessary to pull the medical content residing at the respective medical source. When the patient activates the visual object the process of retrieving the information is performed. The data structure maps each medical data item to a corresponding identifier that indicates where the medical data resides on an external node (data source). For example, the medical data item 1 points to a medical data source 2 identifier. The identifier is extracted from the metadata that was originally sent from the data source node 2, when that data source node 2 dispatched the metadata to the Feed Node 5006 and eventually to the Human-Centric EHR system 5004. So, the identifier stored at the Human-Centric EHR system 5004 allows to reconstruct the location of the source of the data such that it can be retrieved and displayed to the patient.

The table of the identifiers 4702 is dynamically updated as messages are received from the respective data source nodes to notify the Human-Centric EHR system 5006 that new medical information for the patient exists. As a new message is received and it is processed as discussed to extract the identifier of the data location from the metadata, an entry in the index 4700 is created and at the same time an entry in the identifier table 4702 is also created.

In this example, it should be pointed out that no medical data is stored on the Human-Centric EHR system 5004. Only metadata is stored, which in itself conveys no meaningful medical information and in the event of a breach even if the metadata is leaked outside the Human-Centric EHR system 5004, it does not [symbol] se any sensitive information about a patient. Specifically, neither the index 4700 nor the identifier [symbol] 4702 contains personal medical information. The personal medical information resides at the respective medical data source nodes 1, 2 and 3.

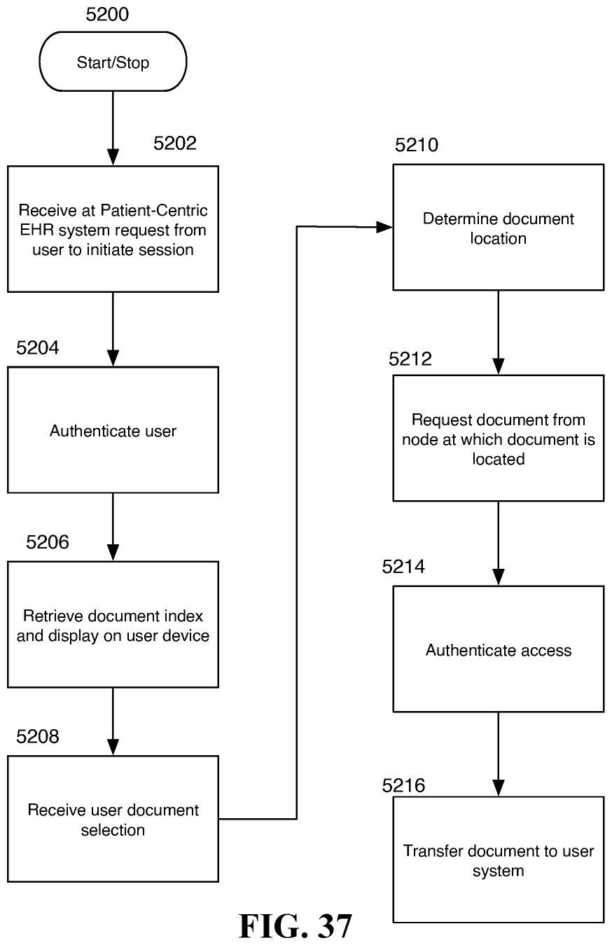

FIG. 37 illustrates the process for a patient or a physician to access medical data. The process starts at 5200. At 5204 the Human-Centric EHR system 5004 receives from the patient system 5002 a request to initiate a session. Typically, if the patient's system 5002 is implemented on a mobile, such as an App, for example, by activating the App on the mobile, a communication is triggered with the server implementing the Human-Centric EHR system 5004, to which the server will respond by an authentication request.

The user authentication is performed at step S204. This is accomplished by the user entering a username and a password combination or a biometric feature such as a fingerprint or face recognition. If the user credentials are recognized by the Human-Centric EHR system 5004, the user profile is loaded, and the GUI would show the metadata-based index of documents that are available for the user to view. This is performed at step S206.

Assume for the purpose of this example that the user selects a document in the list, at step S208. The source of the medical data associated with that selection is retrieved from the user profile at step S210 and the node at which the document is located is determined. As discussed previously, the metadata initially received from the medical data source node defines not only the location of the document at the particular medical data source node, but the location of the document within the entire network, including the location of the node itself. More specifically, the process will extract from the data structure at FIG. 47 the identifier mapped to the user selection and build a medical data read request from a source specified in the data structure.

At step S214, the Human-Centric EHR system 5004 then communicates with the medical data source node by supplying back the data identifier to identify the document requested, though the Feed Node 5006. Since the medical data source node can establish the identity of the requester (Feed Node 5006 or Human-Centric EHR system 5004, that are known to be genuine requesters, the medical data source node will then retrieve the document, render an image of it and the rendered image is then transmitted back through the Feed Node 5006, the Human-Centric EHR system 5004 and ultimately the patient's mobile 5002, where it appears on the screen. Accordingly, the patient can consult the document but there is no actual storage or retention of the medical data at either one of the Feed Node 5006, the Human-Centric [symbol] system 5004 or the Patient's system 5002. So, when the communication session is completed, [symbol] een the Human-Centric EHR server 5004 and the patient's system 5002, no data is retained.

Patient Data Access Management

This section describes additional examples of functionalities allowing a user to control with fine granularity access to medical data in the health record.

From the perspective of the patient, it is important to have control over the privacy of the medical data that is either stored (and retained) in the Human-Centric EHR system or the data that is stored at remote nodes and that is accessed through the Human-Centric EHR system. Typically, the medical information in the Human-Centric EHR system is intended to be consulted by health care professionals to enable them to dispense health care services. It is the practice today to have the entirety of the EHR patient file made available to the health care professional. This is for example, the case of the Dossier Santé Quebec (DSQ). While such an approach has some merit in certain circumstances, such as when the patient is admitted for urgent care at a facility that has no previous record of his or her condition, where it would be useful for the physician to get access to all information in order to be able to make a diagnosis, those instances are rare. In most cases, a physician or any other health care service provider needs less than the entirety of the medical information to dispense high quality services.

Further, the medical information stored or available through the Human-Centric EHR system is very sensitive. The patient may not want one or more health care service professionals to know certain details that are not required for those health care service professionals to do their work. For example, the patient may have been diagnosed with a Sexually Transmitted Disease (STD) and that diagnosis may be embarrassing and something which the patient would like to keep confidential and share only on a need-to-know basis. Accordingly, a mechanism allowing the patient to control with fine granularity who has access to the sensitive medical data, which the present example provides, would be highly useful.

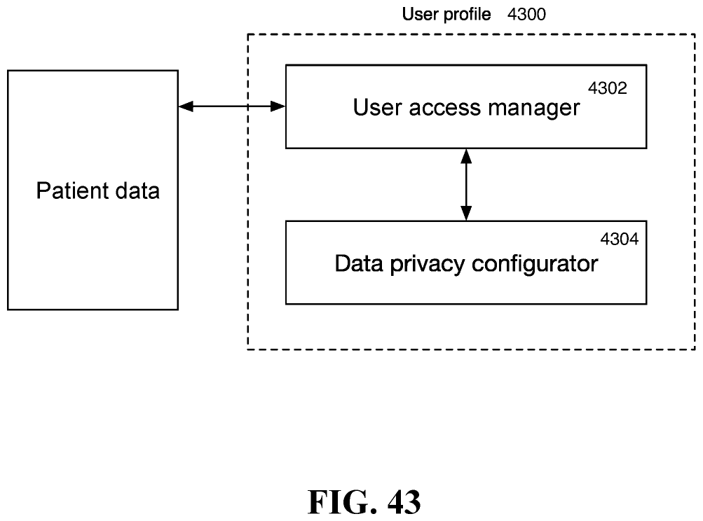

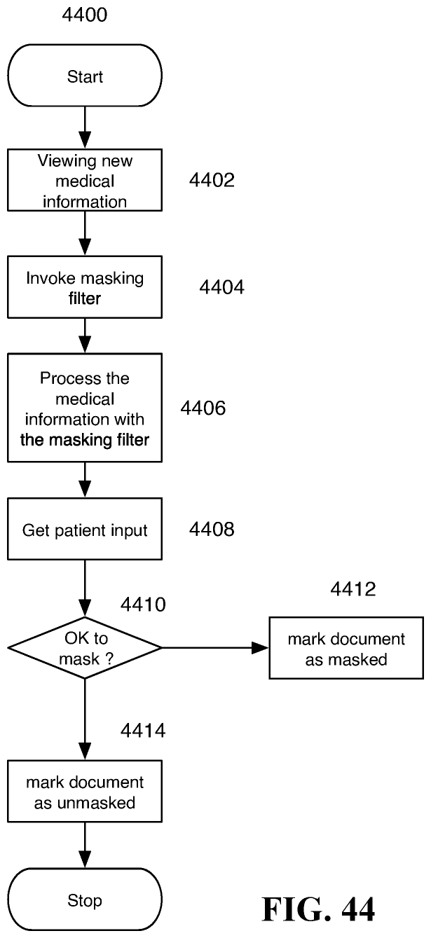

The example of implementation of the invention shown at FIG. 43 is designed to provide this functionality. That figure is a block diagram illustrating some functional blocks of the Human-Centric EHR system, which could be the Human-Centric EHR system 102. Each patient that subscribes to the in the Human-Centric EHR system 102 has a user profile that stores functions and configuration information specific to that patient. The functions and configurations determine how the patient data is accessed or pushed to third parties. [symbol] user profile 4300 has two functions, among others, namely a user access manager 4302 and a data [symbol] acy configurator 4304. The user access manager 4302 holds a list of the individuals that can access medical information about the patient himself and possible dependents, for instance children of individuals for whom the patient is the legal guardian. The data privacy configurator 4304 determines, who among the registered users in the user access manager 4302 has access to what. The combination of the user access manager 4302 and data privacy configurator 4304 form a gateway to the patient data, such that only the information that the patient has agreed to share is actually being shared.

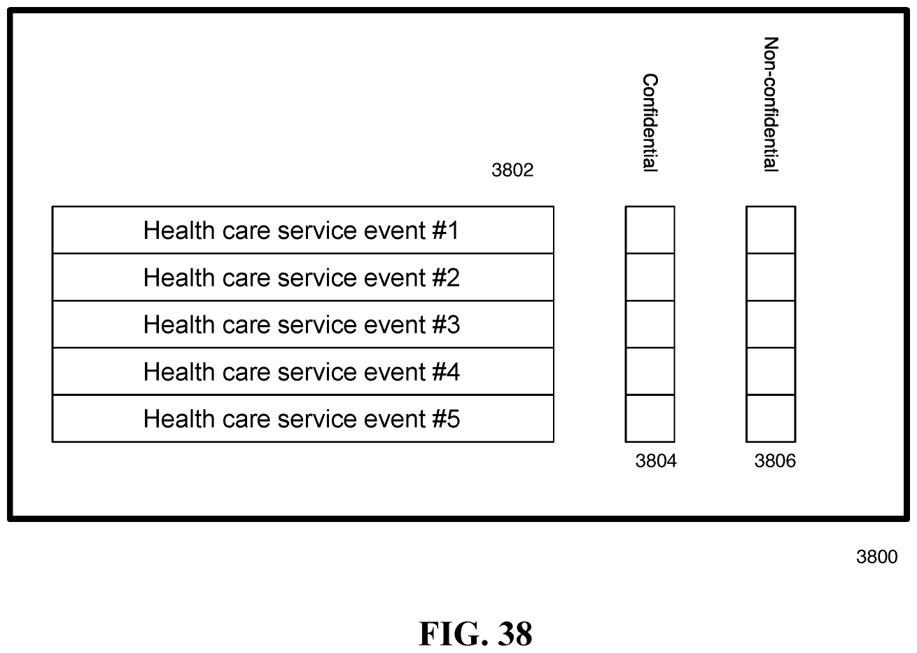

The patient interfaces and controls the data privacy configurator 4304 through a Graphical User Interface (GUI) which has controls allowing the patient to determine how his medical information is accessed. An example of a GUI and the attendant controls is depicted in FIG. 38. The GUI 3800 has a list of health care service events 3802, that reflect the different interactions the patient had with health care service providers. For example, an item in this list could be a blood test result, an imaging procedure, a drug prescription and a surgery procedure among many others.

Beside the list appear two rows of controls, such as check boxes, organized in two groups, namely a confidential group 3804 and a non-confidential group 3806. This allows the patient to designate each health care service event as either a confidential one or a non-confidential one. Accordingly, another user, such as a physician that accesses the medical information will only be allowed to see health care service events that are designated non-confidential.

Objectively, this form of data privacy management requires significant involvement from the patient as the latter needs to update the data privacy configurator 4304 every time a new health care service event is generated. At the same time, it provides a lot of granularity as each health care service event can be individually managed from a confidentiality perspective.

It should also be understood that the above applies to individuals accessing the medical information other than the patient. The patient has full access rights all the time.

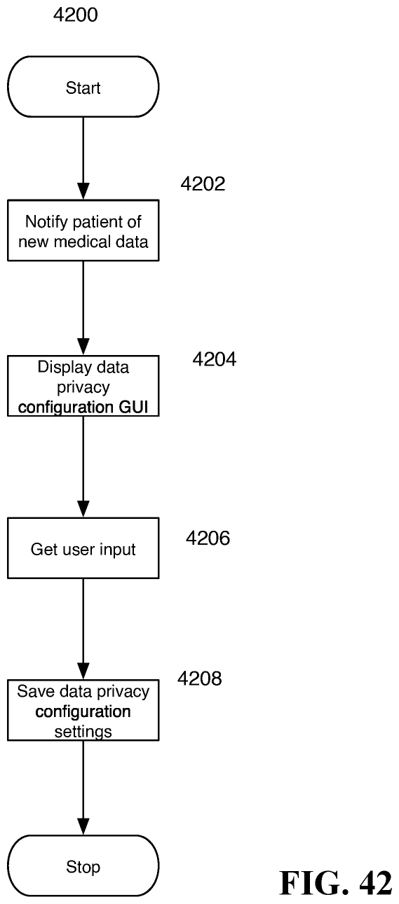

One way to simplify the data privacy management is to invoke the GUI at FIG. 38 every time a new health care service event is recorded in and the patient is notified of that new health care service event. The flowchart of this operation is shown at FIG. 42. The process begins at 4200. At step 4202, the patient is notified of new medical data, as it was previously described, for example by generating a notification on the patient mobile, which requires the patient to open the app and consult the medical data. [symbol] he patient interacts with the app, the GUI at FIG. 38 is invoked (step 4204) and the health care [symbol] ice event highlighted required user input to designate it as confidential or non-confidential, at step 4206.

At step 4208 the new data privacy settings regarding this health care service event are recorded into the data privacy configurator 4304.

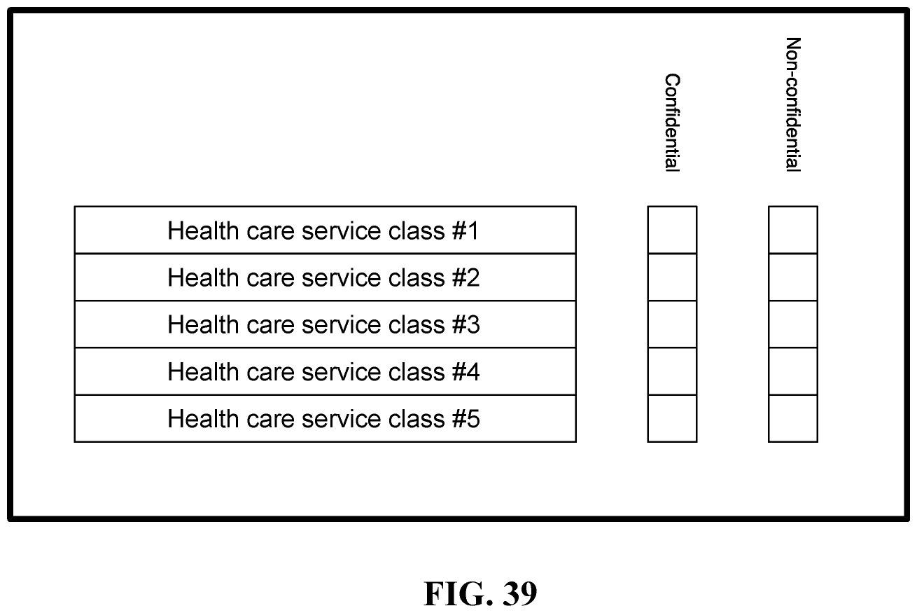

An alternative to the approach described earlier is to group the individual health care service events into classes and designate each class as confidential or a non-confidential one. This option is shown at FIG. 39. Instead of listing individual health care service events, the GUI shows health care service classes that define groupings in which the individual health care service events are classified. In this example, the patient can designate via check boxes whether a particular class is confidential or non-confidential, in which case a user that accesses the medical data will see all the health care service events that are grouped in the particular class.

Once the patient sets his or her preferences in the checkboxes of the GUI at FIG. 39, those settings are stored in the data privacy configurator 4304.

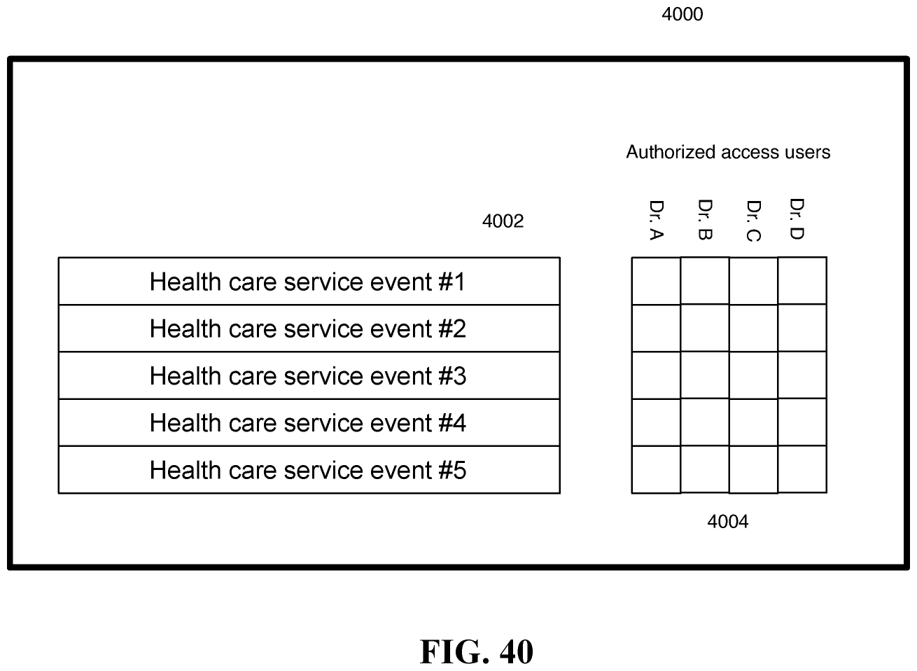

A further refinement of the above approach to data privacy management is shown at FIG. 40. In this example, additional granularity is provided to tailor individual user access rights. In other words, some users may have rights more expanded than others.

The GUI 4000 displays a list of the individual health care service events at 4002 and a list of controls 4004 associated with respective users, which in this example are physicians. The controls 4004, such as check boxes allow the patient to select whether to enable or not enable a particular user to access a particular health care service event. So, if there are certain health care service events that the patient does not want a particular physician to see, the patient can configure that through the controls 4004.

It is to be understood that the authorized access users list is dynamically updated when the list of users in the user access manager 4302 changes. When a new user is added in the user access manager 4302, that change would reflect itself at the GUI 4000.

Since this example manages the data privacy on the basis of individual health care service events, the process described by the flowchart at FIG. 42 can also be followed every time a new health care service [symbol] it is generated. As the patient interacts with the app on his or her profile to see the information [symbol] ciated with the health care service event, the GUI at FIG. 40 is invoked allowing the patient to specify which authorized access user is enabled to see the medical data associated with the health care service event.

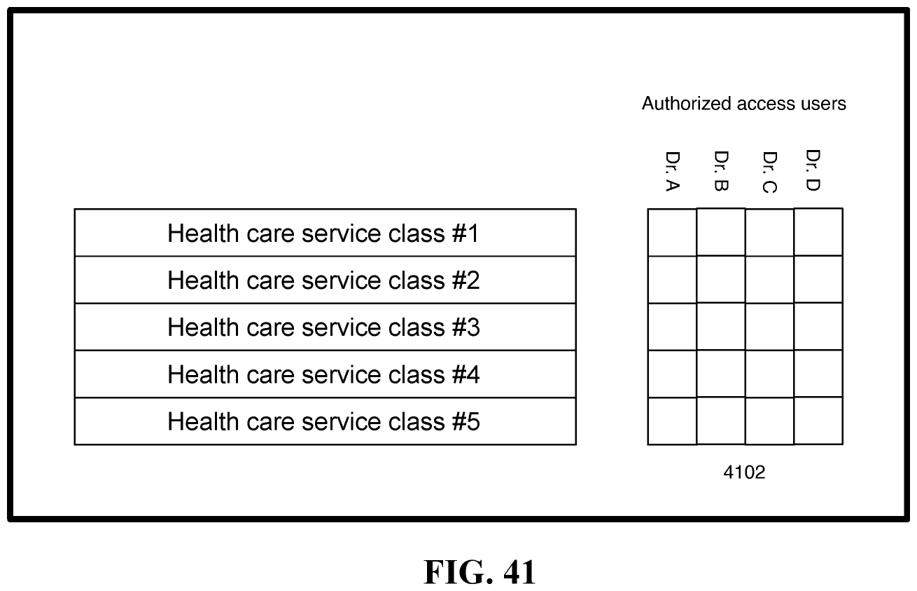

FIG. 41 is another variant where permissions for individual users are granted on a health care service class basis. The GUI at FIG. 41 shows the existing classes of health care service events, allowing through the control group 4102 to indicate which user gets access to which class, with the understanding that once access to a given class is allowed, the user can access each health care service event stored in that particular class.

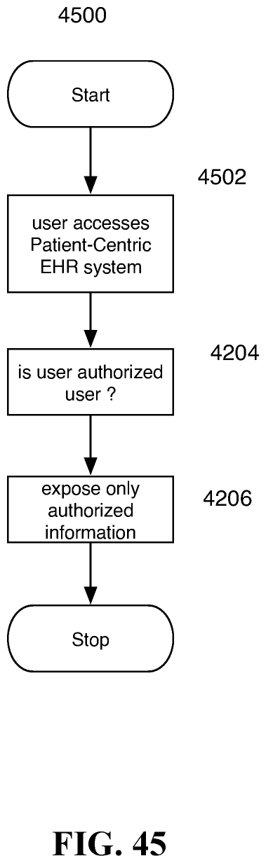

FIG. 45 is a flowchart of the process allowing a user to access the medical information of the patient. This process applies to the example of implementation shown in FIG. 38. The process starts at step 4500. At step 4502 the user logs into the Human-Centric EHR system which requires user verification. While not shown in the flowchart, there could an additional step where the user would designate which file or record the user wants to access. Typically, that would be the case for a physician that treats a number of individual patients having medical information available via the Human-Centric EHR system. So, at that step, the authorized user would identify the patient whose medical data the user wants to access.

At step 4504 the Human-Centric EHR system determines if the user is an authorized user for the particular patient. If there is a match, the user is deemed authorized and access is given according to the data privacy configuration.

At step 4506, the medical data for the patient is modulated according to the data privacy settings and only the information that the patient has authorized is exposed to the user. The information that the patient does not want to expose is not made available to the user. In particular, in the case of the GUIs at FIGS. 38 and 39 the user would be able to see any information that is identified as being non-confidential. In that embodiment, all users that are authorized have the same privileges and see all the information that is designated as being non-confidential. In the embodiments of FIGS. 40 and 41, the filtering operation at step 4506 is performed differently in that it is based on the identity of the authorized user and that user is enabled to see only the information the patient has authorized as far as that user is concerned. [symbol] further control over the access of the information, the user profile can include a log function that [symbol] rds which authorized user has accessed what element of information, along with the date and time. This function provides an additional assurance to the patient and shows what in actuality is happening in terms of data access, in addition to confirming that the data privacy settings work as intended. So, if the patient data contains something that the patient definitely does not want one or more authorized users to know about, the patient can at any time by looking at the log confirm that those users have not accessed that information.

Instead of users taking positive action to access the medical information in the file or record, a push procedure can be implemented when new medical information is available that would send a notification or the medical information itself to all the authorized users that should be informed of the event. Such push procedure is more practical, and it is a proactive way to ensure that treating physicians are kept up to date about patient condition.

The push procedure can send a notification that new medical data is available, where the medical data itself is obfuscated and requires the user to log into the Human-Centric EHR system in order to be able to access the data. Alternatively, the push procedure can be a mechanism to notify the authorized user about the new medical information and provide an access to the information in a facilitated fashion by comparison to the normal access route. An example of facilitation would be to provide a direct link in the notification that the user can invoke to access the medical information.

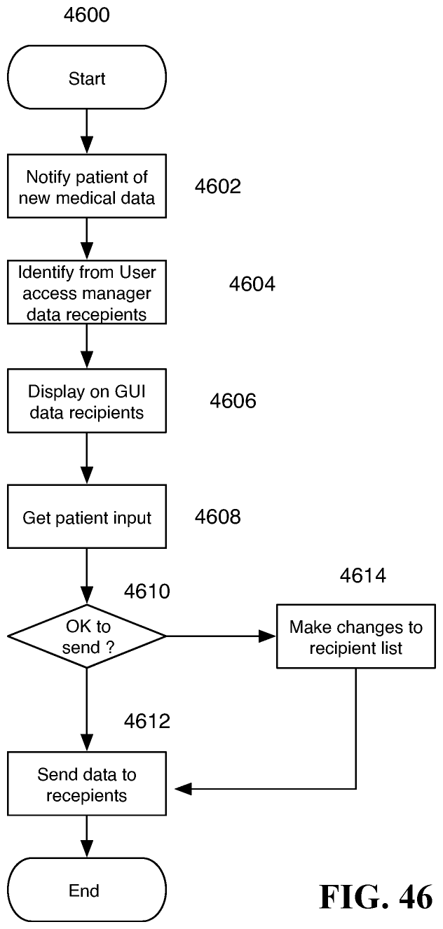

The flowchart at FIG. 46 illustrates the push process in greater detail. The process starts at 4600. At step 4602, the Human-Centric EHR system notifies the patient over the mobile that new medical data is available. As discussed previously, a notification can be sent to the mobile which displays a banner on the display screen indicating that the app requires the attention of the patient. When the patient opens the app and authenticates himself/herself, the patient can then view the new medical information by interacting with the Human-Centric EHR system.

At step 4604, the user profile 4300 extracts from the user access manager 4302 and the data privacy configurator a list of recipients for the notification about the new medical data. The list is built on the basis of the access authorizations that have been previously specified by the patient.

At step 4606, the list of recipients is sent to the patient mobile and displayed on the GUI. This step is optional but preferred because it informs the patient who is about to get the new medical information. [symbol] tep 4608 the patient provides input via the GUI. That input can be a simple confirmation or can be a request to change the list of recipients or to cancel the dispatch. If at decision step 4610 the patient input is to authorize the dispatch, then at step 4612 the Human-Centric EHR system performs it. However, if the patient wants to make changes to the recipient list or cancel the dispatch, that action is carried out at step 4614.

An alternative approach to data privacy management is to use logic that masks or unmasks parts of the medical file on the basis of certain themes, such as for example, nature of the medical information, a time window and locations of healthcare dispensing events, among others. This is achieved by using a filter that provides a certain options to the patient in terms of masking themes and based on the patient selection, the medical data of the patient is processed and placed into a visible category or a non-visible category.

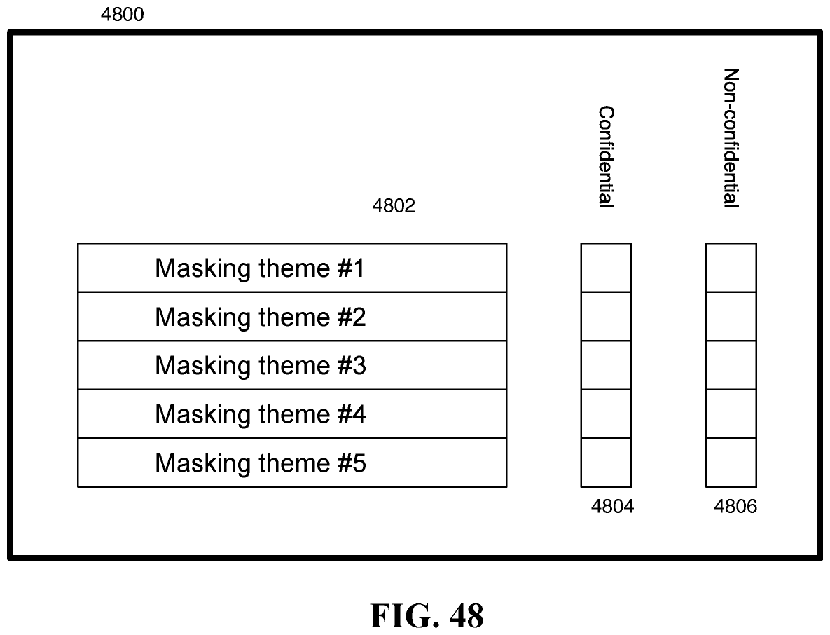

FIG. 48 illustrates the user interface, similar to the user interfaces shown at FIGS. 38 to 41, where the patient can selectively mask information on the basis of certain themes. In this example, the patient selects a particular theme and indicates if the information is confidential or non-confidential. A possible refinement is to provide a more granular specification for whom the mask is effective, such as in FIGS. 40 and 41. For instance if the patient wants to mask information based on theme #1, the patient can specify if the specific authorized user for whom the mask is effective, such as Dr. A, Dr. B, Dr. C or Dr. D or particular care group.

Each theme is defined by parameters. Those parameters identify the medical information to be masked. The identification of what is to be masked and what is not to be masked is performed by processing the medical information through the theme filter.

Examples of themes include:
1) Particular diseases or conditions that the patient wants to mask. For instance, those can be sexually transmitted diseases or screening thereof, psychological diagnoses or treatments and drug use, among others.
2) Time window or time limit during which health care services have been dispensed to the patient.
3) Particular physicians or other health care service providers that have dispensed health care services to the patient.
4) Particular institutions, such as hospitals or clinics, that have dispensed health care services to the patient.
5) Countries or more generally geographic locations where health care services have been dispensed to the patient.
6) Prescribed drugs to treat certain diseases.
7) Clinical research protocols in which the patient was involved.
8) A care group theme.

As indicated earlier, each theme is defined by a set of parameters, which is programmable. The parameters map data items in the medical information of the patient to the theme. The medical data items are indicators or signs that the particular theme exists in the patient medical history. For example, when the patient wishes to mask sexually transmitted diseases, the set of parameters are designed to catch any indicator in the medical history about a sexually transmitted disease. For example, the set of parameters would track diagnoses for STDs, tests for STDs such as test requests and/or tests results and prescribed medication that is used to treat STDs, among others. When a trace of an STD related information is identified, the document containing that trace is masked.

For STDs the tracking can be performed by using a key word search to scan the medical history for terms that are STD related. Prescribed medication is also scanned for drugs that are usually prescribed to threat STDs. Note that since the medical information does not reside on the server of the Human-Centric EHR system 5004, the filter cannot scan the full medical data. Advantageously, the filter is invoked every time the patient or an authorized user will view medical data stored at a remote location. At that time, the data is available and the filter, according to the settings in the patient profile is run. If a certain document is found to contain STD related information that document is marked as confidential or to be masked in the patient profile. Each disease or condition to be masked will have its own set of parameters. When the filter in connection with a particular disease or condition is activated by the patient, the medical information being read or viewed by the patient is screened by each filter to determine if the information is to be masked or not.

A theme such as a time window or time limit are simpler to implement as they operate on the basis of dates. In this case, the patient will indicate the time window or the date limit and every medical information document that is within those date specifications will be masked. [symbol] cular physicians and institutions theme would involve a key word search in the medical information [symbol] entify the documents that may be relevant and mark those as being masked. The country theme can be handled in the same fashion, in other words through a keyword search.

The prescribed drugs theme can be done through a keyword search to identify specific drugs that have been prescribed and that are to be masked. The clinical research protocol theme is handled in the same manner.

The process for the operation of the filter to identify which document will be masked is illustrated by the flowchart of FIG. 44. The process starts at 4400. At step 4402 the patient receives a notification that new medical information is available at a remote node and views the information as per the process described earlier. At step 4404, the masking filter is invoked. This implies processing the medical information that has been sent from the remote node through the parameters of the themes that the patient has activated or enabled via the user interface, as performed at step 4406. The output at step 4406 is list of documents, or items of information in individual documents that constitute traces of certain themes that the patient would like to mask.

At step 4408, the list of the documents to be masked or items of information to be masked are shown or highlighted to the patient via the GUI, such that the patient can approve the selections. If the output of decision step 4410 is “yes” the patient profile is modified to indicate that the documents or items of documents are to be masked (step 4412), otherwise the document remains unmasked (step 4414).

Consent Management

To allow entities that are not involved into providing direct health care services to the patient to access the medical information of the patient, a consent from the patient may be required. In a first example, medical data can be useful for research purposes and research institutions would want to collect medical information from many individuals in order to do analytics to find trends, etc. In this case, the medical data collected from patients would be stripped of nominative information and other non-biological or non-medical information about the individual leaving only medical information and/or general socio-economic information that cannot be traced back to a particular individual. Such de-identified data is then pooled, and analytics can be run on it. [symbol] second example, it may be desirable to perform analytics on the medical data for a particular individual to provide value added advices to that individual, such as a tailored diet or tailored supplements, exercise regimen etc. In this case, the analytics are performed over the medical information of a single individual to produce focused recommendations such as by using Artificial Intelligence (AI). Typically, the analytics are performed by a service that is external to the entity holding the medical data of the patient. Accordingly, for the analytics to be run, the medical data must be exported to the entity, which requires the consent of the patient.

In those two scenarios, among others, patient consent is not a trivial process and requires some degree of management. For example, the consent may be time limited and account for the fact that a patient may change his mind about granting access to an external entity to his or her medical information. In other words, it would be desirable to allow the patient to revoke the consent. Also, the consent should be able to define boundaries about the medical information the patient is granting access over. For instance, the patient may not be willing to share certain medical conditions or events, even in instances where the data being shared is anonymized.

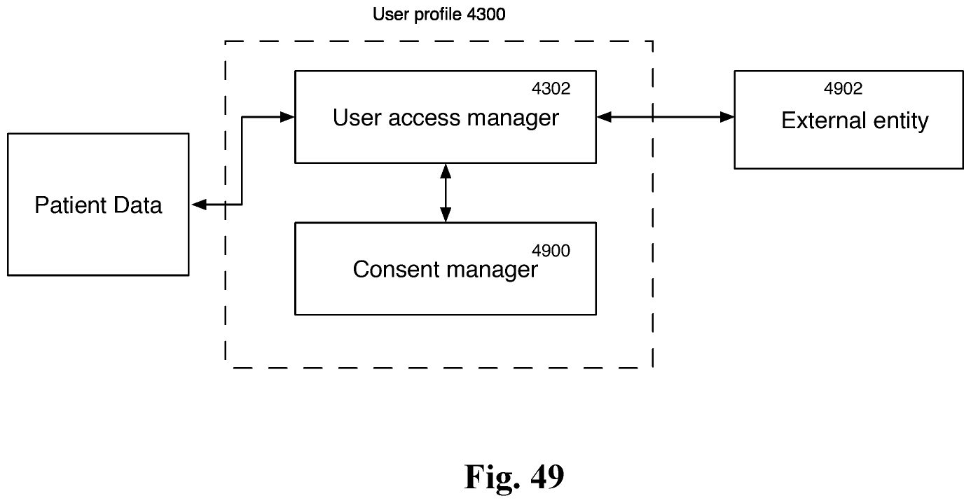

The Human-Centric EHR system that implements the consent management functionality is arranged largely in the same fashion as discussed in the earlier examples, with the exception of the functions shown in FIGS. 49 to 63. With specific reference to FIG. 49 which is a block diagram of the user profile 4300 residing on the server of the EHR system, shows that the user profile 4300 communicates with a patient data repository that is the source of the medical information of the user/patient. The user profile shown in FIG. 49 has two functional modules, among others (not shown) namely a user access manager 4302 and a consent manager 4900. The consent manager provides a series of services focused on consent management to the user access manager. An instance where the consent manager 4900 is invoked is one where an external entity 4902 communicates with the user access manager 4302 to gain access to patient medical data. In that instance, the consent manager 4900 is invoked to perform a consent management operation and authorize access to the patient data consistent with the consent provided by the user/patient.

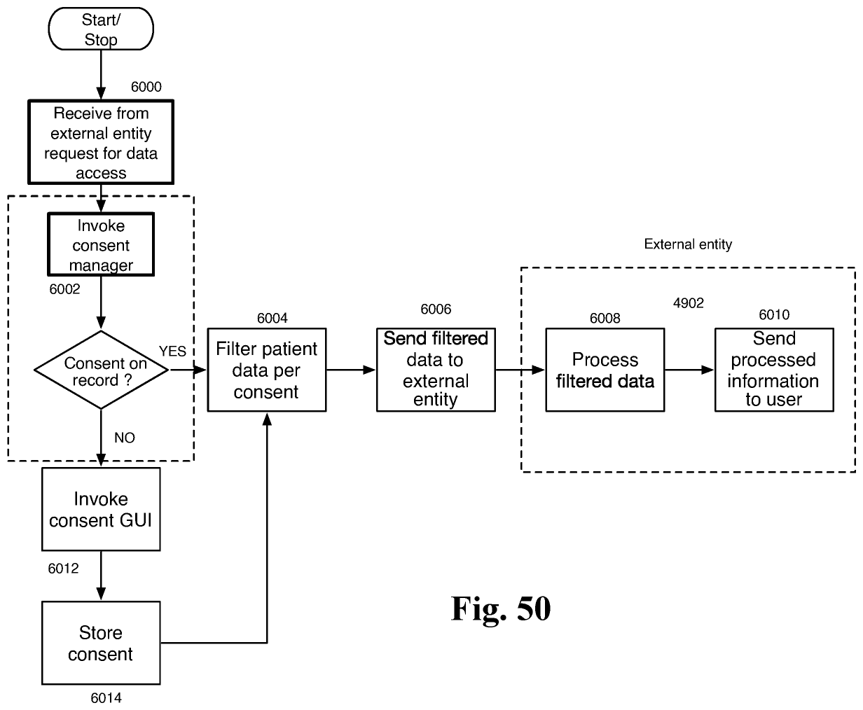

The general process flow is shown in FIG. 50. At step 6000, the Human-Centric EHR receives from the external entity request for data access. Examples of such external entity 4902 include research organizations, whether private or public, or analytics services that are desirous of performing an analytics process on the medical data to extract information that is highly specific to the patient in order to provide some advice, for example advice to improve a condition of the user/patient. [symbol] nples of advice that are related to the well-being of the patient, include:
1. Dietary advice—develop a diet that is specific to the patient and tailored to his or her medical condition as characterized by the medical data.
2. Physical exercise regimen—develop a specific physical activity program tailored to the individual.
3. Cosmetics blends—develop a specific blend of beauty products that are used to care for the face, body and hair to improve the person's appearance.
4. Tailor pharmaceutical regimen or preparations compounding—adjust or substitute ingredients, such as non-active ingredients to better fit the individual.
5. Genetic therapy.

Examples of advice or generally information that is not directly related to well-being, including:
1. Insurance, such as life insurance—an assessment of life insurance acceptability and tailoring life insurance offering to a user/patient. More generally, this can be characterized as a risk assessment for the purpose of a financial or insurance product, where a financial or insurance service provider can view de-identified and highly granular medical data to make a risk assessment that is specific to the user/patient.
2. Granting a visa in locations that prohibit individuals with certain medical conditions.
3. Prior drug or substance use disorder.
4. Work related health suitability.

At step 6002, the user access manager 4302 that can be assumed to run constantly in the background invokes the consent manager 4900 when the request for medical data is received from the external entity 4902. The consent manager 4900 describes collectively the functions that are provided by the Human-Centric EHR to manage consents, such as setting up a consent in relation to a particular external entity, modifying an existing consent or terminating a consent. Specific examples will be provided below to illustrate a range of scenarios. At step 6004, the consent manager 4900 tries to match the request for access to medical data to an existing consent. If an existing consent is found in the user profile 4300, in other words the user has previously authorized that particular external entity to access a portion of the medical data, fully or in part, the medical data is filtered at 6004 according to a filter or mask associated with the particular consent and the filtered data is then sent at 6006 to the external entity. [symbol] external entity receives the filtered data and processes it at 6008. The processing can include running [symbol] tne filtered data through a convolutional neural network trained to perform analytics. After the analysis process 6008 is completed, the results are sent to the user at 6010, via the Human-Centric EHR system or via another channel.

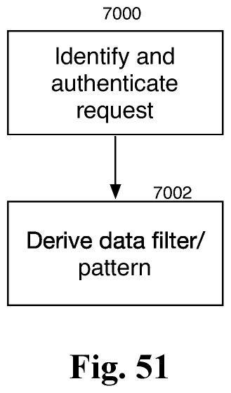

FIG. 51 is a more detailed process flow of steps 6002 and the subsequent decision step, illustrating in more detail the operations performed by the consent manager 4900. After reception of the request to access medical data 6000, the consent manager 4900 tries to determine if there is a consent in place from the user, associated with that particular external entity 4902. This is done at step 7000 and involves carrying out two operations: (1) to identify a consent corresponding to the request and (2) authenticate the request.

Requests can fall generally in two categories, one being one-time request, and the other repeated requests. For one-time requests there is likely not going to be consent on record in the user profile 4300, which will require invoking a mechanism to query the user about setting up a consent, including defining the scope of the medical information that can be exposed. That will be described in detail later. For repeated requests, a consent could be on record and the consent manager 4900 is trying to identify it and authenticate it. Assuming the consent is found, the consent manager will derive from the user profile 4900 at 7002 a filter or pattern that will expose only the portion of the data consistent with the consent.

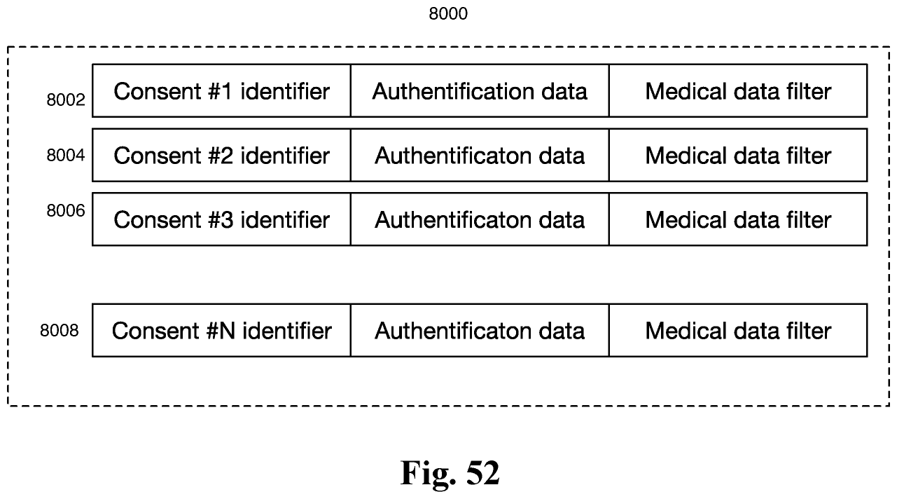

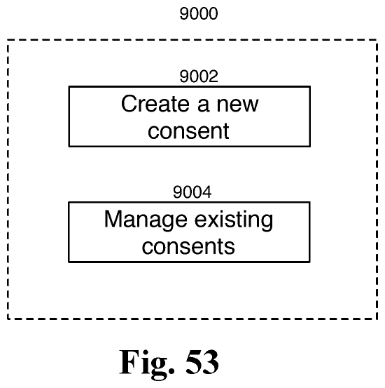

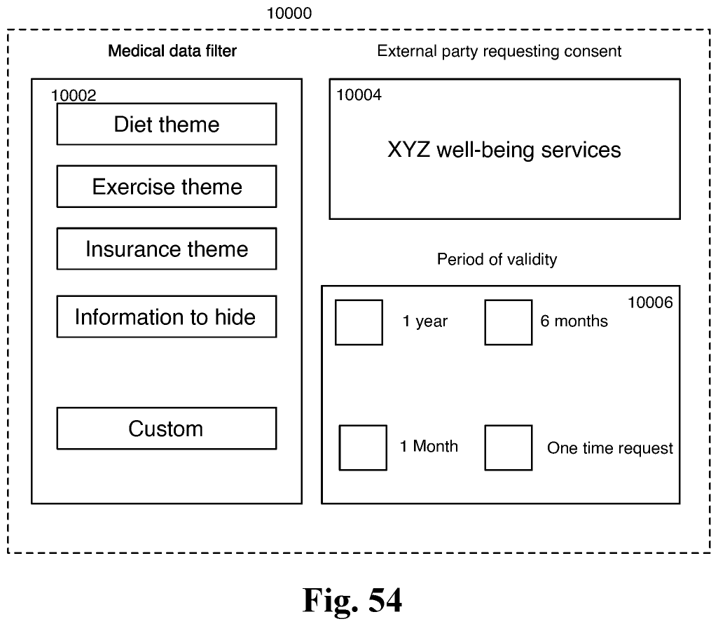

FIG. 52 illustrates the data structure in the memory 8000 of the server implementing the user profile 4300, showing the relevant data elements that make up a consent data element. The memory 8000 holds a plurality of consent data elements 8002, 8004, 8006, 8008. Each consent data element includes an identifier, authentication data, and a medical data filter. The identifier is a unique identification element that distinguishes one consent data element from another. For example, it can be a unique alphanumerical string. The authentication data is used to confirm the identity of the external entity, namely confirms that it is the same entity for which the consent was set up and not somebody else. The medical data filter identifies a subset of the medical data of the user that can be exposed in connection with this request.

The authentication data can be any type of data or mechanism allowing the consent manager 4900 to confirm the identity of the external entity 4902. The request from the external entity would contain two elements of information to identify and authenticate the consent, namely an identifier, and authentication data. If these two elements are present the consent manager 4900 locates the consent data element [symbol] sponding to the identifier and runs the authentication process to determine if it passes or fails. For [symbol] aple, the consent manager 4900 compares the authentication data stored in the consent data element to the authentication data conveyed by the request from the external entity. If both match, the authentication passes, otherwise it fails. The reader skilled in the art will appreciate that the authentication process can be performed differently without necessarily the need to compare locally authentication data.

The medical data filter extracts a portion of the entire medical data set of the user and creates a subset that is then sent to the external entity. The data filter is associated with the particular consent and defines the information that the user is willing to expose in connection with that consent. Note that in some instances, nothing may be sent out as the filter may be designed to block any dispatch of data.

Referring back to the flowchart of FIG. 50, the process path when the decision box is answered NO is to invoke at step 6012 the GUI screen to allow the user to set up a consent. An example of the GUI screen and various controls is shown at FIG. 53. The initial page of the GUI is a welcome screen with two button-like controls 9002 and 9004, the control 9002 being pressed when the user wants to create a new consent, while the button 9004 is pressed to manage existing consents. As it will be described later, the management function allows the user to terminate a consent or to modify it. An example of modification is to change the data filter to change the scope of the medical information being exposed.

Assume the user clicks on the button 9002 to create a new consent. The user is then brought to another page that provides a range of controls to create and define consents, as shown in FIG. 54. The screen 10000 includes three control sub-sets, namely the controls 10002 to define the medical data filter, the information display box 10004 to display the name of the party associated with the consent and the set of controls 10006 to define a period of validity for the consent.

The medical data filter set of controls 10002 provides the user with a set of simple choices in terms of the end use of the filtered data in order to set the appropriate filter. The user sees themes that are simple to understand and would not require the user to parse the entire medical data record to select individual items of information to include or exclude. The range of themes is vast. Examples shown in the drawings include a theme for dietary purposes where the information extracted from the medical data is sufficient to enable the creation of a highly customized diet, and no more. Similarly, the exercise theme is designed to expose only the information that is needed to create an exercise regimen for the user. The insurance theme is designed to extract only the information that an insurance company may need to [symbol] ide an initial quotation for life insurance for the individual and would typically include information [symbol] it key medical conditions of the user that would predict a life span. In another example, the study team could propose its own “filter” for the patient to consider and possibly accept. Note that in such circumstances, the filter could be read only and the patient could either accept or deny the terms but would not be authorized to change the terms.

In a specific example of implementation, the themes are respective sets of medical information categories, such as particular conditions affecting the individual, heart disease, diabetes, high blood pressure, etc. Each theme includes logic that parses the individual entries in the medical record of the user and then populates the categories accordingly. Populating a category may include placing there the entries of the medical data or a simplified version, such as a qualitative assessment of the condition (the patient suffers from hypertension at stage 2 of 4 stages).

The medical data filter definition control also includes a theme of information that is to be excluded (information to hide button). By clicking on it the user is presented with categories of information that are to remain hidden. The selection of the information to hide overrides the other themes. In other words, if a theme is designed to include information about STDs and the information to hide theme specifically indicates excludes STDs, then the particular category of information in the data filter theme will remain empty. While not shown in the drawings, once the user clicks on the button “information to hide” the user is shown yet another screen that lists information elements that can be selectively chosen and that will remain masked. Examples of categories include STDs, psychological conditions, substance use, sex reassignment therapies or psychological procedures, and anything else that the user wants to keep confidential. As per the other themes, the user picks a theme and underlying logic will parse the medical entries to identify those that can be associated with the theme and will mask those.

The user also has a “custom” button that allows to build a custom filter if an otherwise suitable category does not exist in the list. The custom button would bring a list of the entire medical record and the user can individually select the ones to include or exclude.

It will be noted that by default, the information to hide button is set to remove nominative information and any other information of the user. In this fashion, the medical information received by the external entity cannot identify the person associated with the medical information that is being to the external entity 4902. Optionally, a control may be provided, such as a toggle control, to de-identify the data sent out or to include the nominative information. This would make the GUI more user-friendly as it would [symbol] municate clearly to the user the key condition of the data being sent—de-identified or raw data [symbol] iding nominative information.

The display box 10004 shows the identity of the entity making the request. That information is derived from the request itself received by the server of the Human-Centric EHR system.

The set of controls 10006 set the period of validity of the consent. Possible options include one time, one month, 6 months, and one year, among others.

Another option is for the external party to supply a filter that is specific to the request for access. The filter can be a file that is sent by the third party to the Human-Centric EHR system along with the request for access. The filter typically would be read-only and as such not modifiable by the patient. To become active and start filtering the medical data, the filter would require explicit acceptance by the patient. Optionally, the filter may include an explanatory section to explain to the patient what data is being accessed and what data is not being accessed, such that the patient can appreciate the extent of the access being granted.

At the conclusion of the process during which the user supplies the necessary information and makes the relevant selections, the consent manager 4900 creates a consent data item in the memory of the server of the Human-Centric EHR system. The consent data item or consent record includes, as indicated earlier a unique identifier in order to distinguish one consent record from other consent records. The consent identifier may also include the identification of the entity that made the request, such as the name of the entity and other identification characteristics. The consent record further stores authentication data that is generated as a result of a registration process with the external entity 4902, allowing the Human-Centric EHR system to be able to uniquely and securely recognize the external entity 4902, when the later makes future requests for access to medical information. Finally, the consent record stores the medical data filter based on the input by the user.

Referring back to FIG. 50, the act of storing the consent is shown at 6014. At that point the processing goes to step 6004 that will filter the medical data based on the medical data filter in the consent as described earlier. To summarize, the individual medical data entries are parsed to determine which ones belong to a category of information that is to be included in the data sent out, by taking into account the categories of information that are to be specifically excluded. In addition to this categorization process, the data may be further processed to extract features or perform characterization of individual categories, [symbol] ad of sending out medical data entries. An example of feature extraction in a category that is [symbol] ciated with a certain condition, say diabetes, the feature extraction may specify if the user/patient is positive or negative and in the event the user is positive a characterization of the gravity of the situation. Note that the filtering step 6004 can be invoked periodically such as to capture new medical information that is generated over time. If new medical information is identified that is consistent with the filter settings, in other words it is something that fits the consent parameters and therefore should be made available to the third party, that information can be sent out as per step 6006.

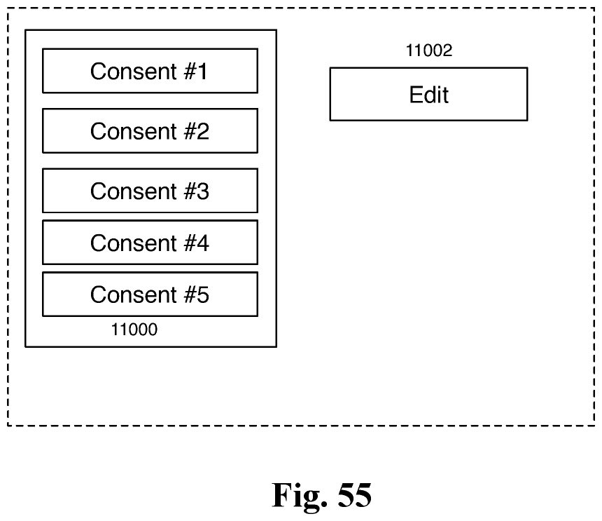

Referring back to the illustration of the GUI 9000, the user has the option to access a manager of existing consents. To access the manager function, the user uses the control 9004 to access the GUI screen shown in FIG. 55. By way of context, the manager of consents is used to control repeated access to the medical data, in particular allowing a third party to periodically access the medical information and get access to updates of that medical information. As it will be described later, the manager also can be used to control access to medical information by third parties that are unspecified and not known to the user. For instance, the user can authorize via a consent access to his/her medical information to third parties for research purposes. Specific examples of those scenarios will be discussed in detail later.

With specific reference to FIG. 55, the GUI presents the user with a depiction of the various consents 11000 that appear as a list in the example. The user can scroll through the list to view all the consents on record. For example, the consents can be identified by the name of the party that has the authorization to access the medical information. For example, the user may see as consent identifier party “ABC”, etc. Alternatively, the consents can be grouped into categories or profiles to make the identification and management of individual consents easier. For instance, the user may be provided with a profile based on the use of the medical information that is provided to a third party. Categories or profiles like research purposes, well-being services, are possibilities. In those instances, the consent manager can be provided with logic to classify the active consents into categories. The categories or profiles can be predetermined categories and the logic can make a decision on the basis of information, such as the name of the external entity to which the medical information is provided. The logic can be trained or otherwise programmed to distinguish between different external entities and place them in the corresponding categories.

Alternatively, a list of consent profiles can be presented to the user that the user can activate or deactivate and that would automatically apply to future requests, thus automating the dispatch of medical data to a third party. For instance, the list may include a profile adapted to medical data use by a research institution. The GUI allows the user to activate or deactivate profiles. When a profile is activated, logic [symbol] process incoming requests and determine on the basis of a keyword search for example, the consent [symbol] ile to associate with the request. Once the association is created, the request is processed as discussed earlier and the consent profile, associated with the request along with the medical data is sent to the third party.

The GUI mechanism provided to display the existing consents is designed to allow the user to view all the existing consents and to identify one or more among them for which a change in parameters is to be made. For instance, assume the consents are presented as a list, the user can highlight the desired one and then click on the control 11002 to edit it. Actuation of this control can bring the user to the GUI shown at FIG. 54 where the user can re-define the medical data filter 10002 or re-set the period of validity. Further to what is shown on the GUI screen of FIG. 54, there may provided a further option to cancel the consent and make further access by the external entity no longer possible.

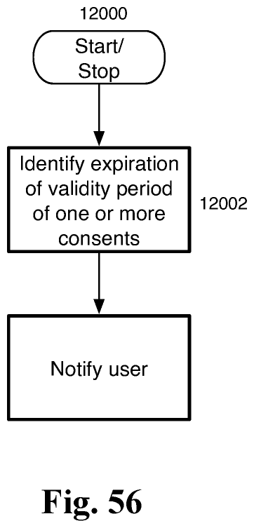

The function of periodically reviewing consents in order to cancel them or renew them may be built into the consent manager functionality. The flowchart at FIG. 56 illustrates this. The process starts at 12000. At step 12002 the consent manager logic identifies among the existing consents those that have a validity period expiring shortly. At step 12004 those consents are brought forward to the attention of the user. That can happen by invoking the GUI shown at FIG. 55 where the consents identified at the previous step are shown and where the user can make a decision, either renew them as such, modify them or cancel them. If the user is unable or unwilling to take action on the consent management, the consent manager logic may be designed to suspend the consents avoid that they go beyond their validity. In this fashion no medical information is sent out to a party that is outside the period of validity specifically indicated by the user originally.

The consent manager can also be designed with a functionality to track the information sent to third parties in order to provide full traceability and be able to establish who accessed what and the consent context that was present at the moment of the access. The tracking information may be made available to the user through a specific GUI screen.

As indicated above, some users may desire to allow controlled access to their medical information to a third party, without necessarily having to authorize the initial access individually. An example is research organizations that use the medical data for medical studies. Users may be willing to freely share their medical data (in a controlled fashion) with one or more entities that will use the data for a specific and well-defined purpose. The difference here with the previous examples is that the recipient of the data is [symbol] nitially identified and known. The recipient can be anyone that fits admissibility criteria. To manage [symbol] type of access to the medical data the GUI of the consent manager is modified to include admissibility criteria. Those criteria can be structured in manner that is simple and intuitive for the user to understand. For example, the admissibility criteria can be based on the purpose of use of the data and may offer the user the following specific choices:
1. Broad research purposes, where the data is used to advance the human knowledge in medicine
2. Research purposes have specific goals, in terms of class of treatment:
   a. Pharmaceutical research
   b. Medical treatment other than pharmaceutical research
3. Research purposes in relation to specific diseases or conditions, for example research in connection with:
   a. Heart disease.
   b. Alzheimer's disease.
   c. Diabetes.
   d. Cancer.

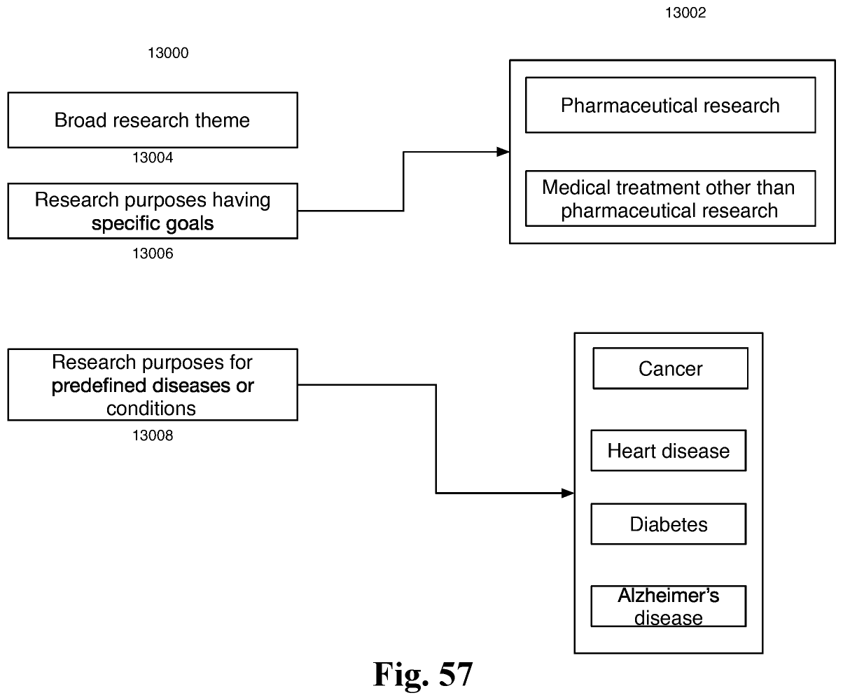

An example of a GUI that would allow the user to make choices and set the admissibility criteria is shown in FIG. 57. Initially, the user sees a first tier options 13000, some of which lead to a second tier options 13002 and possibly to third, fourth or more tier options. If the user activates the control 13004 the selection terminates, and the consent manager stores the selection in the medical data filter which will be processed as it will be discussed later.

Assuming the user makes the choice at the control 13006, the GUI switches to the second-tier options 13002 where the user can select between the two options shown there. Alternatively, if the user selects the option 13008, the user is presented with the second-tier options comprising a list of disease or conditions selections.

In all instances, after the selection operation is completed, the admissibility criteria selection is recorded in the medical data filter. In addition to setting the admissibility criteria it is to be understood that the GUI operates to prompt the user to specify information that the user does not want to share, along with a period of validity, as per the previous examples.

There may be some instances where medical studies may require the research team to communicate with the patient in order to perform specific lab tests interviews, etc. In such instances, the initial request may [symbol] ain instructions to display a message to the user requesting that the user contact information be sent to [symbol] research team such that the research team can in turn reach out to the patient. The instructions can be designed in such a way as to provide on the GUI an option for the user to deny the request, in which case the process aborts.

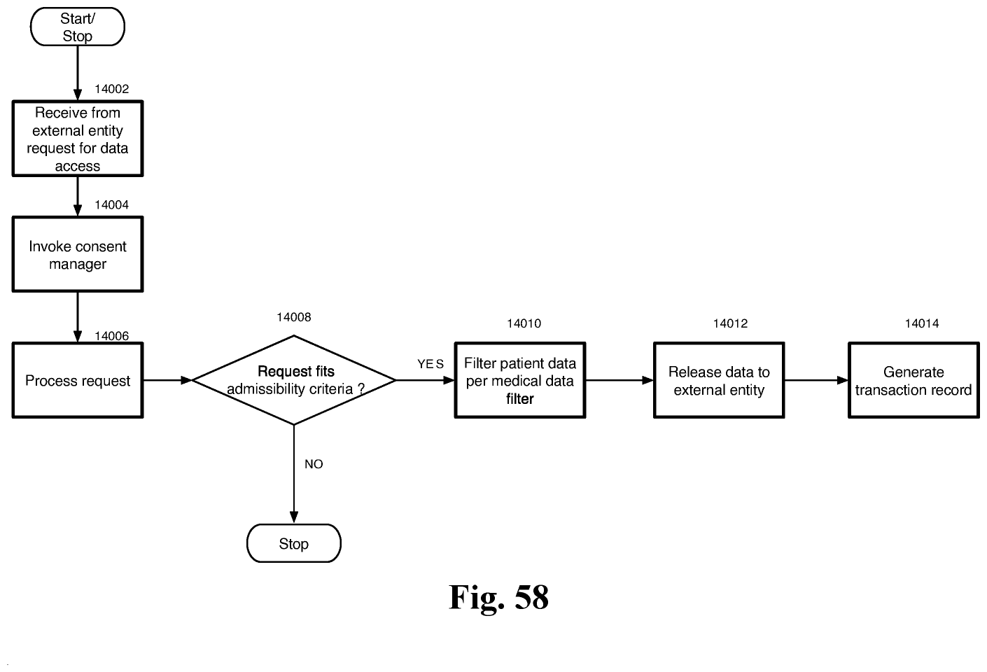

The management of requests of previously unidentified external entities is described in relation to the flowchart of FIG. 58. The process starts at 14000. At step 14002 the access manager receives from an external entity a request to access medical data from the patient. At step 14004 the consent manager is invoked. At step 14006 the request is processed. That step includes determining if a consent is on record with the particular entity generating the request as, for example, the process shown in the flowchart of FIG. 50. Assuming no consent on file exists, the consent manager triggers an agent to determine if the request falls in the admissible categories as specified by the medical data filter.

For example, the agent, which is a logic module, tries to match the request to anyone of the admissible categories specified by the user. Here the request conveys information to define the purpose of the use of the medical data along with the identity of the entity. The request may include keywords and the agent may look at the keywords and on the basis of them determine if the request falls in any admissible category.

If a match between the request and the admissibility criteria is established, as shown by the “YES” answer to the decision box 14008, the medical data is filtered as per the medical data filter settings at step 14010, for instance to strip out the name and other identifying information for the user along with any medical information the user does not want to expose, and the medical information is sent out at step 14012. A transaction record is then generated and stored in the user profile at step 14014. The transaction record has a unique identifier and serves to distinguish that transaction from every other transaction record. In addition to the unique identifier the transaction record includes any other data that is necessary to characterize the transaction, such as time, date, the identification of the external entity and the medical data that was sent. The transaction record provides the traceability discussed earlier. The unique identifier is shared with the external entity by sending it along with the medical data.

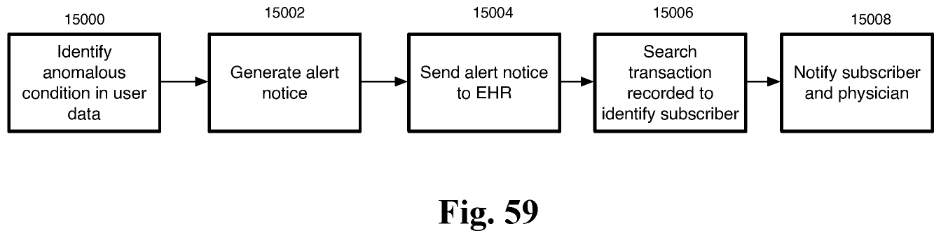

Assuming the request for access fits no admissibility criteria and the decision step 14008 is answered in the negative, the processing stops, and no medical data is sent to the external entity. [symbol] unique transaction record is useful in instances where the external entity, such as a research [symbol] tution while performing the research work finds that the medical data conveys a serious condition and for ethical reasons needs to alert the user. Since no identification of the patient is sent out with medical data, the external entity has no way of alerting the user directly. Accordingly, if such anomalous data is identified, the external entity will send an alert notification such that the Human-Centric EHR system can identify the subscriber associated with the particular transaction, notify the subscriber (user) and optionally notify the physician that provides health care services to the user. The process for sending such an alert notification is shown at FIG. 59.

At step 15000 the external entity identifies an anomalous condition in the medical data of the user, but since the data is de-identified there is no way to know the identify the person and reach that person directly. However, the external entity maintains its own transaction record which contains the unique identifier generated by the Human-Centric EHR system when the medical data was originally sent to the external entity. The alert notification would include the unique identifier along, at minimum, with an indication that anomalous information was found for that user. Optionally, details of the abnormality can be provided, for example by pointing to certain medical parameter that is abnormal. At step 15002 the alert notification containing the above information elements is generated and sent out to the Human-Centric EHR system at step 15004.

At step 15006 the alert notification is received at the Human-Centric EHR system and the server will search the unique identifiers for all the subscribers to find which subscriber is associated to the one in the alert notification. Once the subscriber has been identified the Human-Centric EHR system will notify the subscriber and optionally send a notification to a physician identified in the care support group in the user profile of the subscriber.

In another possible variant to allow the user to share its medical data is to present the user with a list of available projects, such as research projects that could use the medical information. The user can visit websites associated with such projects and proactively send the information instead for the external entity asking for it. Assume the user is presented on a GUI with the list of research projects, the user can select the ones of interest and export the data after the data has been filtered as per the settings in the medical data filter. Note that the external entities can provide their own research project consent document for the user to review and consent to. For example, in instances where the research institution sends out requests to users to gain access to their medical information and eventually to enroll them in research projects, the [symbol] al request may contain the consent information allowing the user to view it and accept it on the GUI. [symbol] example was briefly discussed earlier.

In another variant, a possible option to assist the user in properly setting the admissibility criteria, as explained previously in relation to FIG. 57, is to provide the consent manager with an agent that can guide the user in making choices that are consistent with its medical condition. In other words, while the user may want to offer its medical information for research in relation to particular diseases and conditions, the user data would be of practical usefulness to the research if the user actually suffers from those diseases and conditions. For instance, medical data from someone that has a healthy heart may not be useful to a research project on heart disease. In order to assist the user in making the choices that are consistent with his or her medical condition, the consent manger is provided with an agent that can make a correlation between the medical data of the user and the options offered by the GUI in FIG. 57 to highlight those that are the most consistent with the medical information.

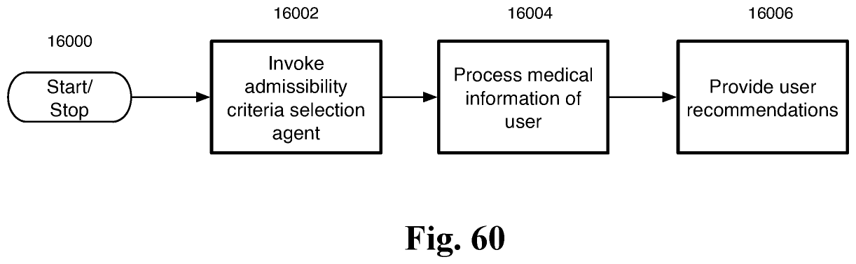

The process is depicted in the flowchart of FIG. 60. The process starts at step 16000. At step 16002 the admissibility criteria selection agent is invoked. The admissibility criteria selection agent is a logic that processes the medical information in the user record to identify diseases or conditions that afflict the user and then match those to admissibility conditions. This occurs at step 16004. In one possible example, the agent will parse the medical information for keywords corresponding to treatment, prescribed drugs or diagnosis associated with a disease or condition in a pre-set number of diseases or conditions the agent is designed to identify. In this example, the output of step 16004 is a list of diseases or conditions the agent has identified in the medical information of the user. At step 16006, the agent tries to match the identified diseases or conditions with choices for the admissibility criteria available to the user. For instance, if there are choices based on diseases or conditions, then those choices that match the diseases or conditions identified in the medical information of the user are highlighted to indicate to the user that his medical data is likely to be most useful for research in those areas. This guides the user in making more relevant selections.

In yet another example of implementation, the Human-Centric EHR system is configured with functionality to allow better matching between research institutions, either private or public that need medical data and human participants to perform fundamental research, either drug or medical device and procedures development. In many instances it is difficult for such research institutions to get access to medical data for statistical research, and even more difficult to get participants involved in clinical trials, especially when the trials require candidates that have a certain rare disease or condition. In other words, [symbol] difficult for research institutions to reach out to candidates who might be interested to participate in [symbol] research work. The typical process for a research institution is to advise hospitals or other care health care centers that treat a large number of patients about research projects and rely on physicians treating patients to determine which ones might fit the admissibility criteria of a certain research project and propose to the patients to participate. The process is long, tedious and in the end misses a number of potential candidates.

The Human-Centric EHR system may be configured to act as an intermediary between on the one hand research institutions and on the other hand a large pool of users in order to automatically perform a screening process and identify individuals who may fit the admissibility criteria of certain research projects, without exposing medical data of the individuals, and notify suitable candidates about the possibility of participation. The process is secure in the sense that no medical information is being exposed, the notification to the users about the possibility of participation in a research project is made in a private and confidential fashion leaving the ultimate decision to each individual as to participate or not. In addition, the process allows to set a financial compensation or other form of compensation in place for the use medical information.

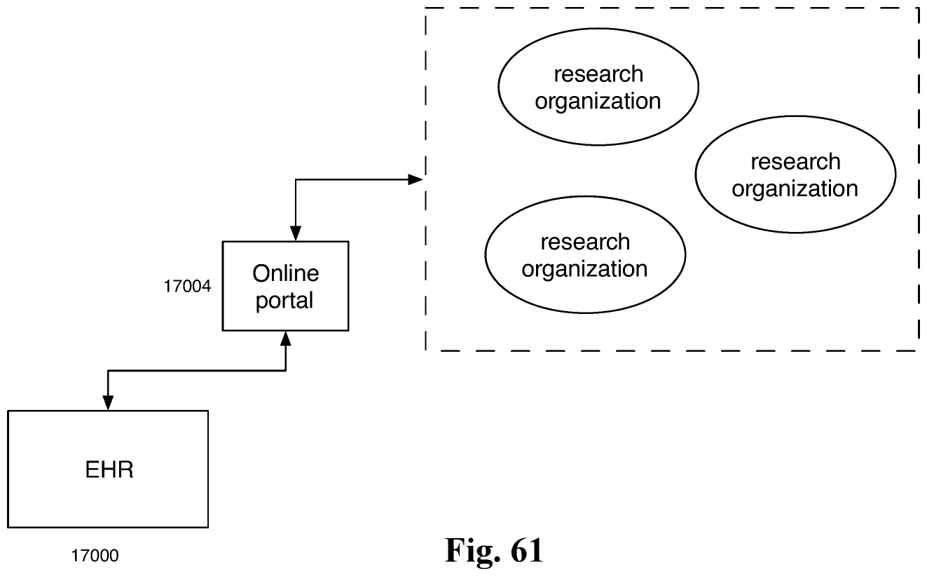

With specific reference to the block diagram of FIG. 61, the Human-Centric EHR system 17000 is provided with a portal where a research institution can send information about research projects. Such research projects may be of several kind, where only the medical information of the user is needed, or where the medical information and additional participation of the user is required. For example, in a clinical trial designed to determine the efficacy and safety of a drug, the admissible participants are required to interact with the research organization that would administer the active compound or a placebo to the user and monitor the response of the human body. Typically, this process requires that the patient visits a clinic and subjects himself to periodic medical examinations over the course a certain time period, typically a number of months.

The research institutions 17002 send to the online portal 17004 information about research projects. The information defines the parameters of the research project, in particular:
1. Medical/physical admissibility criteria—a definition of the medical or physical condition required for admission. The medical admissibility criteria may include, age, race, sex, presence of certain medical conditions which can be further characterized by degree of severity or presence of further physical conditions that can be characterized with any particular granularity. Demographics, including socio-economic status, or geographic localization are other examples of admissibility criteria.
2. Access to medical data only is required or if further engagement with the individual is required and in the latter case the characterization of this further engagement, such as participation in a clinical trial, interview with the research organization or others.
3. Compensation parameters. In other words, is there a possibility for financial compensation or other, the scope and conditions associated with it.

The Human-Centric EHR system 17000 is provided with an agent that receives the information defining the parameters of each research project, tries to match the research project to individual subscribers and when a match is found notifies the individual to determine if the person is interested to participate.

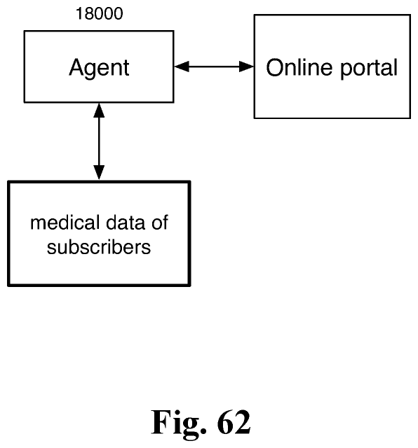

FIG. 62 is a block diagram illustrating this functionality. The agent 18000 is a software implemented function that receives as an input the information defining the parameters of the research project and tries to match those parameters with the medical information of individual subscribers. The information about a particular research project, in particular the admissibility criteria can be structured in any suitable way to enable in turn the agent 18000 to match the information to the medical data of different subscribers. In one example, the admissibility criteria can be structured in terms of categories. The agent is configured to process the medical data of the subscribers and categorize it accordingly and then determine if there is a match into individual categories. In a specific example, the admissibility data may include the following categories:
1. Age—in the range of 20-80
2. Race—any race
3. Sex—any
4. HDL cholesterol levels in excess of 3.1 mmol/L

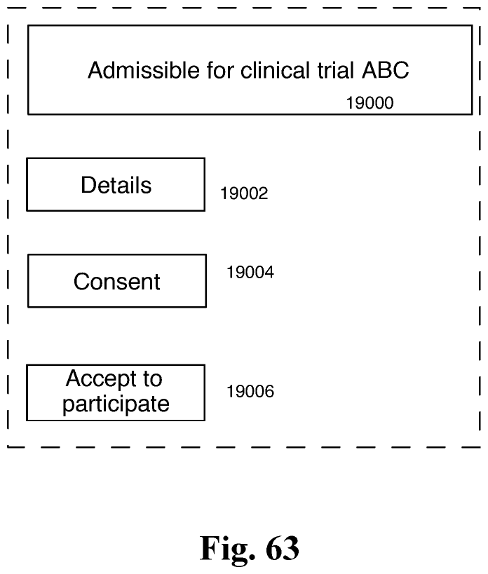

The agent parses the medical information associated with each subscriber to identify those that match the categories above. Matches are identified and a notification is sent to each user who has previously consented to be discoverable for medical research purposes. The notifications can be made through a GUI, as the one shown in FIG. 63. The GUI shows at 19000 the clinical trial or the medical project to which the subscriber is admissible. The display field 19000 would show a range of information including the name of the external entity that is responsible for the project, details about the project and any other [symbol] ble information. The user is provided with a series of controls to manage the acceptance or refusal to [symbol] cipate in the project.

Control 19002 triggers the display of additional information that the user may need to make a decision about his or her participation. For instance, the information may indicate the kind of participation required, such as whether only access to the medical information of the user is required or further involvement is also necessary such as enrollment in a clinical trial. The “details” screen can also indicate if compensation to the user is available and details of that compensation such as the mechanism for payment.

The consent 19004 displays a consent management page the user can consent to have its medical data, either fully or partially released to the external entity. Note that the consent management page may include a functionality to prevent exposing information the user wants to keep private, similar to the control 10002 at FIG. 54.

Finally, the acceptance control 19006, once triggered signifies acceptance to participation by the user. That may include sending the information to which the user has consented or providing the external entity with the contact information of the user such that the user can receive further details about his or her participation.

It should be noted that under any of the above scenarios, the consent information provided by the user is stored in a log that is retained in order to be able to establish for the Human-Centric EHR system legal compliance, if any, for consent management.

Several examples of the utility of the consent management solutions described above will now be provided in the context of immunology certification of an individual.

Immunology Certification

The previous examples of implementation of the Human-Centric EHR system and associated methods and features discussed the benefits of providing a robust consent and control mechanism over the exposure of medical information to a third party. Those examples were done in the context of the [symbol] ery of more traditional health care services, such as the need for a physician, who is not the regular [symbol] ician of the patient to access the medical information in the case of emergency, when for example the patient is travelling and his or her regular physician is not reachable. However, the same technical solutions that allow managing consent and/or control are applicable in a broader context in the society where there exist a need to provide access to some medical information of the user, such as immunology status, for performing social activities such as travel or participation to public events where the user expected to come in contact with a large number of people.

In a first example, an individual presents himself or herself to a venue to take part into an activity. The activity can be social, or professional. An example of a social activity is a concert or a sporting event where the individual either participates directly in the event or takes part of that activity as a spectator. An example of a professional activity is attendance to perform a job where the individual is paid for his or her services. Another example of an activity that could be social or professional is travel. The individual goes to a station, that could be an airport, train station or any other station where the individual uses a vehicle to travel to a destination.

To enable participation in the activity, the organizer requires from the participant an immunology certification, such disclosure of a immunization status in connection with one or more specific diseases. In the context of a global pandemic, the organizer may want to pro-actively manage the participation according to the immunization status of the individuals in order to avoid or limit as much as possible contagion since the participation would physically bring people close to each other. One example of proactive management is for the organizer to deny participation to an individual if the immunization status is either not known or inadequate. By inadequate is meant a known status where the individual has either not been vaccinated to develop resistance to a disease or the immunization is no longer effective. That is to say that the individual has been vaccinated at some point, but the effect of the vaccine may have subsided. This would typically be the situation with diseases where the vaccine triggers an immune response producing antibodies, which progressively fade away, leaving the individual unprotected after a certain period of time. That period of time can be months or years. Accordingly, an inadequate immunization status does not necessarily imply that the individual is unprotected, rather it means that the degree of protection afforded by the vaccine is not known.

When the individual arrives at the venue, an attendant checks the immunization status. The individual opens his or her mobile and opens an app on the mobile that manages the immunization status verification. In this example, the mobile and the app implement the patient's computing entity 106 or [symbol] Access to the app is granted via an authentication process, which can be biometric or via a nip in [symbol] r to establish to the attendant that the individual holding the mobile is the one having the correct access privileges. Without the proper authentication process, someone could use the mobile of another person and impersonate that individual such that the immunization status verification is performed on the wrong individual.

After the authentication process is completed the app communicates with the hand-held scanner of the attendant. An example of such communication is to display on the screen of the mobile a QR code which is read by the scanner. Alternatively, Near Field Communication (NFC) communication can be used, or any other suitable protocol can be used for this scan. After a successful scan, the scanner or backend server to which the scanner is connected will process the information supplied by the mobile, which includes an identification of the mobile or the individual. The identification can be nominative information such as the name of the individual and/or provide an address, social insurance number, driver license number and health card number, among others. Preferably, the identification information does not provide any nominative information. The identification information is used to distinguish the particular individual in an immunization status repository from other individuals in the repository, such that the correct immunization status information will be consulted. For example, the immunization status repository can be implemented by the Human-Centric EHR system 102.

The identification can be static or can be dynamically generated. The latter instance is preferred for security reasons. Accordingly, as the app is opened and after the authentication process is completed, the app communicates with immunization status repository to generate an identifier which is unique for that person and sends it to the app. Accordingly, every time the app is opened, a new identifier is generated for the mobile/individual.

Optionally, the information supplied by the mobile during the scan also conveys an identity or address of the immunization status repository. In implementations where multiple immunization status repositories would exist, it is useful to convey during the scan information identifying the particular repository where the immunization status of that individual can be found. Accordingly, the information sent to the scanner would include sufficient information to enable the verification process to direct the inquiry at the correct location. In other implementations, where there is a single immunization status repository for a geographic area, such as a city, province of country, the network location may not be needed since it would be implicitly known. An example of a large-scale government managed database of medical information is the Dossier Santé Quebec (DSQ) that holds medical information about Quebec residents, [symbol] d implement such immunization status repository. Accordingly, the implementation of the present [symbol] ntion in Quebec or with the knowledge that the individual whose immunization status is being determined is a Quebec resident, would implicitly indicate to the control logic to direct the request to the DSQ.

During the next step, the scanner or backend server communicates with the immunization status repository to inquire about the immunization status of the individual. The inquiry is directed at the address specified during the scan or implicitly identified. The immunization status repository receives the inquiry and processes it. On the basis of the identifier in the inquiry it locates the record of the individual associated with the mobile that was scanned. The immunization status repository then invokes a consent manager for that particular user to determine how the request will be handled. The consent manager operates as discussed in previous examples of implementation. One example is for the consent manager to deny access to immunization status requests. A second example is to enable it fully. A third example is to allow some level of access depending on how the consent manager has been set up by the user. After the consent manager determines what response can be provided to the inquiry, a notification is sent from the immunization status repository to the mobile of the user to notify the user about the request for access received. The notification can provide the user with the following elements of information to help the user determine if the request for access is valid:
1. Identification of the entity asking for access to the immunization status. That can be the name or some general indication of the location where the request came from.
2. The medical information the consent manager, which corresponds to the profile the user has set up previously, has authorized to convey to the entity making the inquiry. That information can be general, such as adequate/inadequate immunization status, but can also include specific elements of information such as:
   a. The brand, type or kind of vaccine the user was vaccinated with.
   b. The number of shots the vaccine requires and the immunization date for each shot.
   c. Estimated date at which full protection was achieved by the vaccine.
   d. Estimated date at which the protection is below a certain level.

After receiving the notification on the mobile the user has the option to authorize the release of information if the user is satisfied that the request for information is legitimate and that the detail of information released is adequate. If the user authorizes the release of the information, the immunization [symbol] is repository will respond to the inquiry and provide the requested information. That response is then [symbol] municated to the attendant, for example it appears on the scanner. The response can be a simple green or red colored block on the display corresponding to an adequate or an inadequate immunization status. The attendant can then clear the user to enter or deny entry accordingly.

The immunization status repository logs the request for information, the information that was released and the authorization given by the user, which is then stored in the user profile to allow the user to track which entity asked for access and what information was provided to that entity and when.

The above provides a general outline of the process that will be better understood with reference to the following more detailed examples.

Airline Travel

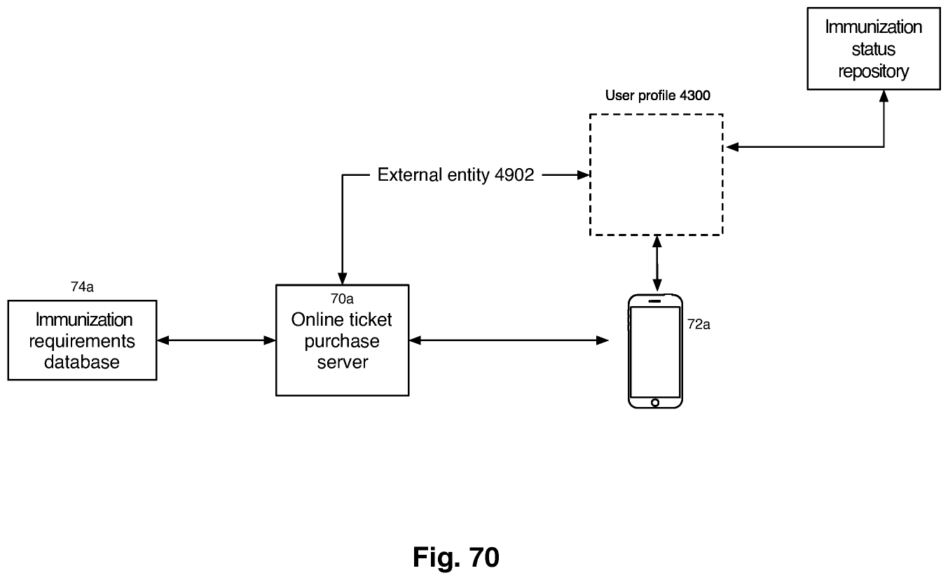

The user makes a purchase of an airline ticket online. FIG. 70 is a block diagram illustrating the various network entities involved in the purchase, which is conditional on the traveler having an adequate immunization status.

The airline company manages the online sale of tickets through a server arrangement 70a. The user accesses the server arrangement 70a via the mobile 72a. The network interaction between the server arrangement 70a and the mobile 72a is an online transaction during which information is exchanged between the server arrangement 70a and the mobile 72a. The online transaction involves providing on the mobile a Graphical User interface (GUI) that would allow the traveler to provide the required information to make the ticket purchase, such as the destination and origin for the trip, the date for the trip, including the outgoing flight date and the incoming flight date, seat selection, luggage information, passport and/visa information and make payment with a credit card or another payment instrument.

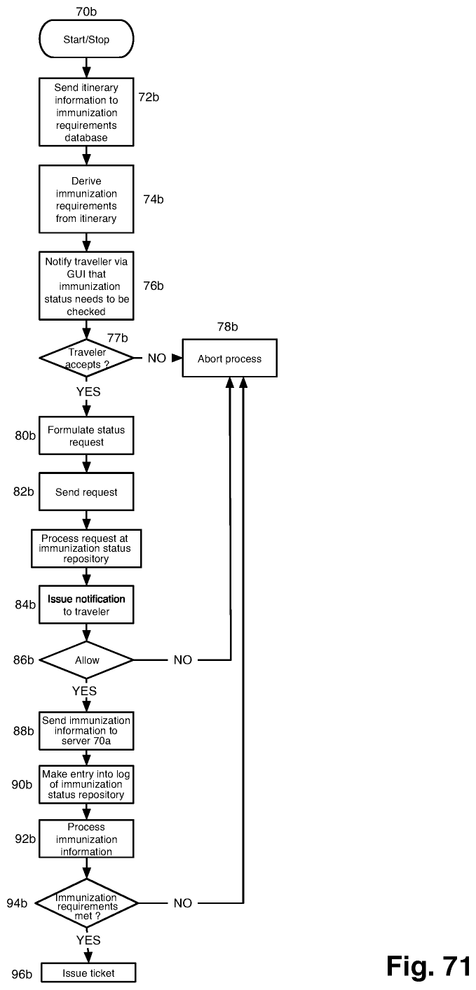

At some point during the transaction but before completing the transaction, the software running on the server arrangement 70a will determine the immunization requirements for the traveler. This process is described in connection with the flowchart of FIG. 71. The process starts at 70b. At step 72b, the server arrangement 70a will dispatch the information about the trip itinerary supplied by the traveler via the GUI on the mobile 72a to the immunization requirements database 74a that will return to the server arrangement 70a a immunization requirement data conveying particulars of the immunization requirements that are required from the traveler for this particular trip. [symbol] e specifically, the immunization requirement database 74a will derive from the itinerary the [symbol] unization requirements, as shown at step 74b. Typically, the immunization requirements are country specific. Some countries may have requirements, and some may not have any, in other words the traveler is not required to demonstrate any particular immunization status. For the countries that have a particular immunization requirement, those requirements may vary. Examples of information that may need to be provided to determine if the requirements are met for a particular country, include:
a. A particular brand, type or kind of vaccine to authorize travel.
b. Confirmation that the number of shots the vaccine requires have been taken.
c. The immunization date for each shot.
d. Estimated date at which full protection was achieved by the vaccine.
e. Estimated date at which the protection is below a certain level.

At step 76b, assuming specific immunization requirements need to be met, the server arrangement 70a will notify the traveler via the GUI at the mobile 72a that his or her immunization status needs to be checked. The GUI is configured to provide the traveler with an option, which can be “yes” or “no”. If the traveler accepts to provide the immunization status information, as shown by the decision step 77b the process continues, otherwise the process aborts.

Since the traveler has already provided nominative information for the ticker purchase, that information may be sufficient for the server arrangement 70a to initiate the process to verify the immunization status. At step 80b the server arrangement will process the itinerary specific immunization requirement information provided by the immunization requirement database 74a to formulate an immunization status request. The immunization status request conveys the immunization information necessary to determine if the specific requirements for the particular trip are being met. The request is sent at step 82b. Assume the EHR system shown in FIG. 49 is the one that manages the patient data of the traveler and acts as an immunization status database. The server arrangement 70a is therefore the equivalent of the external entity 4902 that asks permission to access the medical data of the patient, in this case the immunization information. The request will be handled as discussed previously. Namely, it is received by the user access manager 4302 that invokes the consent manager 4900 to determine if the request should be accepted or rejected. Generally, the process illustrated at FIG. 50 is followed. Here, the process assumes that the user/traveler has a consent on record allowing a third party to access immunization information. [symbol] rring back to the flowchart of FIG. 71, the EHR system will issue a notification to the traveler via [symbol] mobile 72a, at step 84b to notify the traveler that a request has been made to access medical information. The notification would provide the following elements of information:
a. Identity of the entity that has made the request. In this particular example, the entity would be the airline company that is selling the airplane ticket.
b. The particulars of the immunization information that are being released, such as the vaccine brand/kind or type and the vaccinate schedules, among others.

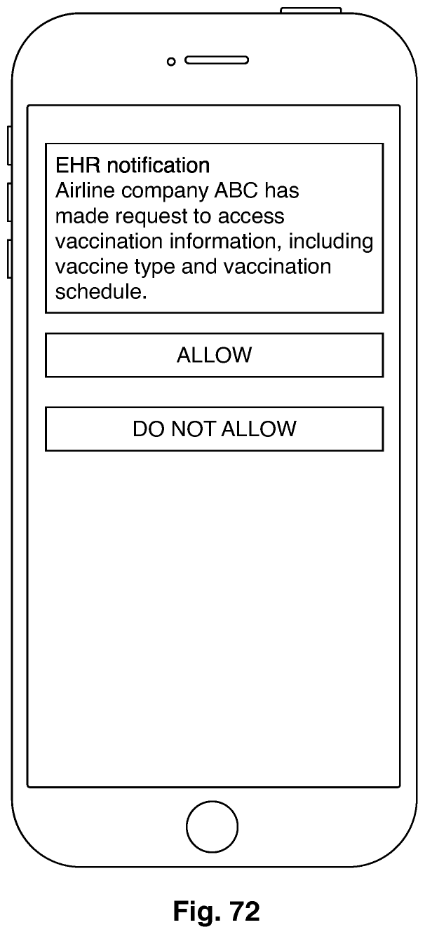

An example of how the notification may look like on the display of the mobile 72a is shown at FIG. 72. The notification embeds an action function, providing the user/traveler with the option via the controls “Allow” and “Do not allow” to authorize the release of the information or deny it.

If at decision step 86b the user/traveler presses the “Allow” button, the information is returned to the server arrangement 70a, as shown at step 88b and an entry is made in the log of the immunization status repository, as shown at step 90b to indicate that the Airline company ABC has been granted access to the immunization information at a particular date and time and the particulars of the information that have been accessed, at step 88b.

However, if the user refuses to release the information by actuating the “Do not allow” button, the process aborts.

At step 92b the immunization information is processed by the server arrangement 70a to determine if the itinerary specific immunization requirements are being met. This is shown by decision step 94b.

If the immunization requirements are met the ticket is issued at step 96b, which would typically include processing payment and making an entry in a database of the server arrangement 70a that a ticket has been issued and that the immunization requirements for the particular trip have been met. On the other hand, if the immunization requirements are not being met, the process aborts.

In a possible variant of the process described earlier, instead of the server arrangement 70a querying directly the immunization status repository (implemented by the Human-Centric EHR system 102) on the basis of the nominative information provided by the user/traveler that identifies the person, the server arrangement 70a can send a message to the mobile 72a such that a communication can be established [symbol] een the server arrangement 70a and the EHR system to complete the immunization information [symbol] mission to the server arrangement 70a. The advantage of this method is that the identity of the individual is confirmed by virtue of the fact that a transaction is ongoing with the mobile 72a and the user/traveler can establish his/her own identity via the mobile 72a with the immunization status repository, accordingly the request for immunization information is necessarily linked to a specific ticket purchase transaction.

In addition, sending nominative information to query the immunization status repository may not be possible in light of regulations that prevent the airline company to use the nominative information for any other purpose but for the ticket purchase.

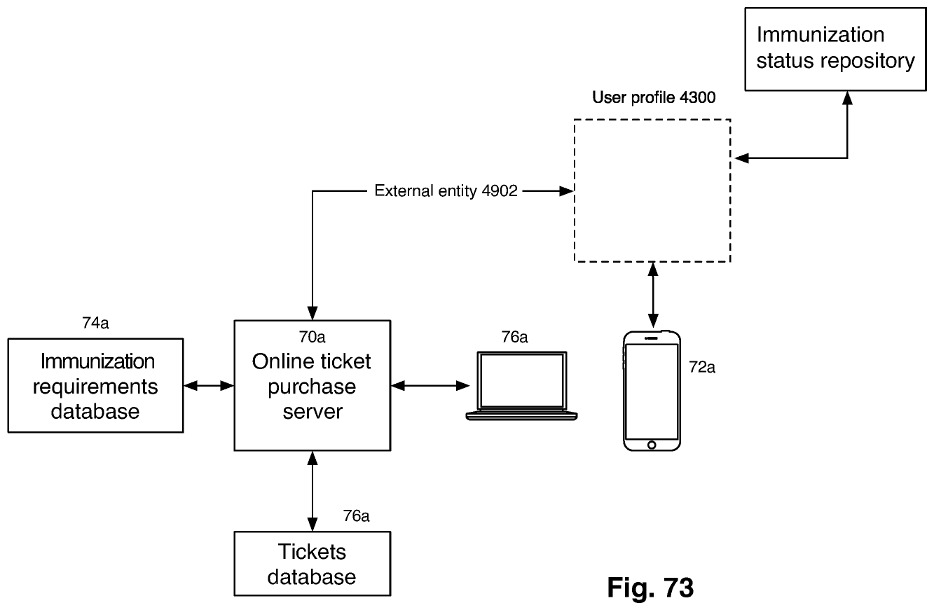

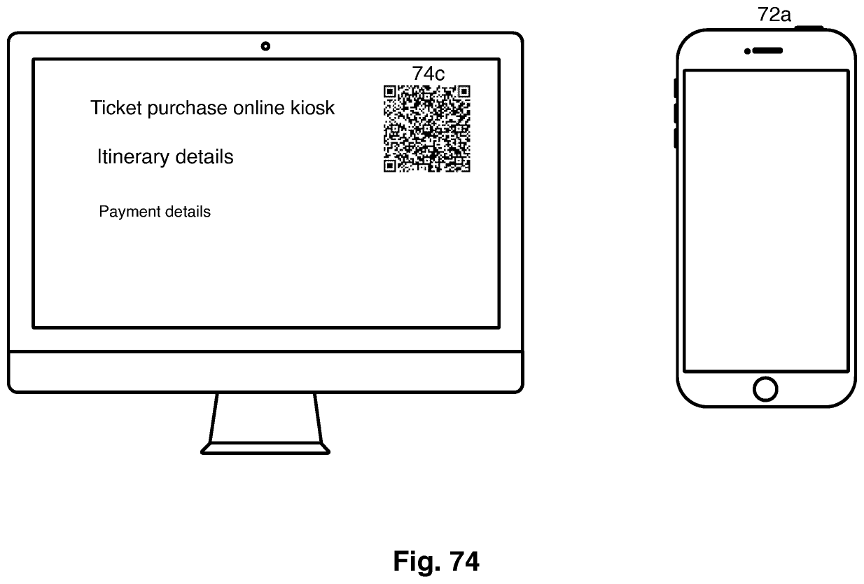

An example of this variant is depicted in FIG. 73, which is a block diagram of the various network entities involved in the process. Similar or identical entities to those shown in FIG. 70 are identified with the same reference numerals. In this variant, the transaction between the server arrangement 70a and the traveler is performed via a computing entity 76a which can be a personal computer. More specifically, the user will conduct the transaction via the personal computer 76a instead of doing it via the mobile 72a. The server 70a will implement the GUI on the personal computer 76a and the user enters the necessary information to perform the purchase of the ticket. When the process reaches the point where the immunization status of the user needs to be provided, the GUI will present on the screen a QR code 74c, shown at FIG. 74 which can be scanned by the mobile 72a. The QR code 74c creates an operational linkage with the Human-Centric EHR 102 via the mobile 72a that acts as the patient's computing entity 106, 5002 programmed with the EHR app, that is assumed to be open in order to scan the QR code 74c. The QR code 74c therefore communicates certain information to the app executed on the mobile 72a, which includes the following:
1. Identification of the ticket purchase session, such that whatever information the immunization status repository will return, it will be routed to the correct session, it being understood that a number of such ticket purchase sessions are being concurrently executed.
2. Identification of the party asking for the immunization related information. In this case that party is Airline company ABC. Further characterization of the request may include the date of the request, itinerary details and any other information.
3. Immunization information that is requested from the immunization status repository, such as the particular brand, type or kind of vaccine to authorize travel, confirmation that the number of shots the vaccine requires have been taken, the immunization date for each shot, estimated date at which full protection was achieved by the vaccine and estimated date at which the protection is below a certain level, among others.
4. Identification or address where the immunization information is to be returned. Typically, this could be a network address associated with the server 70a.

As the QR code is displayed on the screen, the user scans the code with the app which reads the code and directs the information to the immunization status repository. It is to be noted that since the mobile 72a is opened and the app is active, assumes that the necessary authentication processes have been performed at the mobile level and optionally at the app level, such that the request sent via the mobile 72a is in fact authorized by the legitimate user.

The information extracted from the QR code 74c is sent via the app to the immunization status repository and it is processed as discussed previously, in particular as illustrated by the process steps 82b to 96b.

After the ticket purchase is completed, an entry is created by the server 70a in the tickets database 76a which stores active tickets. Each ticket conveys the information about the traveler, itinerary and in this specific case immunization information about the traveler to confirm that the immunization status of the traveler is compliant with the requirements, such as the requirements of a foreign country.

In another possible variant, the ticket purchase process can be designed such that the process does not automatically abort if user is unable to supply to the system the requested immunization information. Accordingly, the system may allow the ticket to be purchased but at the condition the user demonstrates at a later date that he or she meets the immunization requirements. That can be done at a designated kiosk at the airport as it will be described later. Under this variant, the process would be similar to the process shown at the flowchart of FIG. 71, the difference being that if the user answers the decision step about the need to provide the immunization status information in the negative, the process does not abort, but a warning is presented to the user to indicate that while the ticket can be purchased, the user needs to show compliance with the immunization requirements before clearing customs or boarding the plane. If the user accepts the requirements, the ticket is issued and an entry is made in the database 76a, however the [symbol] unization status associated with the ticket is either blank or indicates that the immunization [symbol] mation is missing and needs to be provided.

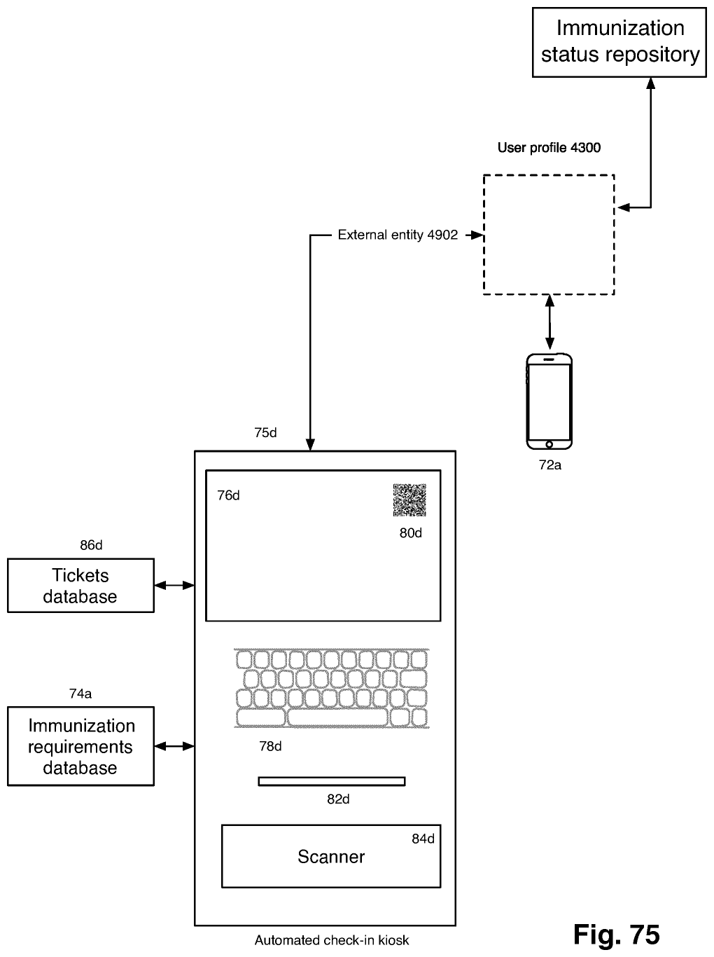

In a different example, the immunization information collection from travelers is not linked directly to the ticket purchase process. That is to say, the ticket is purchased as usual and the traveler is required to demonstrate an acceptable immunization status at the travel station before boarding the travel vehicle, such as the aircraft or train. FIG. 75 is a block diagram showing the arrangement of the various entities of such system.

The system includes a self-service check-in kiosk 75d, such as one for dispensing a boarding pass at an airport and also providing luggage tags. The kiosk 75d is provided with software and communication capabilities to check the immunization status of a traveler. One possibility is for the kiosk to issue a boarding pass only if the immunization status of the traveler is verified, and it is satisfactory. The kiosk has a body with a display 76d on which instructions are displayed and information can be input by the user/traveler. A data input mechanism such as a keyboard 78d is shown, although the keyboard can be integrated into the display 76d if the latter one is a touch sensitive display. A boarding pass and luggage tag dispensing slot 82d is provided to dispense paper-based boarding passes and also luggage tags after a check-in transaction have been completed.

A scanner 84d is provided to scan documents such a passport.

The kiosk 75d is a computer-based device and it will be understood it has a CPU, memory and the necessary interfaces to allow it to communicate with external entities, databases and peripherals. The memory stores software that defines the functionality of the kiosk and in this particular case, in addition to the normal and well understood kiosk functions to enable a travel to perform a check-in operation, a immunization status check.

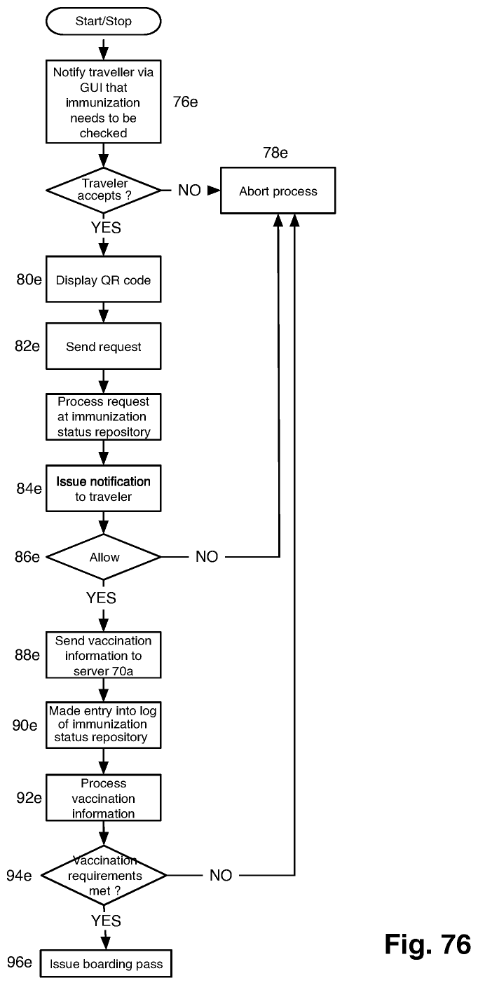

The kiosk 75d communicates with a tickets database 86d in order to be able to perform a check-in transaction. A traveler would typically enter identification information to enable the kiosk 75d to locate the ticket associated with the traveler. The traveler is then required to perform seat selection and provide luggage information. The immunization status determination is shown in relation to the flowchart of FIG. 76, which is similar to the flowchart at FIG. 71. At step 76e the kiosk shows at the display 76d a message notifying the traveler that the immunization status needs to be provided. If the traveler accepts, as per the decision step, the process continues, otherwise the process aborts and no boarding pass is [symbol] d, as per step 78e. At steps 80e the kiosk displays a QR code on the screen which is scanned by the [symbol] mobile 72a via the intermediary of the app. It is to be understood that the immunization requirements database 74a has been previously consulted once the itinerary of the traveler has been obtained and the necessary request for information is encoded in the QR code. At step 82e the information is sent by the mobile 72a to the immunization status repository for processing. At step 84e the immunization status repository sends out the notification to the user mobile 72a for confirmation before releasing any information. If the user allows the release of the information at decision step 86e, the process continues further otherwise it aborts as per step 78e.

If the user authorizes the release of the information, the immunization status repository sends the immunization information to the kiosk 75d at step 88e and makes a log entry at step 90e. The kiosk 75d, upon receiving the immunization information processes it at step 92e. if the immunization requirements are met at decision step 94e the boarding pass issues at step 96e. Otherwise, the process aborts.

It should be noted that while the process has been described in relation to a physical check-in kiosk, the same process would work with electronic check-in, where the check-in operation is performed on a computer and an electronic (not paper) boarding pass is issued, for airplane travel, train travel or other.

As previously indicated, instead of communication via a QR code, other types of wireless communication can be used such as NFC and Bluetooth among others.

It should also be noted that the kiosk implementation discussed above has applications broader than travel. For example, the kiosk may be used to grant access to a location, such as at the entry of a building, elevator access or door access. The user would interact with the kiosk as discussed previously, to convey the immunization information to the computerized entity managing the kiosk operation. The kiosk is designed to control access based on the immunization status. If that status is satisfactory then access is granted, otherwise no access is granted.

In addition to traveling the immunization status of the user can be necessary to access any other venue where several individuals gather. That can be the case of a private social event where the organizers may want to ensure the safety of participants by checking the immunization status of everyone that attends. In the case of occasional events, a kiosk implementation may not be practical, and a mobile app may be provided which works as a kiosk. The app can run on the mobile of the organizer or staff controlling [symbol] ss to the venue, in order to be able to rapidly check the immunization status of attendees and admit [symbol] individuals that have a satisfactory immunization status.

Another utility of such software is for job-related immunization status verification. An employer may want to check periodically the immunization status of employees to ensure that those admitted to the premises are not contagious. For job sites where the kiosk implementation may not be practical, such as construction sites or other venues where a job activity occurs but on a temporary basis, integrating the functionality of a kiosk in the mobile of a manager would allow to check on a regular basis the immunization status of employees.

Such an app is thus practical for occasional use. The app can be downloaded from an app store such as the Apple app store or an Android app store.

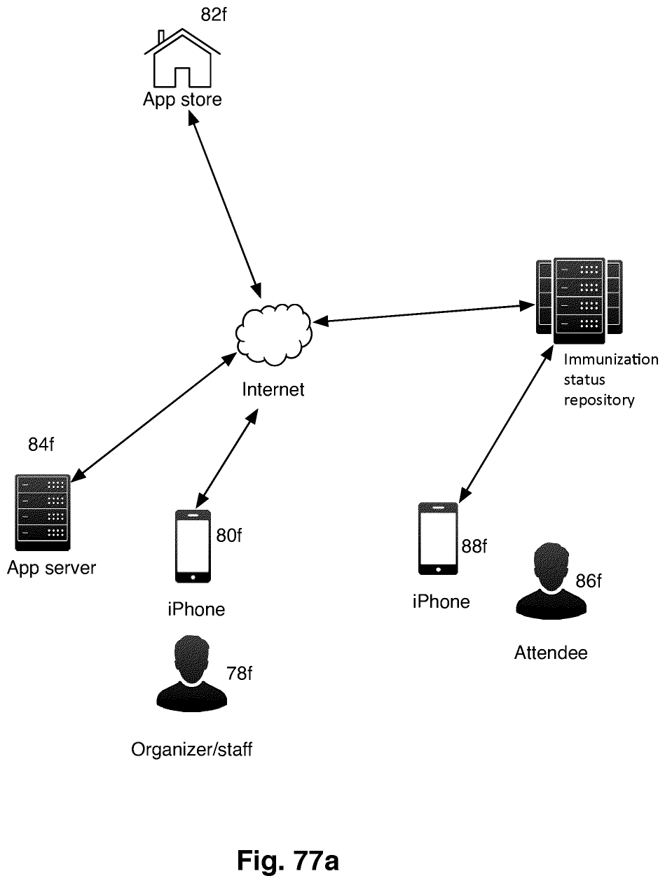

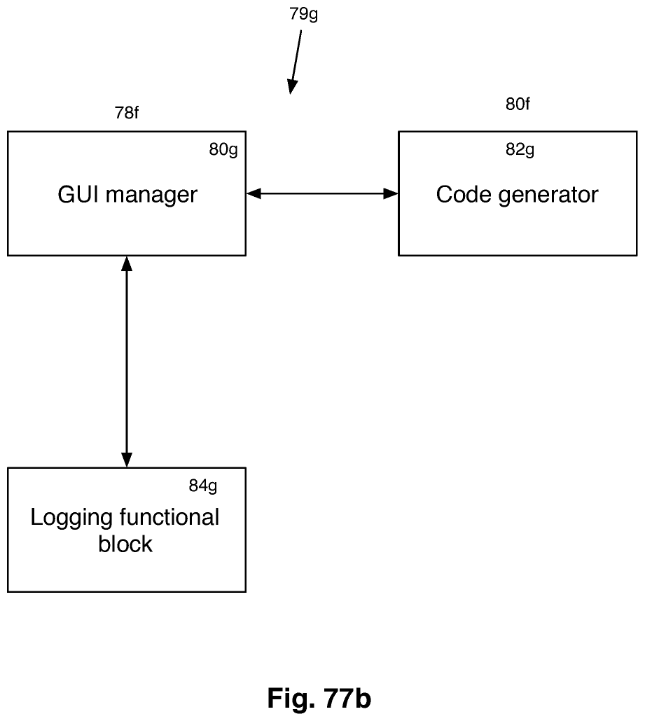

A diagram of the various entities and network interactions is shown at FIG. 77a. The organizer 78f or manager that is responsible to check immunization status downloads on his or her mobile 80f an app from the app store 82f. After the app is installed, it communicates with a server 84f that manages the app operation and interactions with external entities.

In use, as an attendee 86f arrives at the venue. The organizer 78f or manager will greet the attendee and open the app to check the attendee immunization status. The app will generate a QR code on the screen of the mobile 80f. This QR code is similar in function as described in the previous embodiments and is configured to trigger an inquiry, via the mobile 88f of the user of the immunization status of the user via the immunization status repository. To elaborate, as the QR code is shown on the mobile 80f, the user with the mobile 88f opens the app that is linked with the immunization status repository and scans the QR code. The process assumes that suitable user authentication has been performed at the mobile level and optionally at the app level such that the immunization status repository can assume that requests originating from the app on the mobile 88f are from the legitimate user. The QR code contains the request for immunization status which is forwarded to the immunization status repository. The immunization status repository receives the request and will send a notification to the user on the mobile 88f, as discussed in the previous embodiments. If the user authorizes the immunization status release, the immunization status is released and conveyed to the server 84f that in turn sends it to the mobile 80f of the organizer/manager 78f. [symbol] re 77b is a block diagram of the app that is executed on the mobile 80f of the organizer/manager 78f, [symbol] trating its main functional blocks. The app 79g has three main functional blocks including a GUI manager functional block 80g, a communication functional block 82g and a logging functional block 84g.

The GUI manager functional block 80g provides a dashboard allowing the user to set up parameters and to control the behavior of the app. Examples of settable parameters include:
1. Particulars of the immunization status that are to be requested from the attendees. In other words, what level of detail in connection with the immunization status is required. In some instances, such as private social events the organizer may want to ensure that the attendees have some level of immunity. Thus, the immunization status inquiry may be of general nature, only asking if the person has been vaccinated but without the need to provide vaccination details. In other instances, a specific protocol may be in place that requires a more specific knowledge of the immunization status, such as the kind of vaccine, vaccination dates, etc. That protocol can be required by internal policy of a business organization to monitor the immunization status of employees or required by government regulations. Accordingly, the GUI manager functional block 80g can be used to adapt the immunization status inquiry to a range of different situations.
2. Name or identification of the event.

Once the dashboard parameters are set and the app is activated to read the immunization status of an attendee, the communication functional block 82g will communicate with the mobile 88f of the attendee as it was discussed above. Specifically, the communication functional block 82g will generate a QR code. It is to be understood that the QR code is dependent on the settings of the dashboard and will convey the specificity of the immunization status that is requested. The QR code is displayed on the mobile 80f, which in turn is scanned by the mobile 88f of the attendee 86f and processed as discussed previously. The results are transmitted to the mobile 80f and the organizer can then determine if the attendee is fit to be admitted.

Finally, a logging functional block 84g logs the results of the verifications. By logging the results of the immunization status check it becomes possible for the organizer to establish that the immunization status check has indeed been performed, the identity of the individuals checked, and the immunization status results.

In the previous examples of interaction, where the mobile of the user (the individual whose immunization status is being assessed) triggers the transfer of immunization information from the immunization status [symbol] sitory to the requesting party, the direction of communication is from the requester (the party that [symbol] s to know the immunization status) to the user. Specifically, the mobile of the requester generates the QR code which is then read by the mobile of the user (it being understood that other forms of communication such as NFC among others, are also possible). The advantage of this approach (from requester to user) is that it maintains privacy for the user as it is the user that effectively queries the immunization status repository and the requester simply receives results. Objectively, a disadvantage for the requester is that there is no guarantee that the results received from the immunization status repository are those of the user. This could be the case when the user interacts with an automated kiosk where the QR code is generated and could be read by the mobile of a person other than one travelling and checking in as the kiosk has no means of verifying the identity of the mobile that is scanning the QR code. To the extent there is a requirement to authenticate the user that triggers the query of the immunization status repository, a solution is to forward with the immunization results some identification of the user, such as a name that can then be matched to the name in the ticket or passport. If there is a match, then the process could assume that the immunization results provided are those of the individual checking in or purchasing the ticket, otherwise they could be rejected.

A variant is to reverse the direction of the inquiry, where the mobile of the user forwards the necessary identification information to the kiosk which then communicates with the immunization status repository. and receives the results, subject to authorization from the user, in response to a notification sent by the immunization status repository.

It should be noted that the immunization status repository can be configured to contain the immunization status information in a database, or it can be a gateway to other nodes that actually hold the information. In such instance, the immunization status repository contains the mechanism that allows the process to pull the information from the remote source but does not hold any medical information as such.

Medical Testing Reconciliation

This section describes yet another functionality of the Human-Centric EHR system 20000, which in other aspects is analogous to the Human-Centric EHR system 102 described earlier.

The typical medical testing procedure involves multiple steps before the test results are communicated to the patient, a diagnostic is made by the health care professional and eventually a treatment is prescribed. [symbol] e specifically, after the patient sees the health care professional who will prescribe one or more tests, [symbol] patient would typically need to schedule the test at the clinic, lab or a hospital. It is well established that some patients after being prescribed the test may not follow up and actually do it. The consequence is that some medical condition may go unnoticed and further deteriorate over time. Since the responsibility to schedule and present itself to the testing facility rests with the patient, the health care provider has no practical means to pro-actively follow up and remind the patient to take the test.

When the patient goes for testing and the medical test is carried out, the results are rarely available immediately. Some time is necessary for the lab to complete the work and produce test results. Those results then need to be sent to the health care professional such that they are placed in the patient's file and then become part of the patient's electronic record. When test results are sent to the health care professional, a reconciliation needs to be done between the request for the test and the actual test results. It will be apparent that the potential exists for missing test results or incorrectly filing test results. For instance, it is possible that the lab never made the test or misplaced the test results such no results are sent back to the health care professional. Unless a reminder system is put in place, the health care professional would not see the results and the patient would not be advised of the results. Moreover, typical tests, such as blood tests involve multiple individual tests, and it is possible that the technician at the lab forgets to carry out some of the individual tests. Accordingly, the results produced are partial results and they are transmitted to the health care professional that may not realize that some results are missing.

Against this background it will become apparent that the existing testing process is deficient as there are a number of potential failure points in the chain, which creates risk for the patient and also malpractice risk for the health care professional.

In this example, the Human-Centric EHR system 20000 provides a medical test reconciliation and management system to track testing requests and results produced by a testing facility in response to the testing request to reduce the possibility of misplaced or lost information.

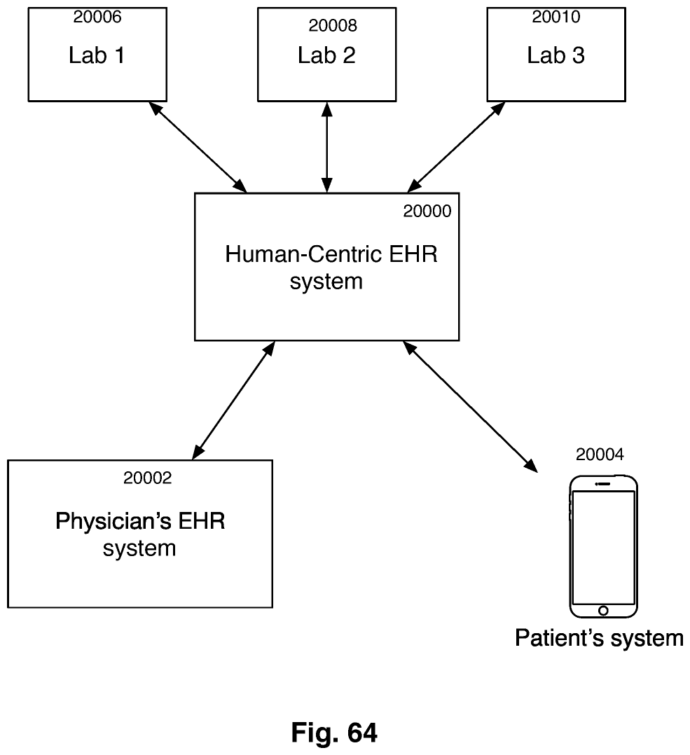

FIG. 64 is a block diagram of the network arrangement including the Human-Centric EHR system 20000. The EHR system 20000 communicates with a patient system 20004, which typically would be a mobile, to allow the patient to receive medical information and also provide inputs. The Human-Centric EHR system 20000 also communicates with the Physician's EHR system 20002, as discussed in previous implementations. In addition, the Human-Centric EHR system 20000 communicates with a plurality of [symbol] ng facilities which are identified as 20006 (Lab 1), 20008 (Lab 2) and 20010 (Lab 3) respectively. A [symbol] ng facility is the organization that provides medical testing services where patients can go get tested and would produce medical results. A testing facility can be a private testing facility that can receive patients at several sample collection locations and would typically have a centralized sample processing site to perform the tests and generate the results. A testing facility can also be a hospital for more complex tests, such as imaging tests.

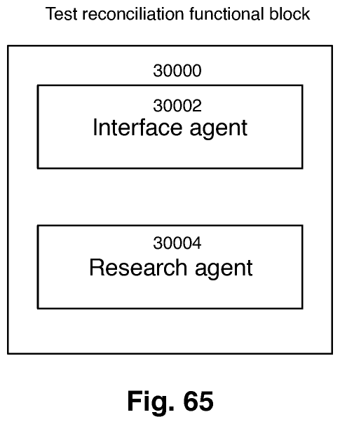

As discussed previously, the Human-Centric EHR system 20000 can be implemented as a server that connects to the various entities shown in FIG. 64 through a communication network such as the Internet. The software executed by the server includes a plurality of functional blocks that include a test reconciliation functional block 30000 shown at FIG. 65. The high-level purpose of the test reconciliation functional block 30000 is to manage the interrelations with the patient and with the testing facilities 20006, 20008 and 20010 in order to provide a tracking function and reduce the possibility of test requests or results being lost. The test reconciliation functional block 30000 includes two functional modules, namely an interface agent 30002 and a research agent 30004. The functionalities of each functional module will be described below.

The interface agent 30002 includes software code that is executed by one or more CPUs of the server to implement an interface with the entities that interact with the test reconciliation functional block, including the patient and the physician among others. The interface allows each entity to input information allowing the test reconciliation functional block 30000 to perform the tracking function and also allowing the various entities to receive information in relation to the tracking function, such as reminders and alerts, among others.

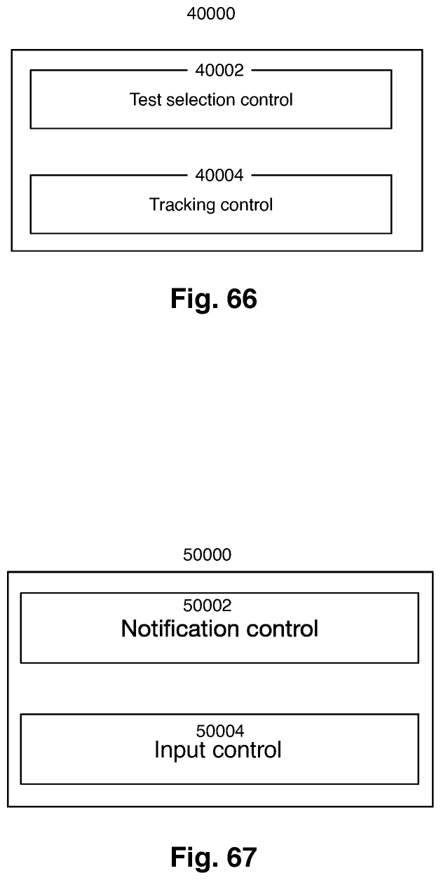

In one example of implementation, the interface agent 30002 implements on the physician's EHR 20002 a Graphical User Interface (GUI) allowing the physician that is prescribing a medical test to supply parameters about the test, in particular tracking parameters. The example of the GUI is depicted at FIG. 66. The GUI when implemented is displayed on a display screen and has information display areas, such as boxes in which information is displayed and controls that respond to user input at which the user, typically the physician will input information. More specifically, the GUI 40000 has two main functional components, namely a test selection control 40002 and a tracking control 40004. The test selection control 40002 allows the user to select the medical test that is required for the patient. In one specific mode of implementation, the test selection control can show on the display screen a list of available tests and provide check boxes or the equivalent input mechanism to allow the user to select a subset of [symbol] idual tests in the range of available tests. Alternatively, the test selection mechanism can be [symbol] nized into themes, based on possible disease conditions, such as STDs, join pain, pulmonary conditions, muscle conditions, etc. In response to input by the user, which is a selection of tests to be performed, the test selection control 40002 produces a test request that identifies the selected medical tests and also associates with the test selection identification information to distinguish this particular test request from other test requests. The identification information may include a unique identifier which does not repeat itself from one test request to the next. Any suitable alphanumeric identifier can be used for that purpose. That identifier can be randomly generated during each transaction (generation of a test request). The identification information may also include identification of the patient such as nominative information or any other suitable information that will allow to uniquely identify the patient for whom the test request is generated.

The tracking control 40004 allows the user to set tracking parameters by making user inputs at the GUI. The tracking parameters include reminder parameters for the patient about having the test done. For instance, if the test is a blood test, the reminder could include a notification to the patient to set up an appointment at a testing facility. Generally, tracking parameters in connection with the patient could include:
1. Whether patient reminder is enabled or disabled.
2. Time delay for a reminder, in other words the time period after which a reminder will be issued.
3. Number of maximal reminders.
4. Enable or disable alerts to the physician if the patient does not follow-up on reminders.

The tracking control 40004 also allows setting tracking parameters for the results of the test. That is to say, once the patient has undergone the test procedure, whether a blood test, an imaging test or any other medical test prescribed, the tracking parameters allow the physician to specify the tracking of the test results. The tracking parameters in connection with the test results include:
1. Whether tracking of results is enabled or disabled.
2. Enable or disable alerts to the physician if tracking results are missing or delayed.
3. Time delay before triggering alerts.
4. Scope/depth of tracking, that is to say whether the tracking operation will look to identify availability of some result to the test or in addition it will perform reconciliation between the test request and the results. Simple (basic) tracking will look for a result but will not try to see if what was done at the lab level corresponds precisely to the test request. For example, if a blood test was prescribed which includes multiple individual tests, the simple tracking will not try to figure out if all the tests in the request have been done. On the other hand, a complex tracking will perform reconciliation and will try to match the tests results to the test request to the individual test level. Simple tracking is suitable for situation where the test request prescribes a single test, such as an imaging test. Complex tracking including reconciliation is suitable for situations where the test request includes multiple tests, which is the case of blood tests.

FIG. 67 illustrates an example of the GUI implemented at the patient's mobile 20004 allowing the user to interact with the reconciliation functional block 30000, in particular receive notifications about prescribed tests results, reminders about the results and provide inputs such as acknowledge that the person has undergone the prescribed tests. More specifically, the GUI 50000 has two controls, namely a notification control 50002 and an input control 50004. The notification control is primarily a display mechanism to provide notifications to the patient, such as notify the patient that a test request has been prescribed by the physician. In a typical doctor visit, the physician would inform the patient that a medical test is required and would provide the patient with a test form in a written form. The notification mechanism would then confirm to the patient the requests and may provide a link where an electronic version of the test form can be retrieved. The notification control 50002 is also used to send reminder notifications in the case the patient has not confirmed that the test has been scheduled.
1. The input control 50004 is used to provide a mechanism for the client to input information. In one form of implementation, that control can be a set of control buttons, such as “yes” “no” to indicate the test status. Note that practically, the controls can be integrated into the actual notification to make it more user-friendly. In this fashion, the notification that appears on the display of the patient's system 20004 integrates the controls allowing the user to input a response, thus presenting to the user a common visual element that displays information and provides the necessary action tools to act in response to the message provided.

It should be noted that the GUI 50000 on the patient's mobile can be implemented via an app installed on the mobile, as it was discussed in previous implementations. In that case, the interface agent 30002 communicates with the app to enable the GUI at FIG. 67.

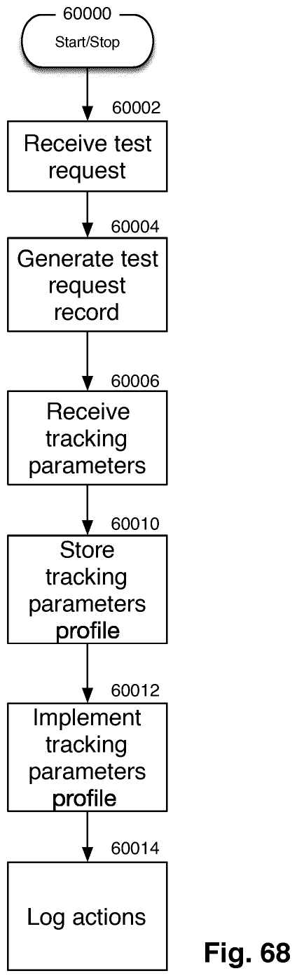

FIG. 68 is a flowchart that illustrates the operation of the interface agent 30000. At step 60000 the process starts. At step 60002, the interface agent 30000 obtains from the GUI at the physician's EHR the [symbol] t that defines the test request. As indicated previously, the test request defines one or more medical [symbol] for the patient. At step 60004, a test request record is generated. The test request record is a data group that combines the information defining the one or more medical tests and also identification information. The identification information has a unique identifier which is automatically generated when the test request record is created. The unique identifier can be a string of alphanumerical characters or be in any other form that uniquely distinguishes the test request record from other test request records for that particular patient or for other patients.

At step 60006 tracking parameters are received via the GUI, in particular via the tracking control 40004. On the basis of the tracking parameters a tracking profile is created that is stored in the server machine readable storage and linked to the test request record. Assume for the purpose of this example that the tracking parameters profile require reminders to be sent to the patient, define the reminder period to one week and enable alerts to the physician if the patient has not done the test after a number of reminders or a time frame, say a month.

At step 60012 the tracking parameters profile is implemented. That would involve starting a timer to measure the different time periods set and generate notifications that are sent to the patient via the notification control 50002, which would typically interact with the app on the mobile 20004. An example of notifications stream to the patient is as follows:
1. Immediately following the patient visit, a notification is sent to the mobile 20004 indicating that medical tests have been prescribed with a link or an attachment to a document that lists or details the tests to be performed. The patient can use this information to schedule the test at the lab or hospital. The input control 50004 is enabled to allow the patient to indicate that his or her test has been scheduled.
2. If the patient indicates that the test has been scheduled at the input control 50004, the reminder timer of the tracking parameters profile is stopped, and no further reminders are sent.
3. However, if no indication is received from the input control 50004 that the test has been scheduled, a reminder is sent after the period of time stated in the tracking parameters profile. The reminder is a notification sent to the mobile 20004 which reminds the patient to schedule the test and enables or maintains enabled the input control 50004 to indicate that the test is scheduled or has been done.
4. Step 3 is repeated the number of times specified in the tracking parameters profile. For example, the profile can specify that three reminders should be sent and no more. After three reminders have been sent the reminder functionality is completed and no more reminders are sent.
5. If the reminders cycle above is exhausted without any confirmation having been received from the patient, an alert is sent to the physician, if the alert function is enabled in the tracking parameters profile. The alert is a notification that would appear on the display of the physician computer or mobile device that would prompt the physician to take an action, such as schedule a personal call with the patient to further remind the patient as to the benefits of taking the prescribed test.

At step 60014, the actions taken by the system, in particular the tracking parameters profile, the reminders sent, and the actions taken (or not taken) by the patient are logged. The logging constitutes a record allowing to establish that in addition to prescribing a medical test to the patient, the physician followed up with diligence to remind the patient a reasonable number of times to take the test.

The research agent 30004 implements a tracking functionality after the test has been carried out and results are in principle available. The research agent implements logic that can search lab results and tries to match them to stored test request records and optionally reconcile the results with the test specified in the request.

In a first example of implementation, Lab1 20006, Lab 2 20008 and Lab 3 20010 that generate test results send those results to the server 20000 that stores those results in the machine-readable storage. The results may be structured in a number of possible ways. One possibility is to store the results as individual files such as pdf files where the information stored can be interpreted by the logic of the research agent 30004. Other ways or formats to store the information are possible. In this form of implementation, test results are pushed by the Labs (Lab1 20006, Lab 2 20008 and Lab 3 20010) to the server 20000. This assumes that the Labs would conduct some form of filtering before pushing the test results to send only those that are associated with patients serviced by the server of EHR system 20000. In other words, the Labs would not push test results that are not associated with patients that are not serviced by the EHR system 20000.

The processing includes for each active test request record, an attempt to identify test results that match the test request record. Note that test request records can have two possible states, that is; active or not [symbol] e. Test request records that are not active are those for which a test result has been identified [symbol] iously. Accordingly, unless test results have been matched to a test request record, the test request record is by default active.

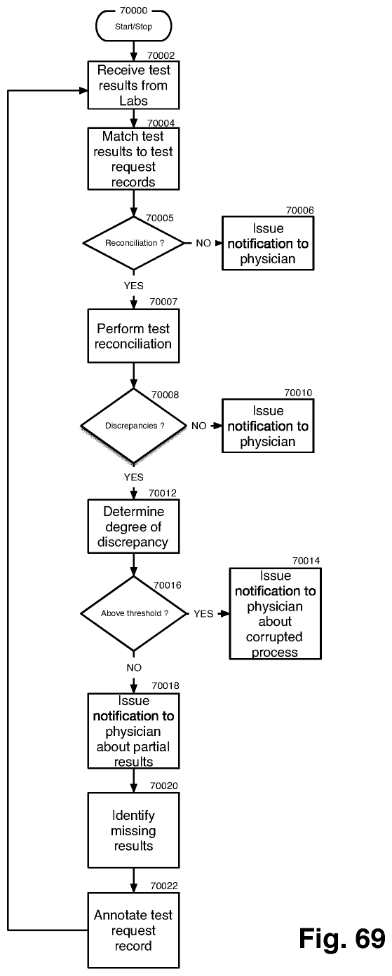

The process is illustrated by the flowchart at FIG. 69. The process starts at 70000. At step 70002 the Labs push test results to the server 2000. For each active test request record the research agent 30004 tries to match a test result. The parameters of the matching operation are defined by the tracking parameters linked to the particular test result request. Accordingly, the matching operation will be different form one test result request to another insofar the tracking parameters specify different processing.

At step 70004 the research agent matches test results to test result requests on the basis if identification information. Typically, that operation is a multi-factor match. A first factor is the identifier of the test request record which is unique and a match at that level would indicate a definitive match. However, it is possible that the Labs may not have included the identifier of the test request record in the test results and it is preferable to try matching also on basis of additional factors such as information about the patient. A match at the level of the identifier or the patient identification information is considered a match between the test result and the test request record.

Once a match is found, decision step 70005 determines if a reconciliation between the requested tests and the performed ones is necessary, according to the tracking parameters profile. In the negative the process performs step 70006 which includes issuing a notification to the physician to indicate that test results associated with that particular test request record have been obtained, such that the physician can look at the test results and communicate those to the patient, according to the process described earlier in the patent application, including sending a notification to the mobile of the patient.

If on the other hand reconciliation is required per the tracking parameters profile, the process continues with step 70007, which carries out reconciliation between what has been prescribed in terms of test and what test has actually been done. This is especially useful for blood or other tests where multiple individual tests are prescribed. The research agent 30004 will process the test request record to identify the individual tests that have been requested. This initial sub-step may be done on the basis of the test identification in the test request record, either test codes or plain language to construct a list of requested tests. The subsequent sub-step is to process the test results to generate a list of performed tests. Again, the research agent 30004 may look at the test results for test codes or plain test description, using [symbol] uage processing based on keywords and text classification techniques among others. The requested [symbol] f tests and the performed list of tests are then compared.

At decision step 70008 the process determines if discrepancies exist. A discrepancy will exist if the list of requested tests does not fully match the list of performed tests. If no discrepancy exists, step 70010 is performed where a notification is issued to the physician to indicate that test results have been received and that they are complete.

However, if discrepancies exist, further processing is performed. First the degree of discrepancy is determined at step 70012. For vast discrepancies, such as none or only a few of the requested tests have been performed, an alert is raised. The alert is channeled through the interface agent 30002 and would be sent as a notification to the physician's EHR to indicate to the physician that something wrong with the test has happened and that his or her intervention is required.

Smaller discrepancies could be normal. For instance, the research agent 30004 may not have correctly interpreted the tests designations in the test request record or in the test results for one or more tests. Another possible explanation is that sometimes test results performed in response to a single request are delivered in multiple batches of results. Some tests may take longer to complete by the lab and may be available later than some other tests.

Practically, one approach is to define a discrepancy threshold. If the discrepancy is above a certain level, that is an indication of a corrupted process and an alert is issued. Otherwise, if the discrepancy is below the threshold, that is considered an indication that results are missing, in other words the test results are partial test results. As shown at the process step 70016, which is a decision step, the process determines the degree of discrepancy and if it is above the threshold, the process branches to step 70014 to issue a notification to the physician that the process is corrupted and manual intervention is necessary. The notification can convey the test results and the also the results of the analysis of step 70007 and step 70012 to facilitate the process.

If the discrepancy level is below the threshold, as shown at step 70018 a notification is issued to the physician to indicate that partial results are issued. Optionally, at step 70020 the process identifies the tests that have been requested and for which results have not been received yet and includes that information in the notification. At step 70022 the missing test information is linked to the test request record such that a new iteration of the reconciliation process would only be done on the missing results. It [symbol] be noted that once step 70022 is completed, the process loops back at step 70006 to process a new [symbol] h of test results received from the Labs.

Note that the process integrates timers to ensure that the processes or sub-processes time out instead of running indefinitely, for example to indicate that results are missing after a preset time period. More specifically, there is a first timer set once the test request record becomes active, that is the requested test has been carried out. Typically, that timer can be set to a period of one week. For one week the process shown at FIG. 70 runs and looks for results. If no results are found and the process times out, an alert is sent to the physician to indicate that results are missing. The physician can then decide to take appropriate action.

A second timer is provided to manage partial results. For instance, once the occurrence of partial results is identified, such as at steps 70016 and 70018, indicating that it is likely that additional results will be received, the timer starts. The second timer is likely to be set to lesser time interval than the first timer, say a few days for the additional results to arrive. After this period times out, an alert notification is sent to the physician.

Note that although not shown in the flowchart of FIG. 69 a logging sub-process is performed to log the various activities, in particular the tracking and the reconciliation activities such as to be able to establish at a later date what was actually done and identify the alerts and notifications that have been sent as a result of the processing.

Drug Prescription Management

The Human-Centric EHR system 102 that was described above according to a range of possible implementations constitutes a platform allowing managing drug prescriptions to facilitate the transmission of the prescription to a patient, facilitate the purchase of prescribed drugs and also follow-up with the patient to allow the physician to track whether the patient is taking the medication and whether side effects are occurring that may require some remedial action, such as discontinuation of the medication or substitution of a different medication.

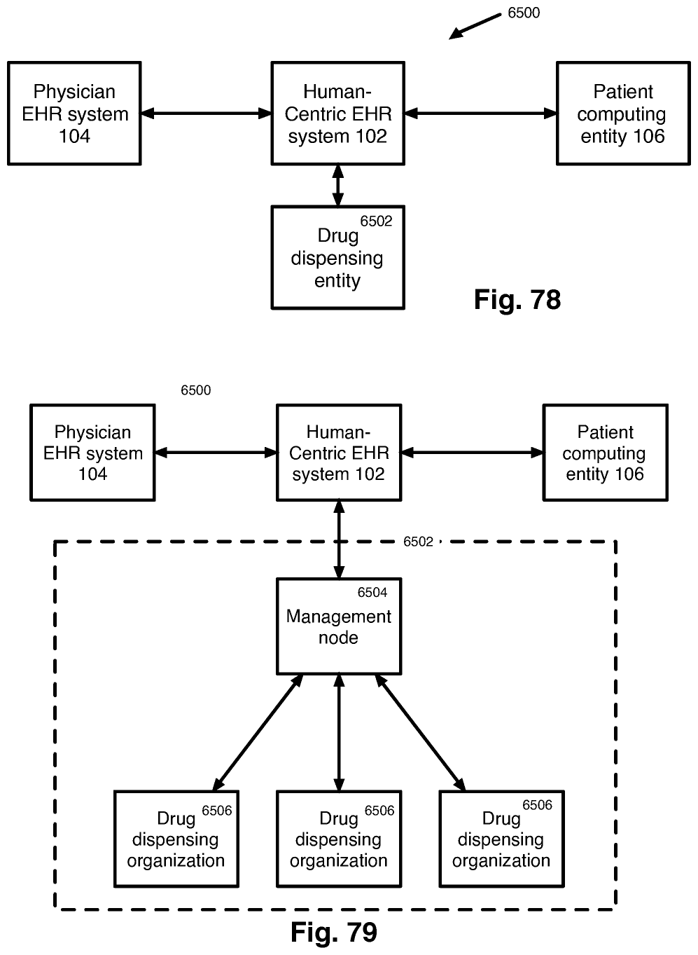

FIG. 78 is a block diagram of a network arrangement 6500 illustrating a number of network entities that interoperate to provide services to the patient for drug prescription management. Specifically, the network arrangement includes a Physician EHR system 104, a Human-Centric EHR system 102 and a patient [symbol] puting entity 106. Further, the network arrangement includes a drug dispensing entity 6502 in [symbol] munication with the Human-Centric EHR system 102. The drug dispensing entity 6502 generically refers to a single organization or to a group of organizations that can dispense drugs to fulfill a drug prescription and optionally provide ancillary services and products to the patient. A single organization can be a pharmacy for example. A group of organizations can be a group of pharmacies. Such a group of pharmacies can be a pharmacy chain, where individual pharmacies are linked to one another by a computer system that manages the chain. An illustration of a multi-organizational drug dispensing entity is illustrated in FIG. 79. The drug dispensing entity 6502 has a management node 6504 that communicates with the Human-Centric EHR system 102, and in turn communicates with the individual drug dispensing organizations 6506. In the example shown, three drug dispensing organizations are shown, it being understood that fewer than three of more than three can be provided.

In a practical example, the drug dispensing entity 6502 is a pharmacy chain that has individual pharmacies at different locations throughout a city, a province or a country. The pharmacy chain has a central management location, which is associated with the management node 6504. The central management location controls the server arrangement that communicates with the Human-Centric EHR system 102, it being understood that each drug dispensing organization would also be associated with a corresponding server that forms a node in the network arrangement 6500. In most types of implementations, the drug dispensing entity would operate its own private network and communications with the Human-Centric EHR system 102 would occur via the management node 6504. Accordingly, in this example of implementation the drug dispensing entity 6502 defines its own private network, which is distinct form the network arrangement 6500. Both networks are configured to communicate with each other such as to be able to provide a functional interoperation to provide prescription drug services to the patient.

Note that while FIGS. 78 and 79 make reference to a single drug dispensing entity 6502, the network arrangement 6500 can communicate with multiple drug dispensing entities 6502, where each drug dispensing entity 6502 operates in its own private network. In this example, each drug dispensing entity 6502 can be a different pharmacy chain and as such the patient has more than one option, in terms of pharmacy brand that he or she can deal with for prescription drug dispensing services.

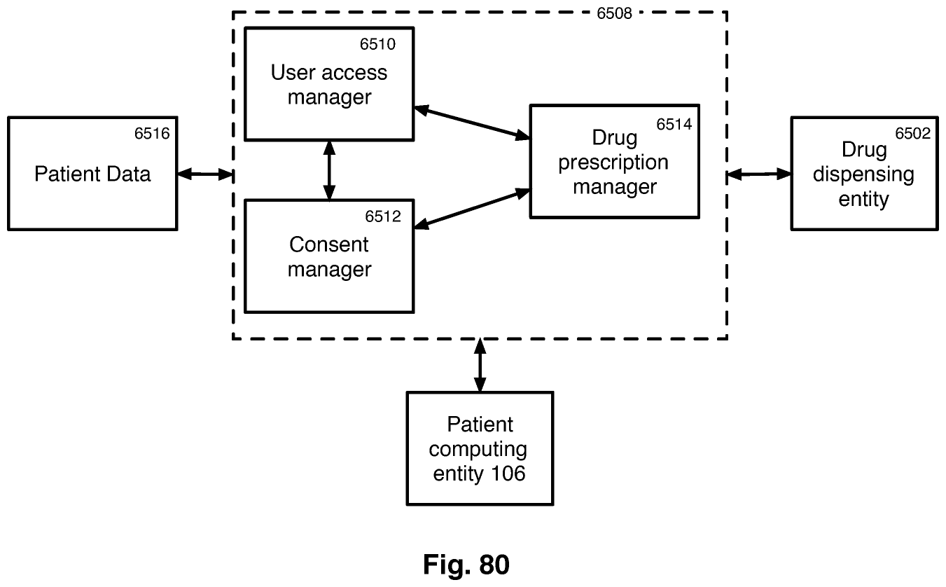

FIG. 80 is a block diagram illustrating software implemented functional blocks that are part of the Human-Centric EHR system 102. It should be noted that FIG. 80 does not show the software [symbol] itecture of the Human-Centric EHR system 102 in its entirety. It depicts only the elements that are [symbol] ssary for the implementation of the drug prescription management function in this example.

More specifically, and as described earlier the Human-Centric EHR system 102 has a multiplicity of user profiles associated with different users or patients. Each user profile holds parameter settings that reflect individual patient preferences in terms of customization of the different functionalities that the Human-Centric EHR system 102 provides. In FIG. 80, the user profile is generically designated by 6508 and it is associated with a number of software implemented functionalities to implement drug prescription management. In the specific example shown, those functionalities are built on top of the user access manager 6510 functions and the consent manager 6512 functions, described previously.

The additional functionalities included in the user profile 6508 to provide the drug prescription management include a drug prescription manager that interfaces with the patient, via the patient computing entity 106 and also interfaces with the drug dispensing entity 6502. Generally, the drug prescription manager 6514 provides an interface allowing the patient to customize the functionality of the drug prescription manager 6514, for example define the scope of interaction that a drug dispensing entity 6502 can have with the patient. In a specific example, the scope of interaction may define the range of services and products the drug dispensing entity 6502 can provide to the patient and/or the patient data that the drug dispensing entity 6502 can see.

The scope of interaction is expressed with parameters, examples of which are discussed below. Once those parameters are defined, the prescription drug manager 6514 will modulate the interactions with the drug dispensing entity 6502 accordingly, for example limit access to only those parts of the patient data that the patient has authorized to be exposed and nothing more. It will be noted that the drug prescription manager 6514 interoperates with the user-access manager 6510 and with the consent manager 6512 which are gateways to the patient data. For instance, the drug prescription manager parameters can be set through the consent manager user interface 6512. In this fashion, access to patient data by external entities is managed through a unified GUI that covers the entire spectrum of service scenarios for the patient. Alternatively, the drug prescription manager 6514 can have its own interface to allow the patient to set the necessary parameters such as to customize the functionality of the drug prescription manager 6514 as desired.

Generally, the drug prescription manger 6514 provides two levels of services. There is an internal level of services that do not involve an external entity, such as the drug dispensing entity 6502. Typically, the [symbol] nal level of services includes interactions with the physician that has prescribed the drugs to the [symbol] nt. And there is also an external level of services that deal primarily with interaction involving the drug dispensing entity 6502.

The internal level of services are functionalities that are mainly intended to track events that occur after a physician has prescribed a drug to the patient such as for example, track whether the prescription has been fulfilled, in other words, is the patient taking the drug as prescribed. Another event or condition that the internal level of services track is suitability of the prescribed drug, such detecting the occurrence of undesirable reactions to the drug and in the affirmative alerting the physician. The internal level of services thus supports the patient in terms of (1) compliance with the prescribed drug regimen and (2) maintain patient engagement to identify undesirable reactions to a prescribed drug that may require discontinuance of the drug or substitution with a different drug. An ancillary benefit to providing this level of services is liability management on the physician side by being able to log communications such as reminders sent to the patient and thus establish engagement with the patient after prescription of a drug.

The external level of services, which involve the drug dispensing entity 6502 are intended to facilitate the commercial procurement of the prescribed drugs and also the provision of ancillary services and products the drug dispensing entity 6502 may want to provide to the patient. The drug prescription manager 6514, however manages this interaction to avoid uncontrolled marketing materials and requests flooding the patient. Examples of both levels of services are provided below.

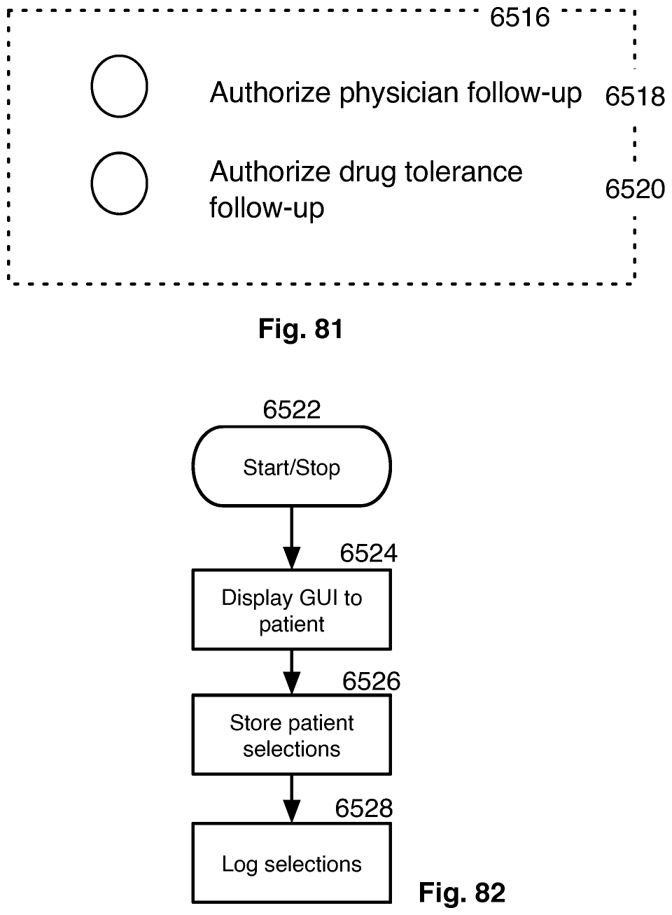

FIG. 81 is an example of a GUI which can be invoked by the drug prescription manager 6514 allowing the patient to define the scope of the internal class of services. For convenience, the GUI can be integrated into the user access manager 6510 or the consent manager 6512 or can be a stand-alone GUI that is associated with the drug prescription manager 6514.

The GUI 6516 has a display area holding a number of controls allowing the patient to define the parameters of the internal level of services. In this example, the controls are in the form of radio buttons, but it will be understood that any other suitable GUI control allowing to set the patient preferences can be used. The controls include a first radio button control 6518 allowing the patient to specify whether physician follow-ups after a drug prescription has been issued are allowed. That selection would apply to follow-ups to ensure the patient is fulfilling the prescription and taking the medication. That selectable parameter can have a default value, which is in the affirmative, in other words physician follow-up is enabled but the patient may not want that and can change it such as follow-ups are not allowed. The GUI [symbol] also has an additional control 6520 which relates to follow-ups regarding drug tolerance which are [symbol] iries made to see if the drug is tolerated adequately by the patient. Again, the setting can have a default value, which is to enable the inquires, but that setting is changeable if the patient so desires.

The flowchart at FIG. 82 illustrates the process for setting the parameters of the GUI 6516. The process starts at 6522. At step 6524 the GUI 6516 is displayed to the patient on the patient's computing entity 106. It will be recalled that access to the Human-Centric system 102 by the patient, when the patient's computing entity 106 is implemented by a mobile requires validation to ensure the person accessing the Human-Centric EHR system 102 is the actual patient. In the embodiment where the mobile 106 uses an app which communicates with the Human-Centric EHR system 102, the patent would typically perform authentication at the mobile level, which can be biometric, or password based to unlock the mobile and preferably another authentication at the app level to enable the app. The authentication at the app level can be biometric or password based. Once authentication is completed and the app is unlocked, the user can access the GUI 6516 via the settings menu.

At step 6524 the user sets the parameters via the GUI controls and closes the GUI. The parameters as modified by the patient are communicated to the server of the Human-Centric EHR system 102 and stored in the user profile, at step 6526. At step 6528 the patient selections are stored in the system log of the Human-Centric EHR system 102 to keep a record of the settings changes such that the system behavior at some later date can be explained. For instance, if a patient decides to deny follow-up services and omits to fulfill the prescription of a certain drug, which has downstream consequences for his or her health, the log will be able to establish that the absence of follow-up is at the express directive of the patient.

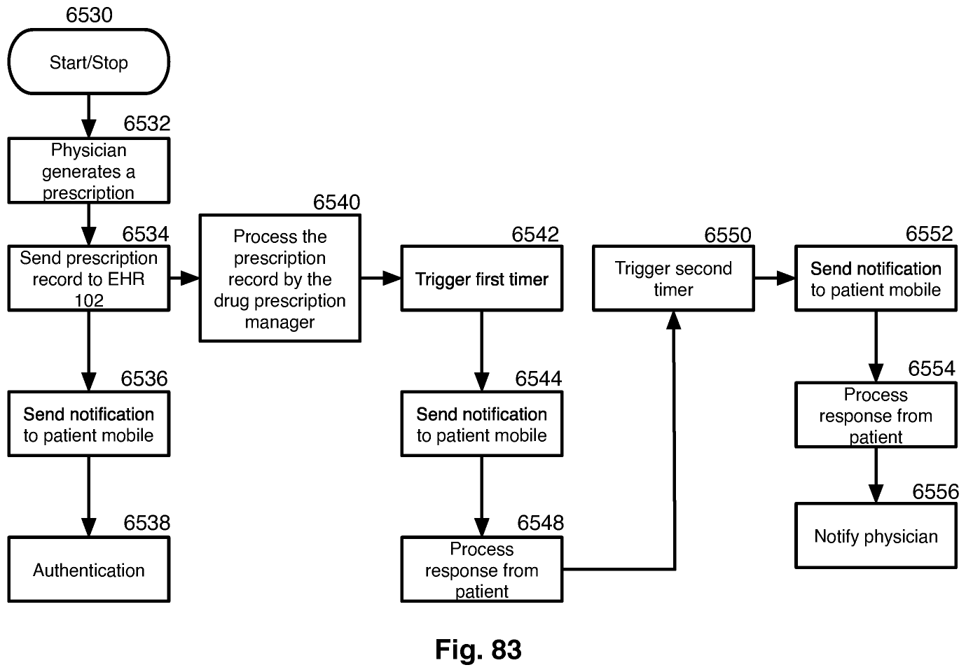

FIG. 83 illustrates a flowchart depicting the steps of an interaction involving a physician and a patient which involves the internal level of services in the context of a drug prescription. For clarity, that interaction does not involve the drug dispensing entity 6502.

The process starts at 6530. Assume that the patient is having a consultation with the physician, which can either be in person or virtual. The physician prescribes a drug to the patient and generates a prescription. The physician uses the prescription generation tool at the physician EHR system 104. This is shown at step 6532 which involves the generation of an electronic drug prescription record that is sent to the Human-Centric EHR system 102 and stored in the patient data, as identified by step 6534. Since this is a new event in the medical information of the patient, the patient will be notified about it, as described earlier and as per step 6536. If the patient computing entity 106 is a mobile, the Human-Centric EHR [symbol] m 102 will generate a notification via the communication channel established between the server of [symbol] Human-Centric EHR system 102 and the app on the patient mobile 106. Accordingly, the mobile will display on the screen a notification indicating that the app requires user attention. As discussed previously, the type of information that is shown on the notification can be determined via user settings in the app. It is preferred to not provide any medical information for privacy purposes.

After the notification appears on the mobile screen, the patient will unlock the mobile via biometric or password authentication and open the app, which preferably also requires separate authentication at the app level. This authentication process is identified by step 6538. At this point, the prescription record is accessible to the patient and the patient can view it and optionally communicate it to a third party, via the controlled information sharing process described previously. The third party may be a pharmacist that will fulfill the prescription. Specifically, the pharmacist may be directed via a link to the server of the Human-Centric EHR system 102, that will expose the prescription record, via a QR code, as discussed previously.

Note that the act by the patent to access the prescription record via the app is reported back by the app to the server of the Human-Centric EHR system 102, and that activity is logged in the system central log, which will be able to establish that the prescription record was delivered to the patient and the patient acknowledged it by accessing the prescription record.

The drug prescription record contains drug prescription information. The structure and format of the drug prescription record can vary. In one example, the drug prescription record can be a pdf file and provide an image of a drug prescription document listing the one or more medications prescribed to the patient. In this example, the drug prescription information is simply an image of a document. Optionally, the drug prescription record can convey semantic information, that identifies via a code or other identifier that the record contains drug prescription information. This is a useful approach in managing the drug prescription record by the drug prescription manager 6514, in particular allowing the drug prescription manager 6514 to distinguish prescription drugs from other types of medical information that are uploaded to the Human-Centric EHR system 102. In such example, the drug prescription manager 6514 processes the entire flow of medical information associated with the patient that is uploaded to the Human-Centric EHR system 102, to filter out the drug prescription records on the basis of the semantic information. Alternatively, the drug prescription manager can be configured to perform a scan of the medical information flow to identify records that are prescription drug related. Such scan can be a key word-based scan, for example. [symbol] processing of the drug prescription record by the drug prescription manager 6514 after reception by [symbol] ne Human-Centric EHR system 102 is shown by a branching out process thread, which starts by the initiation of the internal level of services, in particular the identification by the drug prescription manager of the new prescription record. This is shown at step 6540. At step 6542, the drug prescription manager 6514 will trigger a series of notification processes, depending on the settings specified by the patient at the GUI shown at FIG. 81. Assuming the patient has authorized physician follow-up to ensure the prescription has been fulfilled and then drug tolerance follow-ups, the drug prescription manager will trigger a first timer at step 6542 to send at step 6544 a notification to the patient to inquire if the patient has fulfilled the prescription. The notification process is as described earlier, where the notification is shown at the screen of the mobile and the patient is authenticated as at step 6538 to view the notification.

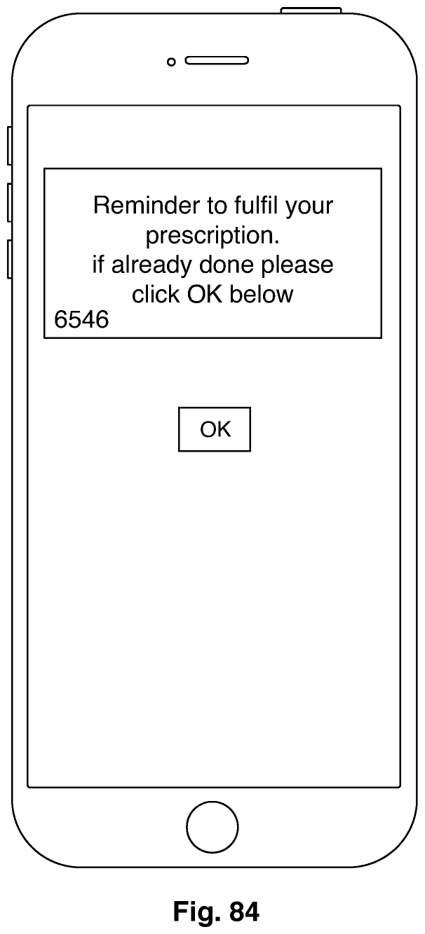

FIG. 84 is an example of what the notification could look like after it is opened on the display of the mobile, after successful user authentication. The notification 6546 includes a response mechanism, in the form of a button “OK” allowing the patient to indicate that the prescription has been fulfilled. At step 6548 the Human-Centric EHR system 102 receives the response from the notification, at step 6548. If the response is “OK” or the equivalent, the timer triggered at step 6542 is stopped and the patient response is recorded in the system log to establish that a follow up was done and the patient acknowledged that the prescription was fulfilled and presumably the prescribed drug is taken.

On the other hand, if no response is provided to the notification or the patient responds via the notification that the prescription is not yet fulfilled, the response is logged and the timer at step 6542 is re-set, such that another notification can be sent out later. In that case, steps 6544 and 6548 are repeated. The number of times the notification is sent out can vary. Note that if after several attempts the patient still does not acknowledge that the prescription is being fulfilled, a message can be sent to the physician's EHR system 104 to notify the physician.

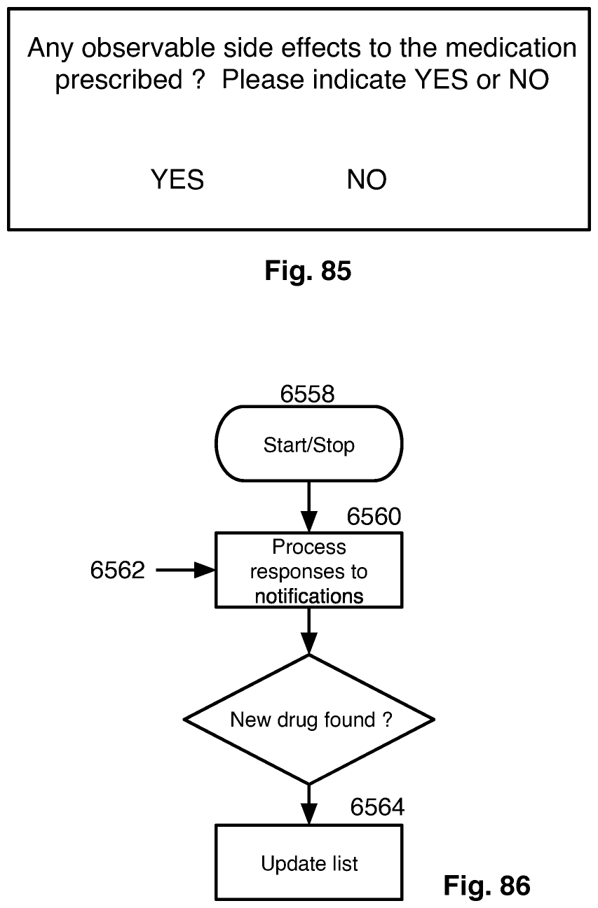

In the event the patient acknowledges that the prescription has been fulfilled, the drug prescription manager triggers a second timer at step 6550 for the purpose of a follow-up to see if the patient has any abnormal reactions to the prescribed medication. The notification about possible adverse effects is sent at 6552. An example of a notification is shown at FIG. 85. For clarity and simplicity, the mobile of the patient is not shown. This example of notification is generic, in the sense it asks a general question about possible side effects the patient may be observing and provides a response mechanism, in the form of a “YES” and “NO” answer. Although not shown specifically in the flowchart of FIG. 83, it will be [symbol] rstood that the patient can access the notification via an authentication process identical to the one [symbol] tified by step 6538.

At step 6554 the response to the notification is processed. If the answer is “NO” no action is taken. Otherwise, a message is sent to the physician via the physician's EHR system 104 to follow-up with the patient as shown at step 6556.

In the event the patient does not provide a response steps 6550 and 6552 repeat a predetermined number of times and all events are recorded in a log, as indicated previously.

In a possible variant, the notification sent at step 6552 is adapted to the particular medication prescribed instead of being a generic one. That is to say that the questions about the possible side effects are specific to the medication prescribed. The questions are thus more focused and likely to elicit a more meaningful response from the patient. In this variant, the drug prescription manager 6514 includes a notification generator module that generates questions in dependence of the particular drugs that have been prescribed by the physician. The drug generator module, which is software implemented, has two main units, one being provided to extract from the prescription record the particular drugs prescribed by the physician in a machine readable form and the other mapping drugs to possible side effect questions, which are drug specific. The first unit therefore receives at an input the drug prescription record and performs a processing such as a keyword-based processing to identify the different medications that have been prescribed to the patient and provides the list so extracted to the second unit that maps each drug on the list with possible side effects. That mapping is obtained from a database, not shown in the drawings. The database thus returns a list of possible side effects and the notification is built by the notification generator module to include the side effect questions that are sent to the patient. The responses received by the patient are processed as discussed previously and sent to the physician if any side effects are noted.

In a further variant, the prescription drug manger performs historical tracking of the patient responses to the notifications to avoid asking questions about drugs for which it has been established that no negative reaction exists. This process is described by the flowchart of FIG. 86. The process starts at 6558. At step 6560 responses to notifications received from the patient at 6562 are received by the prescription drug manager 6514 and processed against the list of drugs in the patient profile against which no adverse reaction has been recorded. Accordingly, if the patient is prescribed a new drug and at input 6562 the patient indicates that no adverse reaction is observed, that drug is placed on the list in the patient profile at step 6564. Thus, by virtue of this process, the list is continuously updated, and it holds all the [symbol] ications that the patient has taken in the past or is currently taking for which no adverse reactions [symbol] been recorded.

When a prescription record is generated, before a notification is sent out by the prescription drug manager 6514, the list stored at step 6564 is retrieved and compared to the drugs in the current prescription record. If no new drugs are found, in other words the prescription record only lists drugs for which the list shows that no adverse reaction exists, then no notification will be sent out. In this fashion, the process will only send out notifications for newly prescribed drugs.

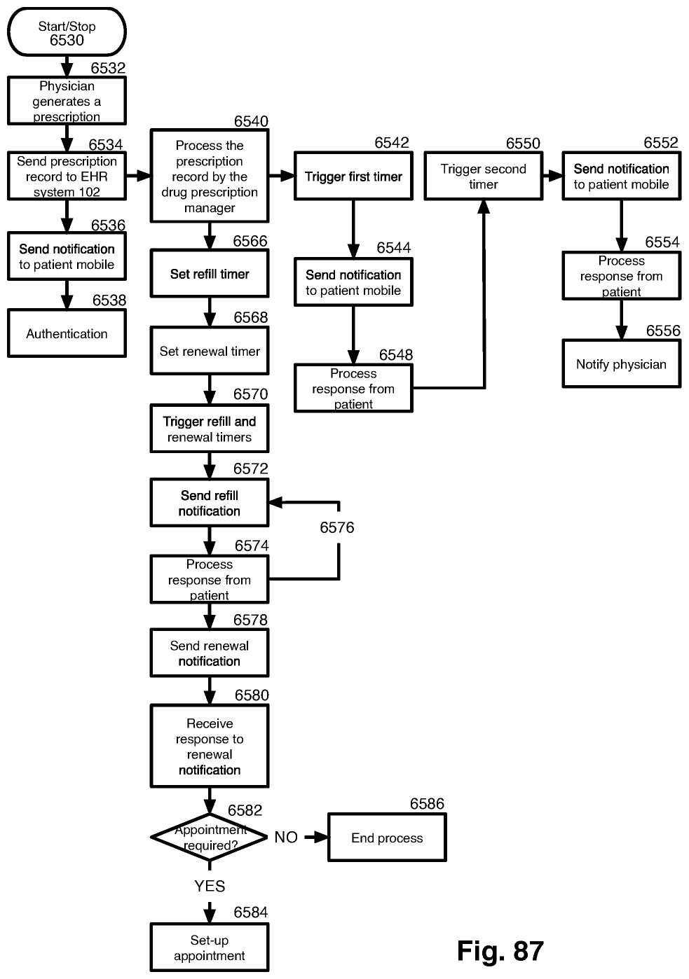

Another possible service at the internal level is the management of drug prescription reminders. The reminder service can provide two types of reminders. A first reminder to indicate to the patient that the prescription is due for a refill. Typically, refills are done on a monthly basis and such reminder would be issued at or about the estimated time period at which the refill is due. A second reminder is to indicate that a prescription renewal is due and thus avoid a situation where the patient runs out of medication. Specific examples of both reminders are provided below. The flowchart at FIG. 87 is an extension of the one of FIG. 83 and illustrates the process steps that occur to implement both reminder mechanisms. Also note that the GUI 6516 can be modified to take into account the reminders by providing controls to indicate if the patient authorizes reminders for refills and reminders for renewals.

The process steps to implement the refill reminders branches out of the step 6540, where the drug prescription manager 6514 receives the drug prescription record. At step 6566, the timer is set which can have a selectable duration, which by default can be of 30 days, that is the typical refill period. At step 6568 a renewal timer is set, again of selectable duration, which typically would be of 12 months, which is the default renewal period for a prescription. Both the refill timer and the renewal timer are triggered as a result of the interaction occurring at steps 6542, 6544 and 6548. Once the patient advises that the drug prescription has been fulfilled, the refill and the renewal timers are triggered at step 6570.

After the refill timer expires, a refill reminder is sent at step 6572. The notification can be similar to the notification shown at FIG. 84, in that it conveys a response mechanism allowing the patient to acknowledge that a refill has been ordered or will be ordered shortly. The response from the patient is processed at 6574. In the event no response is received from the patient to the notification, additional notifications can be sent and eventually a notification can be sent to the physician to determine if his or her involvement is required. The loopback arrow 6576 depicts the repetition of this process at each refill cycle, such as every month. [symbol] r the drug prescription renewal period expires, in this example 12 months after the issuance of the drug prescription, the renewal timer triggers a renewal notification at step 6578. The behavior of the renewal notification is different from the one of the refill notification, which is mostly automatic. The renewal notification can be a stand-alone notification that does not require a response from the patient and is sent to indicate to the patient that a meeting with the physician, either in person or by video conference is necessary for the physician to issue a renewal. The intent of the notification is to prompt the patient to make an appointment with the physician. Note that all those interactions are logged at the system level, therefore even though no response is required from the patient at the notification, it can be established by the log that the notification was sent and the patient viewed it, hence he or she was made aware of the necessity to renew the drug prescription.

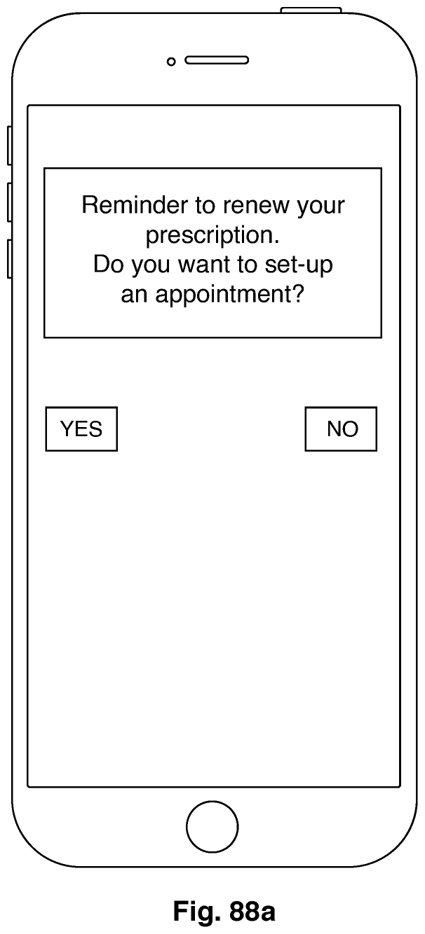

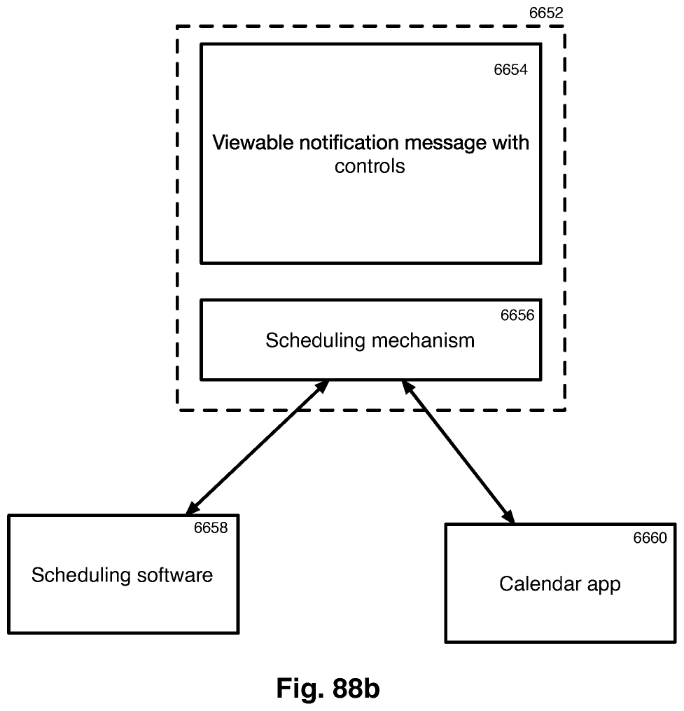

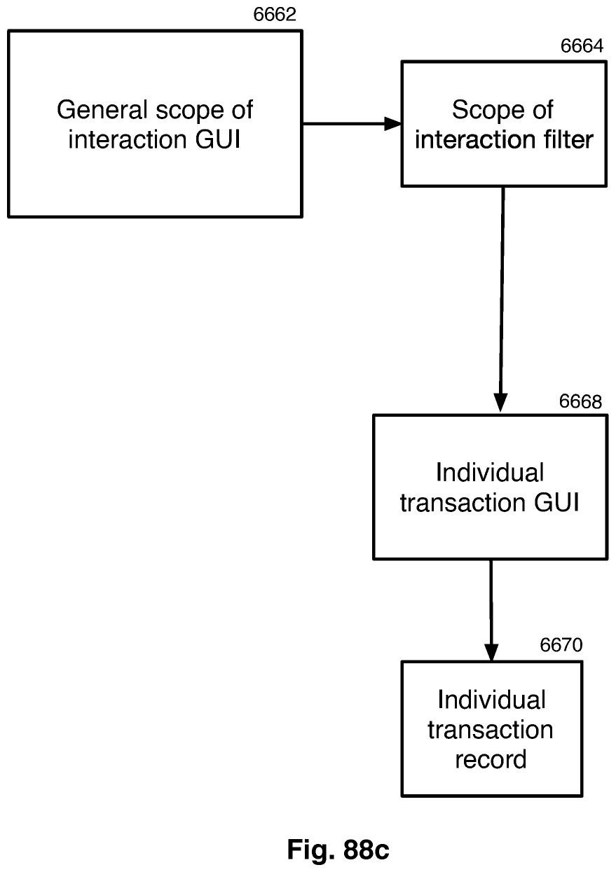

Optionally, the reminder can include a scheduling mechanism to set an appointment with the physician. An example of such notification is shown at FIG. 88a. The notification includes control buttons, YES and NO. If the patient answers, NO, nothing happens. But if the patient answers YES, that triggers an appointment setting process, which can include opening up a calendar and providing scheduling parameters, such as possible dates and times during which the physician is available, extracted from a schedule of the physician obtained from the physician EHR system 104.

FIG. 88b illustrates the structure in terms of functional blocks of the renewal notification. The notification 6652 includes a viewable area 6654 that contains a message for the patient and also user operable controls that the patient can selectively activate. In this example the controls can be the buttons YES and NO as shown in FIG. 88a. The notification also includes a scheduling mechanism 6656 which is responsive to the controls in the visible area 6654. If the patient answers NO, the scheduling mechanism 6656 does nothing. However, if the patient answers YES, the scheduling mechanism 6656 will perform a series of interactions with scheduling software 6658 at the physician EHR system 106 end, to obtain scheduling information, in term of possible dates and times where an appointment with the physician is possible and forwards those to the patient for selection. That particular interaction can be performed as a sequence of individual notifications, where the YES answer to the initial notification would trigger the internal interaction to obtain the scheduling information that is delivered to the mobile 106 as a second notification showing in its visible area 6654 the scheduling information, such as a list of dates and times. The patient can select anyone of those dates and times simply by pressing on the selected option, which would in turn trigger the underlying scheduling mechanism 6656 to communicate the selected option to the scheduling software at the physician EHR system 106 end such that the selected [symbol] and time is entered in the physician calendar. Optionally, the scheduling mechanism 6656 can also [symbol] act with a calendar app 6660 on the mobile 106 to enter the selected date and time in the calendar of the patient.

Referring back to FIG. 87, step 6580 illustrates the processing of the response to the notification. If decision step 6582 determines that an appointment is requested by the patient, the process sets-up the appointment at step 6584. Otherwise, the process ends at 6586 while logging the activity that the patient was reminded of the necessity to renew the prescription but declined an appointment with the physician to do so.

As indicated briefly above, the external level of services involves the drug dispensing entity 6502 that can fulfill the drug prescription and also provide ancillary services to the patient, which are customized to what the patient wants. Accordingly, the patient can specify the level of engagement with the drug dispensing entity 6502 which can be as simple as the purchase of the prescription drugs to a more elaborate level of engagement where the patient receives offers for additional products and services that may be specifically related to the drug prescription or of more general nature.

Referring back to the block diagram at FIG. 80, the drug prescription manager 6514 is configured to implement a GUI allowing the patient to define the desired scope of interaction with the drug dispensing entity 6502. The GUI is configured with user operable controls, not shown, allowing the user to specify a range of parameters. Generally, the parameters fall in broad categories such as:
1. A global category where the patient can authorize or not authorize an interaction with the drug prescription entity 6502—That parameter, if set in the affirmative (authorize) would enable the drug dispensing entity, through a controlled communication mechanism, to engage the patient via the app on the mobile 106. Otherwise, if the patient choses to set this parameter to “not authorize”, the drug dispensing entity 6502, is not able to communicate with the patient via the app.
2. A category of products or services that the drug dispensing entity 6502 is authorized to offer that are in direct relation to the drug prescription, such as:
   a. Home delivery services of the prescribed drugs.
   b. Product offers or services supplementing one or more of the prescribed drugs. For example, a known side effect of a prescribed drug may be dryness of the skin. In that example, the drug dispensing entity 6502 may want to offer a moisturizing cream against the skin dryness, along with the prescribed drugs.
   c. Prescription related notifications such as refill reminders.
3. A category of products and services that are unrelated to the drug prescription, such as general marketing offers.

When the user interacts with the GUI and sets the different class parameters as desired, the settings are stored in a general scope of interaction filter that is part of the user profile 6508. That filter is invoked when a prescription is delivered to the patient computing entity 106 to modulate the patient interaction with the drug dispensing entity 6502 according to the preferences of the patient.

This is illustrated by the block diagram at FIG. 88c. The general scope of interaction GUI 6662 outputs a general scope of interaction filter 6664 that defines the preferences of the patient as to its interaction with the drug dispensing entity 6502 in terms of broad classes or themes set via the GUI 6662. In this fashion, repeated individual transactions with the drug-dispensing entity 6502 are streamlined, for example by removing options as to products and services that can be offered by the drug-dispensing entity 6502 that the patient does not want. Specifically, the scope of interaction filter 6664 will condition an individual transaction GUI 6668, which is triggered when a drug prescription transaction is to be performed. As it will be described below, once a drug prescription record is delivered to the patient mobile 106, a transaction GUI 6668 is triggered to ask the patient what specific services are requested from the drug dispensing entity 6502 in relation to the particular drug prescription. The transaction GUI 6668 that the patient will see, is dependent on the selections made on the general scope of interaction GUI 6662. The options provided by the transaction GUI 6668 are constrained to the classes of products and services specified at the general scope of interaction GUI 6662.

The interaction between the patient and the transaction GUI 6668 produces a transaction record 6670 that defines the specific services and products the patient wants from the drug dispensing entity 6502 in the context of a particular transaction. For instance, at every drug prescription refill, a specific transaction record 6670 is produced.

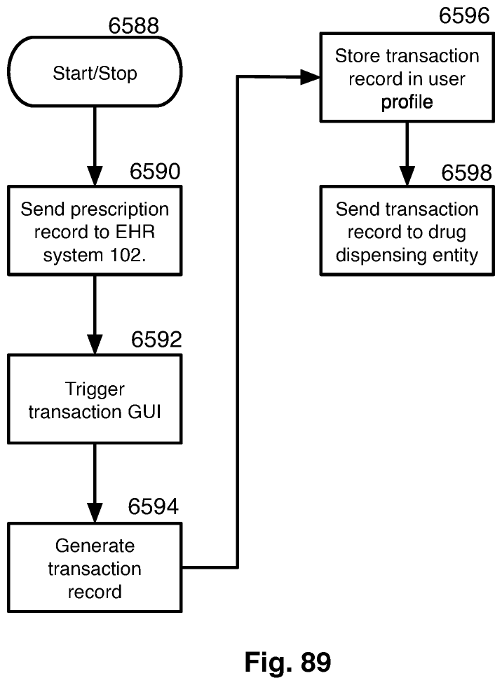

A more specific example of the overall interaction is depicted by the flowchart at FIG. 89. The process starts at step 6588. At step 6590 the patient receives a prescription record from the physician EHR system 104. It will be understood that step 6590 encompasses steps 6534, 6536 and 6538 described [symbol] er, in particular in relation to the flowcharts of FIGS. 83 and 87. At step 6592 the delivery of the [symbol] ription record prompts the drug prescription manager 6514 to trigger a transaction GUI 6668 at step 6592, which is conditioned by the interaction filter 6664 as discussed, which will enable or disable choices on the transaction GUI 6668 depending on the settings of the general scope of interaction GUI 6662.

In a first specific example, the interaction GUI 6662, hence the interaction filter 6664 are set to disable an interaction with the drug dispensing entity 6502. Practically, the steps 6590 and 6592 are performed in the background and the transaction GUI 6668 is not enabled in the sense it is not shown to the patient allowing him or her to make selections for ancillary services as no interaction with the drug dispensing entity 6502 is allowed. That example would correspond to a situation where the patient prefers to fulfill the drug prescription by visiting a pharmacy in person.

In a second specific example, assume the patient has set the global scope of interaction GUI 6662 to enable interaction with the drug dispensing entity 6502 but that interaction is constrained to products and services in direct relation to the drug prescription. In other words, products and services of general nature and not specifically related to the drug prescription are not authorized. In this example, at step 6592 the patient is presented with a transaction GUI 6668 providing a list of options that the patient can choose from within the scope of products and services related to the drug prescription.

Optionally, the transaction GUI 6668 provides a selection mechanism allowing the patient to select a drug dispensing entity 6502 among several possible drug dispensing entities 6502. Recall, that in practice the network arrangement shown in FIGS. 78 and 79 would include multiple drug dispensing entities 6502, such as different brands of pharmacy chains. Accordingly, the entry point in the selection process defined by the transaction GUI 6668 would include a list of the available drug dispensing entities 6502, asking the patient to make a selection.

After the selection of a drug dispensing entity 6502 the selection of product and services is made at the transaction GUI 6668. The choices can be displayed as a list with associated controls allowing the patient to pick and choose the desired ones. For example, the transaction GUI 6668 can present the list of the following items along with selection controls:
1. Home delivery service (yes or no)
2. Products and services complementary to the drug prescription (yes or no)
3. Refill reminders (yes or no)

Note, in relation to the refill reminder services such services are already provided by the EHR system 106, however the patient may prefer getting reminders from a particular drug dispensing entity. Also, both reminder services may be enabled at the same time.

In a third example, all the available products and services are enabled, including the class of product and services unrelated to the drug prescription. The interaction with the transaction GUI 6668 is generally similar to the previous example, the difference being that an additional page is provided to list the additional choices in the category of products and services not directly related to the drug prescription.

Note that in response to the selection of one or more options at the transaction GUI 6668 additional GUI pages can be presented to prompt the patient to supply additional information that the selected drug dispensing entity 6502 may need to perform the selected product and services. For example, if the patient wants home delivery of the prescribed drugs, address information, information about a payment instrument that can be charged by the drug dispensing entity 6502 for the delivery and for the prescription drugs and in the instance the patient has insurance, insurance policy specifics to allow the drug dispensing entity to process the cost of the prescription directly with the insurer, may need to be provided. Some of that information may already exist in the user profile 6508, some may not. Once the necessary information is provided by the patient all the patient input data is stored into the transaction record 6670, as shown at step 6594 and sent to the selected drug dispensing entity 6502. The transaction record 6670 could include the drug prescription record, either as a file that contains the drug prescription information, such as an image of the drug prescription, or a link to a network location where that information can be found. The drug prescription manager 6514 also generates a unique identifier for the transaction record allowing communications from the drug dispensing entity 6502 to be linked to the transaction record 6670. Preferably, the identifier is anonymous in the sense it does not convey any nominative information, phone number, email address, etc. of the patient. In this way, confidentiality of the patient is maintained to the extent possible.

The transaction record 6670 is stored in the user profile 6508 at step 6596, to provide a record of a request made for products and services at the drug dispensing entity 6502, such that communications originating from the drug dispensing entity 6502 to the patient, in response to the request, can be correctly linked to the patient user profile 6508. Also, the transaction record can be used as a filter to constrain or eliminate [symbol] munications from the drug dispensing entity 6502 that are inconsistent with the settings of the general [symbol] e of interaction GUI 6662 and the transaction GUI 6668.

Step 6598 identifies the transmission of the transaction record 6670 to the drug dispensing entity 6502.

Once the drug dispensing entity 6502 receives the transaction record 6670 it performs the services requested, including fulfilling the drug prescription and delivering the drugs to the home of the patient, if that service has been requested. Communications with the patient happen through the Human-Centric EHR 106. Specifically, the drug dispensing entity 6502 sends a message through the Human-Centric EHR 106, which includes as an identifier, the identifier in the transaction record, such that the Human-Centric EHR 106 can match it to a user profile and process the message. The processing can be performed as any other communication sent to the patient, including triggering a notification on the user mobile 106 to prompt the user to interact with the app, where the message from the drug dispensing entity 6502 can be viewed after authentication of the patient has been performed.

Optionally, the user profile may be provided with a filtering agent (not shown in the drawings) to control the information that the drug dispensing entity 6502 is sending to the patient. The filtering agent is designed to process the messaging that the patient is receiving from the drug dispensing entity 6502 to avoid communications that are outside the scope of the interaction specified by the patient, in particular offers for services or products that have not been selected by the patient. The filtering agent uses the transaction record 6670, in the sense it filters the messaging on the basis of the information contained in that record. For example, the filtering can be done on the basis of keywords. If the messaging contains keywords which are inconsistent with the filter settings, the messages are rejected, and they do not reach the patient.

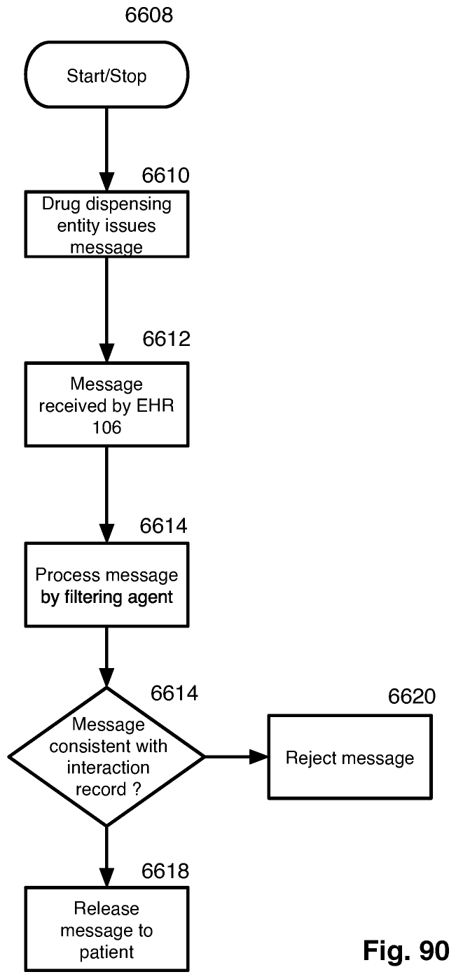

This process as illustrated by the flowchart at FIG. 90 starts at step 6608. At step 6610 the drug dispensing entity 6610 sends a message to the patient via the EHR system 106. The message contains the identifier created earlier via the process illustrated at FIG. 89, allowing the logic at the Human-Centric EHR 106 to identify the user profile of the patient among the user profiles of other patients that subscribe to the services of the Human-Centric EHR 106. The message is received by the Human-Centric EHR 106 at step 6612. At step 6614 the message is processed by the filtering agent. If at decision step 6616 the filtering agent finds the message to be consistent with the settings of the transaction GUI 6668, the message is released to the patient at step 6618, otherwise the message is rejected at step 6620. [symbol] possible variant, the drug prescription management systems and methods described above can be [symbol] ited for other medical related services provided by entities that are external to the Human-Centric EHR system 106. Laboratory services or imaging services are such examples.

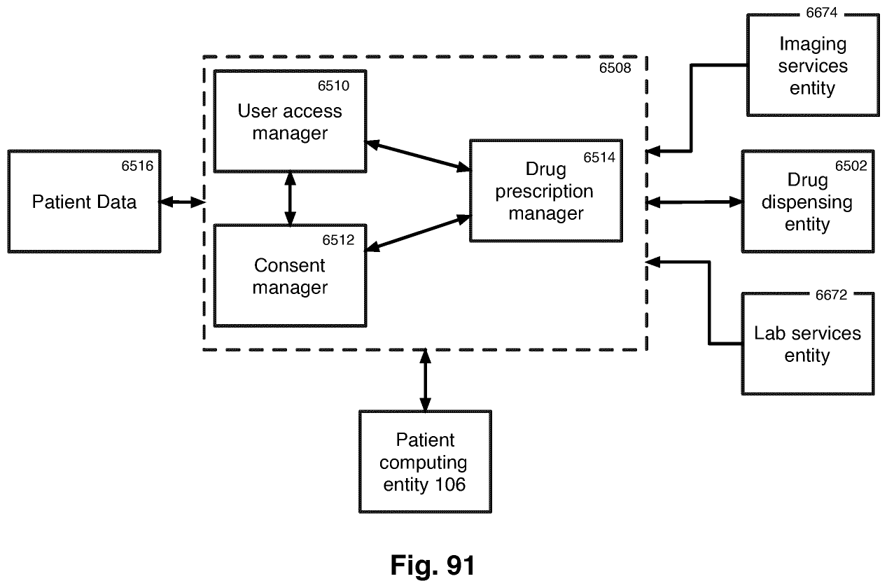

FIG. 91 is a block diagram of a network arrangement similar to the one described previously in connection with FIG. 80, now showing additional external entities providing laboratory services and imaging services to the patient. Specifically, in addition to the drug dispensing entity, the Human-Centric EHR system 106 also communicates with a lab services entity 6672 and with an imaging services entity 6674. For simplicity, one lab services entity 6672 and only one imaging services entity 6674 are shown, it being understood that multiple such service entities an connect to the Human-centric EHR system 106.

The lab services entity 6672 would typically provide medical testing services, such as diagnostic testing that is prescribed by a physician to detect, diagnose or monitor diseases or conditions of the patient. A specific example would be a blood test. Similarly, the imaging services entity 6674 provides medical testing services by providing a picture of the inside of the human body using X-ray emissions, ultrasound, radio waves and radiation, among others.

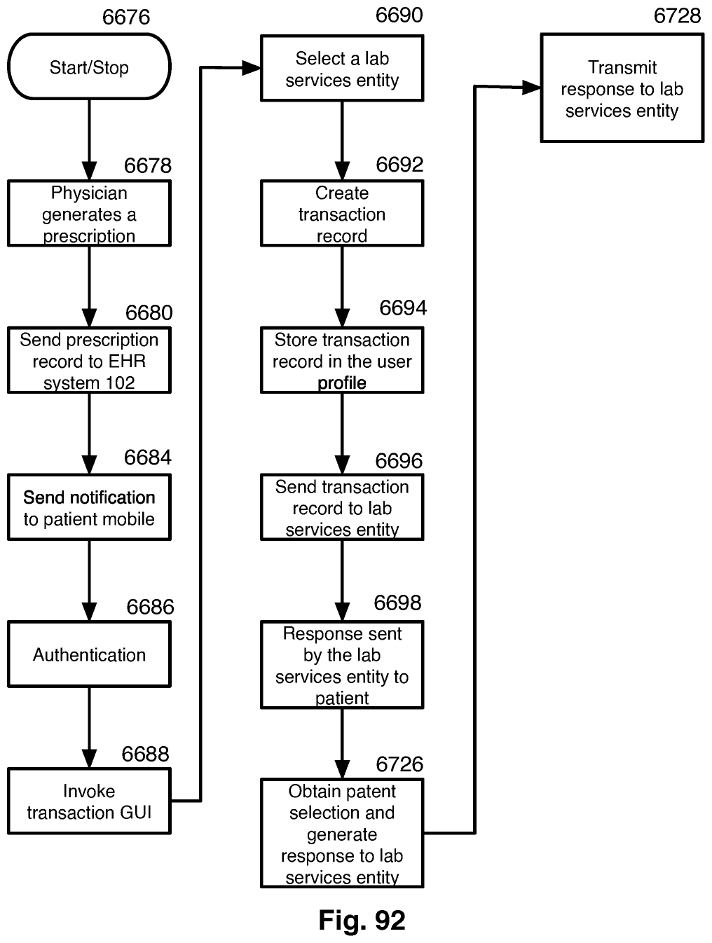

FIG. 92 is a flowchart of a process illustrating the typical interaction when a patient is provided with a requisition for a medical test and needs to schedule an appointment with either one of the lab services entity 6672 or the imaging services entity 6674.

The process starts at step 6676. At step 6678 the physician generates a requisition for a medical test, which can be either a lab test or an imaging test. Assume for the purpose of this example that the test is a blood test. At step 6680 the requisition record for the blood test is sent to the Human-Centric EHR system 102. The requisition record can include an image of the requisition form showing a list of available blood tests, with the requested tests being checked. Alternatively, or in addition to the image, the requisition conveys semantic information about the requested tests making the computer processing of the request easier, such as machine identification of the individual tests being requested. At step 6682, the Human-Centric EHR system 106 receives the requisition record and generates a notification to the patient to notify the patient that the requisition record is deposited and can be accessed via the app. The access to the requisition record requires user authentication at the mobile level and optionally at the app level, as shown at step 6686. [symbol] n the patient accesses the requisition record on the mobile 106, a transaction GUI 6688 is invoked at [symbol] 6688. The transaction GUI will present the patient with a list of entities that can perform the medical tests specified in the requisition. At step 6690, the patient selects a lab services entity 6674 in the list. At step 6692 the Human-Centric EHR system 106 logic creates a transaction record that packages the medical test request and assigns a unique identifier to the transaction record. At step 6694 the transaction record is stored in the user profile and at step 6696 the transaction record is sent to the selected lab services entity 6674. At step 6698 the transaction record is processed by the lab services entity 6674 and a response is sent to the patient via the Human-Centric EHR system 106, as shown at step 6698, by linking the identifier in the response to the identifier in the transaction record. For example, response includes a list of possible locations where the patient can go to collect a blood sample.

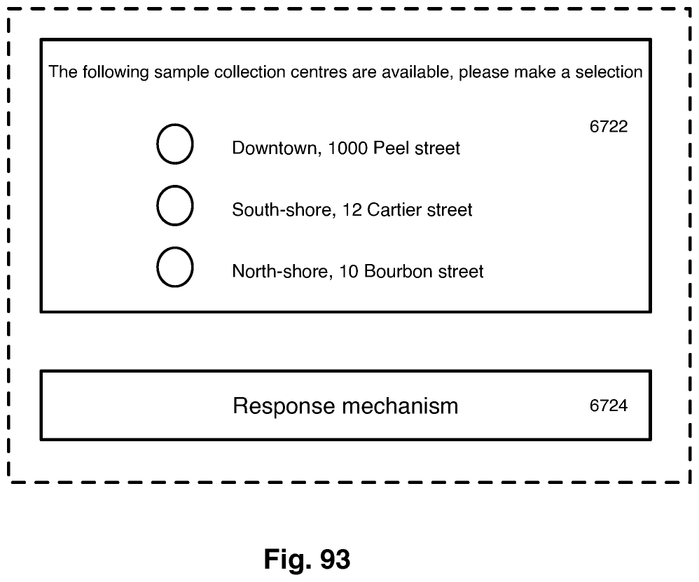

An example of the notification is depicted in FIG. 93. It has a viewable area 6722 that illustrates information that can be viewed by the patient with controls allowing the patient to make a selection and an underlying response mechanism 6724 to generate a response to the lab services entity 6672 via the Human-Centric EHR system 106, where the response conveys the patient selection. Referring back to the flowchart of FIG. 92, step 6728 illustrates the transmission of the response to the lab services entity 6672. As a further step, shown at 6730, the lab services entity 6672 can send a further notification, using the same process described previously, to schedule an appointment at the selected collection center. In that example of implementation, the notification would include a notification structure similar to the one shown in FIG. 88b that includes a scheduling mechanism to schedule the appointment.

Drug Prescription Reconciliation and Security Management.

The ability to provide a drug prescription to a patient in electronic format is practical and enables a series of value-add services to the patient, as discussed in the previous section “Drug prescription management”. In one example discussed above, the patient is provided with an image of the drug prescription, such as a pdf file of the prescription that can be viewed on the mobile of the user and transmitted as such to the pharmacist for fulfillment. Objectively, a potential risk of this approach is the possibility of misuse of the prescription, especially if narcotics have been prescribed. For instance, an individual can make copies of the prescription and try fulfilling the prescription several times at different pharmacies.

To avoid this potential problem the invention provides a drug prescription reconciliation and security management function which is integrated into the Human-Centric EHR system 102 that is designed to communicate drug prescription information to the patient via the patient computing entity 106 such as to [symbol] m the patient that a drug prescription has been issued and what medication has been prescribed but [symbol] information provided is not enough to allow the user to fulfill the prescription. For instance, the information provided may lack prescription validation data which makes the drug prescription official such that a pharmacy can fulfil it. The validation data can be a physician signature, a physician stamp or any other data element establishing that the drug prescription is genuine. Without such drug prescription validation data present, the information provided to the patient does not constitute a valid drug prescription and cannot be legally fulfilled by a pharmacy.

The drug prescription reconciliation and management function communicates the drug prescription with the validation data to the pharmacy selected by the user, as discussed above, such that the pharmacist can fulfill the prescription. The dispatch of the drug prescription to the pharmacy is managed and accounted for to avoid sending the drug prescription to multiple pharmacies in uncontrolled fashion. For example, a single dispatch event is allowed.

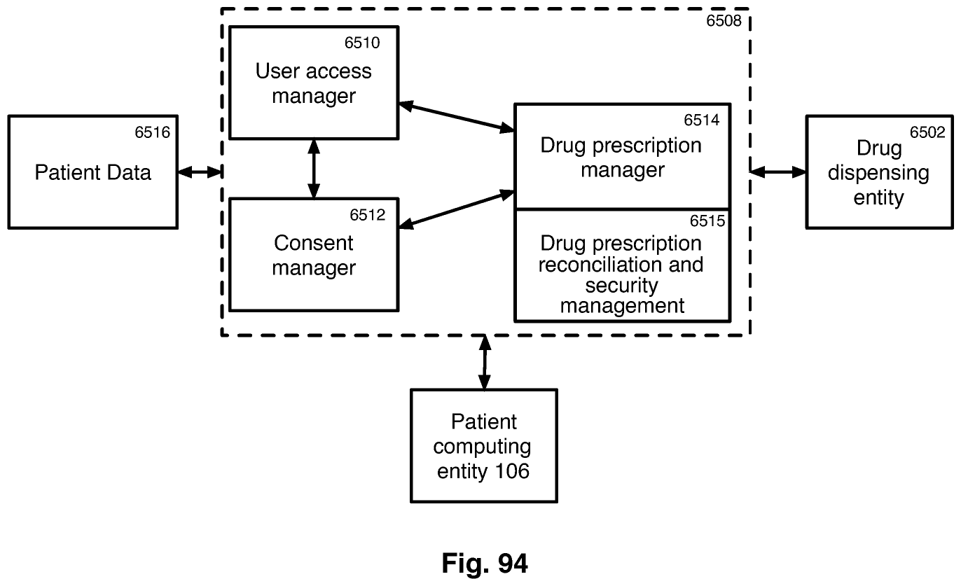

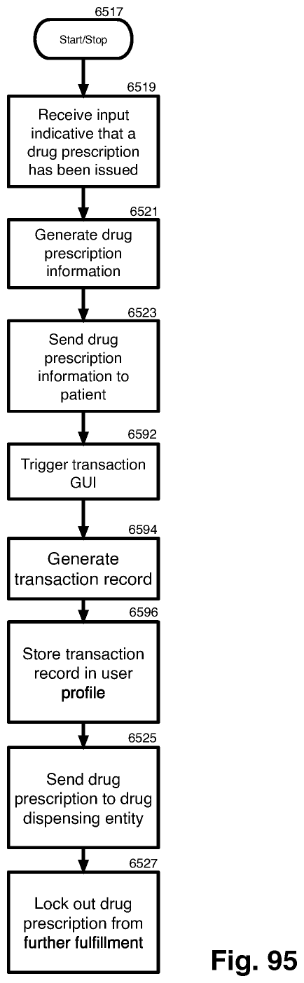

A specific example of implementation of the drug prescription reconciliation and security management function will now be described in connection with the drawings. Referring more particularly to FIG. 94 which shows a block diagram of the various functionalities associated with the user profile 6508, the drug prescription manager functional block 6514 also implements the drug prescription reconciliation and security management function. That function is identified at 6515. The drug prescription reconciliation and security management function 6515 manages the transmission of the drug prescription to the patient on the one hand and the transmission of the drug prescription to the drug dispensing entity 6502 (pharmacy) after the patient has selected the pharmacy that will be fulfilling the drug prescription, on the other hand. The process implemented by the drug prescription reconciliation and security management function 6515 is illustrated by the flowchart at FIG. 95.

The process starts at 6517. At step 6519 the drug prescription reconciliation and security management functional block 6515 receives as an input data or a signal indicative that a drug prescription has been issued for a particular patient. At step 6521, the drug prescription reconciliation and security management functional block 6515 will generate on the basis of the drug prescription issued a drug prescription information document or record that is sent to the patient, at step 6523, as per the processes discussed earlier, namely by triggering the display of a notification to the mobile of the patient and then making the drug prescription information document available to the patient subject to successful authentication at the mobile level and optionally at the app level. [symbol] hat point, the patient can continue the transaction regarding the drug prescription with the drug [symbol] ensing entity 6502, generally as described in relation to the flowchart of FIG. 89, in particular the transaction GUI is shown at step 6592, the transaction record is generated at 6594 and the transaction record is stored in the user profile at step 6596. Note steps 6592, 6594 and 6596 are the same in both flowcharts in FIGS. 89 and 94.

At step 6525, the drug prescription reconciliation and security management functional block sends the drug prescription to the selected drug dispensing entity 6502 in a format that allows the pharmacist to fulfill the drug prescription. That is to say, the drug prescription, which is part of the transaction record, includes the validation data authenticating the drug prescription, such as the signature of the physician, a physician stamp or any other validation element.

In a practical example, assume the physician produces the drug prescription as a pdf file which contains a list of the prescribed drugs and a physician signature and stamp. When the pdf file is received by the drug prescription reconciliation and security management functional block 6515, the pdf file is processed to extract the list of the prescribed drugs and that extracted list constitutes the drug prescription information which is made available to the patient via the mobile. That is to say, the patient will be able to view via the app on the mobile a list of prescribed drugs but there will be no validation data that would allow this list to be considered as a valid drug prescription and to be fulfilled at a pharmacy. So even if the patient prints or exports this list out of the app it cannot be legally fulfilled.

However, once the transaction record is generated as per step 6594, the original pdf file which is available to the drug prescription reconciliation and security management functional block 6515 sends the authentic drug prescription to the drug dispensing entity 6502 for fulfillment. The transmission of the drug prescription can be done in several ways. For instance, the transmission can be a fax transmission, as currently pharmacies accept drug prescriptions sent by fax for fulfillment. Accordingly, in this mode of implementation, the transmission of the drug prescription is done over a different communication channel than the remainder of the transaction record. Alternatively, the drug prescription can be sent as an encrypted file where the pharmacy has the decryption key such that the transmission is a protected transmission. Also note that the system log of the EHR system 102 is marked to note the successful transmission of the prescription to the drug dispensing entity 6502 in order to lock out this particular drug prescription from being fulfilled again by the patient. This is shown at step 6527. In a specific example, the drug prescription is marked by a flag or otherwise to be recognizable by the system as being fulfilled and will not enable the patient to generate another transaction with the drug dispensing entity 6502. [symbol] tically, this can be accomplished by disabling the transaction GUI in relation to this particular drug [symbol] ription. Accordingly, while the drug prescription information is still available for the patient to view on the app of the mobile, accessing the drug prescription information page will no longer trigger the transaction GUI.

As indicated previously, the advantage of this approach is to avoid or at least reduce the possibility of fraudulently obtaining prescription drugs by maintaining the authentic drug prescriptions in a safe enclave defined by the joint domains of the EHR 102 and the drug dispensing entity 6502. In this fashion, the patient has never access to the authentic drug prescription, and the fulfillment transaction is not directly accessible to permit manipulations of the authentic drug prescription. At the same time the patient is fully informed about the particulars of the drug prescription, namely the drugs being prescribed.

1. A method to manage exposure of confidential user information in a user data record to a third party, the method comprising:
a. providing an electronic record system managed by a server arrangement, the server arrangement being configured to interact with a user device associated with the user,
b. implementing with the server arrangement a data privacy configurator including a graphical user interface (GUI) allowing the user at the user device to specify data access parameters, the data access parameters distinguishing between first data in the user data record that the user is willing to expose to the third party from second data in the user data record that the user is not willing to expose to the third party, and
c. in response to a request received at the server arrangement to communicate user data from the user data record to the third party, processing the user data record according to the data access parameters to allow the third party to access the first data while precluding the third party to access the second data.

2. The method as defined in claim 1, wherein the GUI includes user operable controls allowing the user at the user device to selectively designate the second data in the user data record as masked to preclude exposure of the second data to the third party.

3. The method as defined in claim 2, wherein the user operable controls are capable to selectively acquire a masked state and an unmasked state in relation to the second data, wherein in the masked state the data privacy configurator precludes exposure of the second data to the third party and in the unmasked state the data privacy configurator enables exposure of the second data to the third party.

4. The method as defined in claim 1, wherein the data privacy configurator is configured to store data privacy configuration settings associated with a plurality of third parties to respective data access parameters, and in response to a request from a particular third party of the third parties, deriving from the data privacy configuration settings the data access parameters associated with the particular third party and processing the request of the particular third party according to the derived data access parameters.

5. The method as defined in claim 4, wherein the data privacy configurator allows the user to set data access parameters independently for different third parties desirous to have access to the user data.

6. The method as defined in claim 1, wherein, in response to an update to the user data record including an addition of a data item to the user data record, prompting the user at the user device to set data access parameters at the GUI in relation to the data item.

7. The method as defined in claim 1, wherein the user data record includes a plurality of data items, the data privacy configurator being configured to define data access parameters in relation of a set of data items of the plurality of data items, wherein the data access parameters apply globally to the data items in the set.

8. The method as defined in claim 7, wherein the data privacy configurator implements a plurality of selectable masking themes, the data privacy configurator further implementing logic for processing the plurality of data items to derive a sub-group of data items of the user data record associated to the selected masking theme and set the data access parameters for the data items of the sub-group to a common masked state or unmasked state.

9. The method as defined in claim 1, wherein the user data record is a medical record and holds medical information about the user who is a patient.

10. A computer-implemented method of establishing a cohort to conduct research, the computer-implemented method comprising:
storing in computer-readable memory:
health records about patients; and
information indicative of (i) consent of respective ones of the patients that respective ones of the health records about the respective ones of the patients be accessible for the research and (ii) parameters regarding the consent of the respective ones of the patients;
receiving, with at least one processor, a request from a third party related to the research and indicative of at least one criterion for the research;
identifying, with the at least one processor, individual ones of the health records about individual ones of the patients that meet the at least one criterion for the research and that correspond to the parameters regarding the consent of the individual ones of the patients; and
transmitting, with the at least one processor, data included in the individual ones of the health records to the third party for conducting the research.

11. The computer-implemented method of claim 10, wherein the parameters regarding the consent of the respective ones of the patients include a period of validity of the consent of the respective ones of the patients.

12. The computer-implemented method of claim 10, wherein the parameters regarding the consent of the respective ones of the patients include a definition of information included in the respective ones of the health records that the respective ones of the patients consent to making accessible.

13. The computer-implemented method of claim 10, wherein the parameters regarding the consent of the respective ones of the patients include a type of research project for which the respective ones of the patients consent to making the respective ones of the health records accessible.

14. The computer-implemented method of claim 10, comprising anonymizing, with the at least one processor, the data included in the individual ones of the health records such that the data included in the individual ones of the health records is anonymized before the transmitting.

15. The computer-implemented method of claim 10, wherein the at least one criterion for the research includes at least one of a disorder, an infection, a disability, and an injury.

16. The computer-implemented method of claim 10, wherein the at least one criterion for the research includes a medical test.

17. The computer-implemented method of claim 10, wherein the at least one criterion for the research includes a willingness to physically participate in the research.

18. The computer-implemented method of claim 10, comprising recording in the computer-readable memory information indicative of transmission of the data included in the individual ones of the health records to the third party for traceability.

19. The computer-implemented method of claim 10, comprising, in response to the research identifying an anomalous condition in the data included in a given health record of the individual ones of the health records that is about a given patient of the individual ones of the patients, notifying the given patient of the anomalous condition.

20. The computer-implemented method of claim 10, wherein the third party is a pharmaceutical company.

21. The computer-implemented method of claim 10, wherein the third party is a biomedical company.

22. A system for establishing a cohort to conduct research, the system comprising at least one processor and computer-readable memory and being configured to:
store:
health records about patients; and
information indicative of (i) consent of respective ones of the patients that respective ones of the health records about the respective ones of the patients be accessible for the research and (ii) parameters regarding the consent of the respective ones of the patients;
receive a request from a third party related to the research and indicative of at least one criterion for the research;
identify individual ones of the health records about individual ones of the patients that meet the at least one criterion for the research and that correspond to the parameters regarding the consent of the individual ones of the patients; and
transmit data included in the individual ones of the health records to the third party for conducting the research.

23. Non-transitory computer-readable storage media comprising instructions executable by at least one processor of a computing entity for establishing a cohort to conduct research, the instructions being configured to cause the computing entity to:
store:
health records about patients; and
information indicative of (i) consent of respective ones of the patients that respective ones of the health records about the respective ones of the patients be accessible for the research and (ii) parameters regarding the consent of the respective ones of the patients;
receive a request from a third party related to the research and indicative of at least one criterion for the research;
identify individual ones of the health records about individual ones of the patients that meet the at least one criterion for the research and that correspond to the parameters regarding the consent of the individual ones of the patients; and
transmit data included in the individual ones of the health records to the third party for conducting the research.
